(19) United States
(12) Patent Application Publication
(10) Pub. No.: US 2022/0301710 A1
(43) Pub. Date: Sep. 22, 2022

(54) DETERMINING CAUSES OF DISEASES SUCH AS CANCER, USING MACHINE LEARNING ANALYSIS OF GENETIC DATA

(71) Applicant: The Johns Hopkins University, Baltimore, MD (US)

(72) Inventors: Cristian Tomasetti, Baltimore, MD (US); Bahman Afsari, Baltimore, MD (US)

(21) Appl. No.: 17/616,740

(22) PCT Filed: Jun. 5, 2020
(86) PCT No.: PCT/US2020/036327

§ 371 Date: Dec. 6, 2021

Related U.S. Application Data
(60) Provisional application No. 62/858,007, filed on Jun. 6, 2019.

(51) Int. Cl. G16H 50/20; G16H 70/60; G06N 5/00; C12N 15/10

(52) U.S. Cl. CPC G16H 50/20; G16H 70/60; G06N 5/003; C12N 15/102

(57) ABSTRACT

This document describes technology that can be used for detecting an etiological factor of a disease in a subject having the disease, training data is received that includes data objects each recording i) a disease label, ii) at least one corresponding mutational signature, and iii) corresponding etiological tags. A first set of features based on single nucleotide mutations and a second set of features based on dinucleotide mutations are generated. A machine learning model is trained on the first set of features and on the second set of features. A classifier is generated that is configured to: operate by receiving a new-genomic-data-object, the new-genomic-data-object specific to the subject having the disease; and generate, from the new-genomic-data-object, a etiological-classification for the new-genomic-data-object, the etiological-classification indicating a corresponding etiological factor that matches one of the etiological tags.

### CROSS-REFERENCE TO RELATED APPLICATIONS

This application claims the benefit of U.S. Patent Application Ser. No. 62/858,007, filed on Jun. 6, 2019. The disclosure of the prior application is considered part of (and is incorporated by reference in) the disclosure of this application.

### TECHNICAL FIELD

This document describes technology that can be used for detecting an etiological factor of a disease in a subject having the disease.

### BACKGROUND INFORMATION

Etiology is the study of causation, or origination. More completely, etiology is the study of the causes, origins, or reasons behind the way that things are, or the way they function, or it can refer to the causes themselves. The word is commonly used in medicine, (where it is a branch of medicine studying causes of disease) and in philosophy, but also in physics, psychology, government, geography, spatial analysis, theology, and biology, in reference to the causes or origins of various phenomena.

Machine learning (ML) is the scientific study of algorithms and statistical models that computer systems use in order to perform a specific task effectively without using explicit instructions, relying on patterns and inference instead. It is seen as a subset of artificial intelligence.

### SUMMARY

Etiological factors can be detected for various diseases, including cancers. For example, over the past decade a personalized approach for cancer diagnosis and treatment has evolved to include both genotypic and phenotypic characteristics of patient specific tumors. The identification and characterization of “driver DNA mutations” has been a critical aspect of defining cancer beyond tumor origin and morphology. These driver mutations have created an entirely new approach for development of targeted therapeutics such as Keytruda/PDL1 biomarker (DNA mismatch repair deficiency), Vitrakvi/NTRK gene fusion, and Rozlytrek/NTRK genetic mutation. However, linking biologically relevant “DNA mutations” to actionable and effective outcomes and development of new strategies to deliver “precision, personalized, preventive medicines” goals requires analyzing molecular data which deciphers the “history and footprints” of carcinogen forces, specific driver mutations but also global mutational signatures. This document provides supervised, machine-learning techniques that can identify signatures, called SuperSigs, that can have immediate applications for both prevention and therapy selection. For example, the methods described herein can enable the combination of knowledge about local molecular features (e.g. hot spot “driver mutations”) with global landscape features (e.g. the mutation rate of Cytosine to Adenine representing global damage to the DNA by carcinogens) to determine the optimal treatment choice or the probability of survival of a patient.

As demonstrated herein the SuperSigs technology described herein, contrary to current unsupervised and/or local feature approaches, can be used to enable precision medicine, by assigning patients to different cancer treatment regimens based on their mutational history. Availability of highly curated database signatures as a basis of defining the driving causes of mutations can enable clinicians to adopt a genome-wide holistic approach towards patient management by integrating endogenous, environmental, and inherited factors that are underlying the deadly “mutational DNA signatures”: a highly curated database of “mutational DNA signatures” created through the combination of thousands of human genome sequences with highly sophisticated analytical and mathematical algorithms to establish the footprints that lead up to the transformation of genes.

In one aspect, this document features methods for detecting an etiological factor of a disease in a subject having the disease. The methods can include, or consist essentially of, receiving training data that includes data objects each recording i) a disease label, ii) at least one corresponding mutational signature, and iii) corresponding etiological tags. The methods can include generating a first set of features based on single nucleotide mutations. The methods can include generating a second set of features based on dinucleotide mutations. The methods can include training a machine learning model on the first set of features and on the second set of features. The methods can include generating, from the machine learning model, a classifier that is configured to: operate by receiving a new-genomic-data-object, the new-genomic-data-object specific to the subject having the disease; and generate, from the new-genomic-data-object, a etiological-classification for the new-genomic-data-object, the etiological-classification indicating a corresponding etiological factor that matches one of the etiological tags. The methods can include receiving the subject's genome. The methods can include generating, from the subject's genome, a subject-genomic-data-object for the subject. The methods can include detecting an etiological factor for the subject by providing the subject-genomic-data-object to the classifier. In addition to the methods, computer-readable media, systems, devices, and software may be used.

In some aspects, the first set of features are possible substitutions of single nucleotides of a group consisting of C>A, C>G, C>T, T>A, T>C, and T>G.

In some aspects, the first set of features are defined using a pyrimidine of the mutated Watson-Crick base pair.

In some aspects, a third set of features is generated based on trinucleotide mutations, wherein training the machine learning model further comprises training the machine learning model on the third set of features.

In some aspects, a fourth set of features is generated based on all mutations, wherein training the machine learning model further comprises training the machine learning model on the fourth set of features.

In some aspects, training of the machine learning model comprises organizing the features into a partition tree that includes layers of nodes, each node representing a particular type of mutation and each child of the node representing possible mutations that are a type of mutation in the particular node.

In some aspects, the training of the machine learning model further comprises pruning the partition tree by removing a pruned node and all other nodes that are children of the pruned node.

In some aspects, the training of the machine learning model comprises selecting some, but not all, of the nodes as candidate nodes to be used for candidate testing; and testing the candidate nodes to generate first-phase candidate nodes.

In some aspects, training of the machine learning model further comprises:

generating second-phase candidates by, for each particular first-phase candidate node, adjusting a value for each parent node that is also a first-phase candidate node, the adjustment being based on the particular first-phase candidate node; selecting, as a second-phase candidate, a first-phase candidate with a remaining value above a threshold value.

In some aspects, training of the machine learning model further comprises generating final candidates by combining second-phase candidates of training data that did have a particular tag with training data that did not have the particular tag.

In some aspects, hypermethylation and hypomethylation are considered similarly and independently.

In some aspects, the disease is a cancer.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A and 1B show supervised versus unsupervised mutational signatures. A) The various cases in which the supervised and unsupervised approaches can be compared. B) Example of randomly generated signatures. The distribution of weights of each signature is approximated by a segmented line to simplify its depiction.

FIGS. 2A and 2B show age signatures. A) Examples of age signatures. All features of an age signature are contained in the pie chart (IUPAC notations: B=not A, D=not C, H=not G, V=not T, W=A or T, S=C or G, M=A or C, K=G or T, R=A or G, Y=C or T). The average percentage of mutations belonging to a certain feature, out of the total number of somatic mutations, is listed under the feature's name. B) Accuracies of tissues' predictions. Each tissue is represented by a point, which depicts the prediction accuracies of the unsupervised approach (x-axis coordinate value) versus the supervised one (y-axis coordinate value). The great majority of points lie above the line, indicating the greater accuracy of the supervised approach.

FIGS. 3A and 3B show environmental, DNA polymerization or repair, and other factors' signatures. A) Some examples of signatures. All features of a signature are contained in the pie chart (IUPAC notations: B=not A, D=not C, H=not G, V=not T, W=A or T, S=C or G, M=A or C, K=G or T, R=A or G, Y=C or T). The average percentage of mutations belonging to a certain feature, out of the total number of somatic mutations, is listed under the feature's name. B) Comparison of prediction accuracies between supervised and unsupervised approaches. Each tissue is represented by a point, which depicts the prediction accuracies of the unsupervised approach (x-axis coordinate value) versus the supervised one (y-axis coordinate value). The great majority of points lie above the line, indicating the greater accuracy of the supervised approach.

FIGS. 4A and 4B show the tissue dependence of the signatures. A) Smoking signatures in different tissues. All features of a signature are contained in the pie chart (IUPAC notations: B=not A, D=not C, H=not G, V=not T, W=A or T, S=C or G, M=A or C, K=G or T, R=A or G, Y=C or T). The average percentage of mutations belonging to a certain feature, out of the total number of somatic mutations, is listed under the feature's name. B) Distances of smoking and aging signatures for different tissues. Multidimensional scaling plot (MDS). A point represents each signature. The closer two points are, the more similar their corresponding signatures are.

FIG. 5 shows mutational signatures of obesity in kidney (KIRP) and esophageal (ESCA) cancer patients. All features of a signature are contained in its pie chart (IUPAC notations: B=not A, D=not C, H=not G, V=not T, W=A or T, S=C or G, M=A or C, K=G or T, R=A or G, Y=C or T). The average percentage of mutations belonging to a certain feature, out of the total number of somatic mutations, is listed under the feature's name.

FIGS. 6A and 6B show example data that can be used when detecting an etiological factor of a disease. For example, the data can be generated by one or more computing processors, stored in computer memory, transmitted across a data network, etc. The data can be stored in one or more datastores accessible by local or remote clients for the purposes of reading, writing, etc. during process described in this document.

A training data object 600 can include data objects (e.g., rows in a table) that record i) a disease label, ii) at least one corresponding mutational signature, and iii) corresponding etiological tags.

Mutation features 602 can include data objects (e.g., rows in a table) that record features and one or more associated values for these features. Various mutation features may be associated with different kinds of mutations. For example, some mutation features 604 may be based on single nucleotide mutations (e.g., possible substitutions of single nucleotides of a group consisting of C>A, C>G, C>T, T>A, T>C, and T>G, and/or defined using a pyrimidine of the mutated Watson-Crick base pair). For example, some mutation features 604 may be based on dinucleotide mutations. For example, some mutation features 604 may be based on trinucleotide mutations. For example, some mutation features 604 may be based on all mutation types. Other types of mutations may be possible.

A genomic data object 604 can include variables for genes and non-genetic values. An etiologic factor classifier 606 or classifiers can receive a new genomic data object 604 and generate and etiologic classifications 604. The etiologic classifications 604 can indicate a corresponding etiological factor that matches one of the etiological tags.

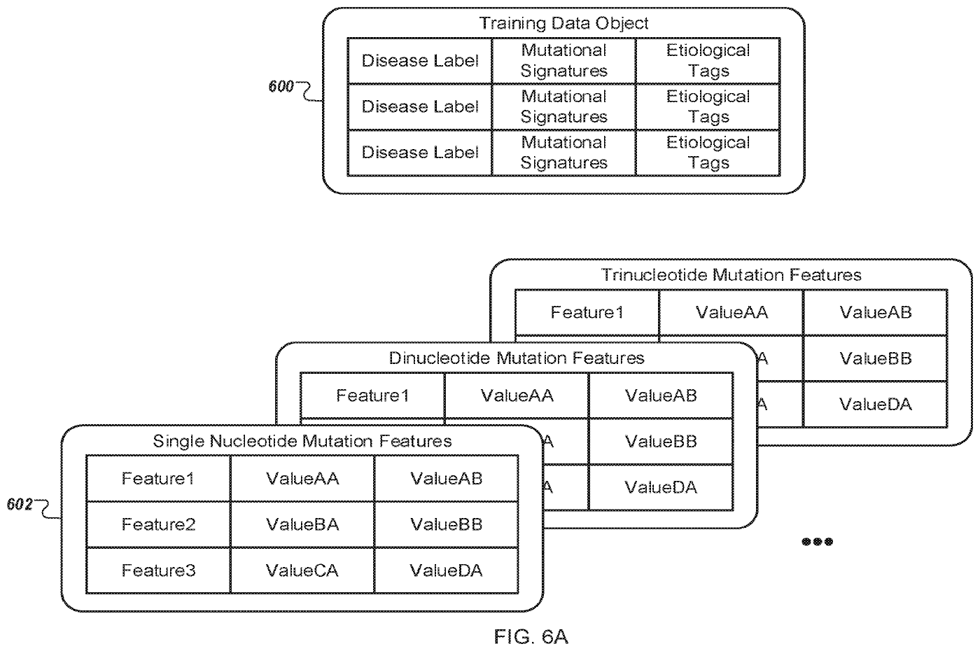

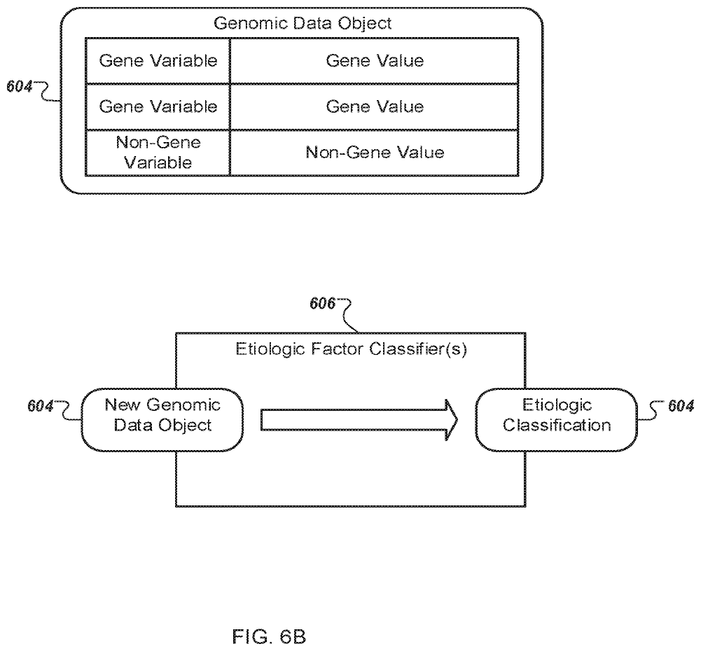

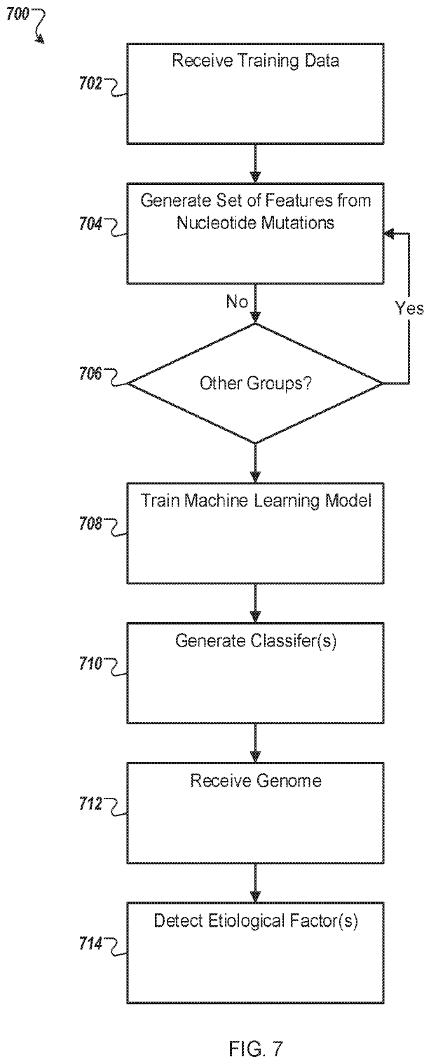

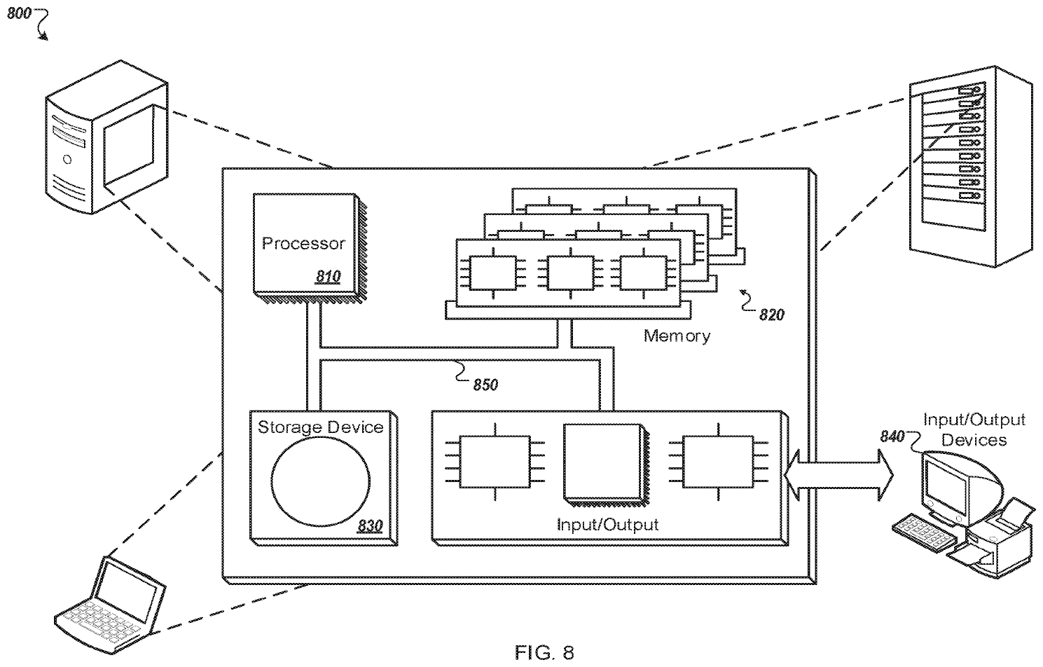

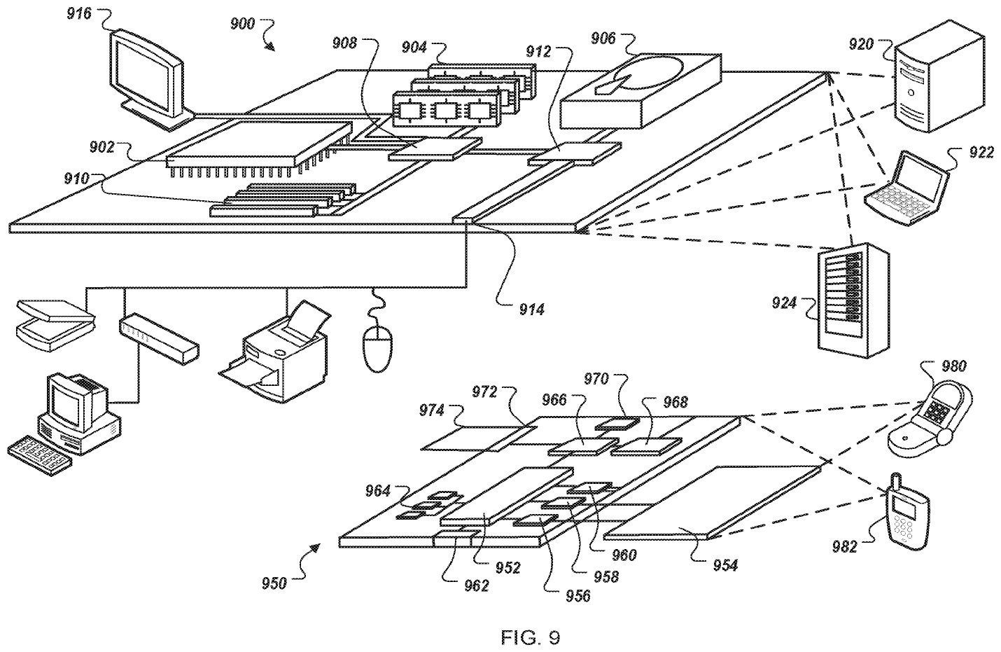

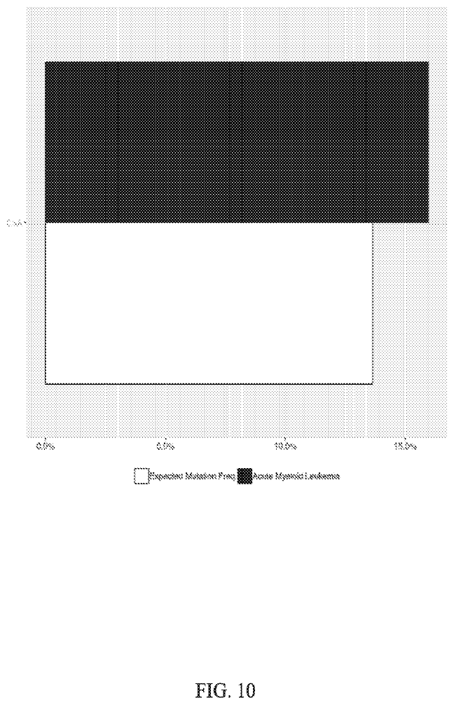

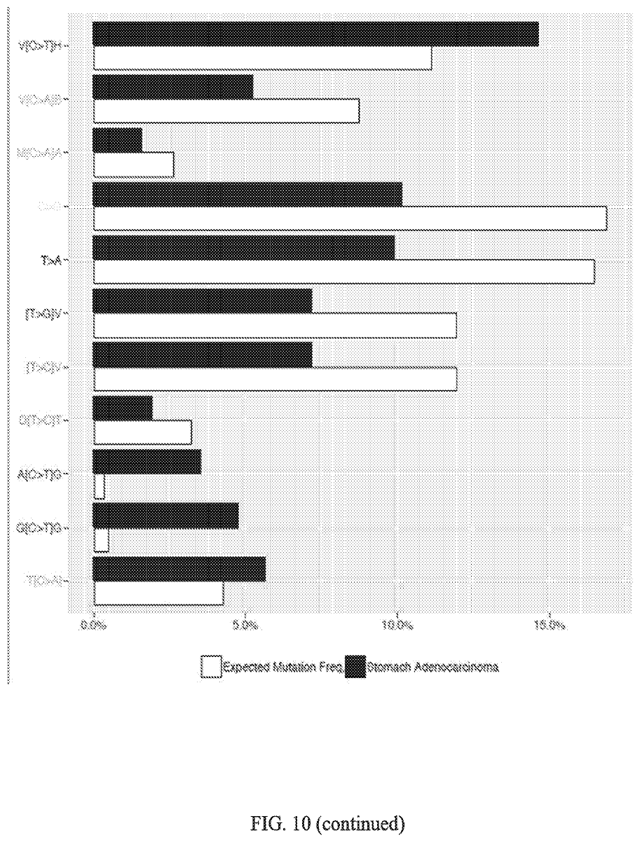

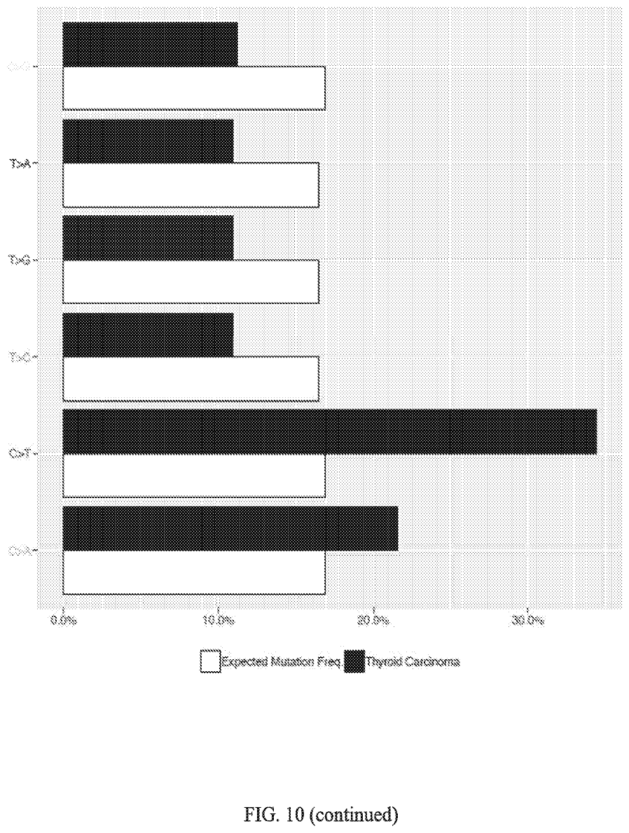

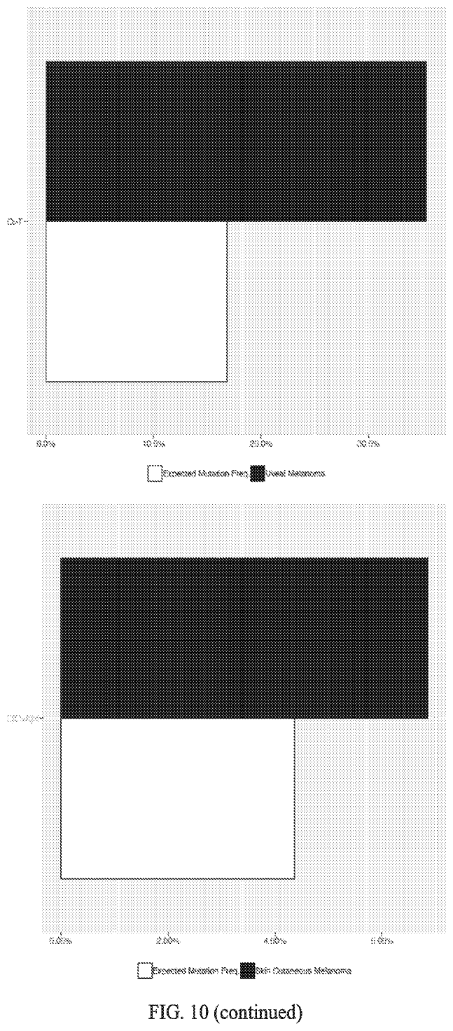

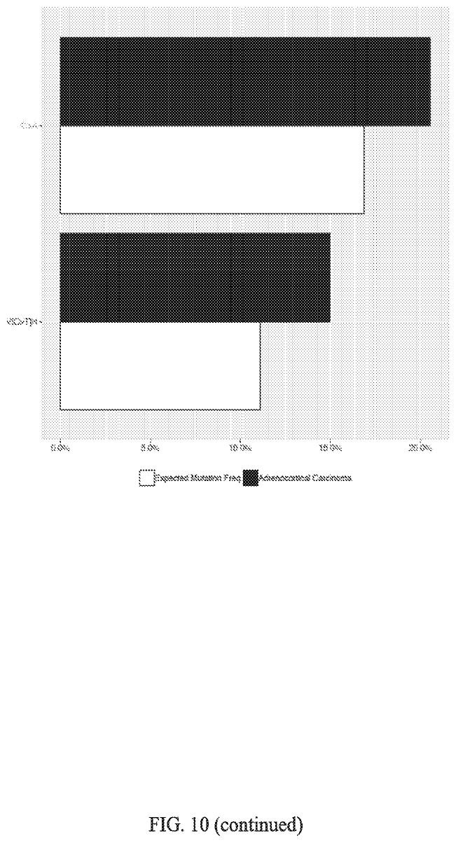

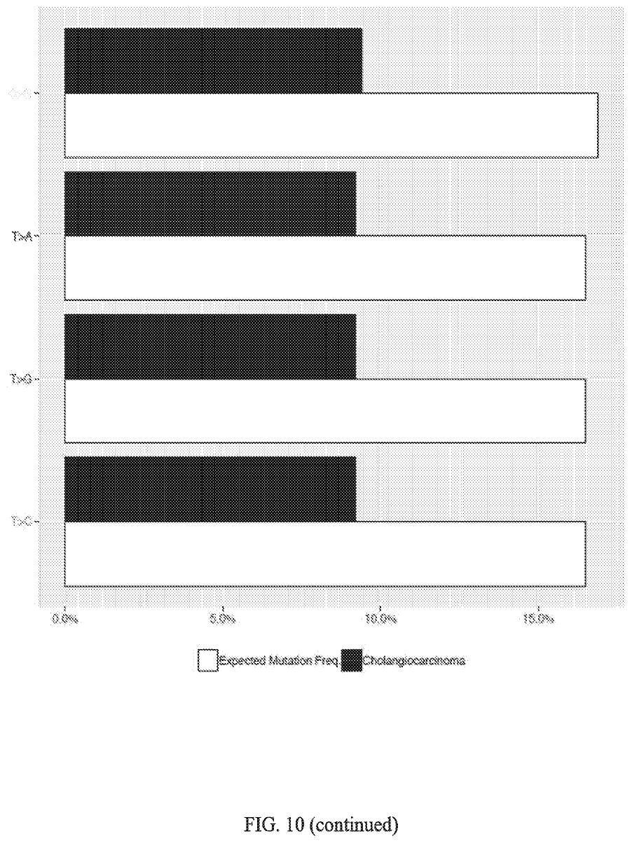

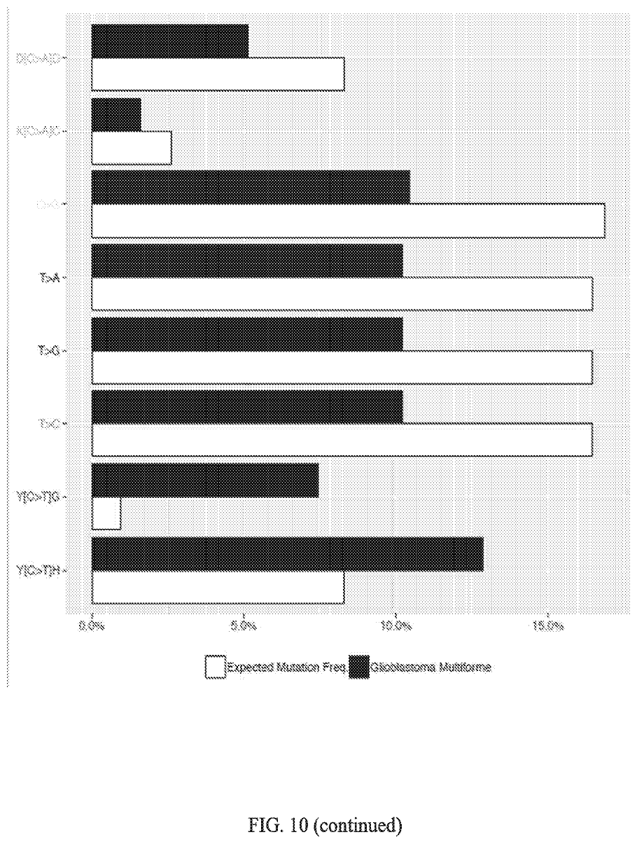

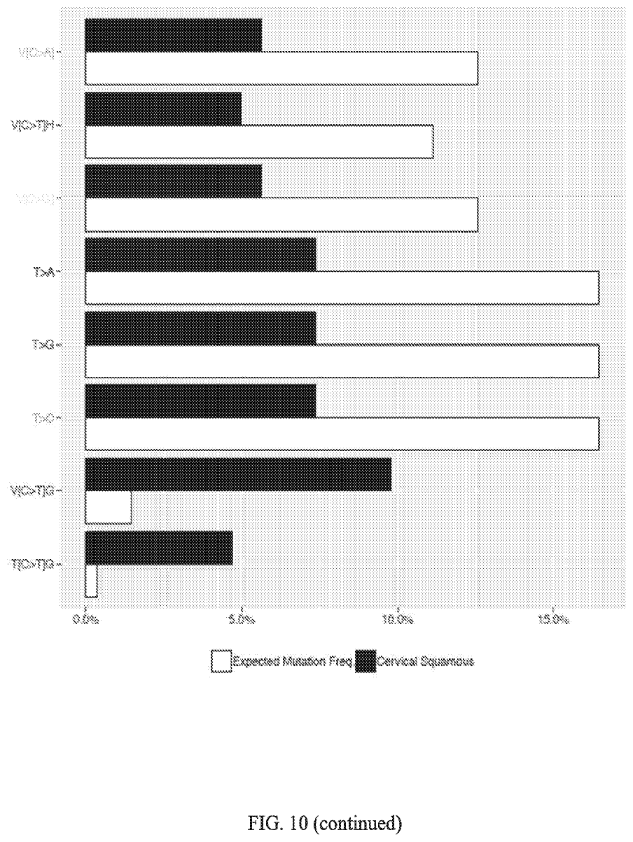

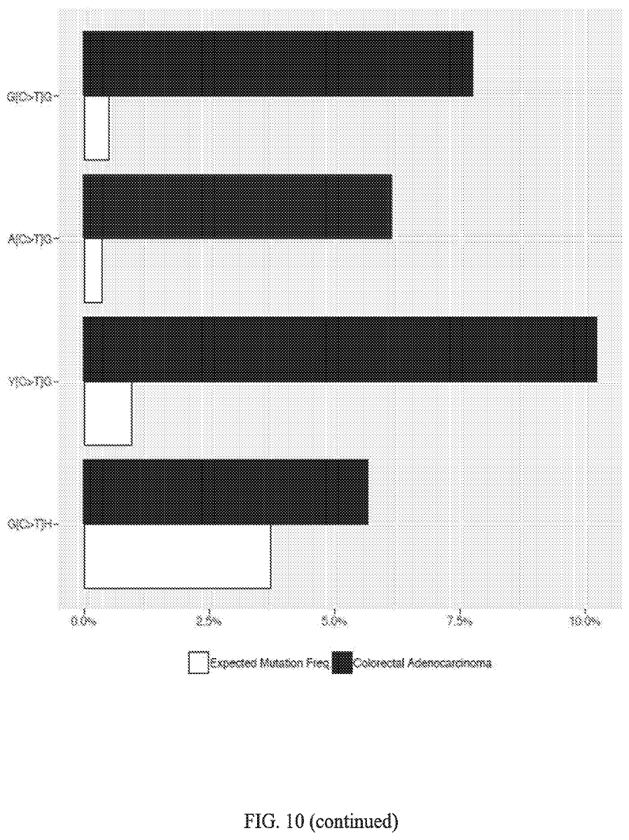

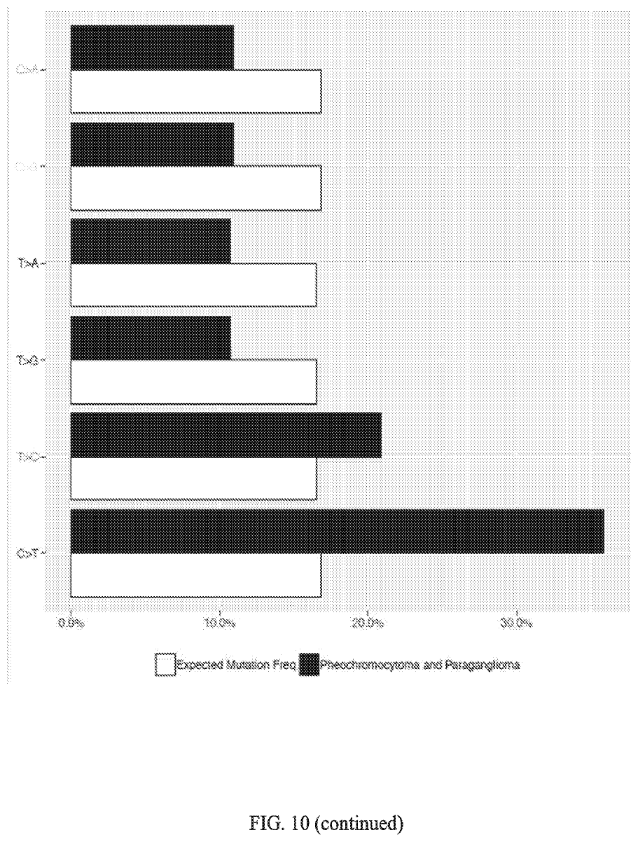

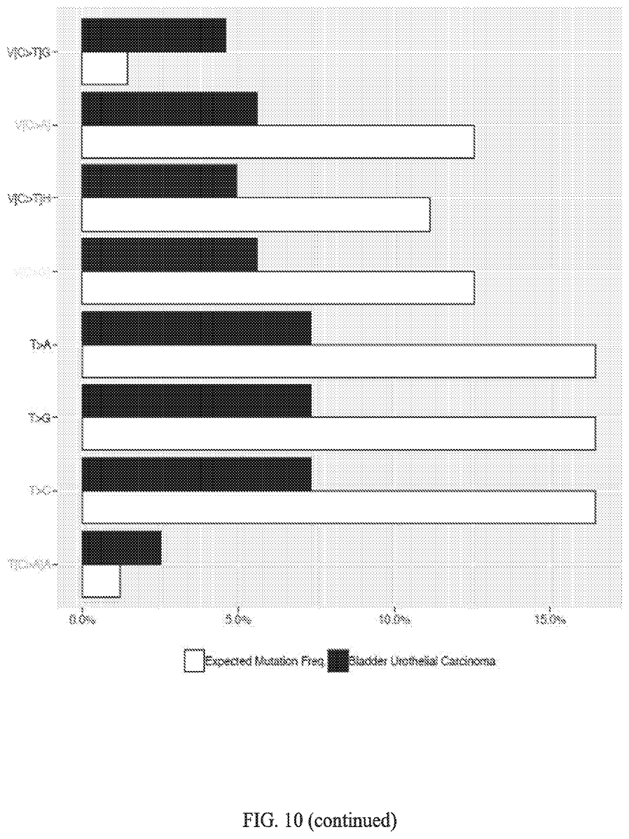

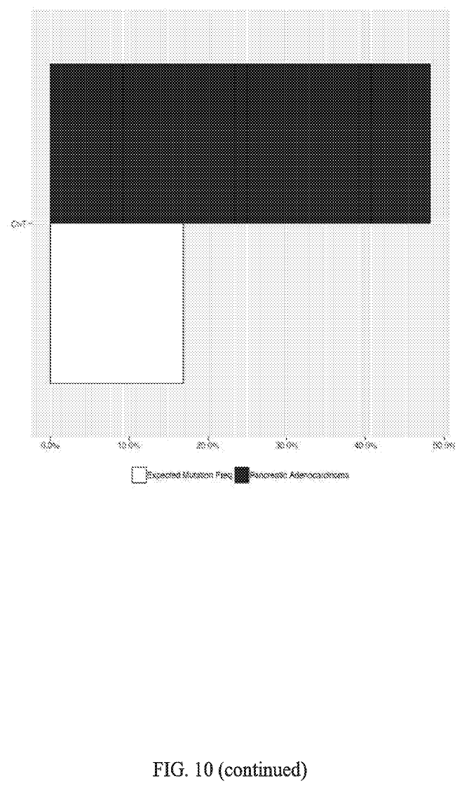

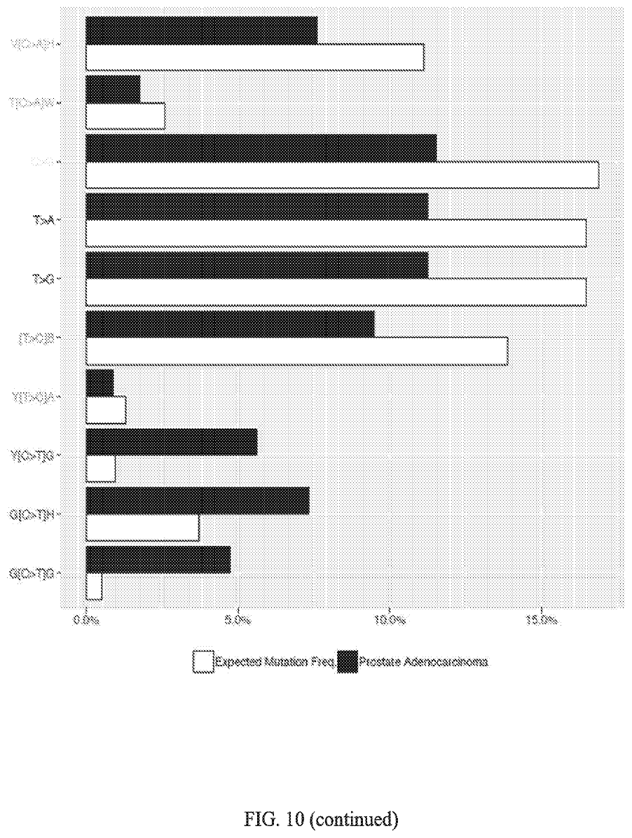

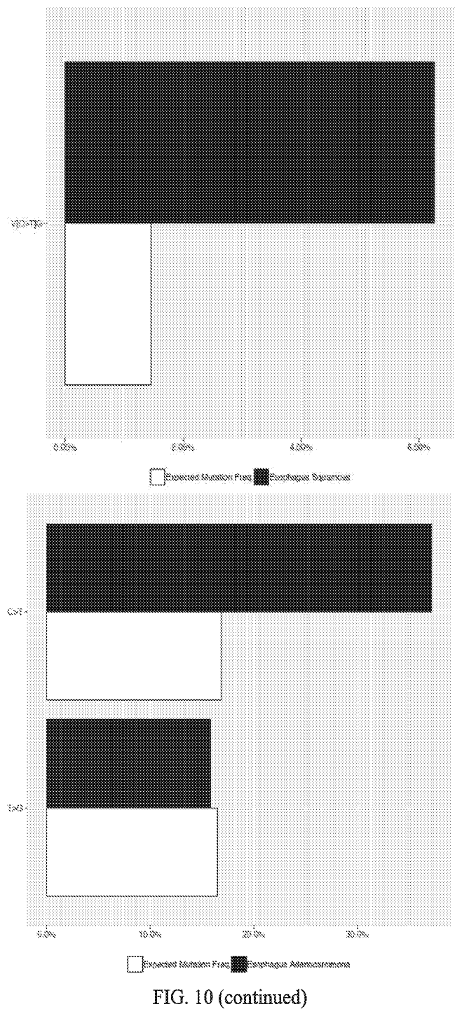

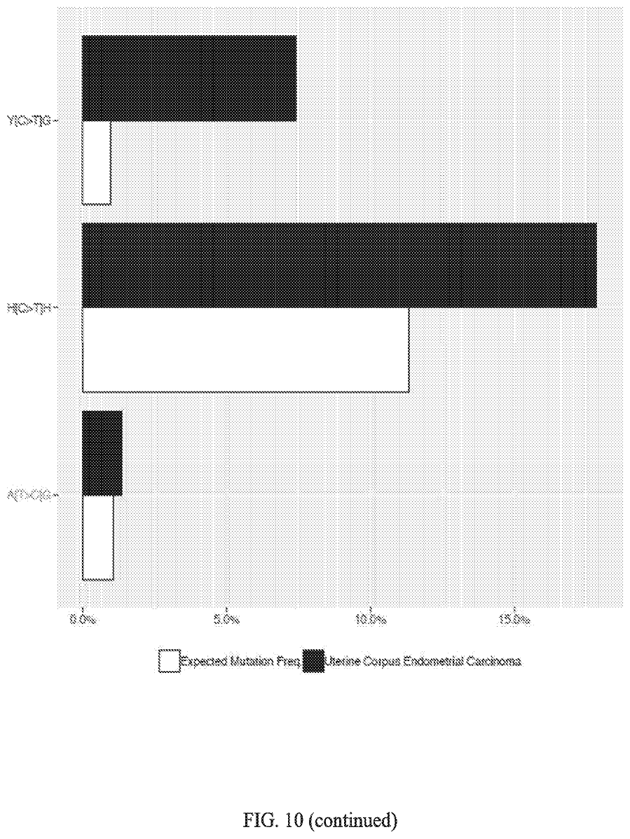

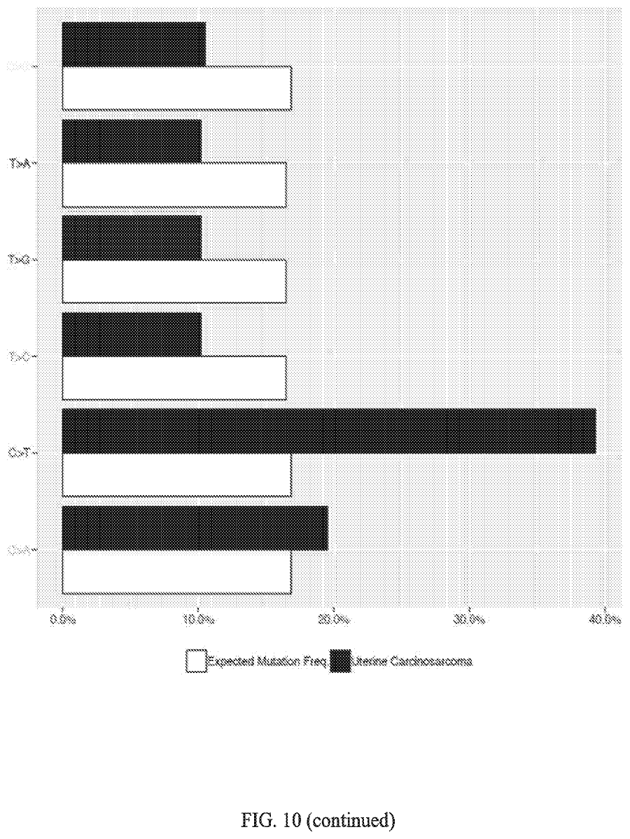

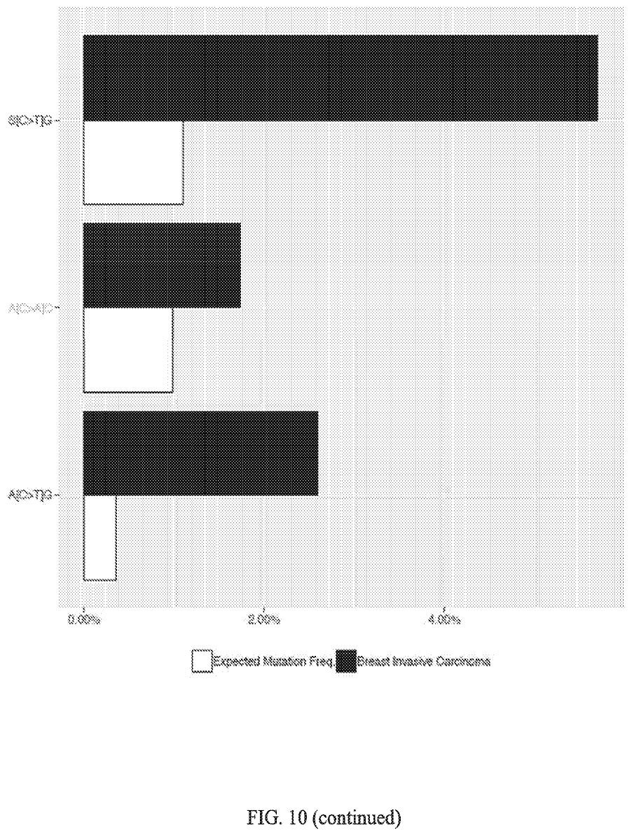

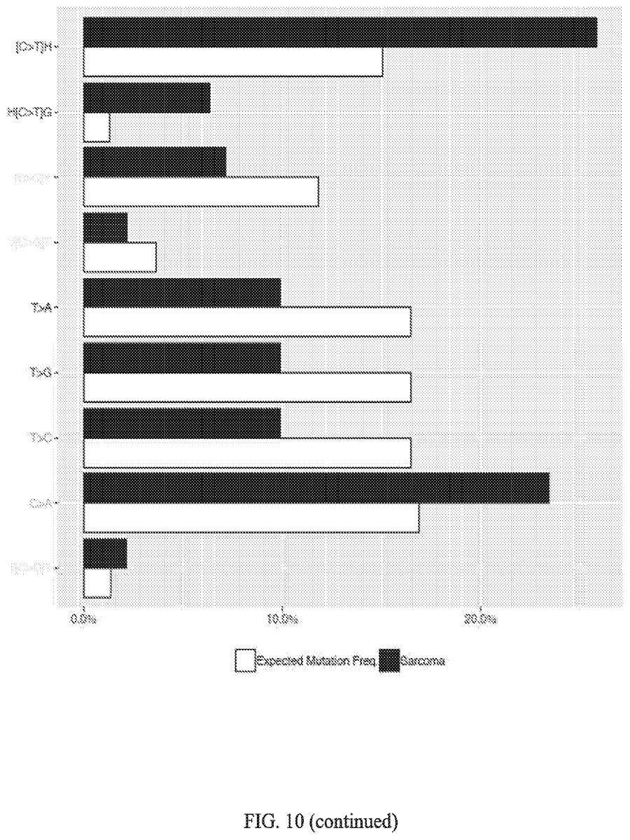

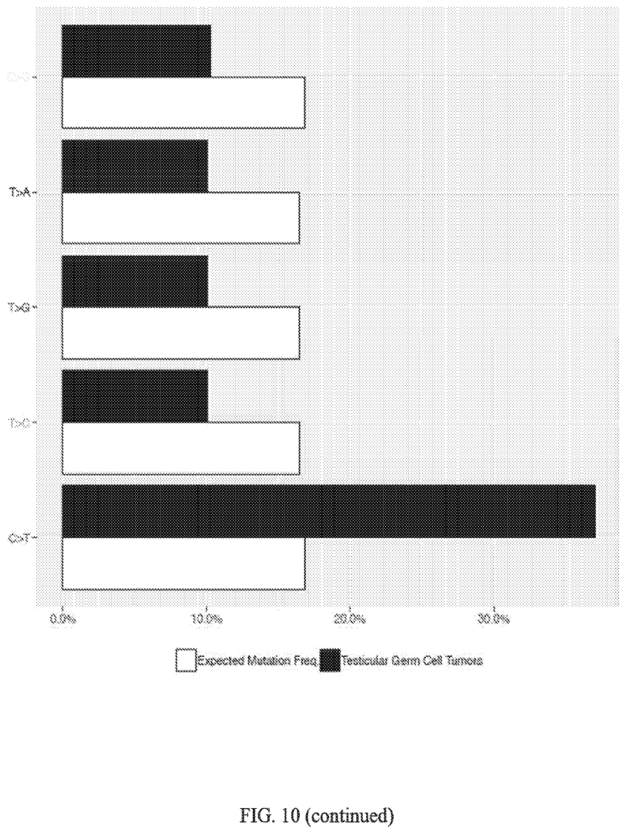

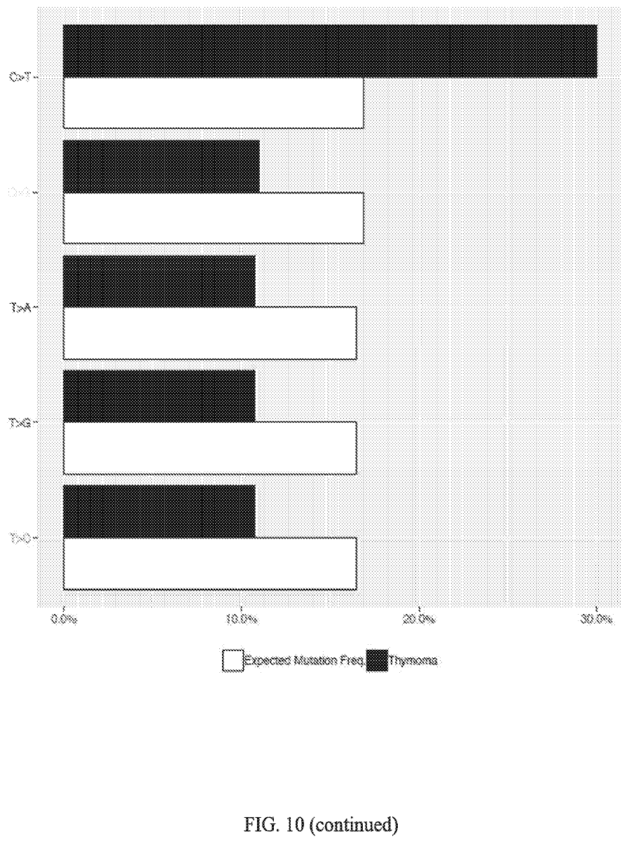

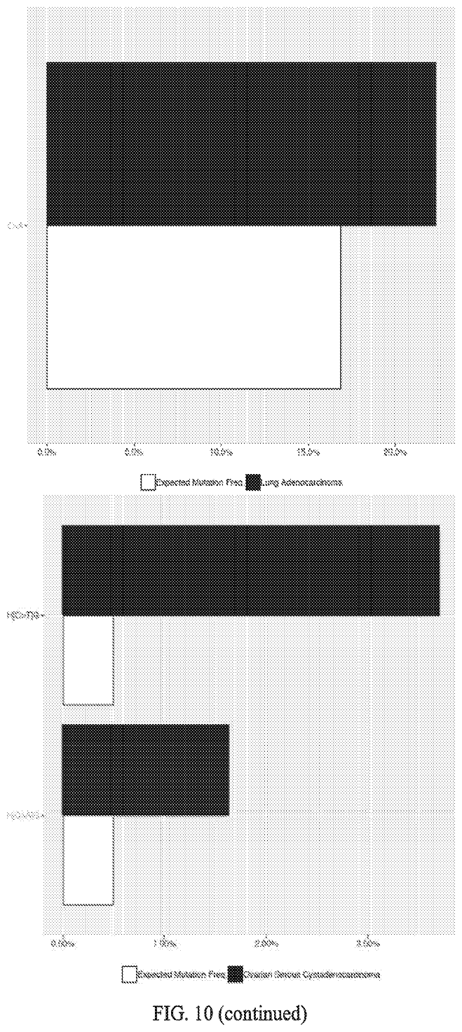

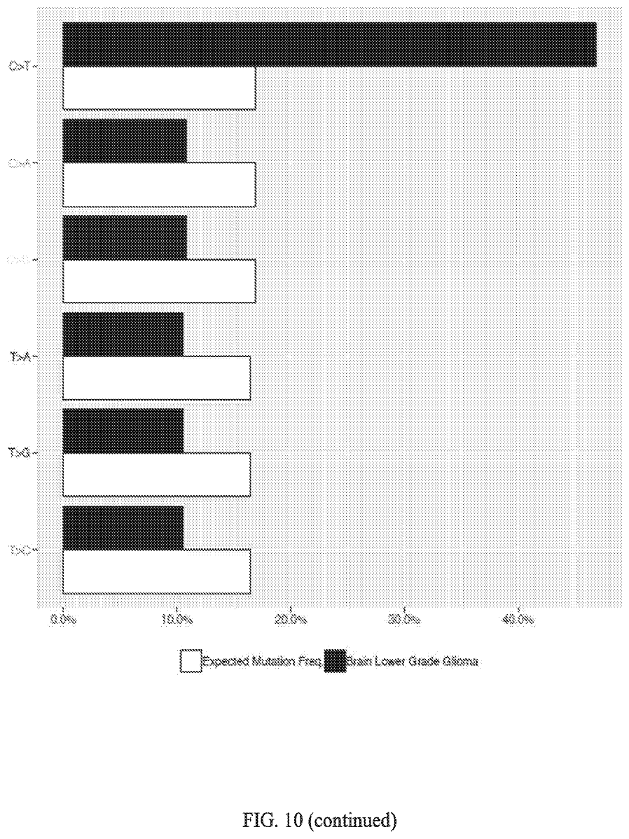

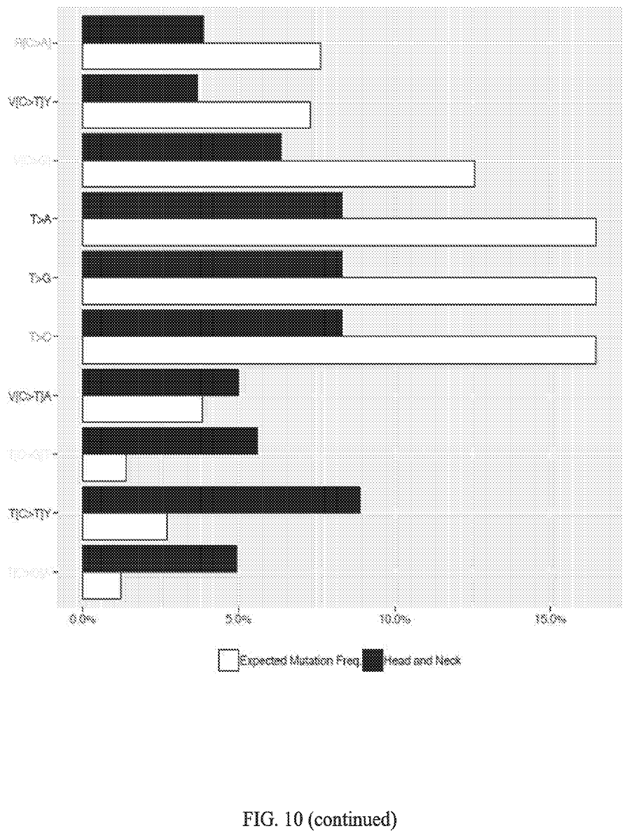

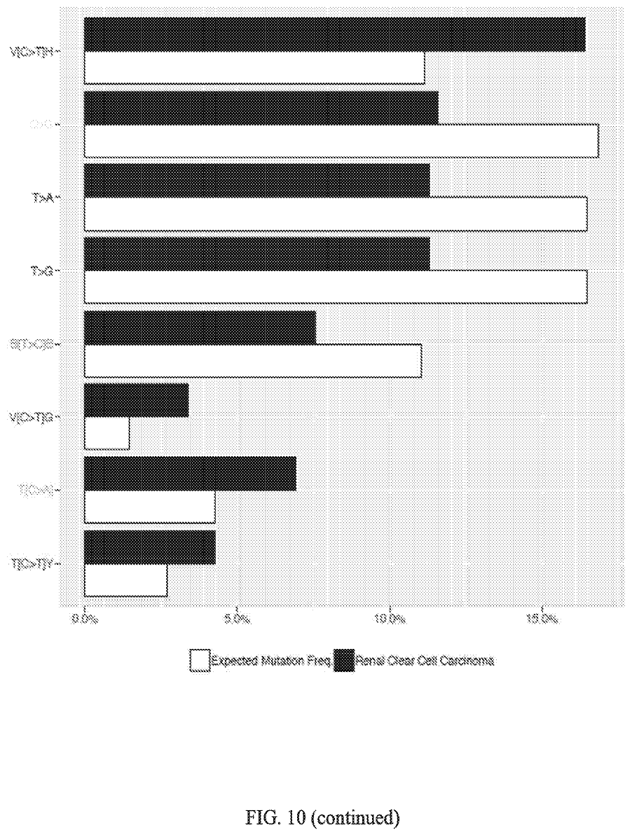

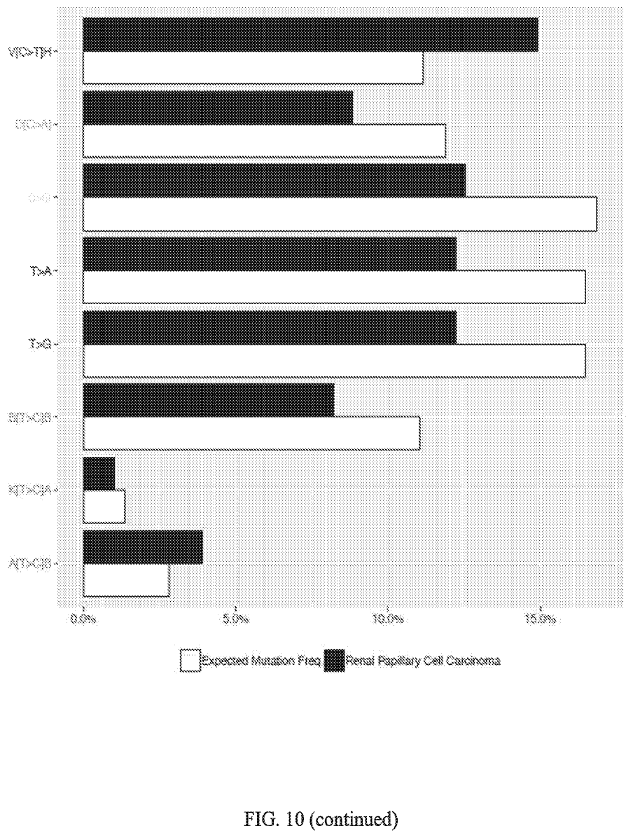

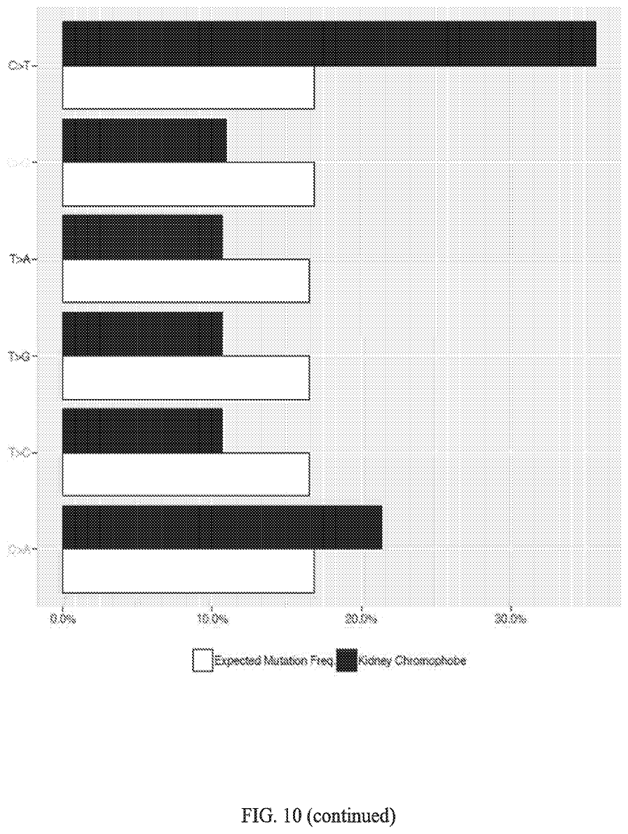

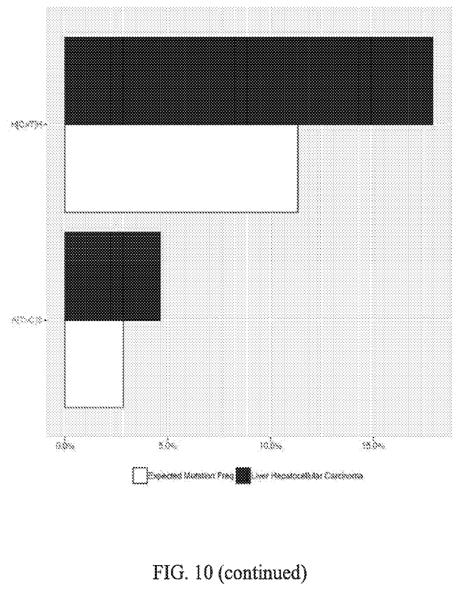

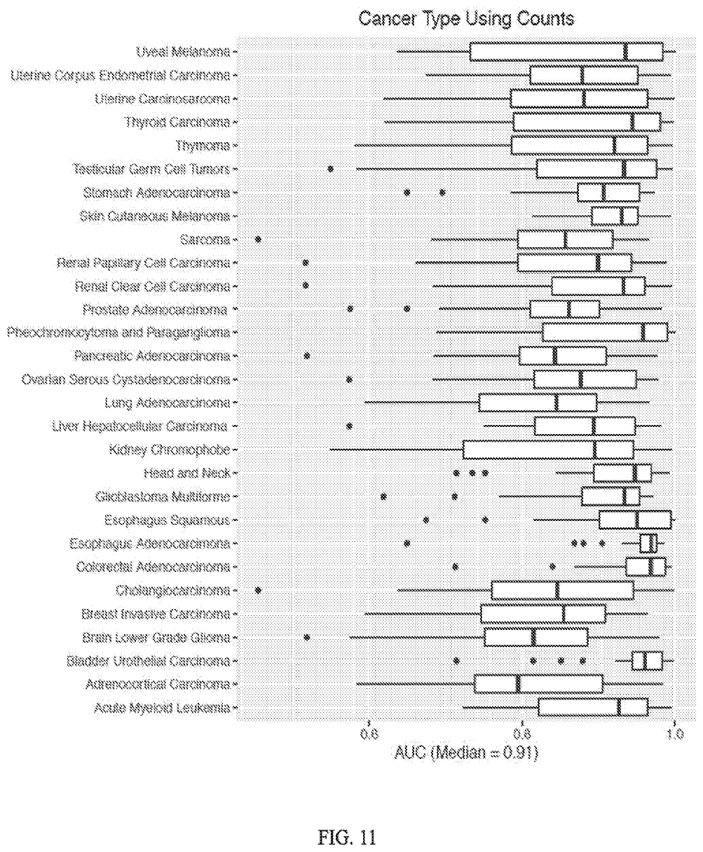

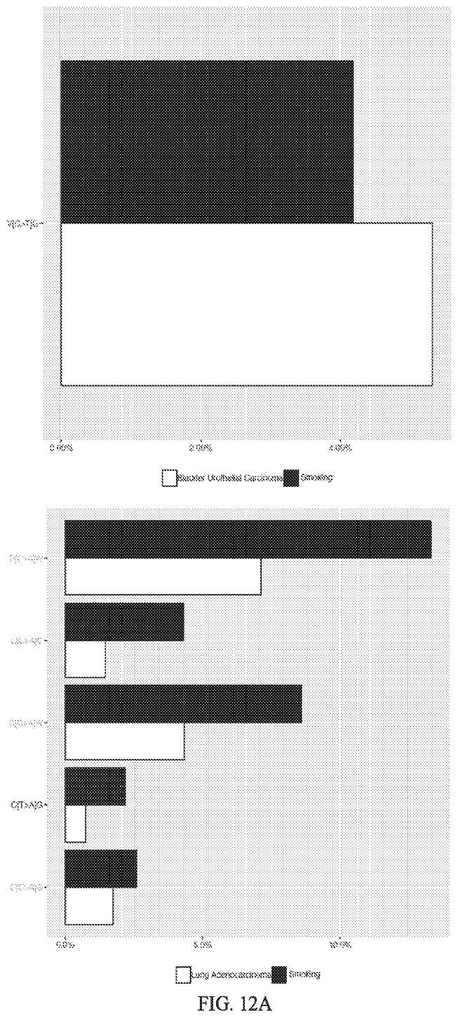

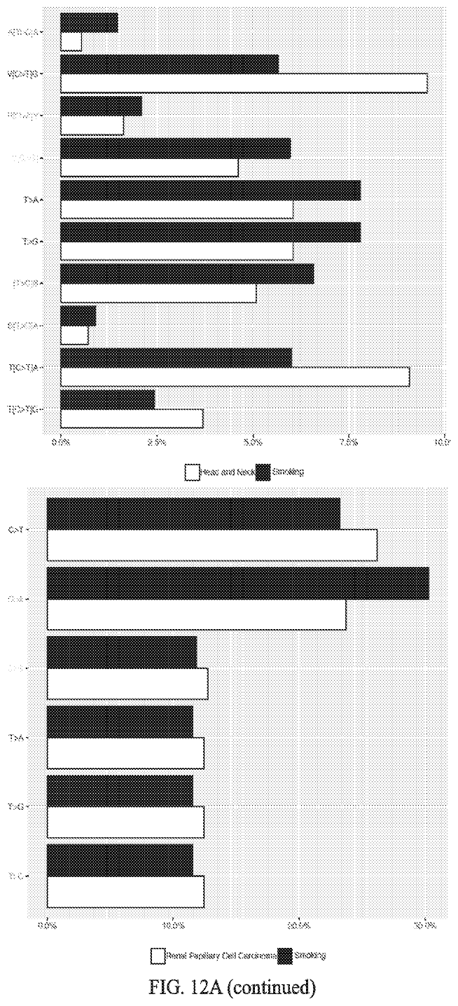

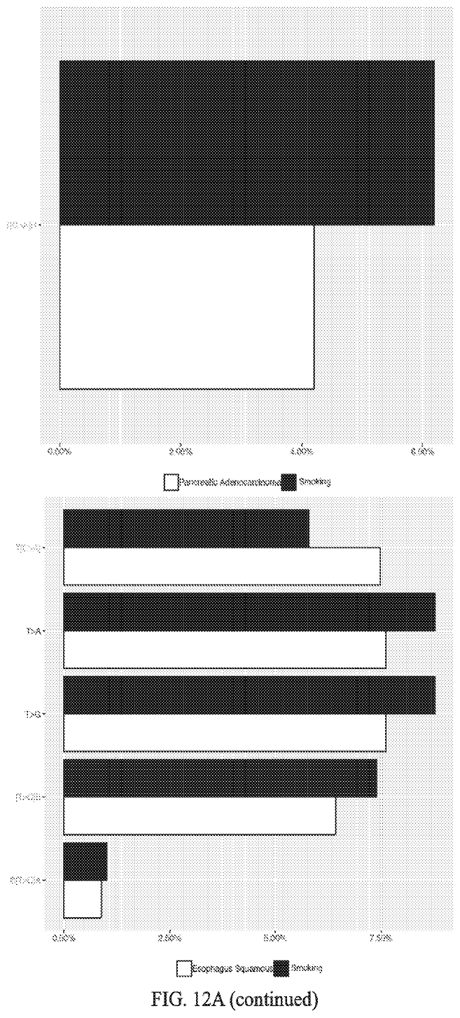

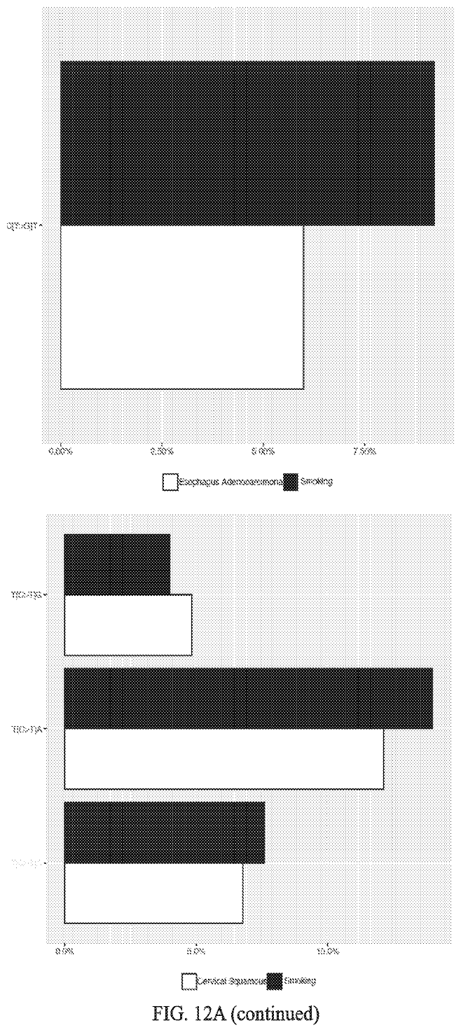

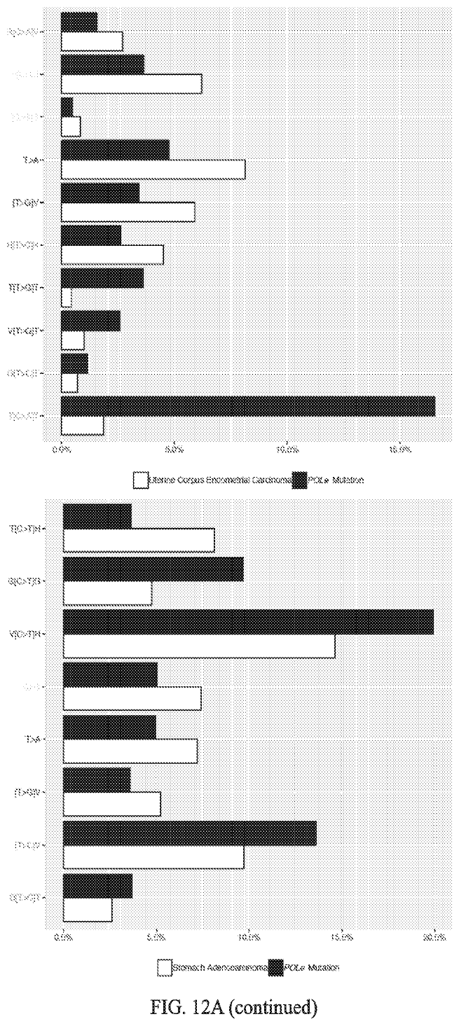

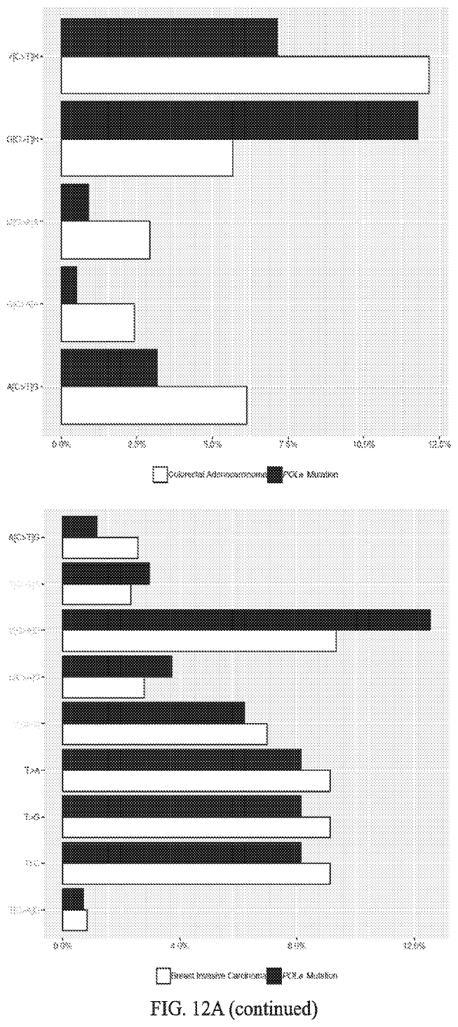

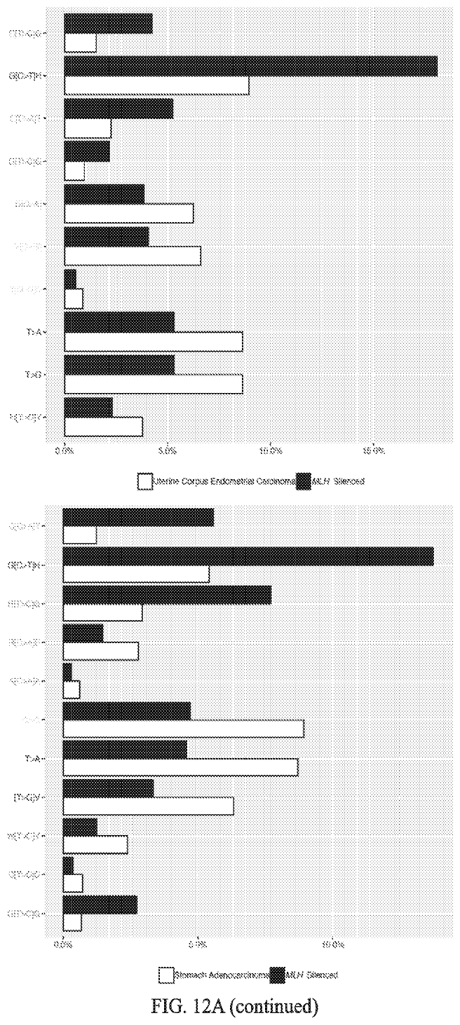

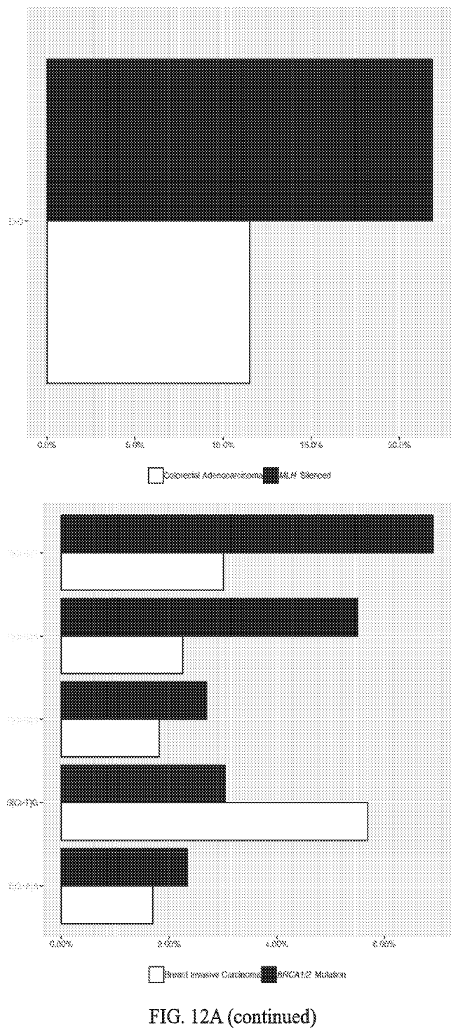

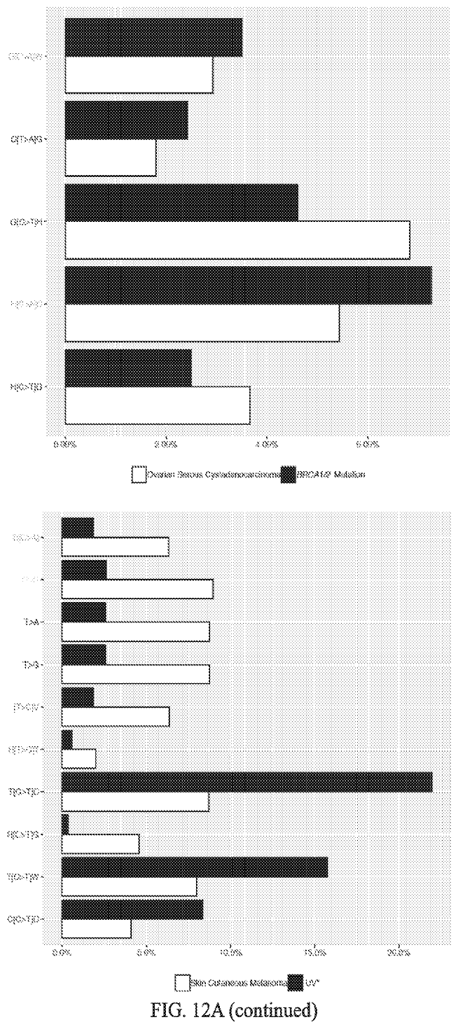

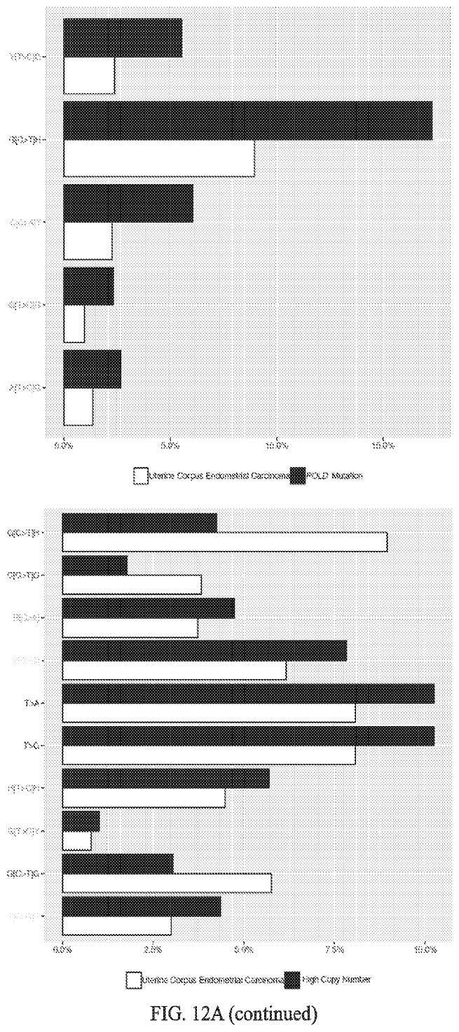

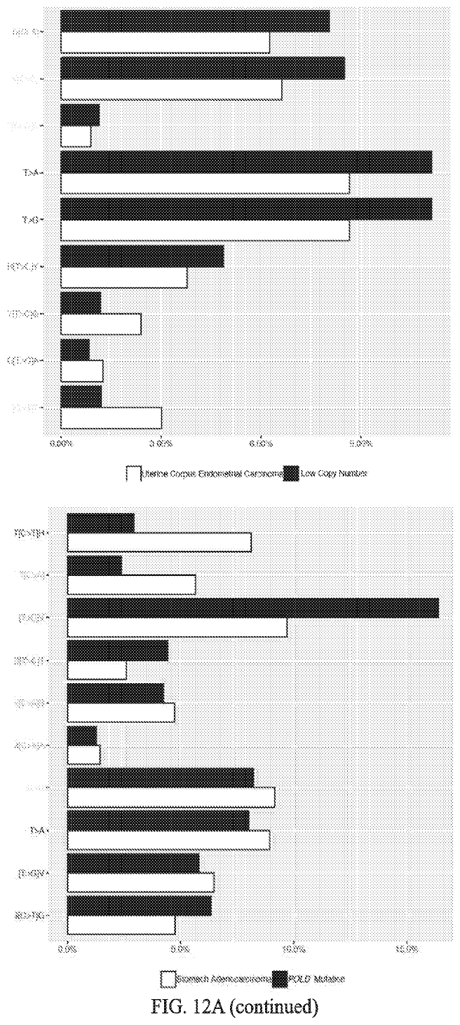

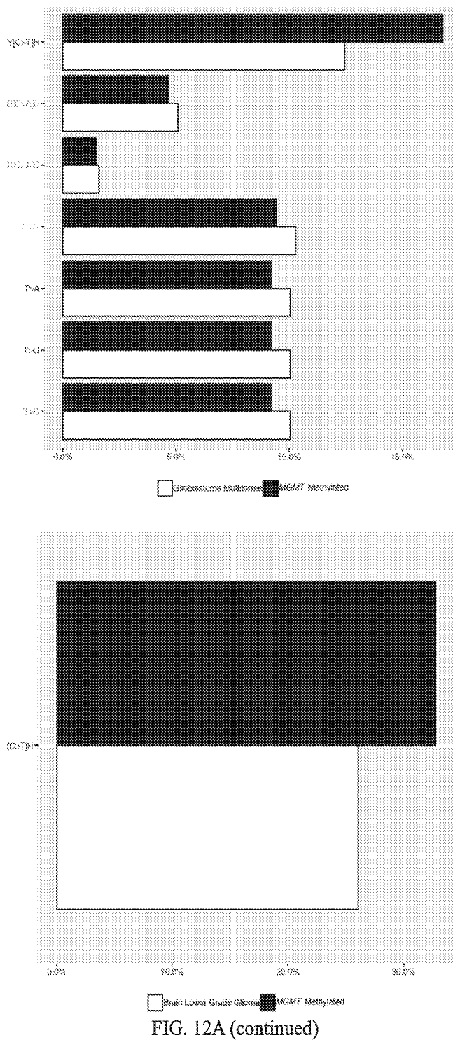

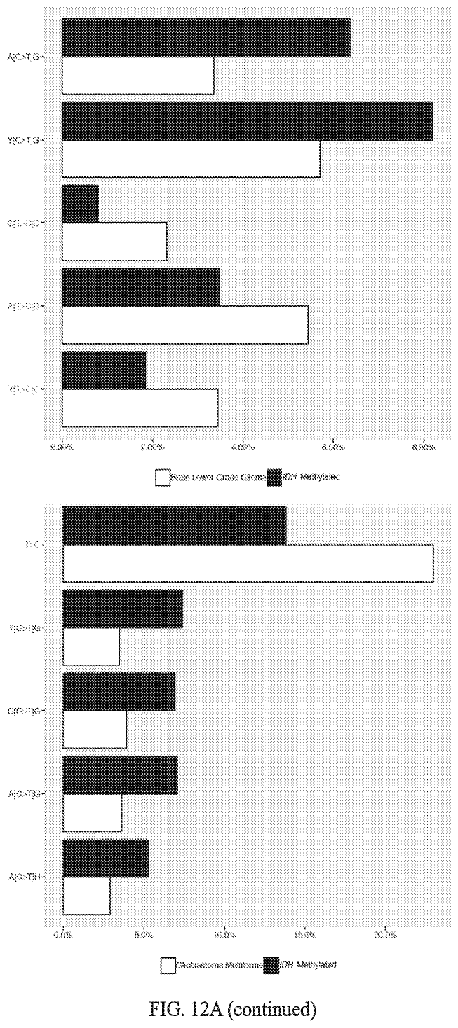

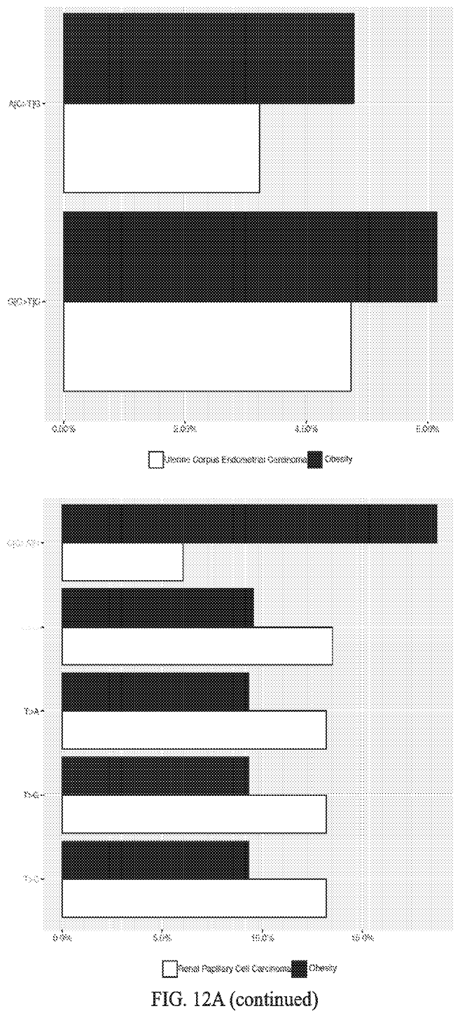

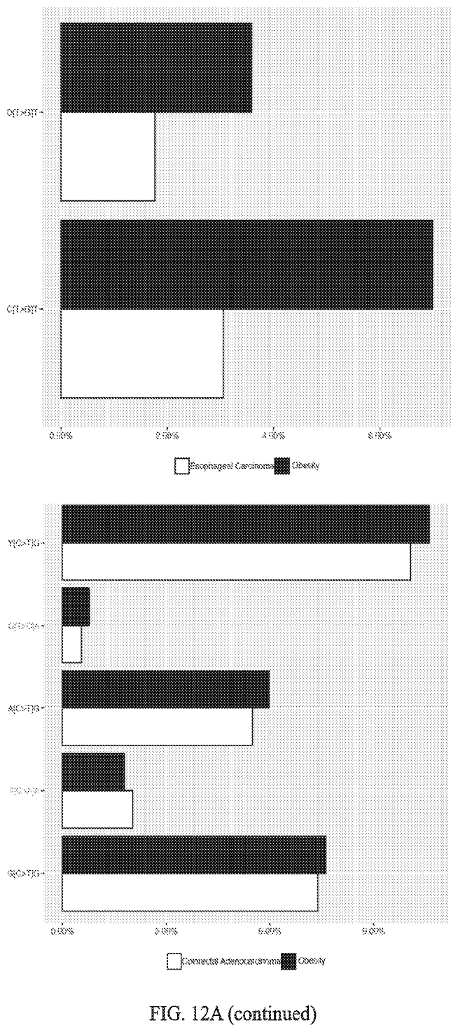

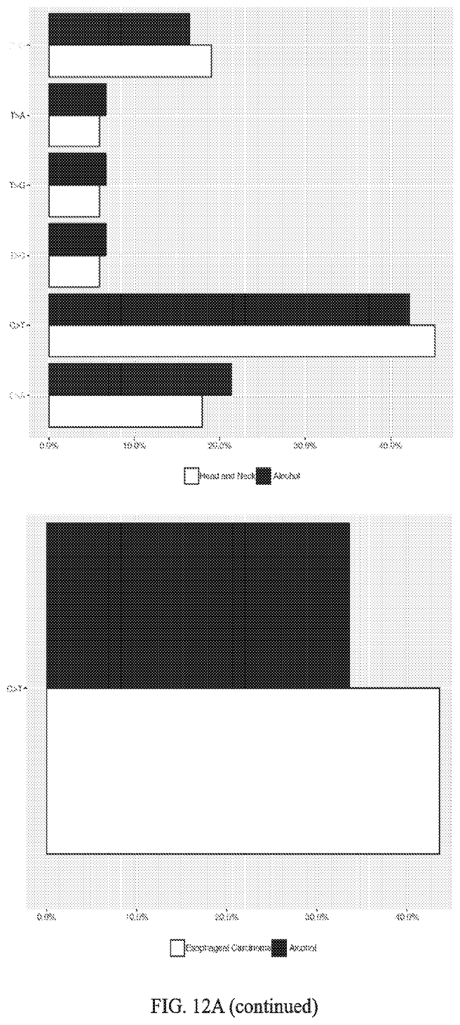

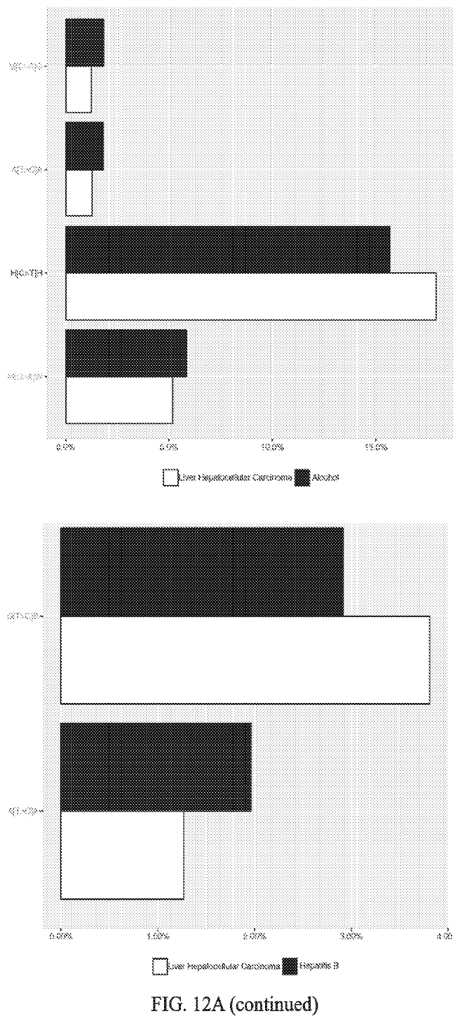

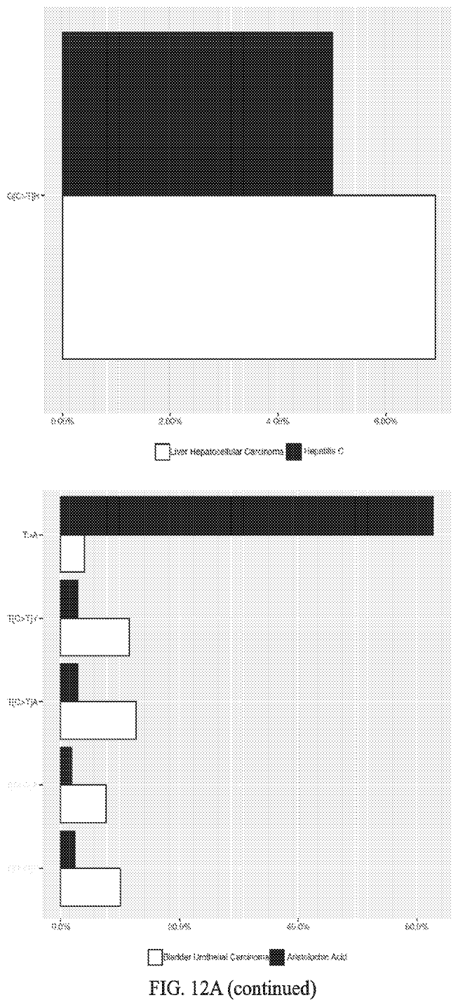

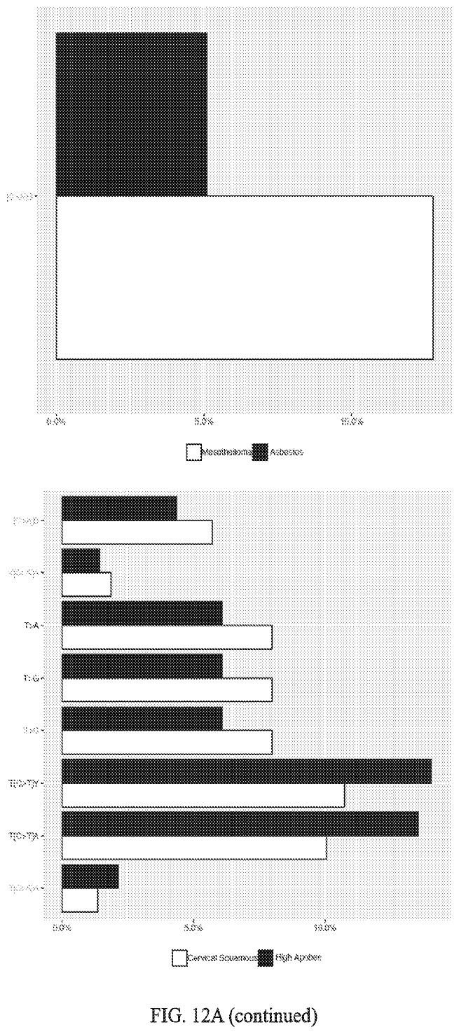

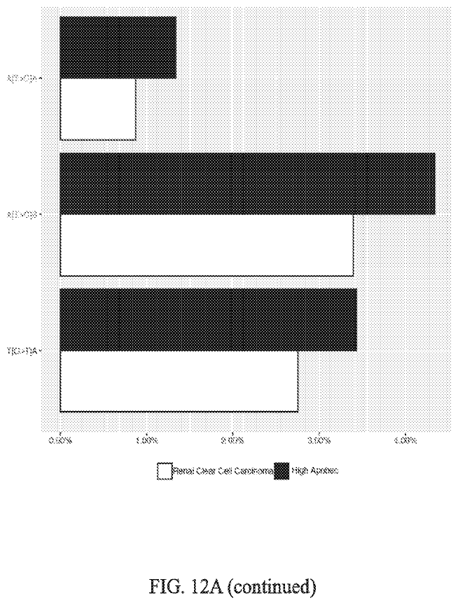

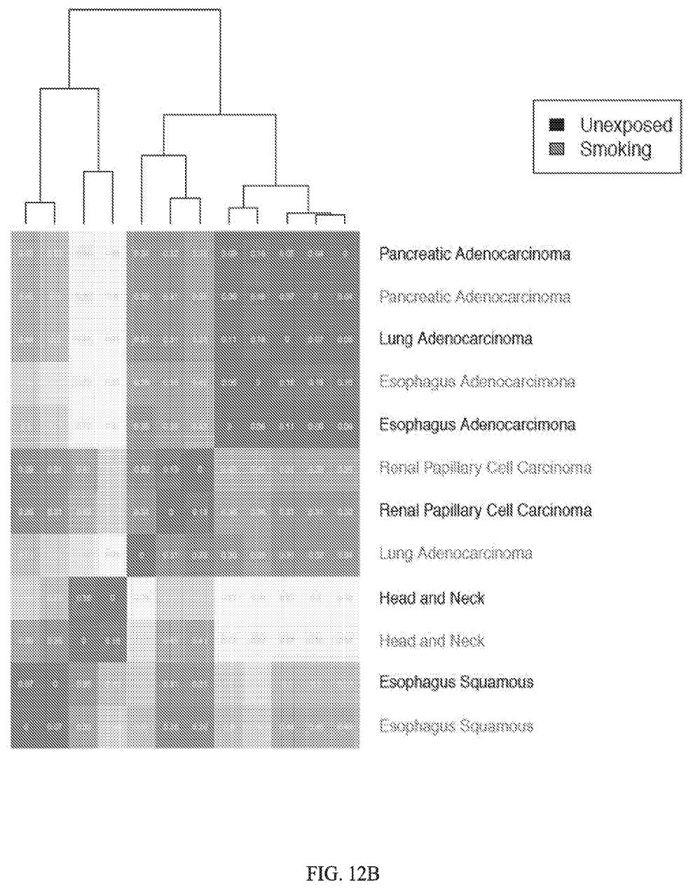

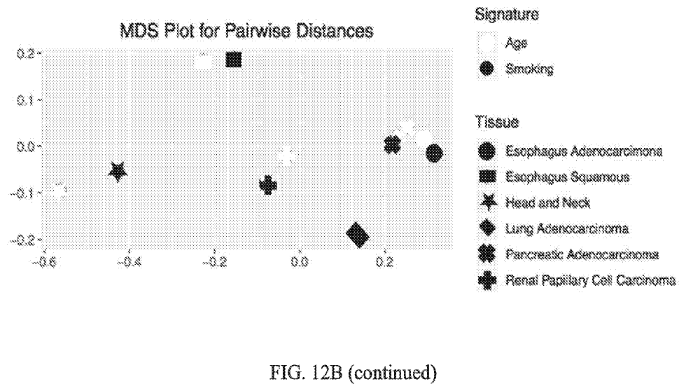

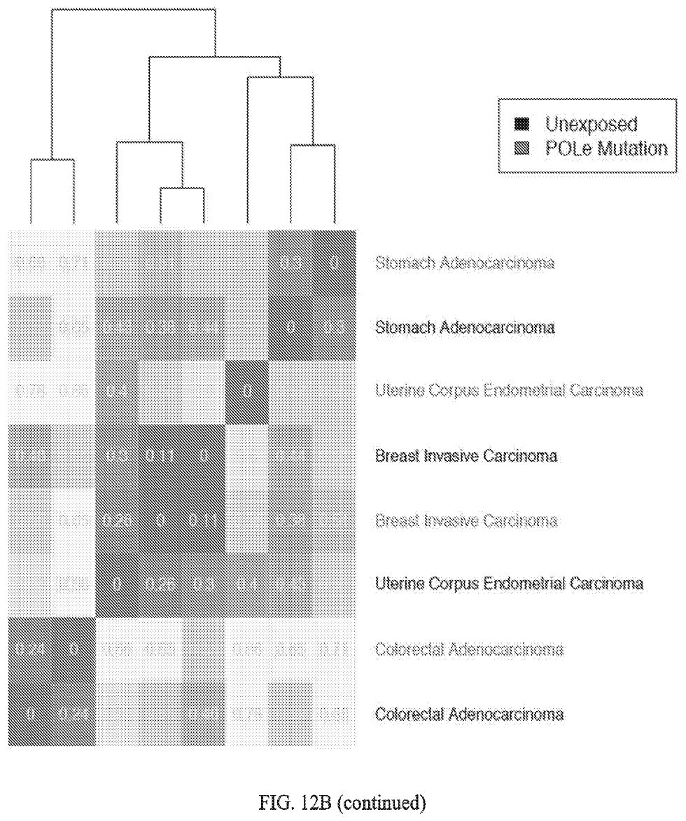

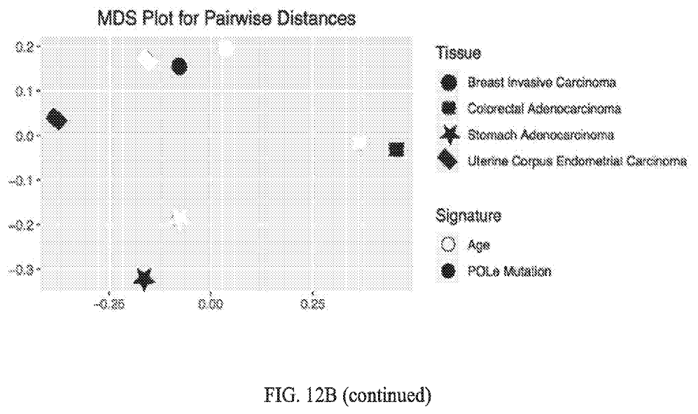

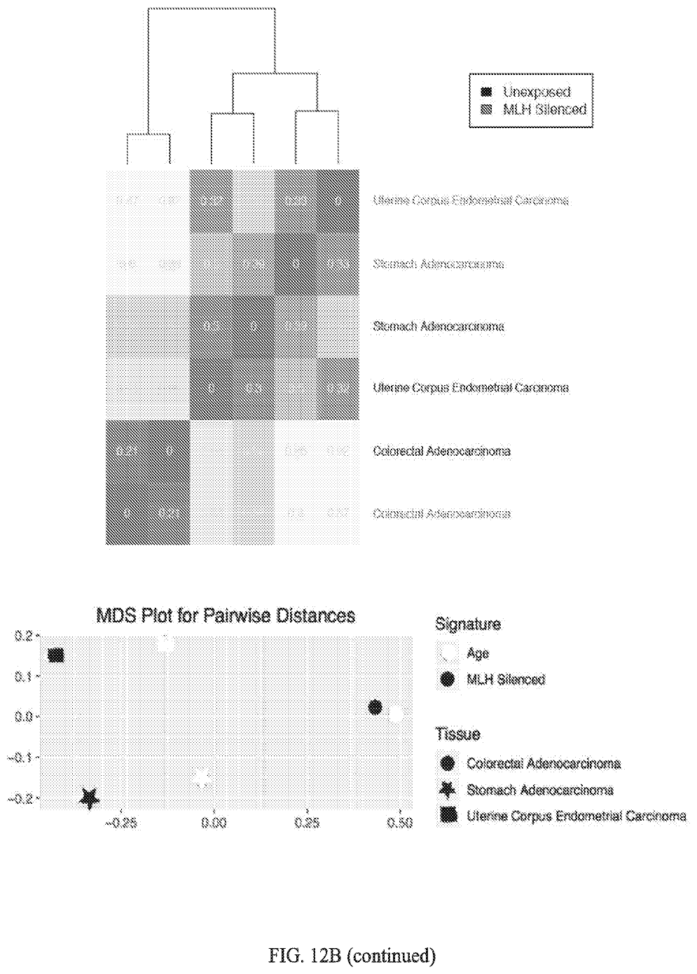

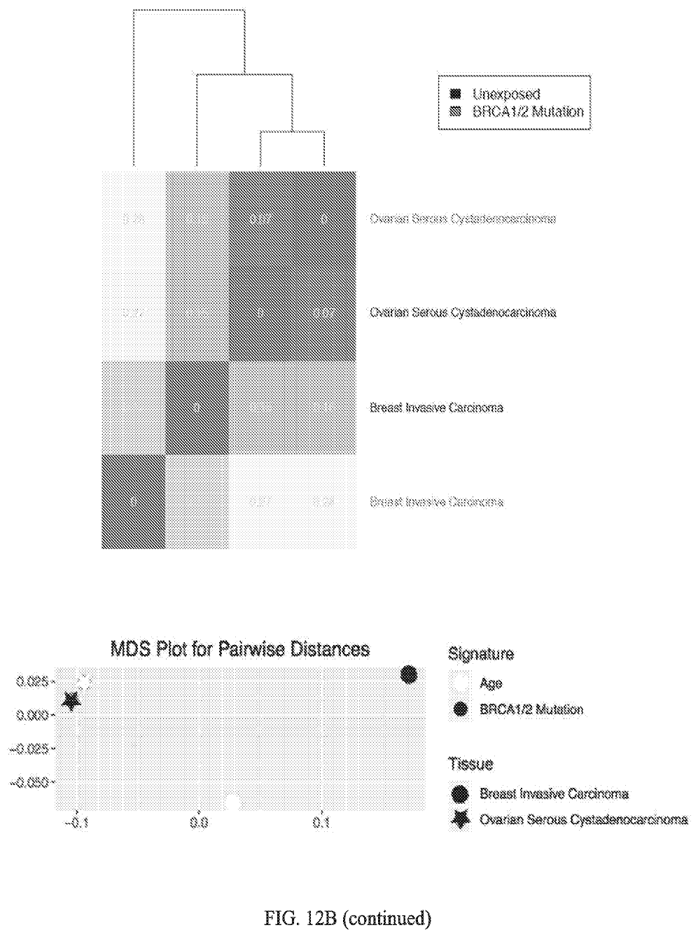

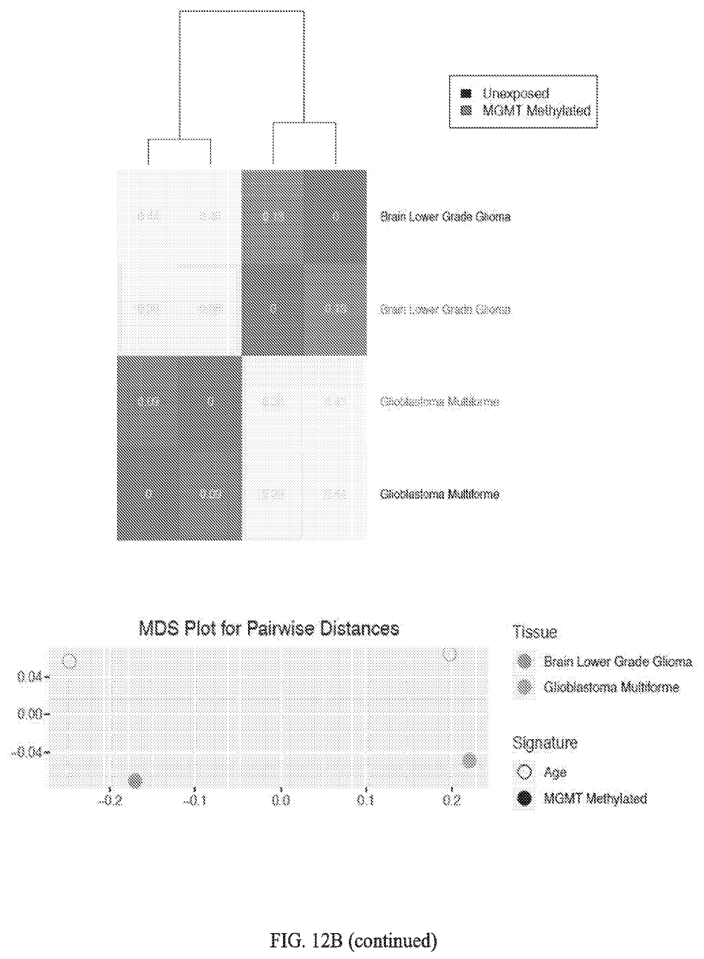

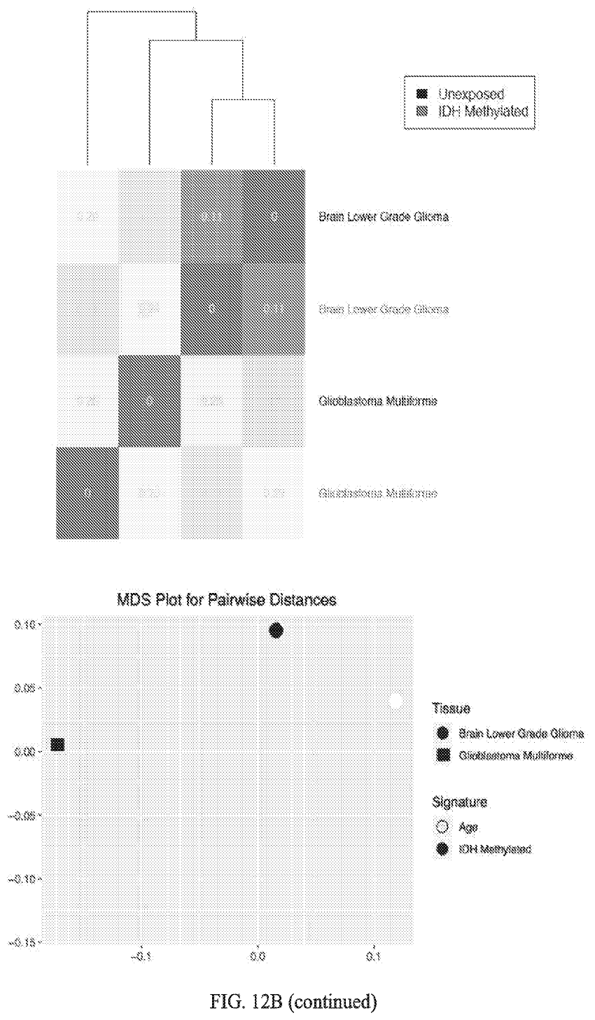

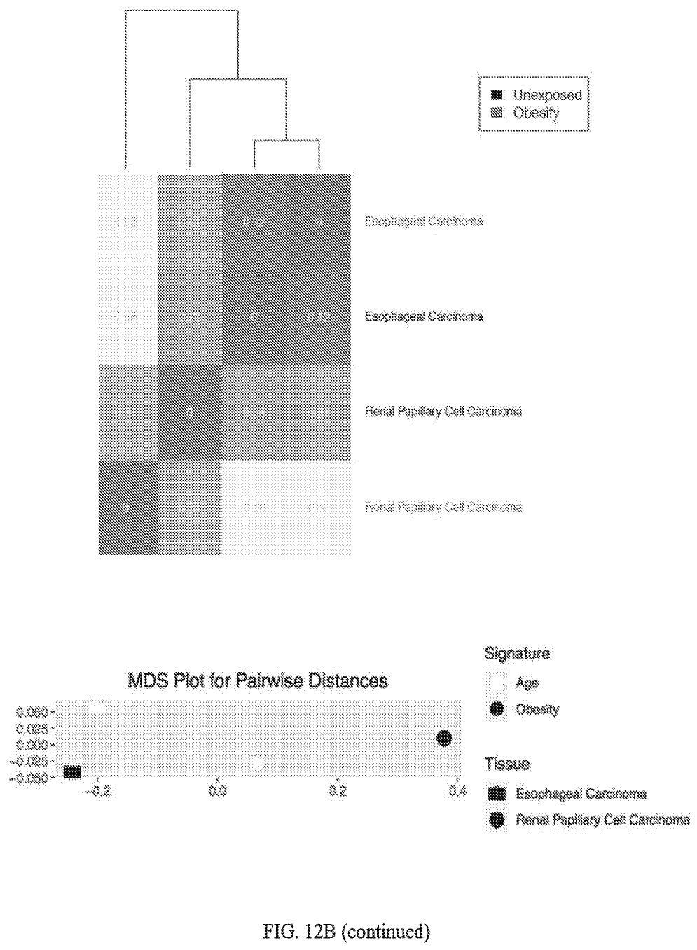

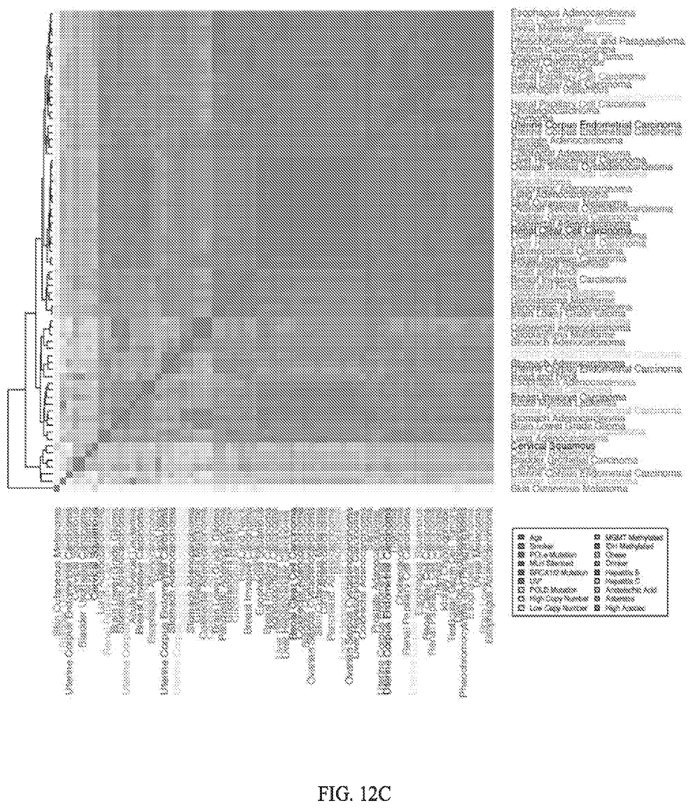

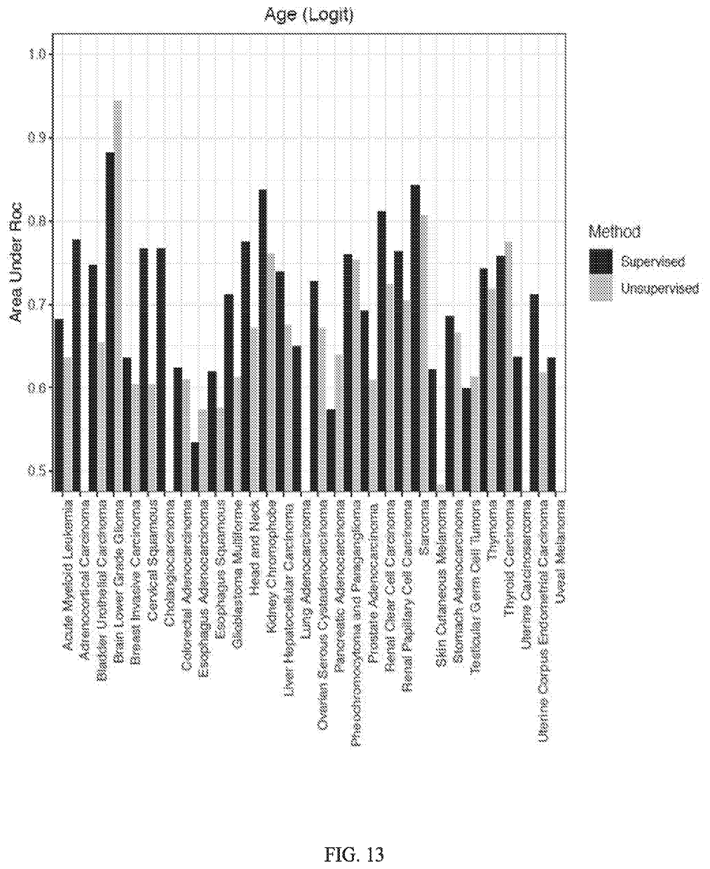

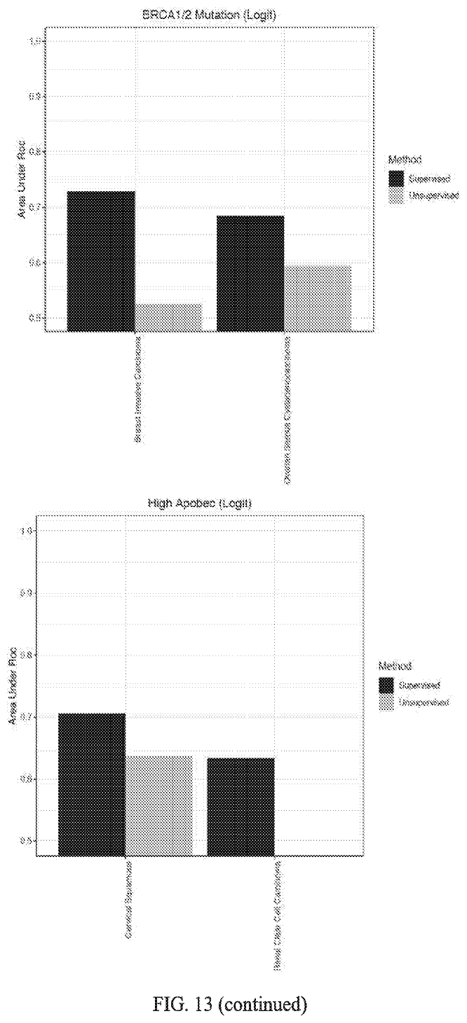

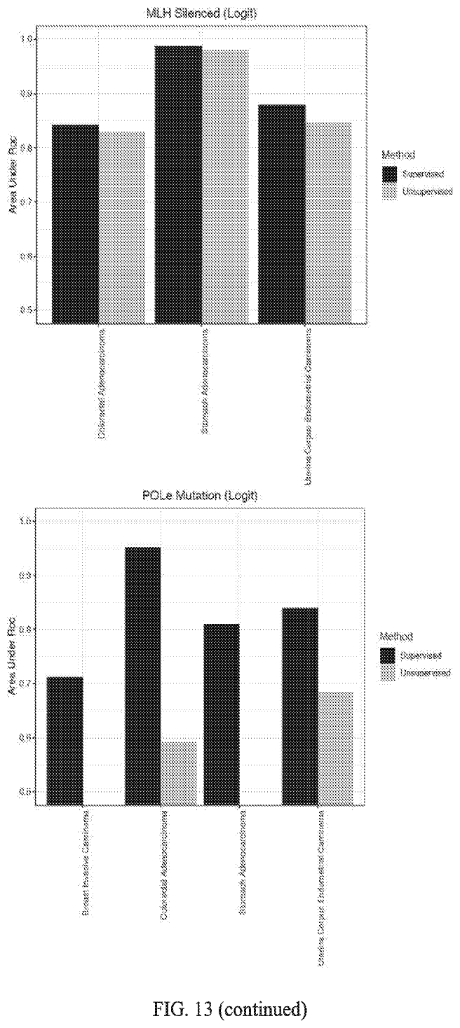

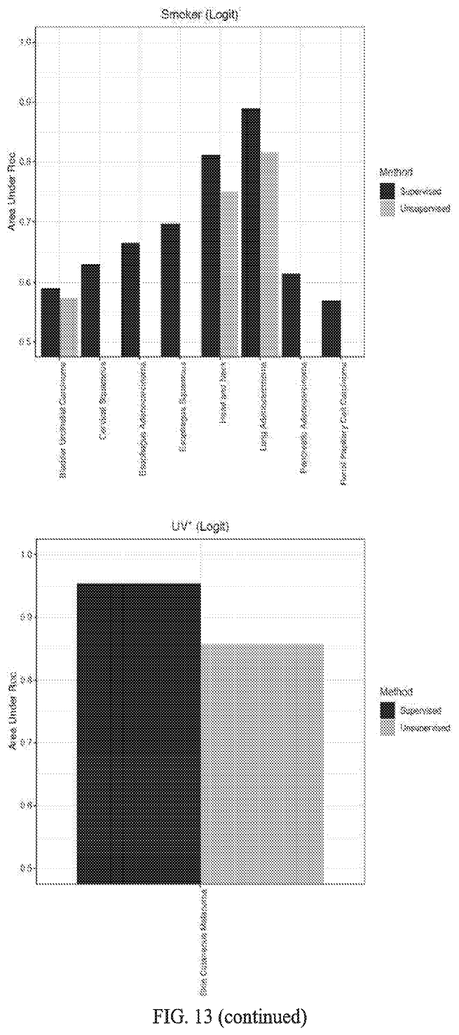

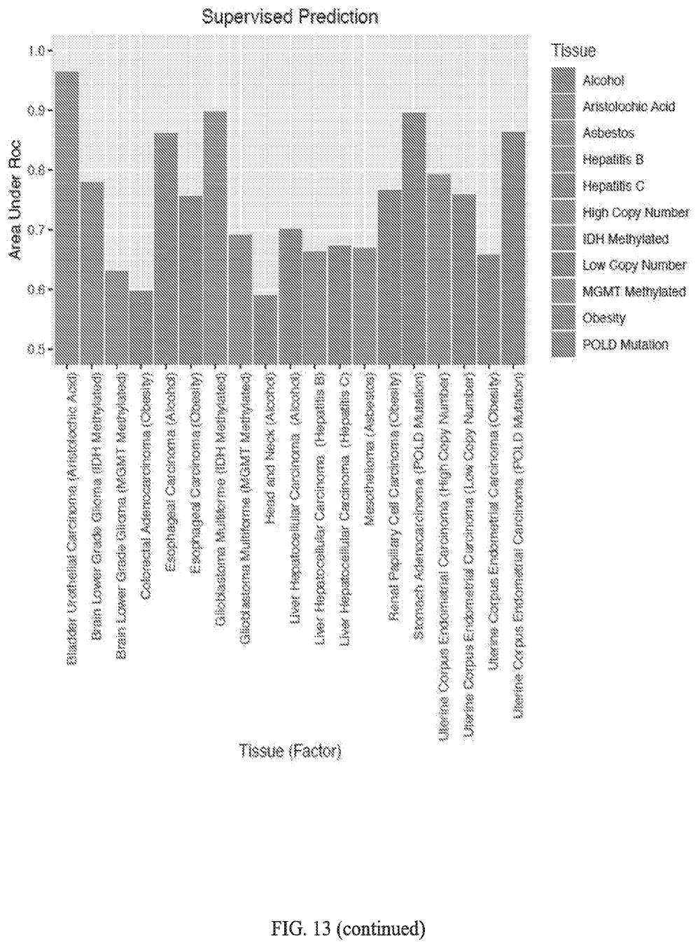

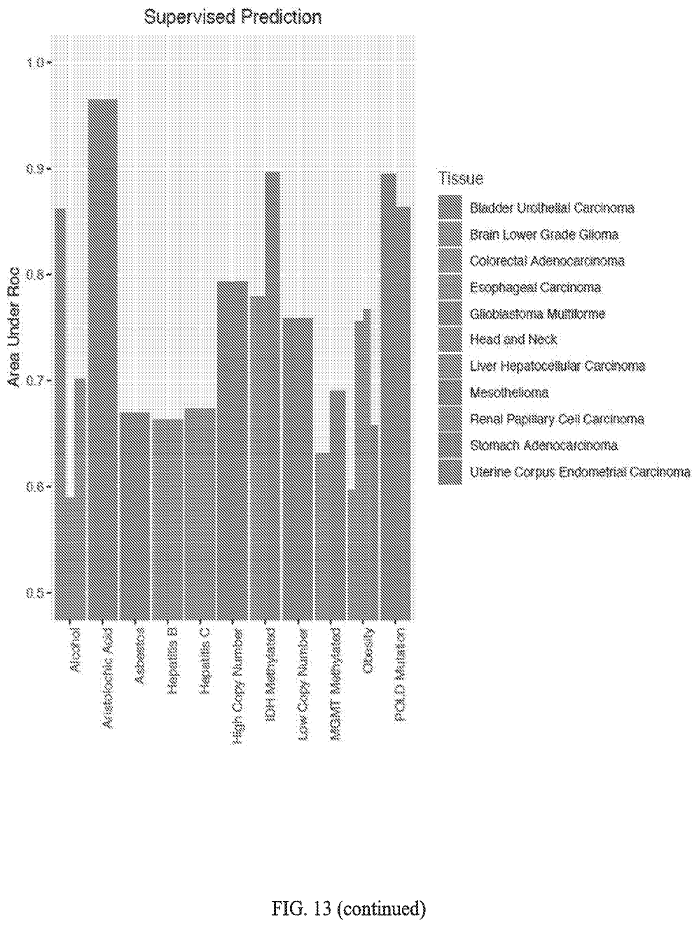

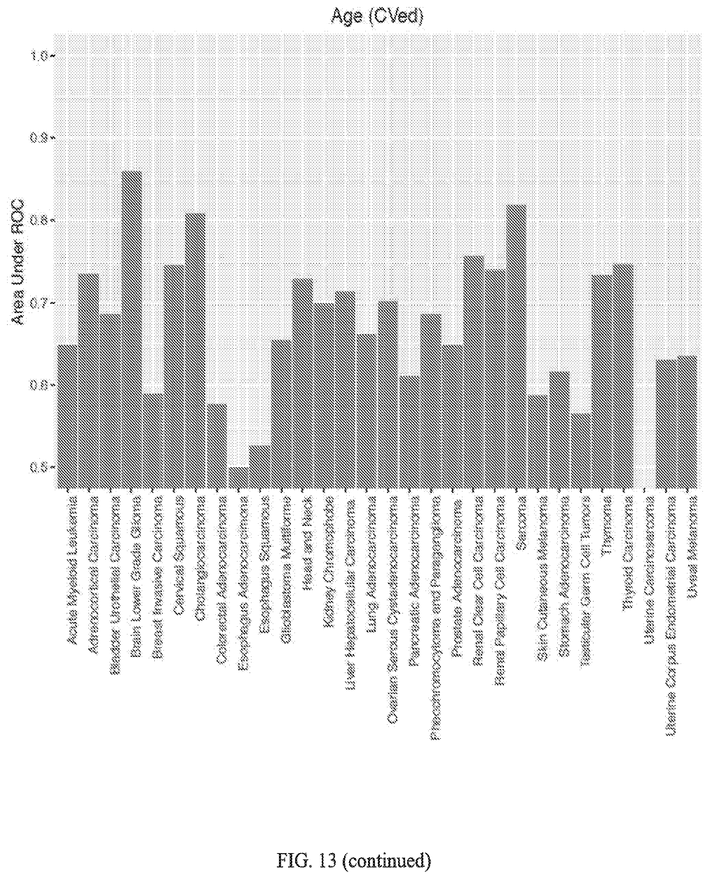

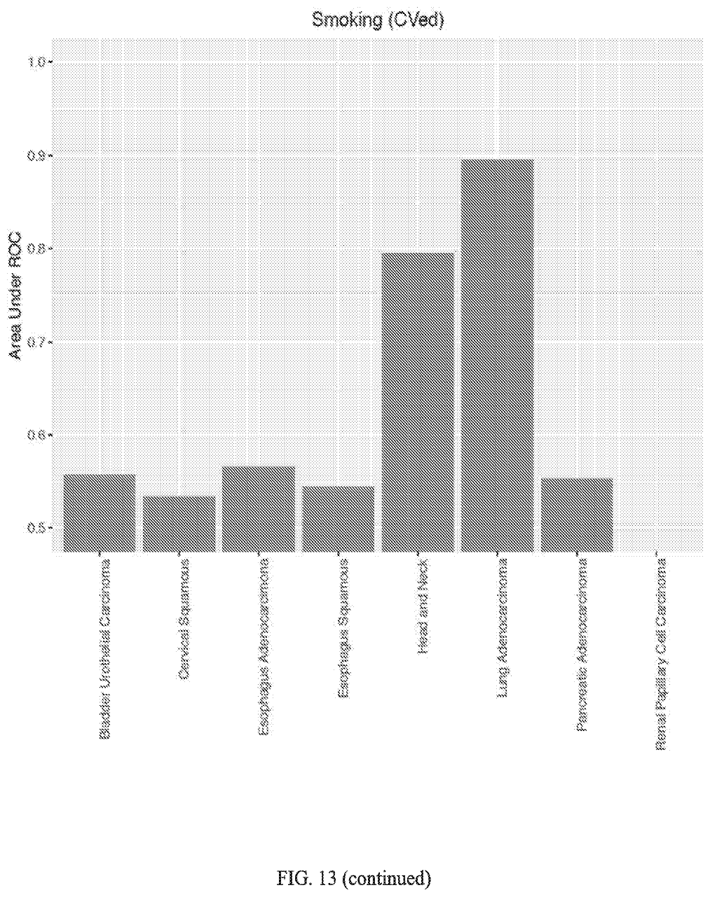

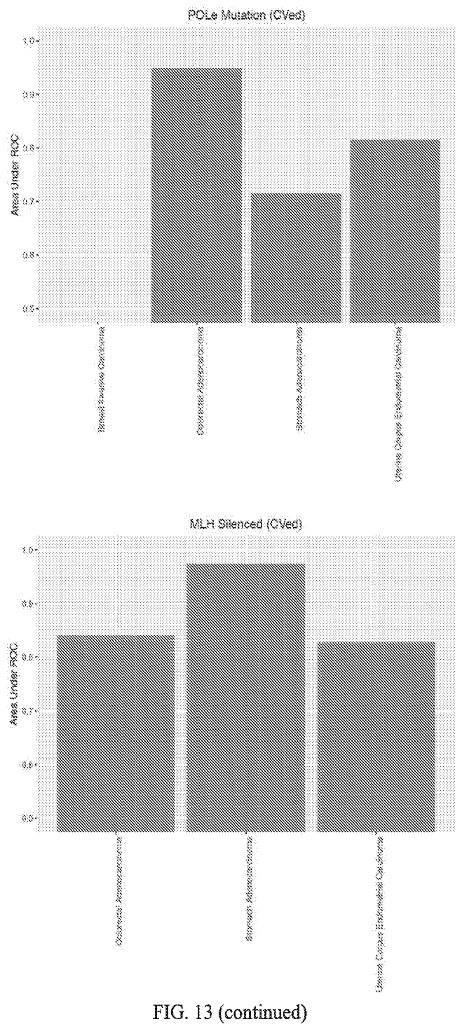

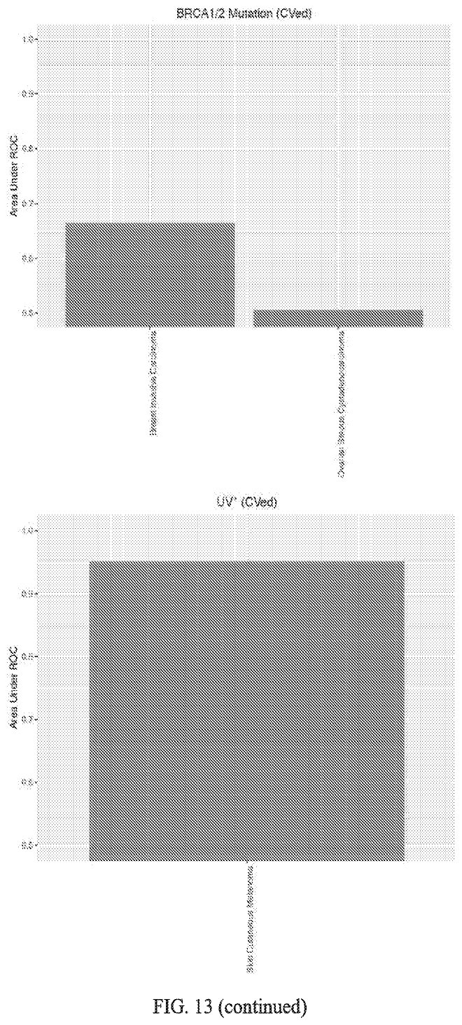

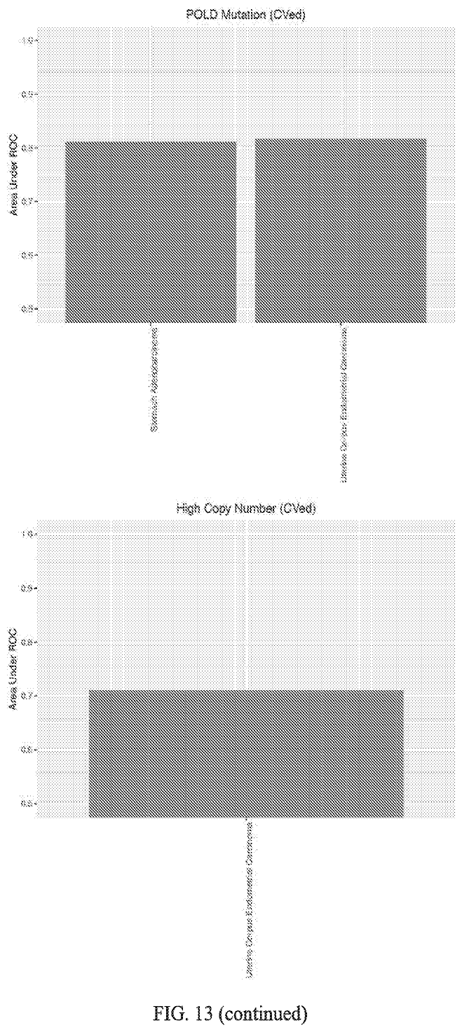

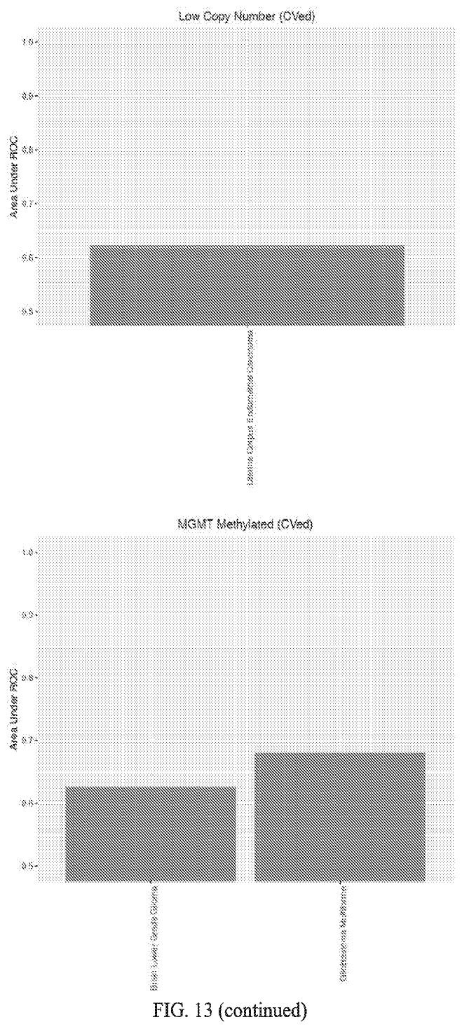

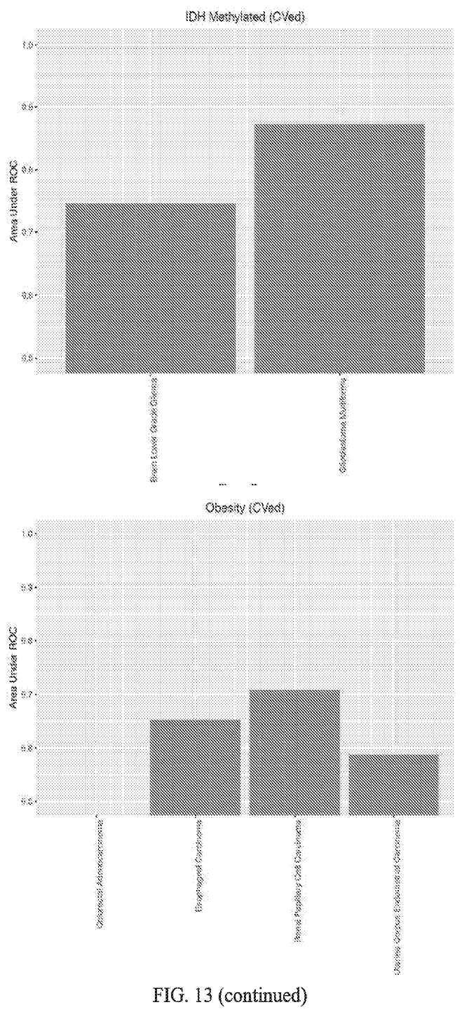

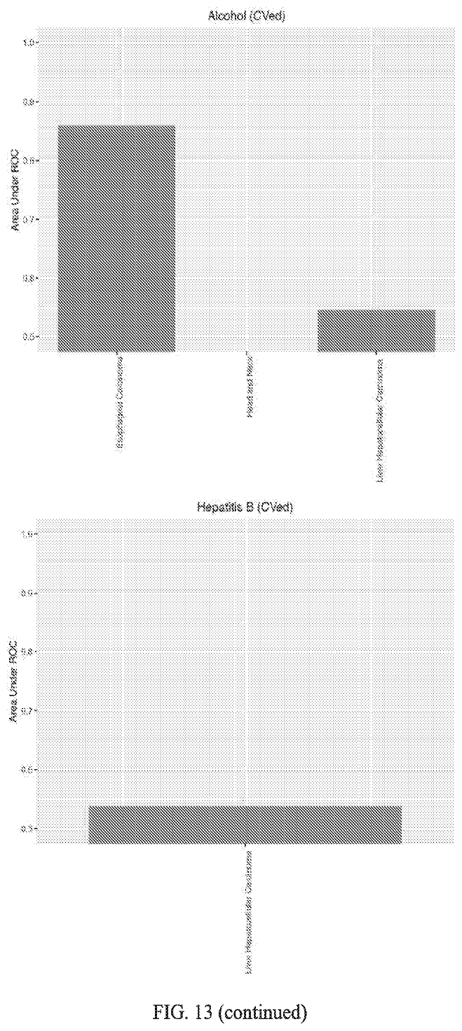

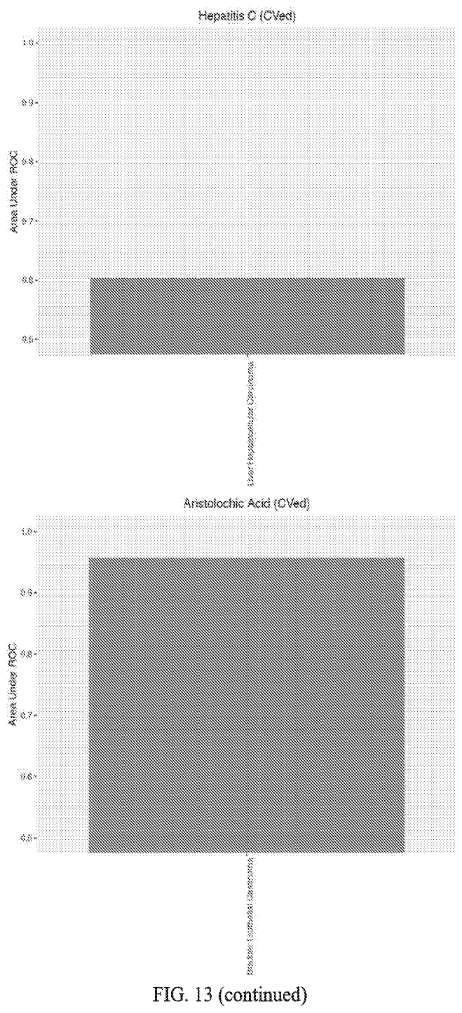

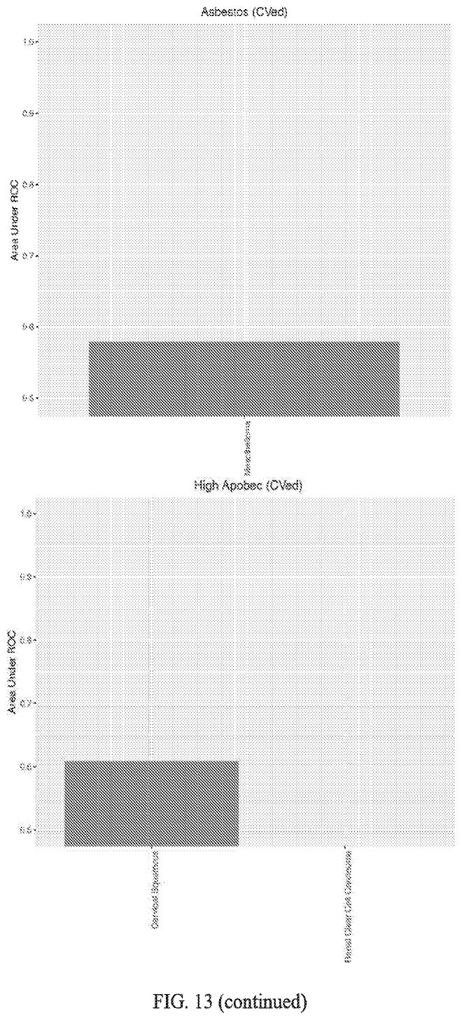

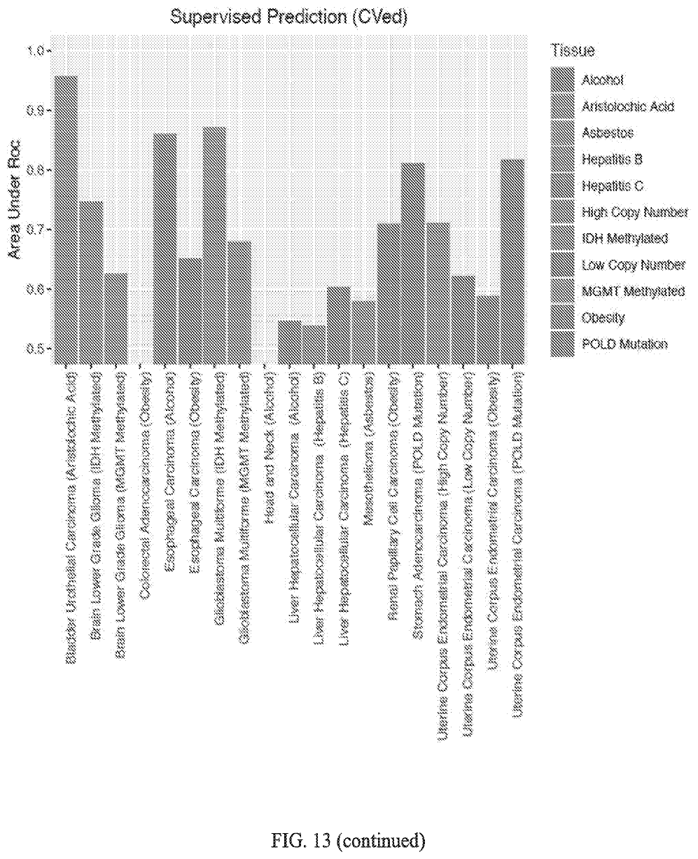

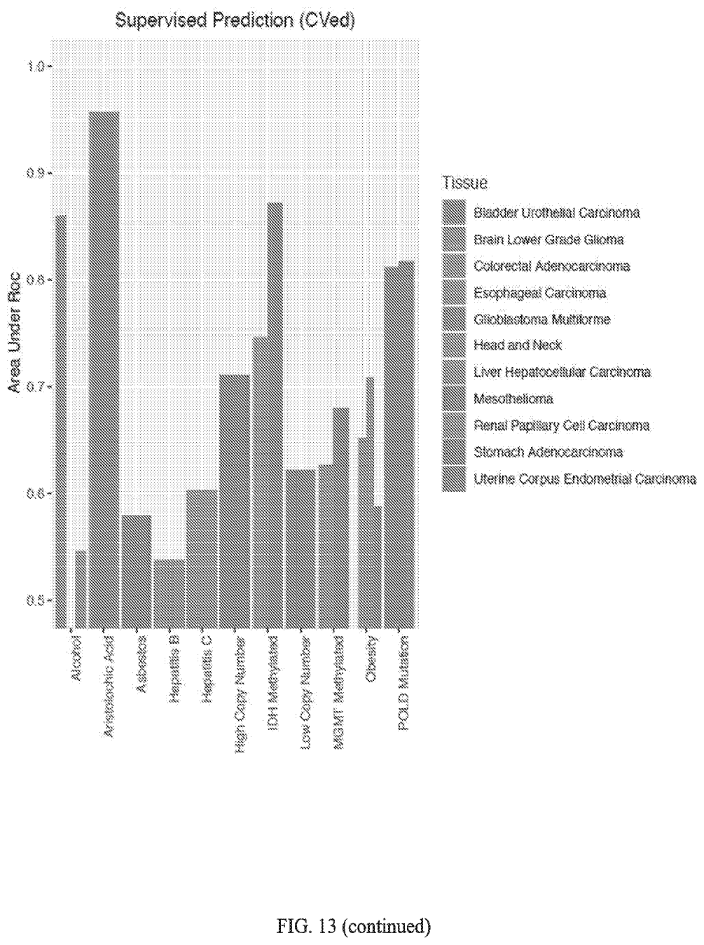

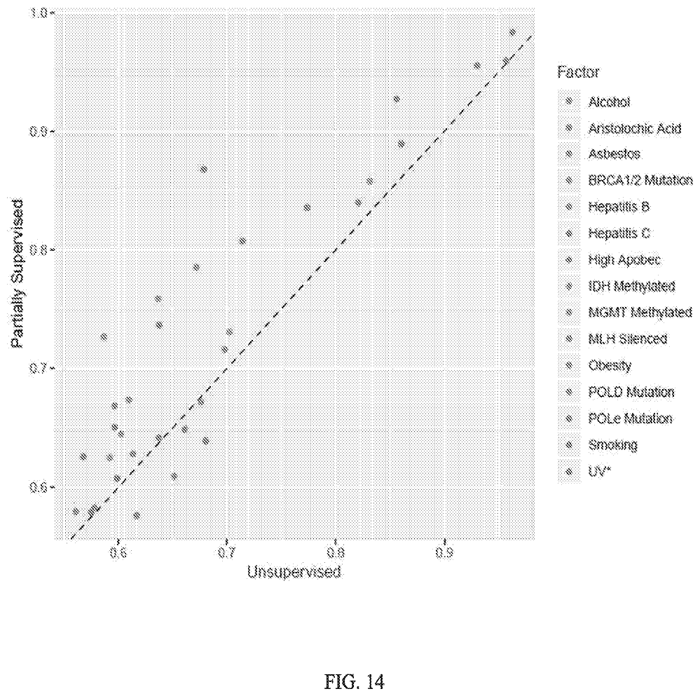

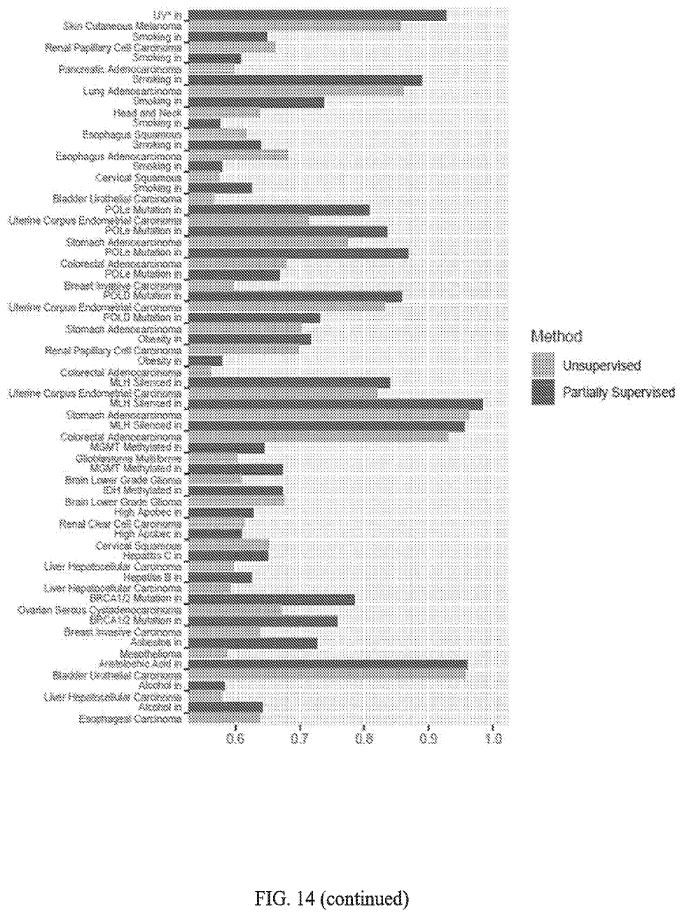

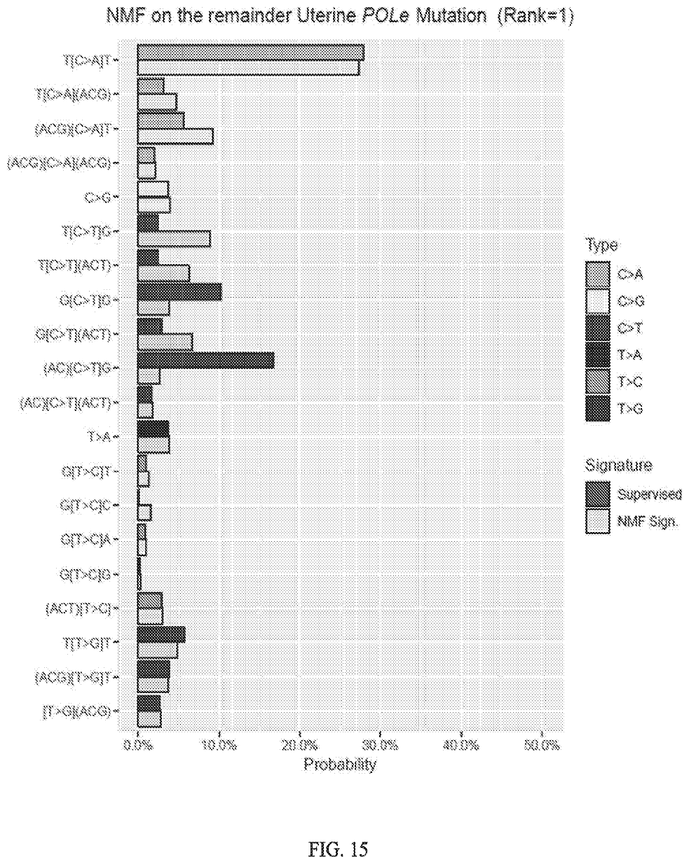

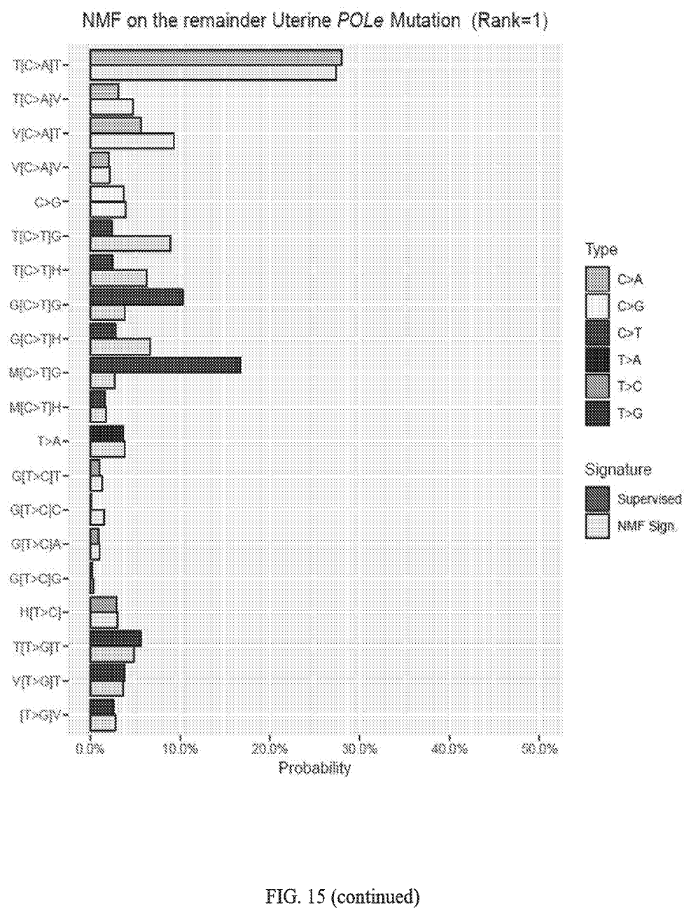

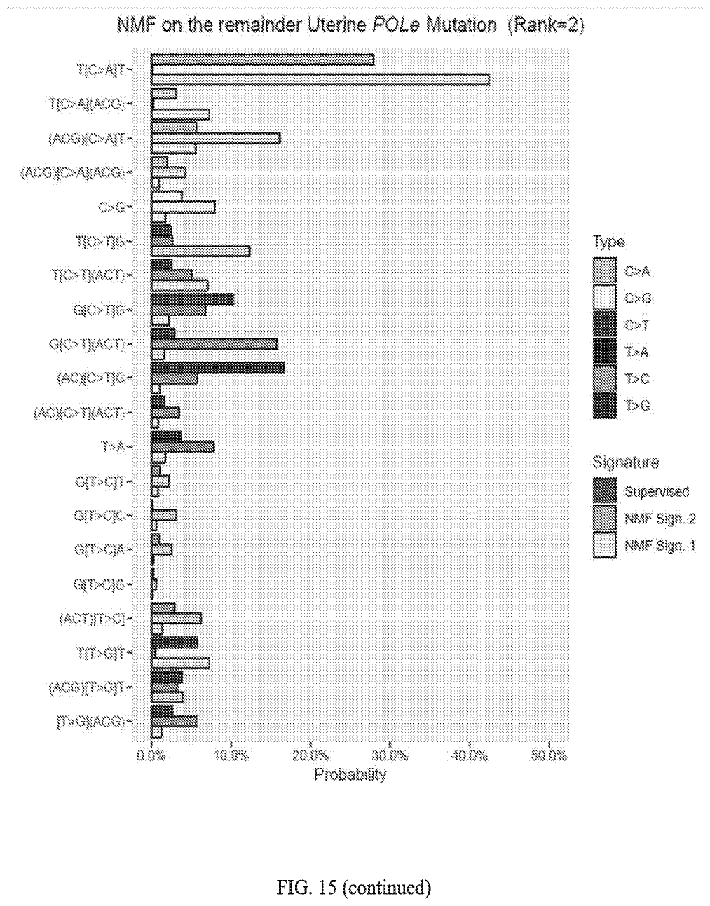

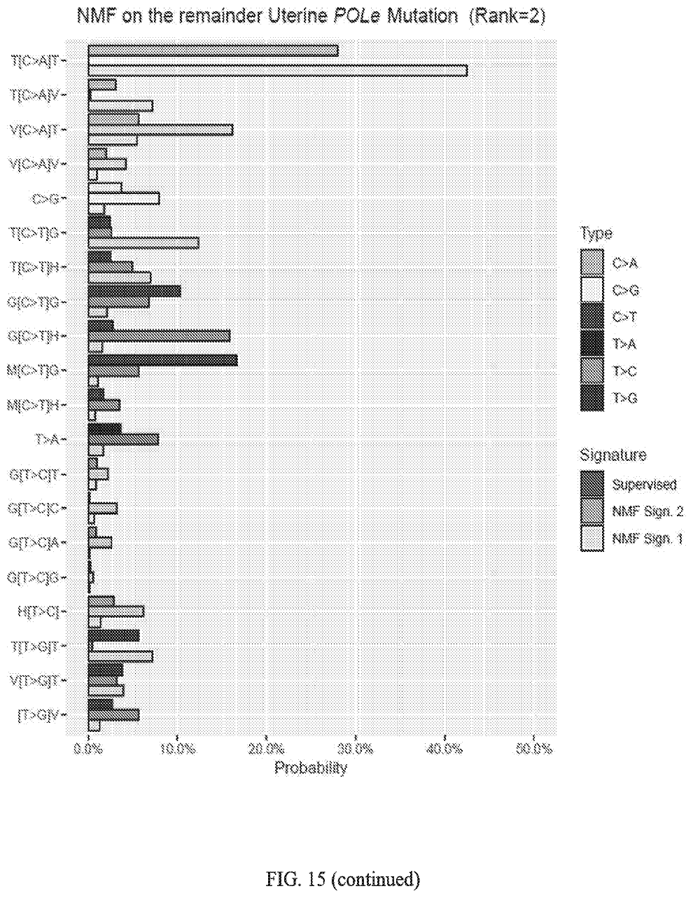

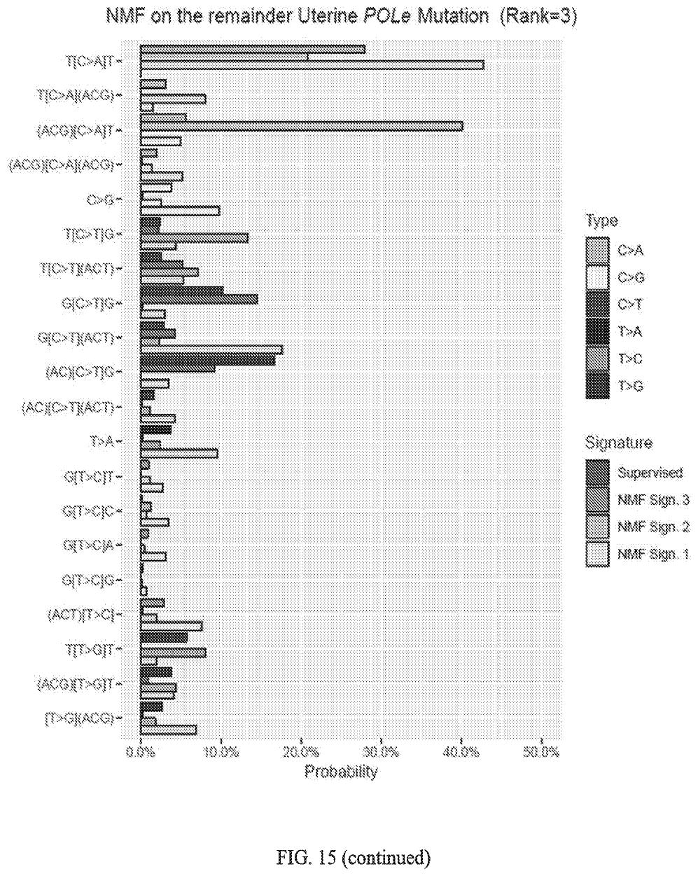

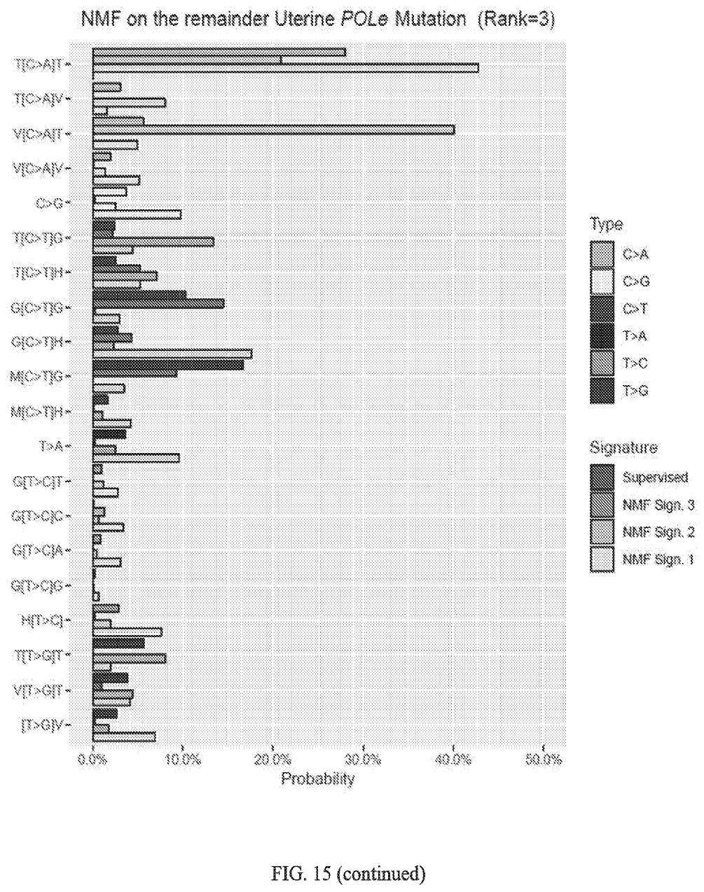

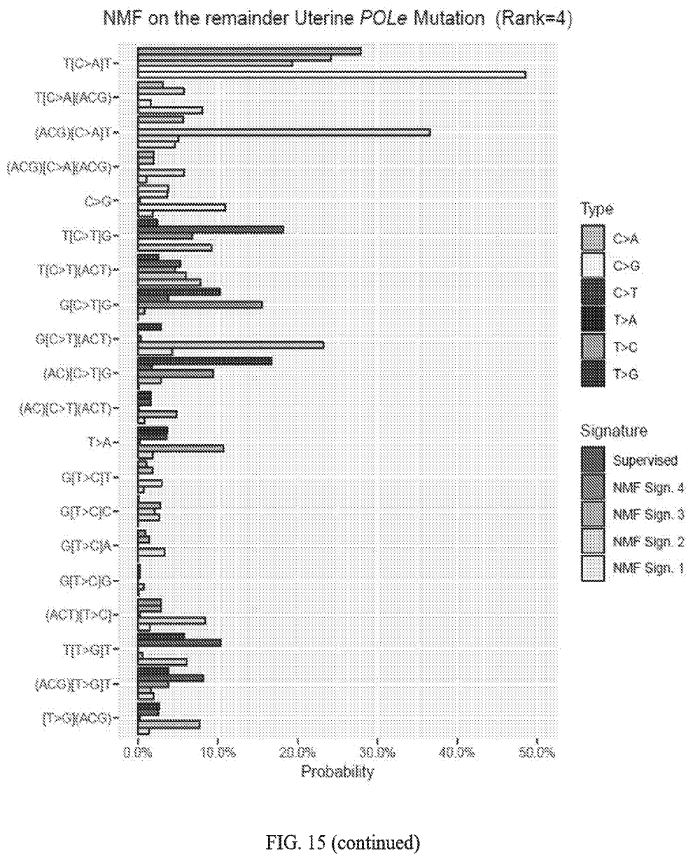

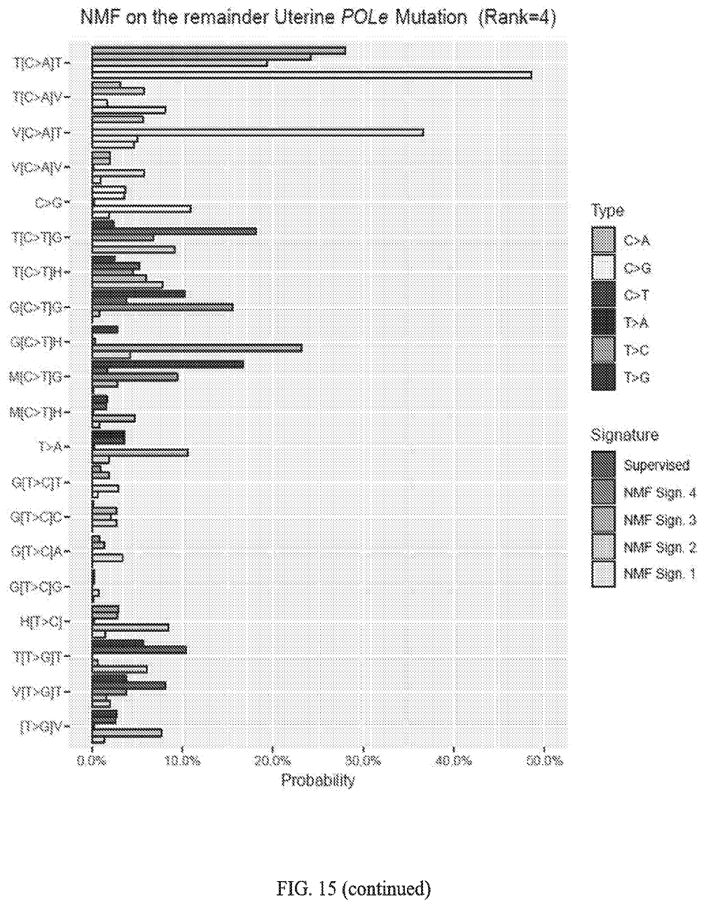

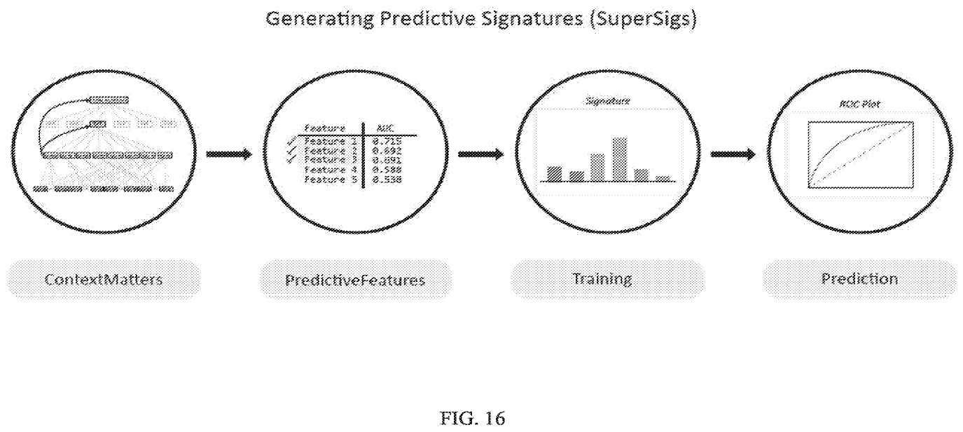

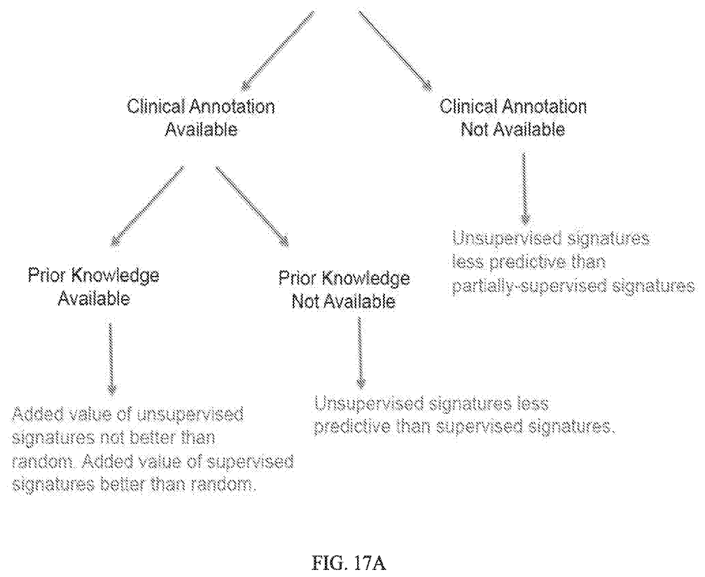

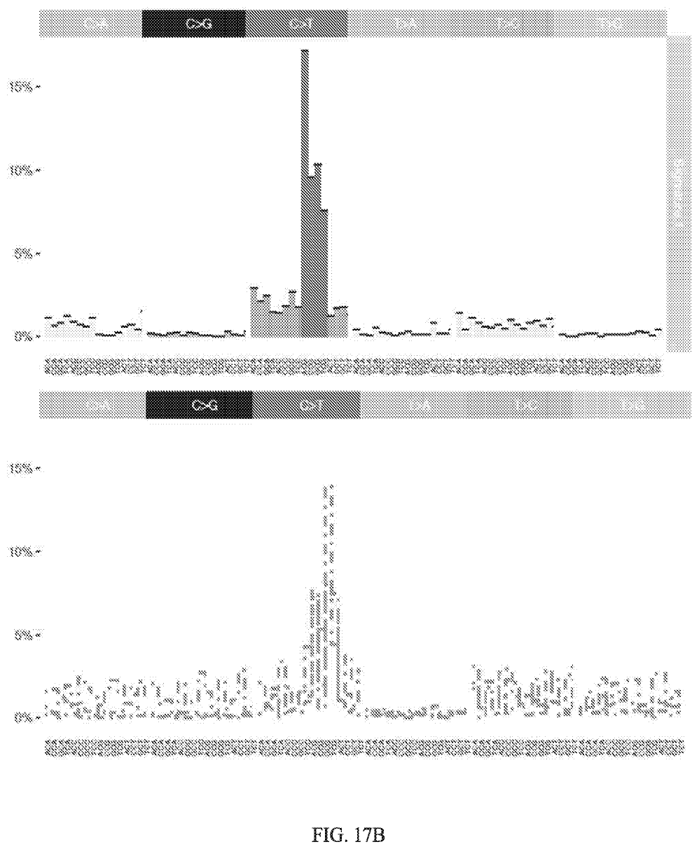

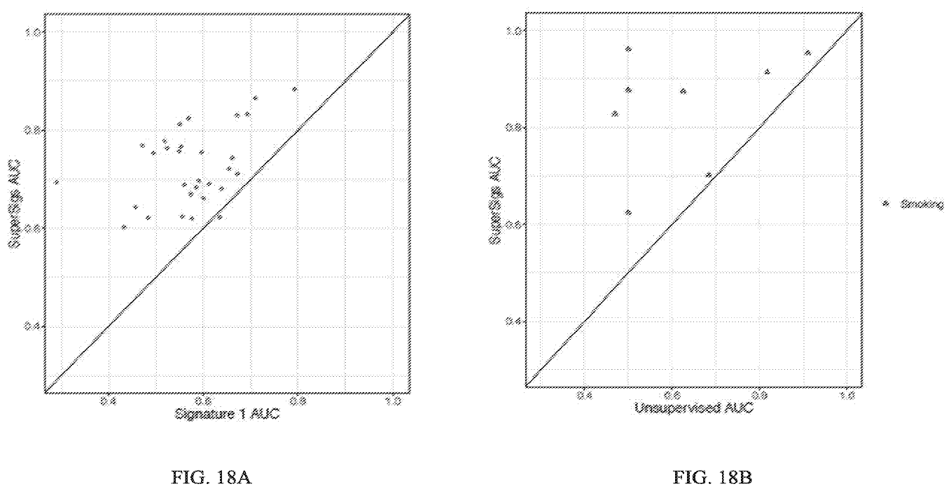

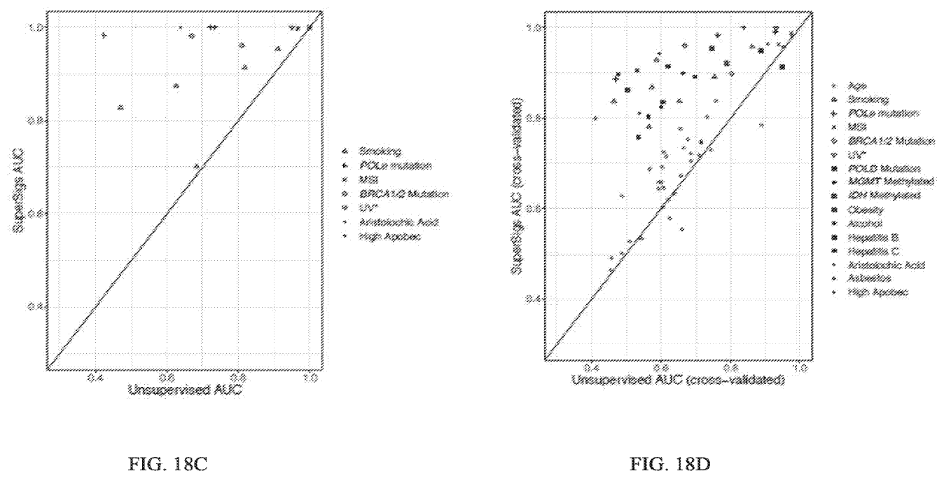

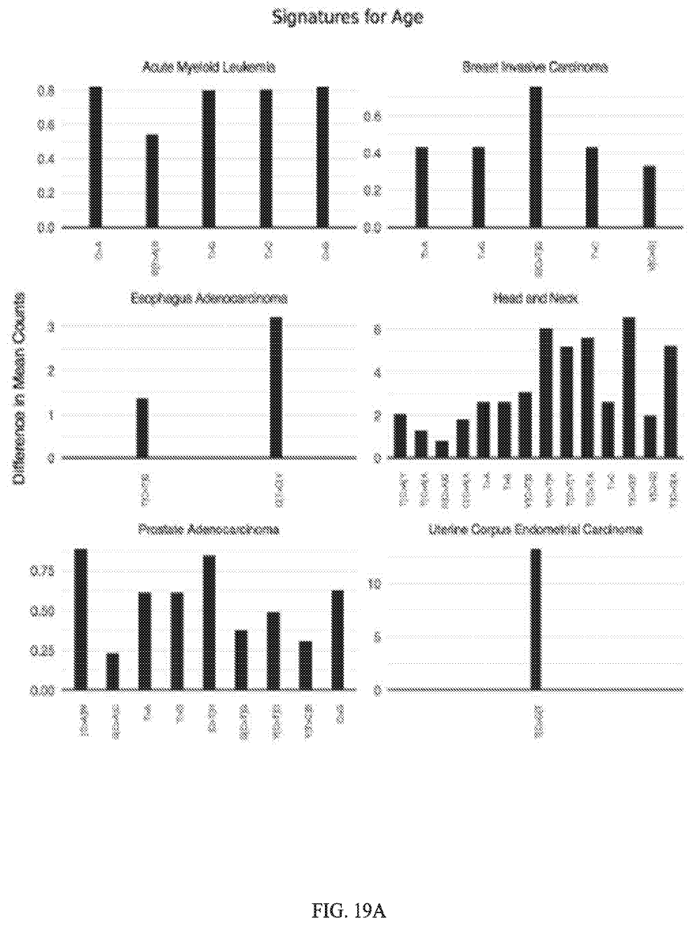

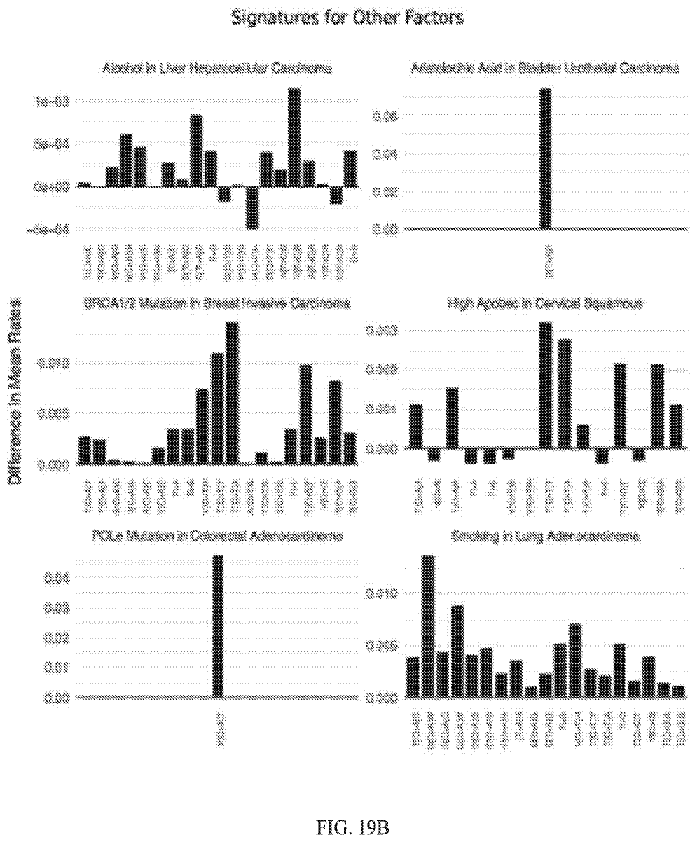

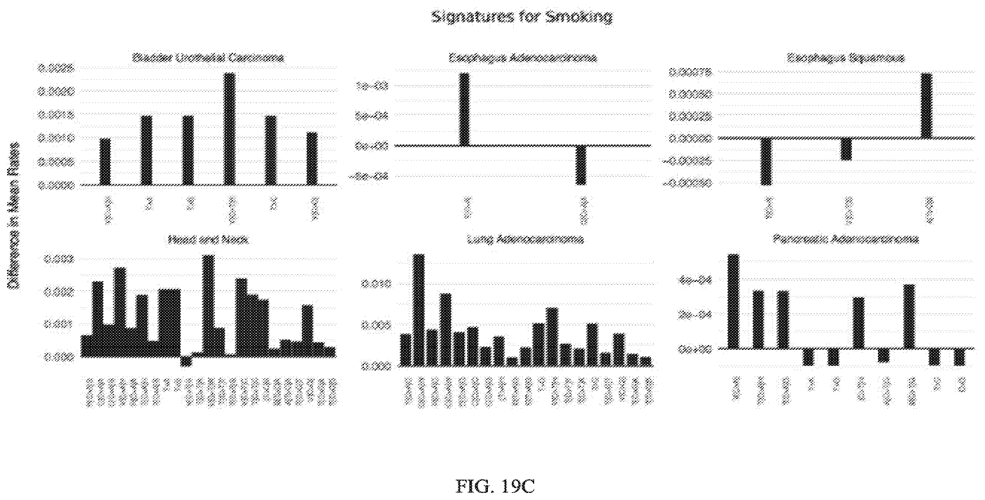

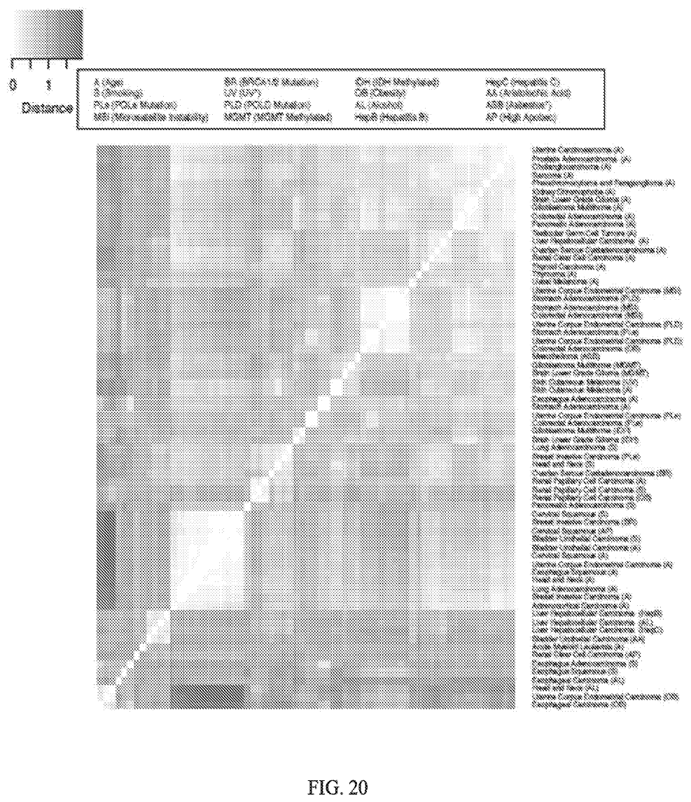

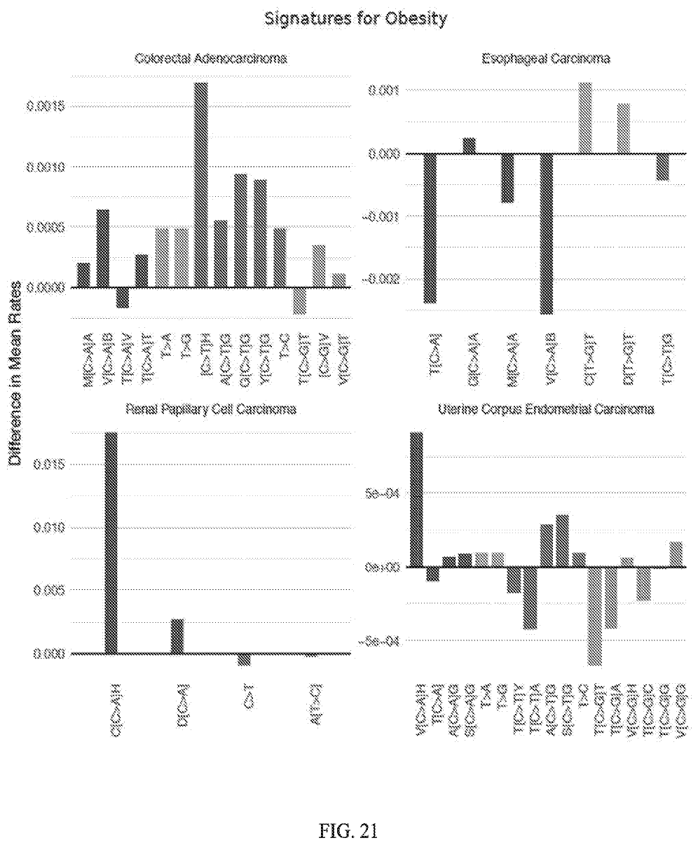

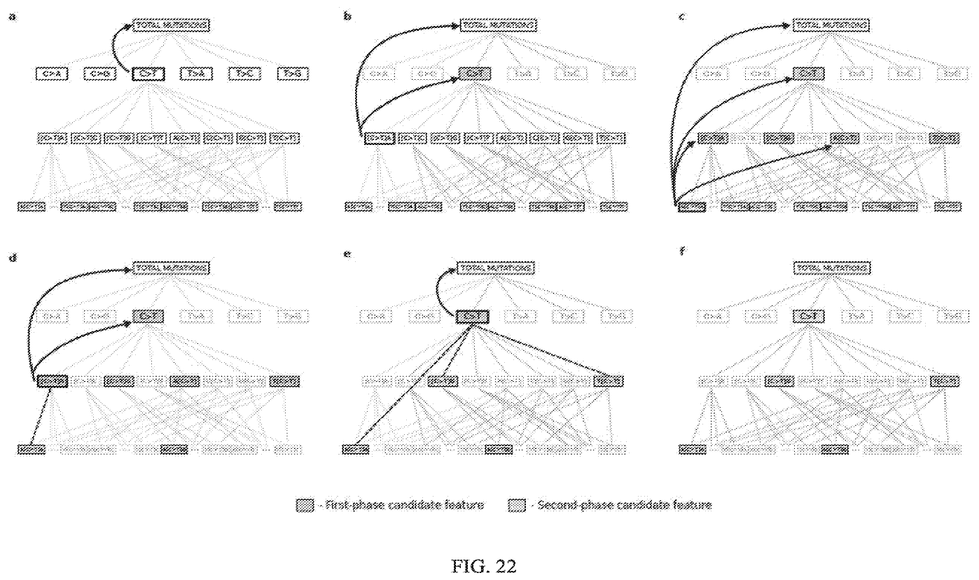

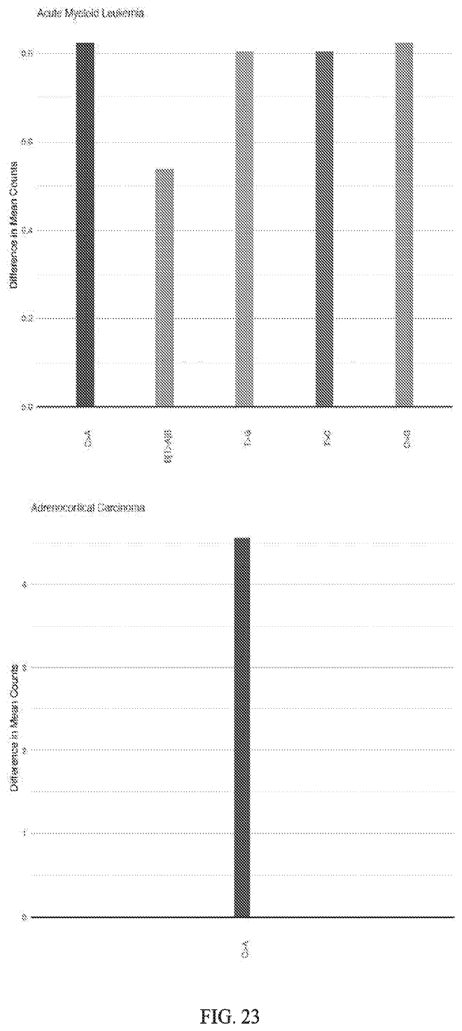

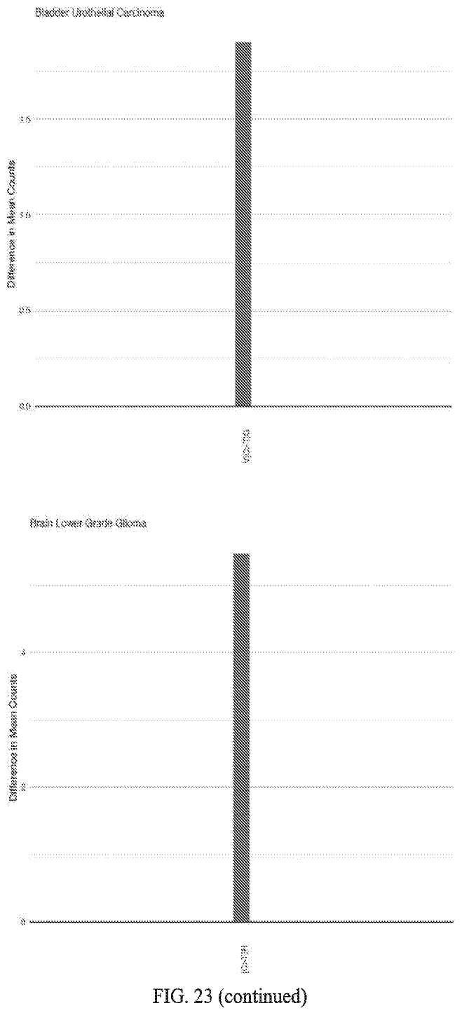

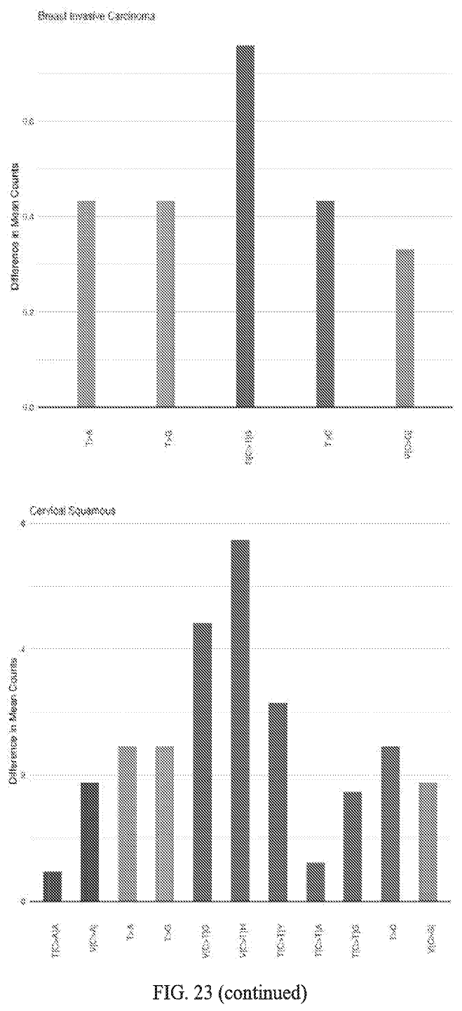

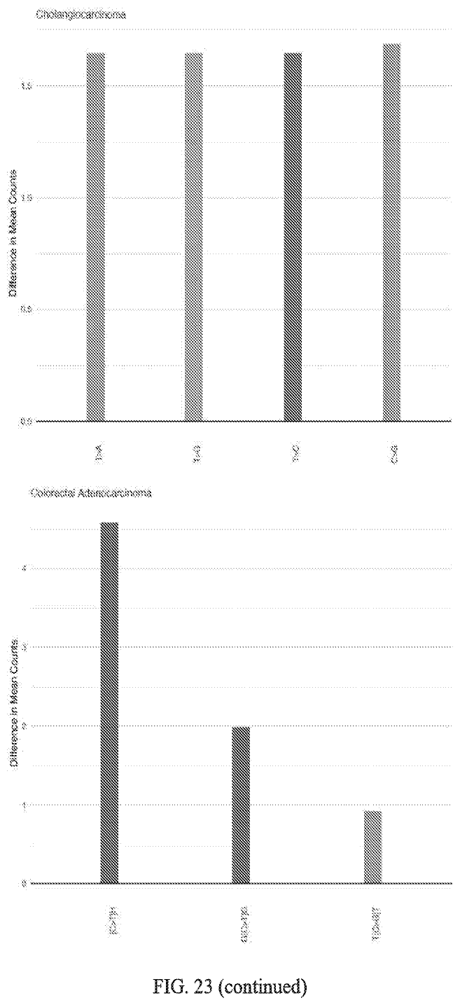

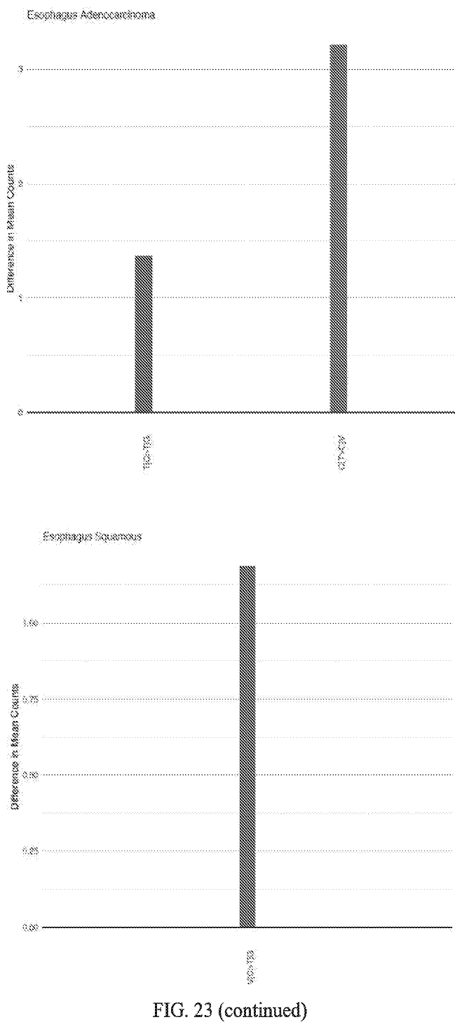

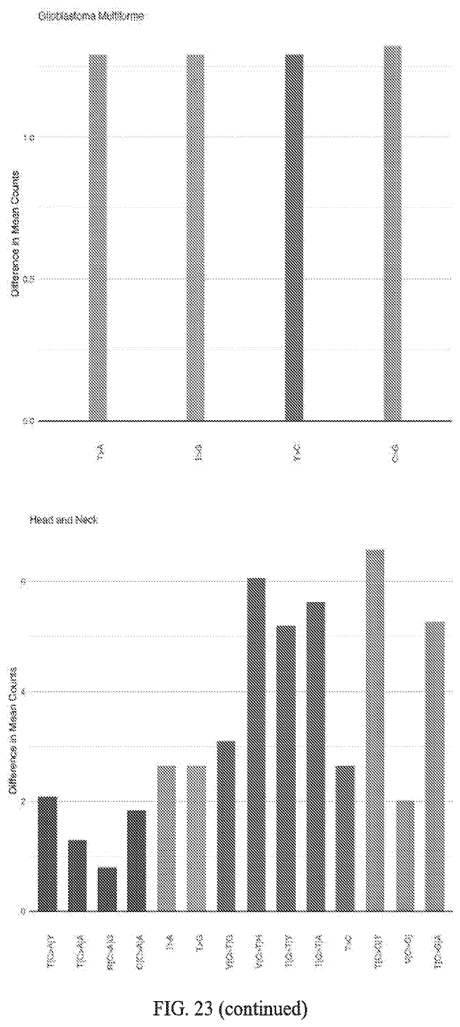

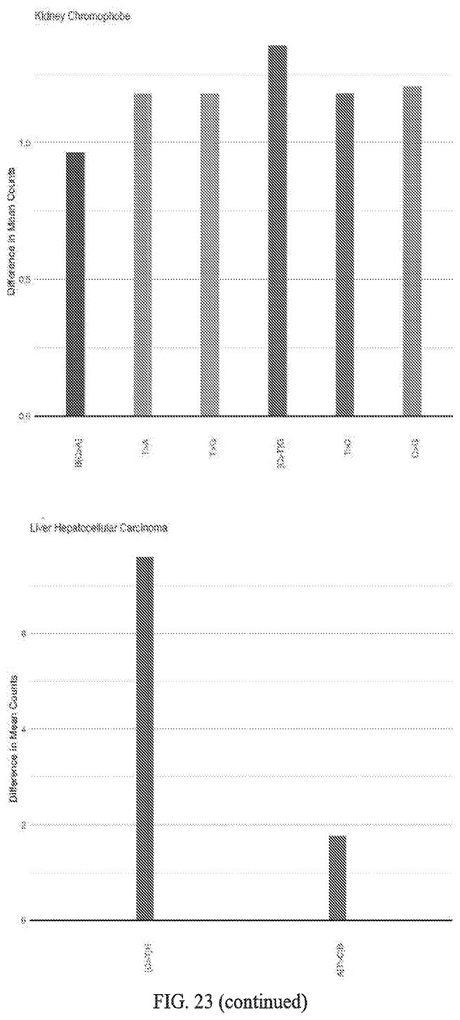

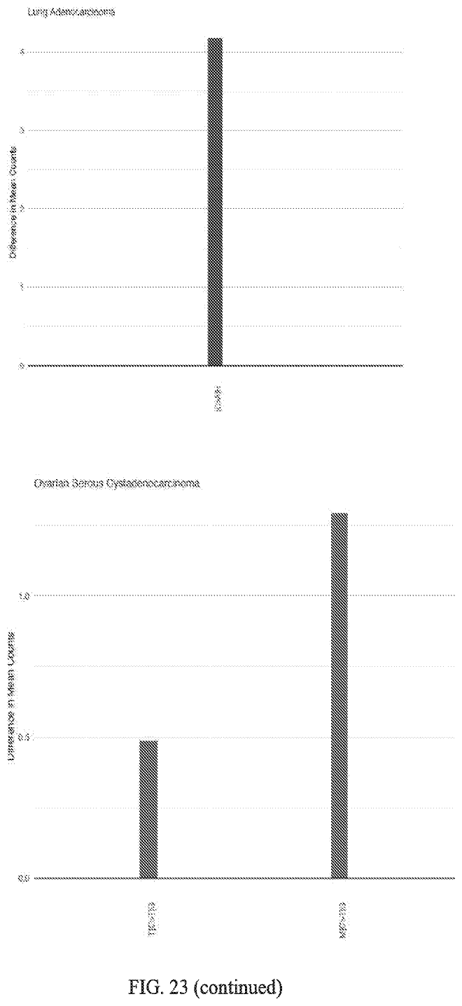

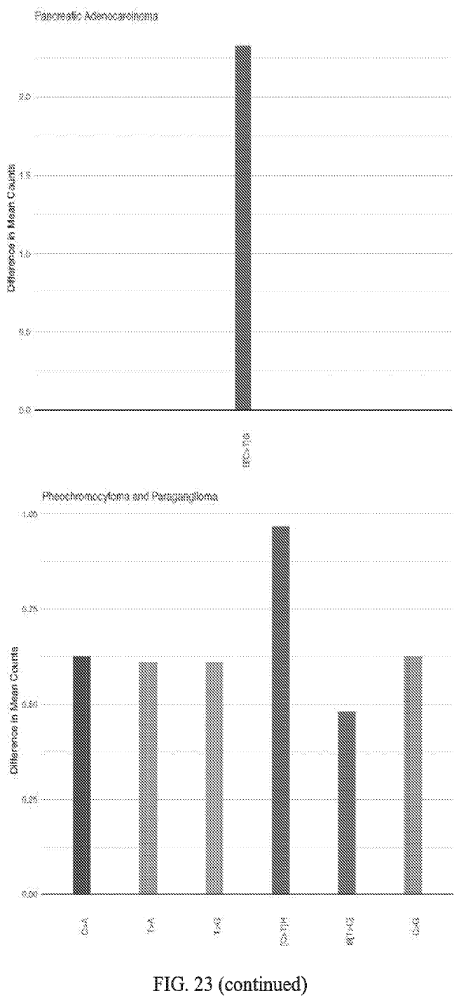

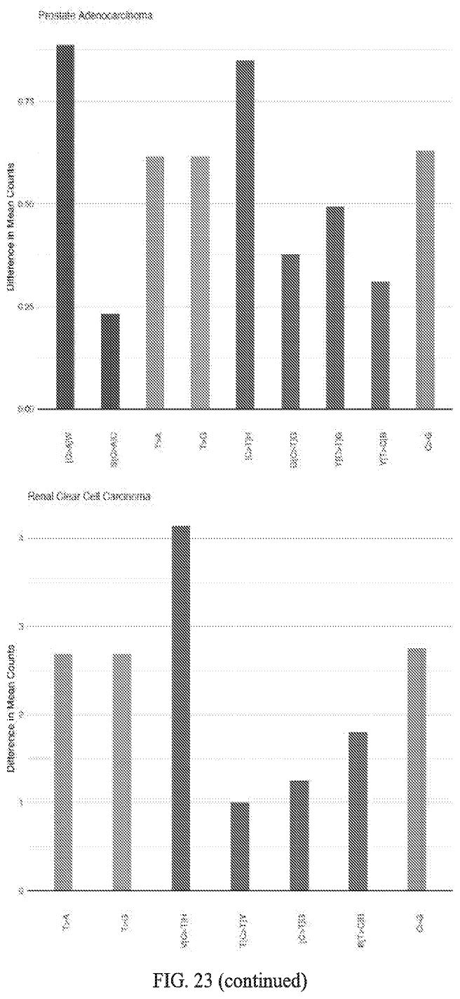

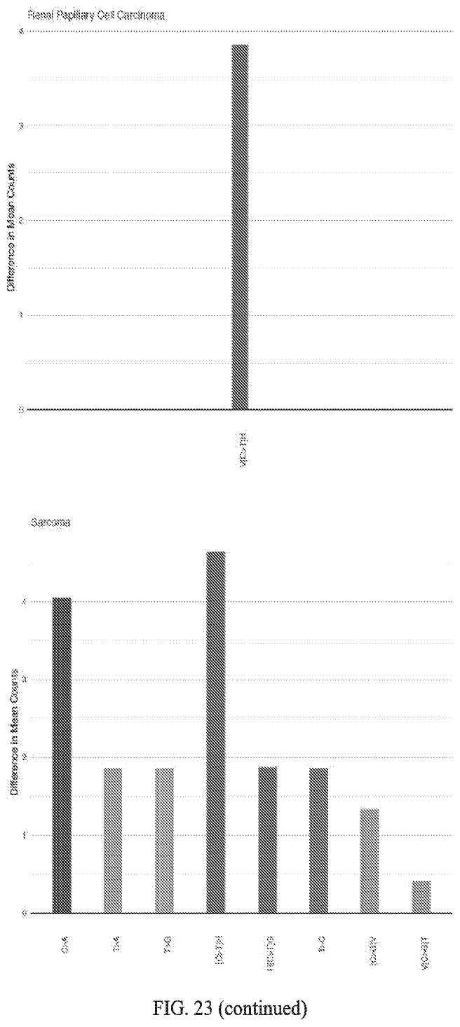

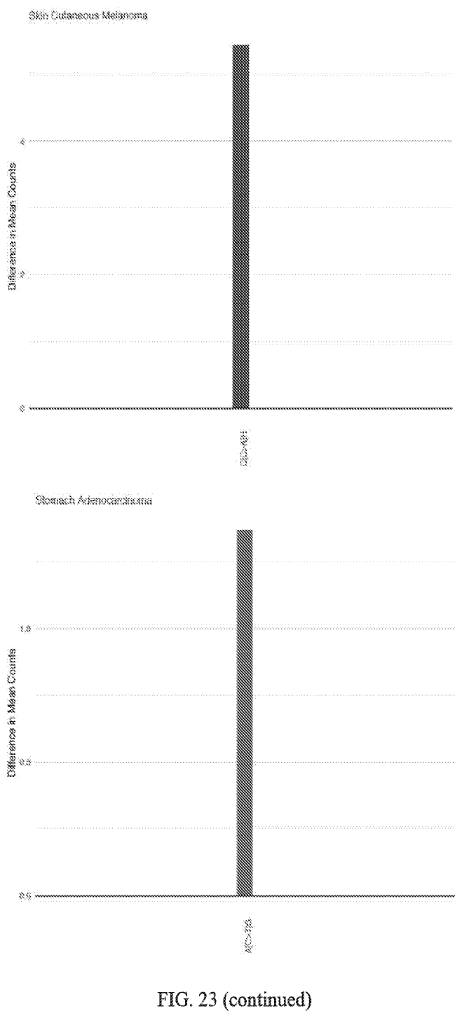

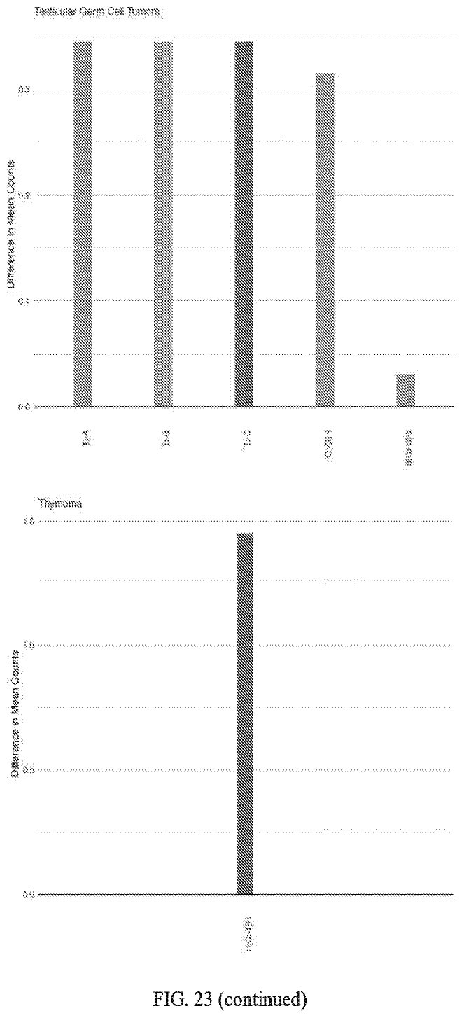

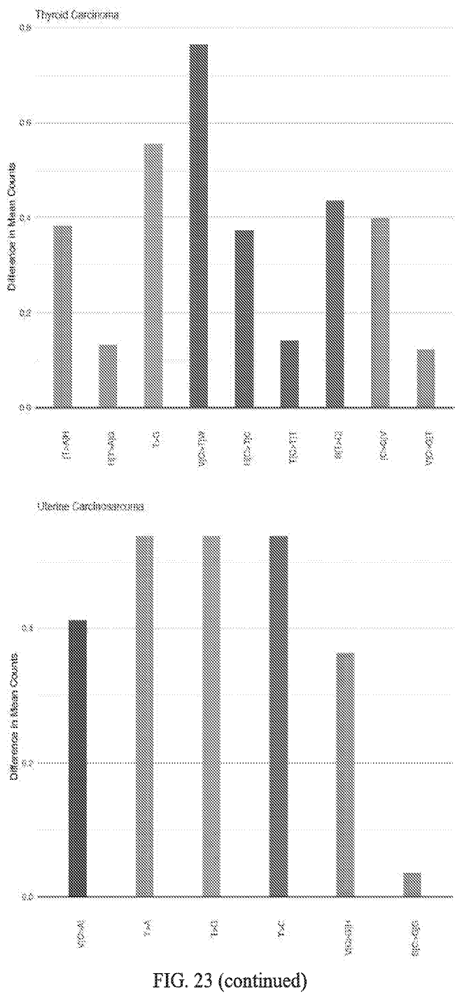

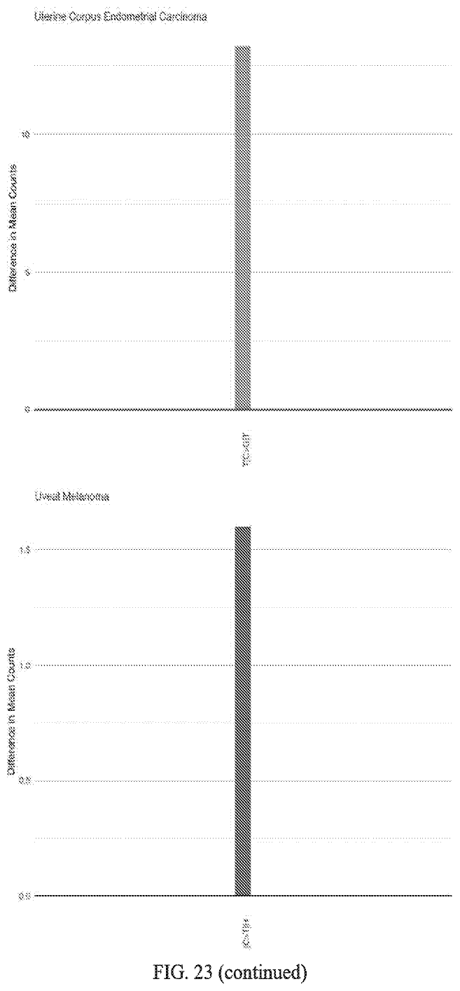

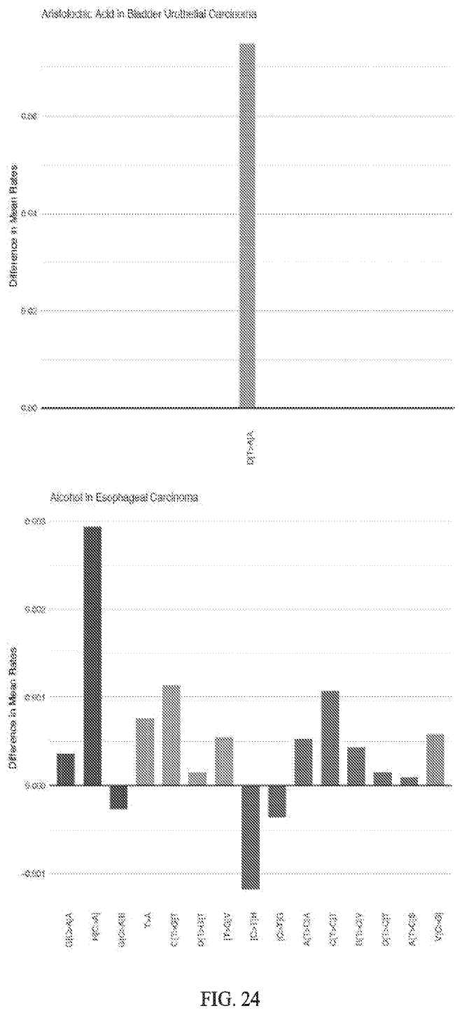

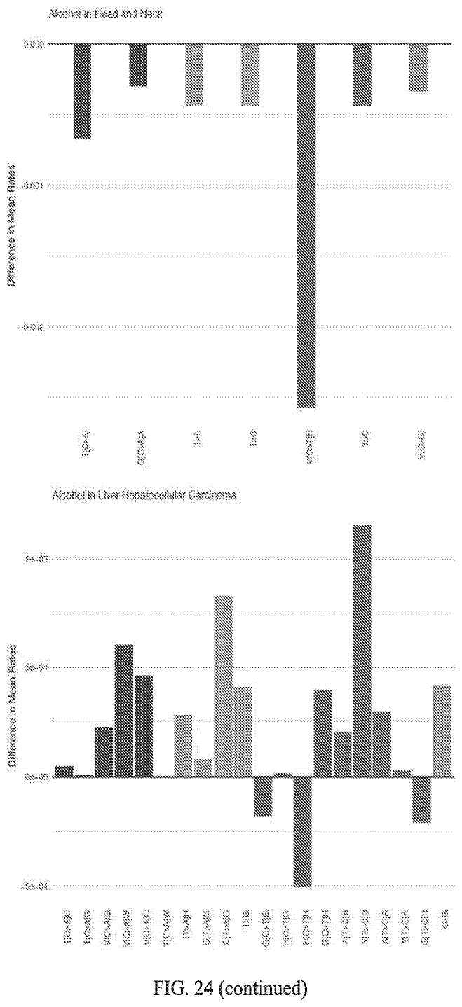

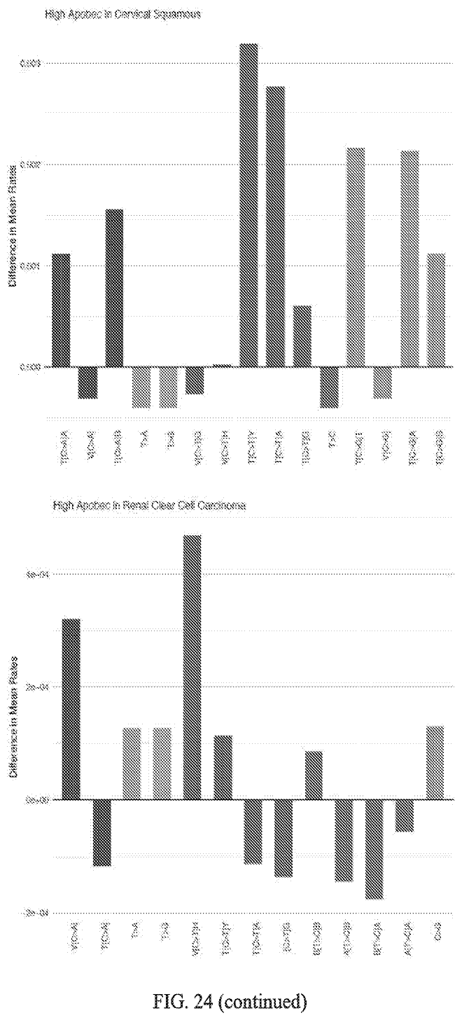

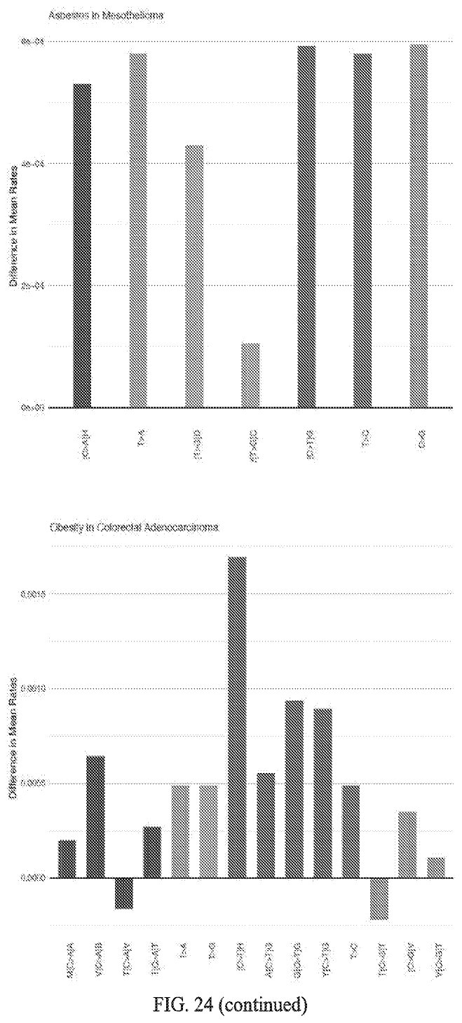

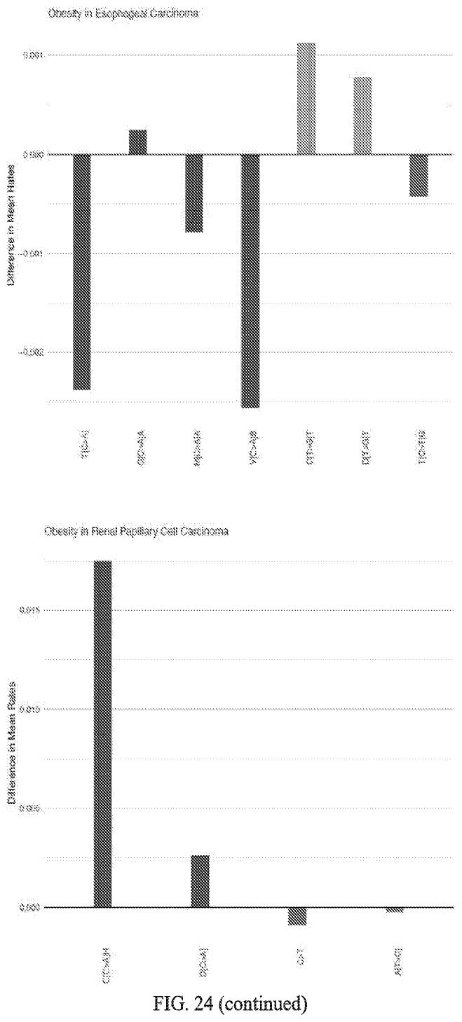

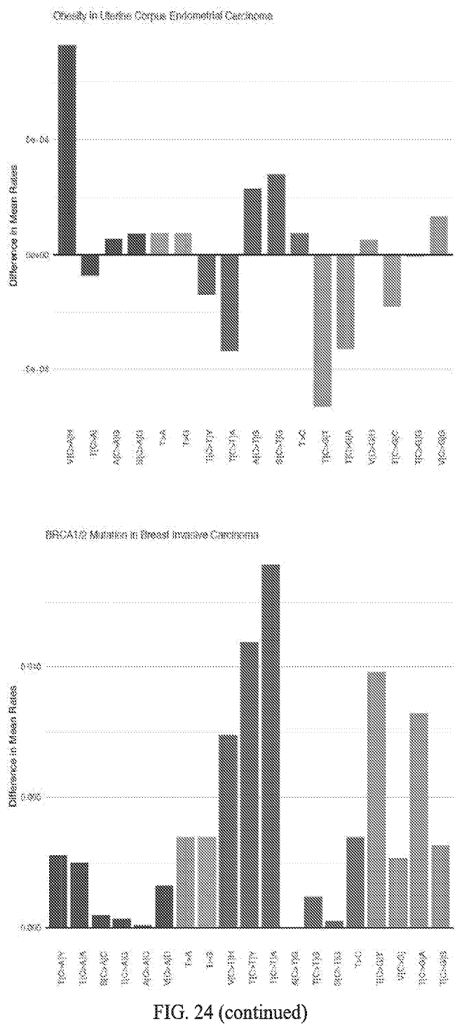

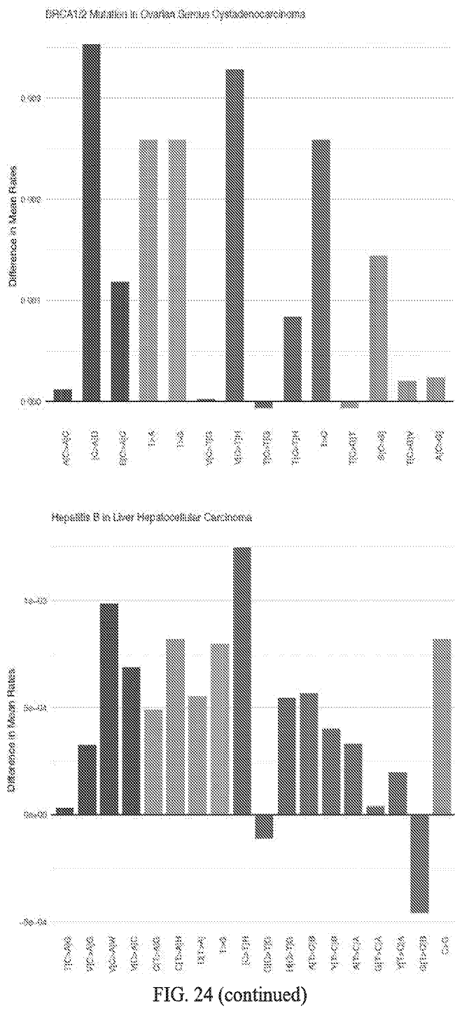

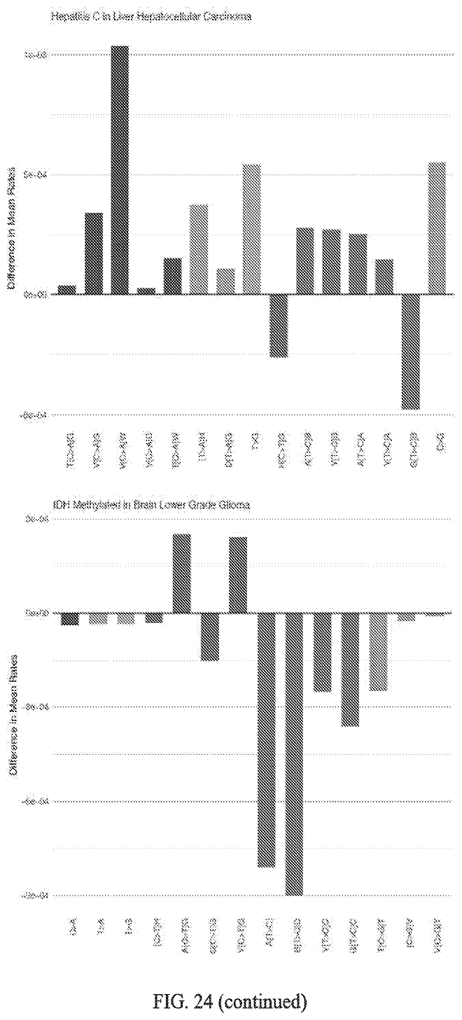

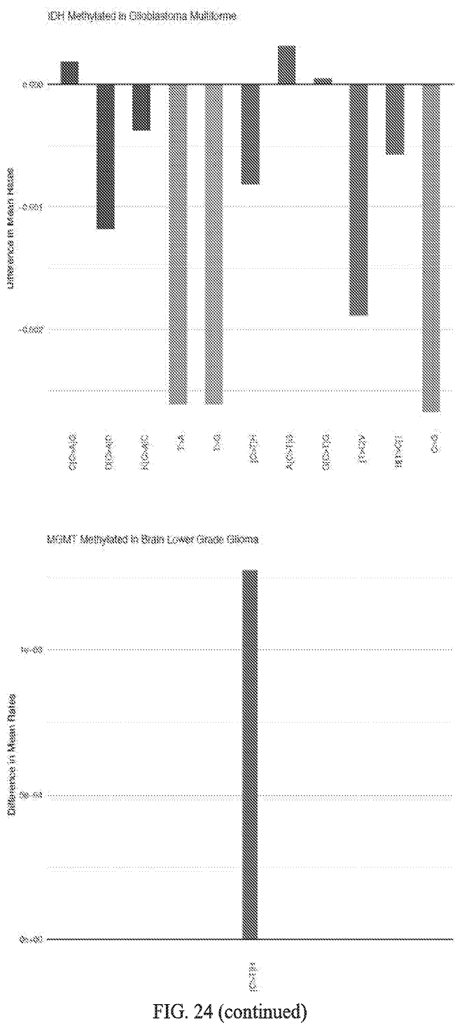

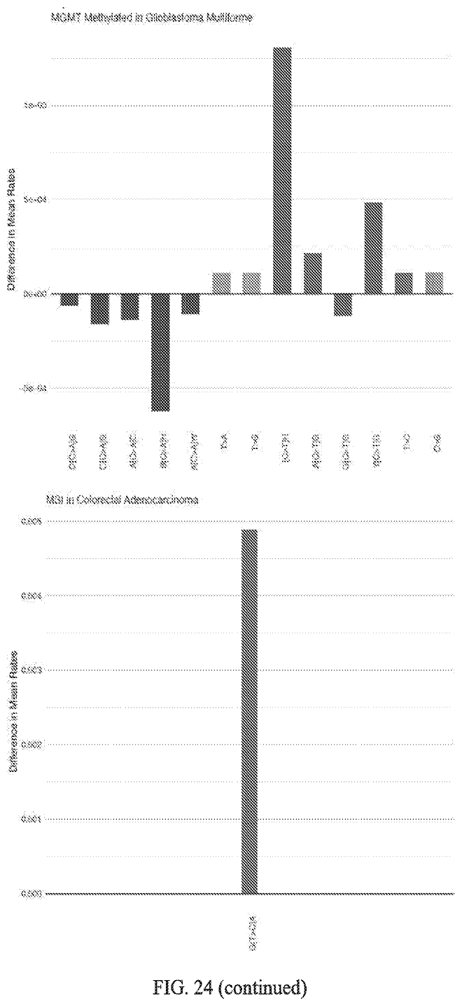

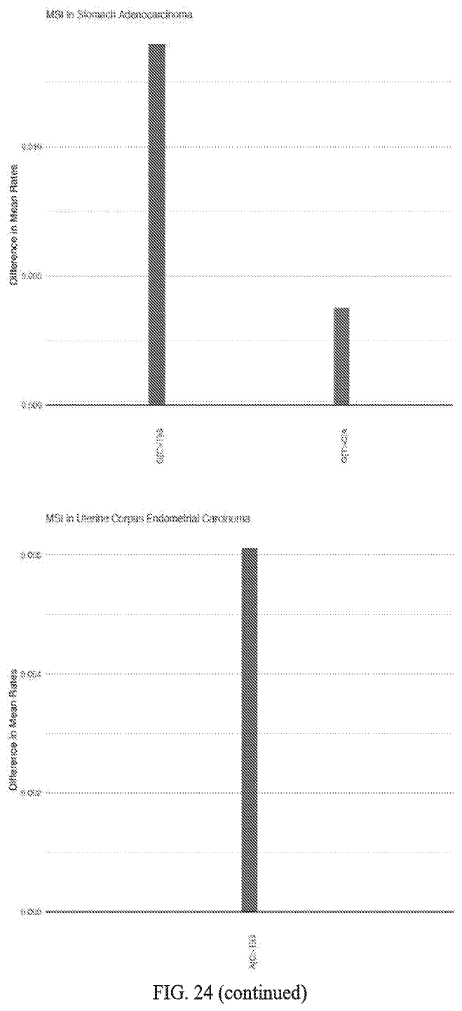

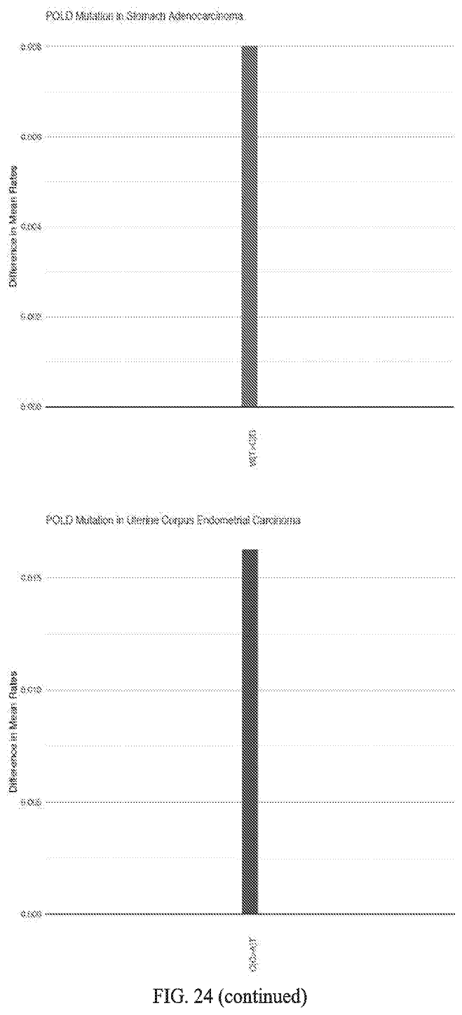

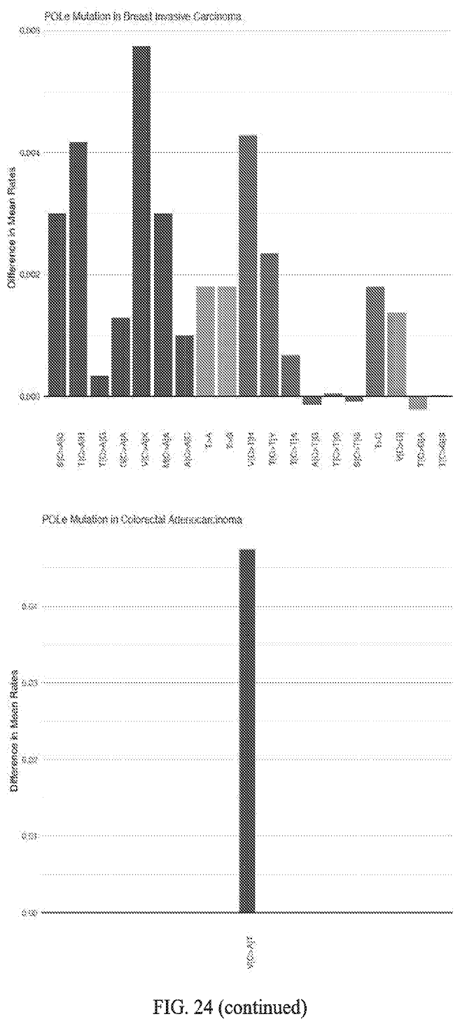

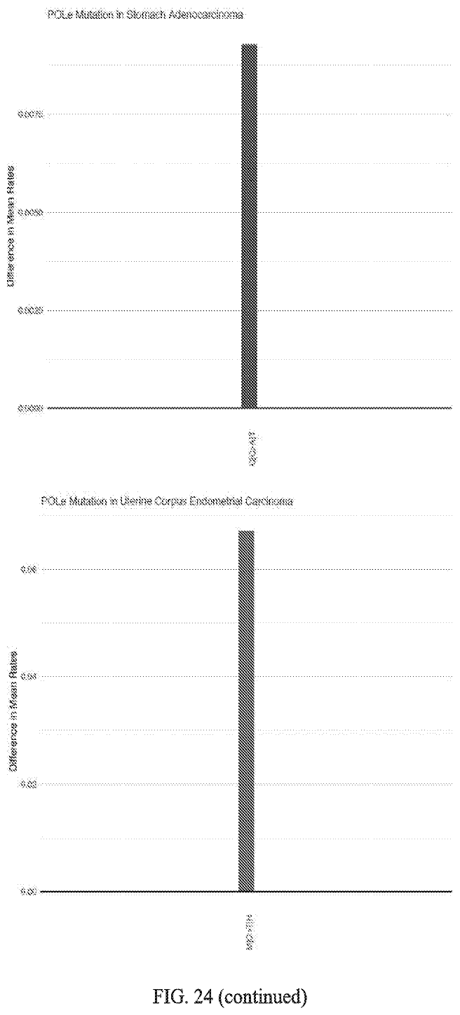

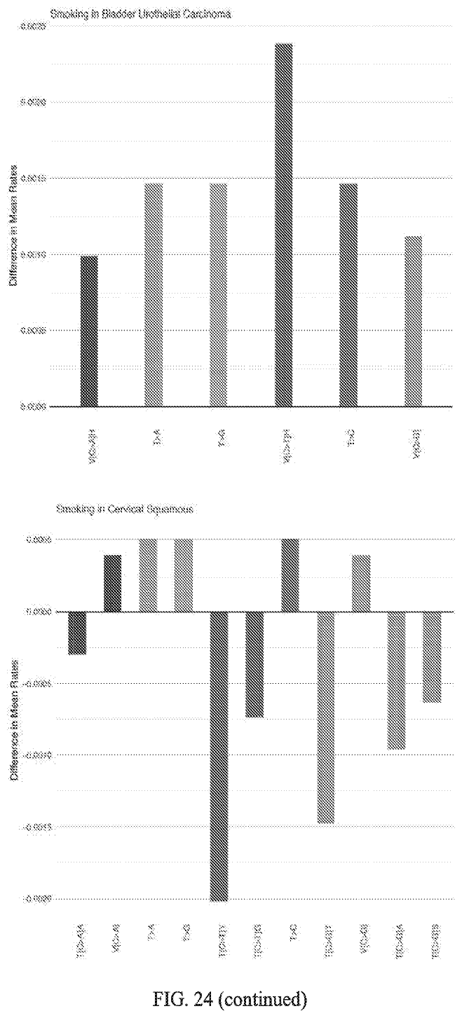

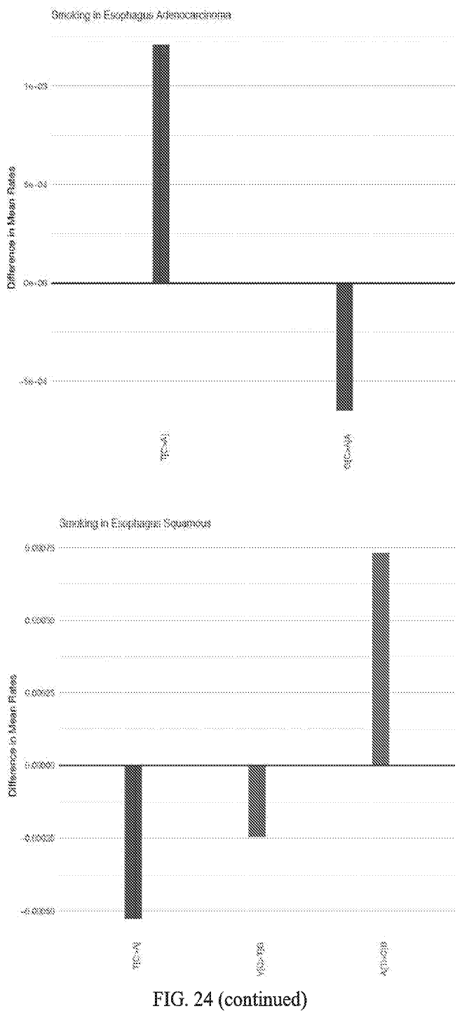

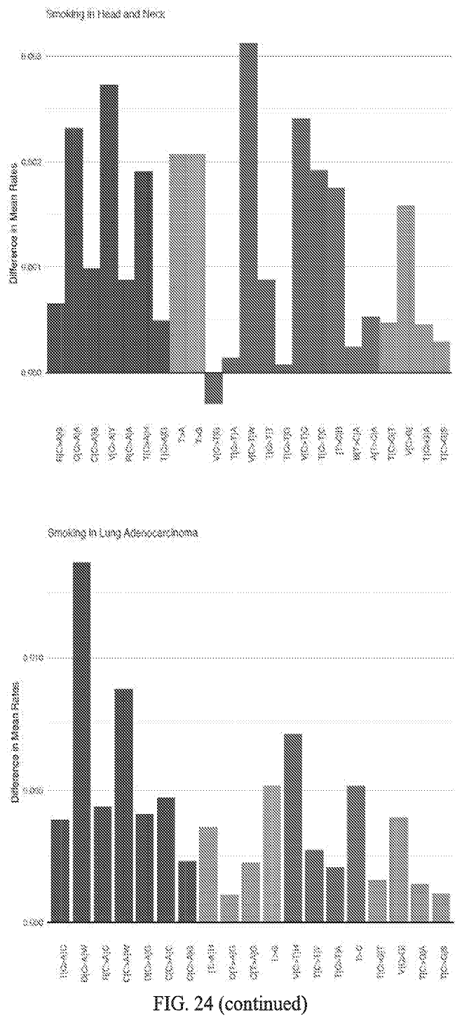

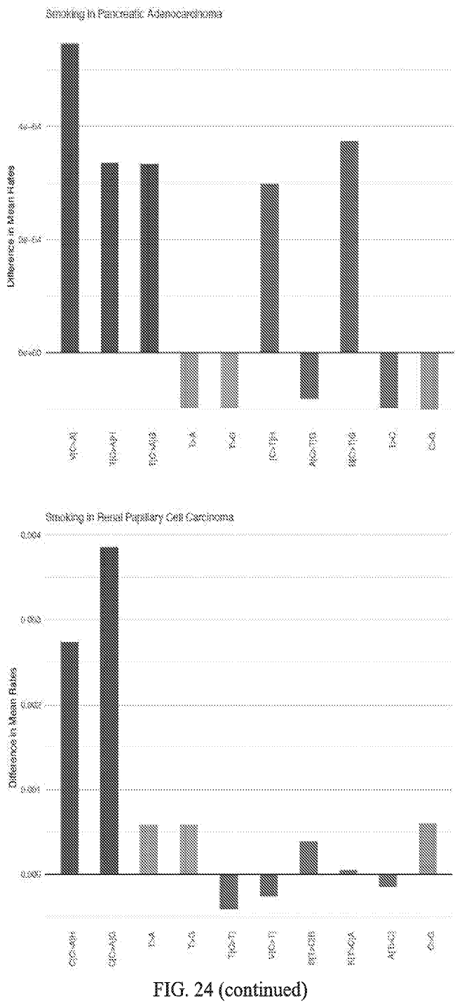

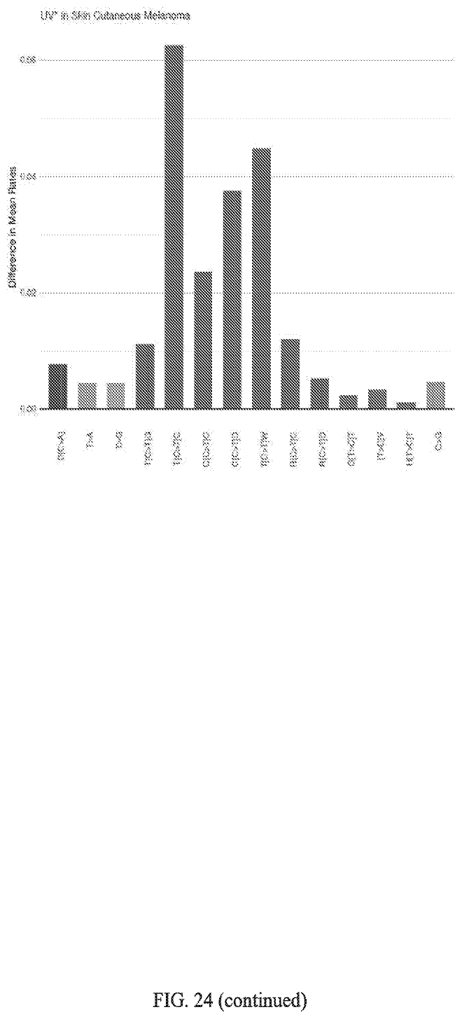

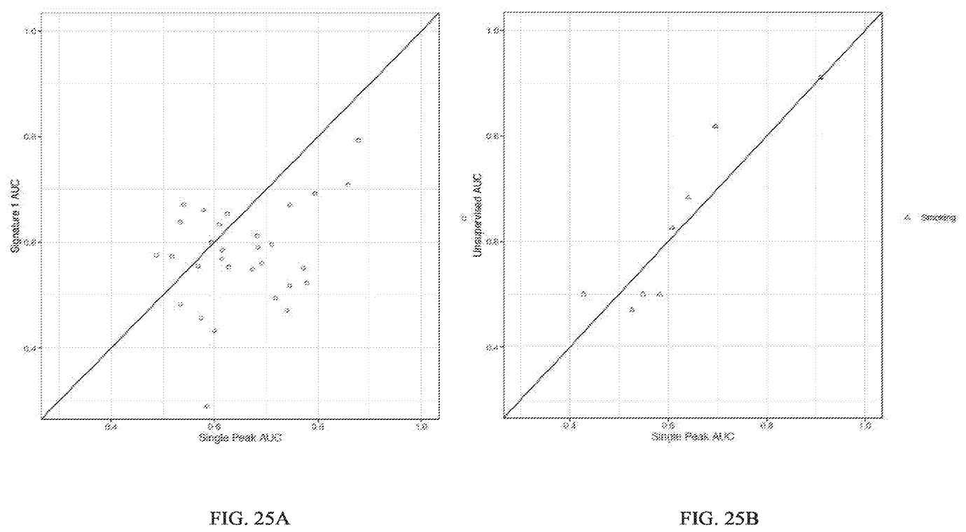

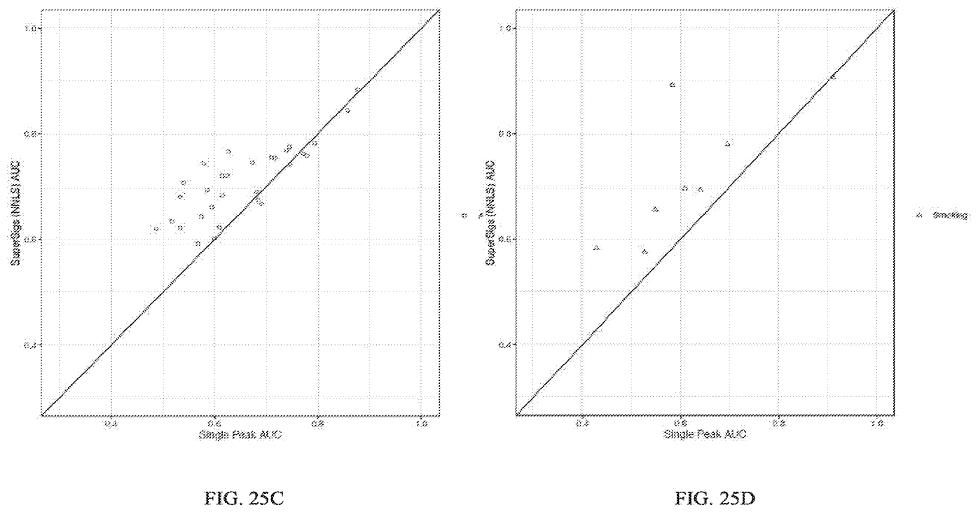

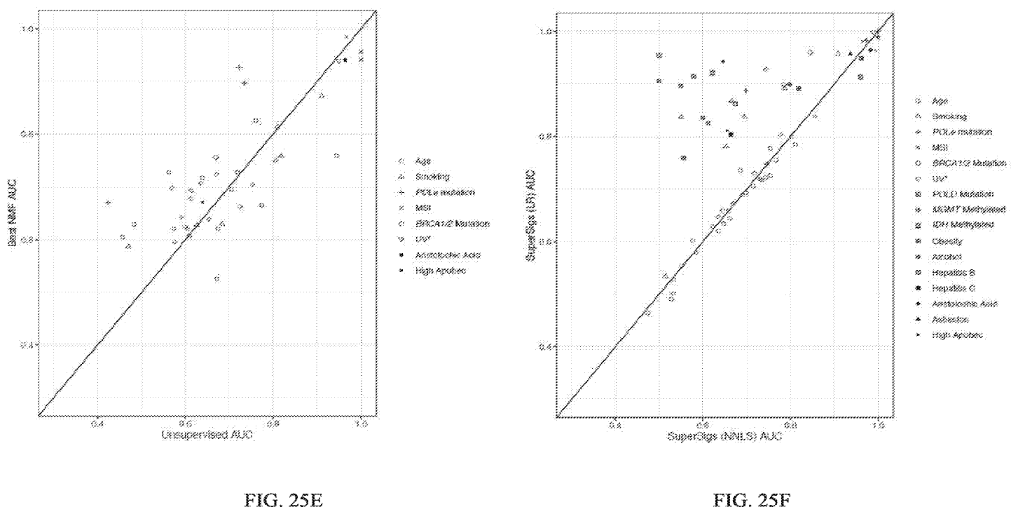

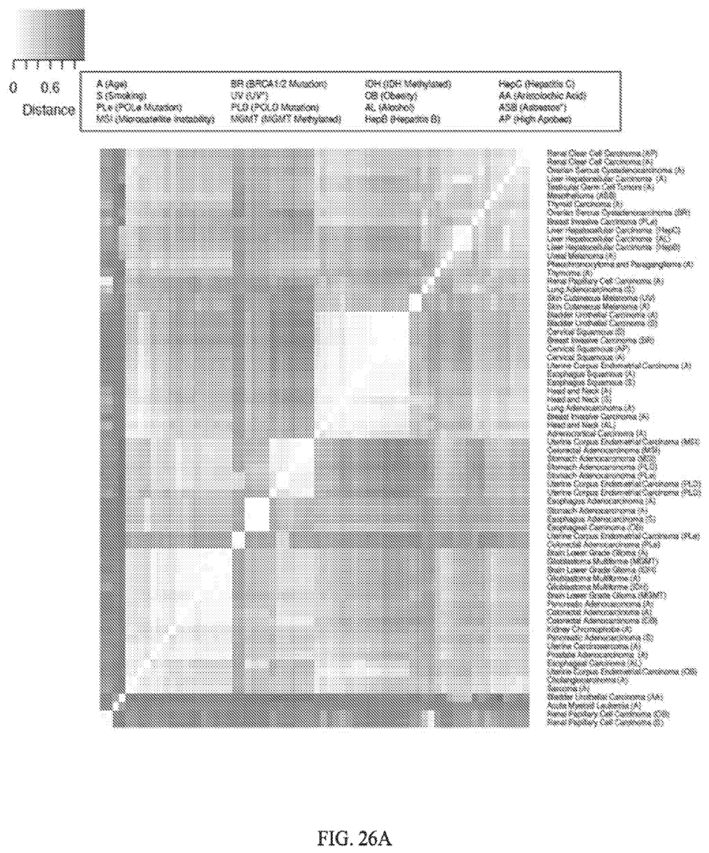

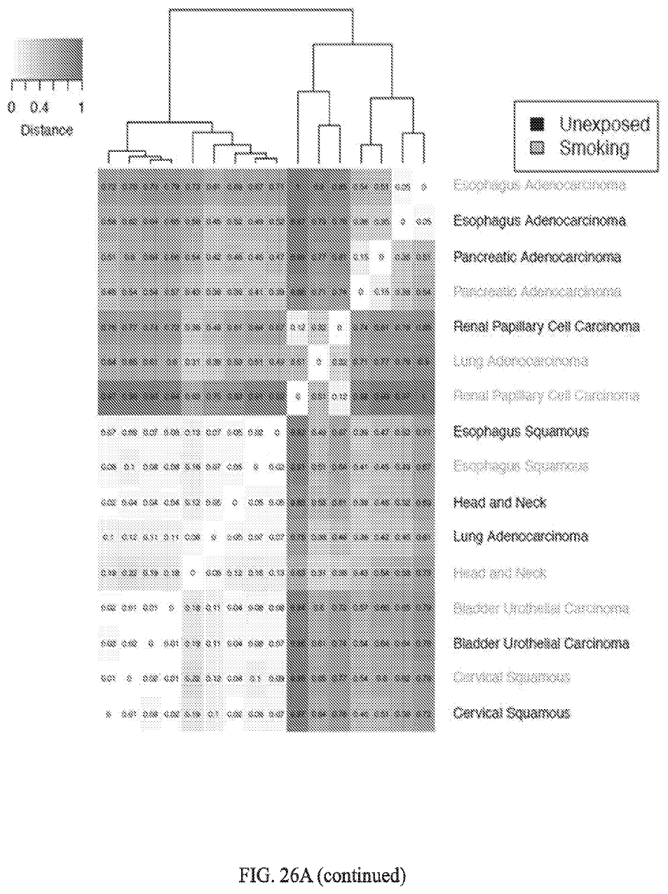

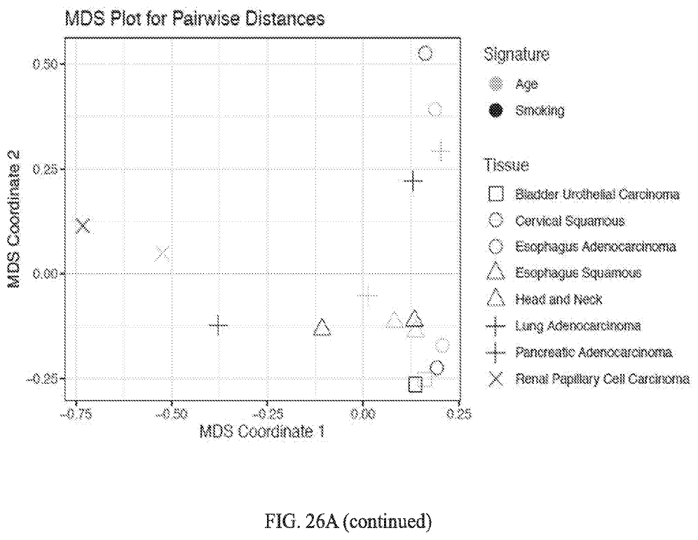

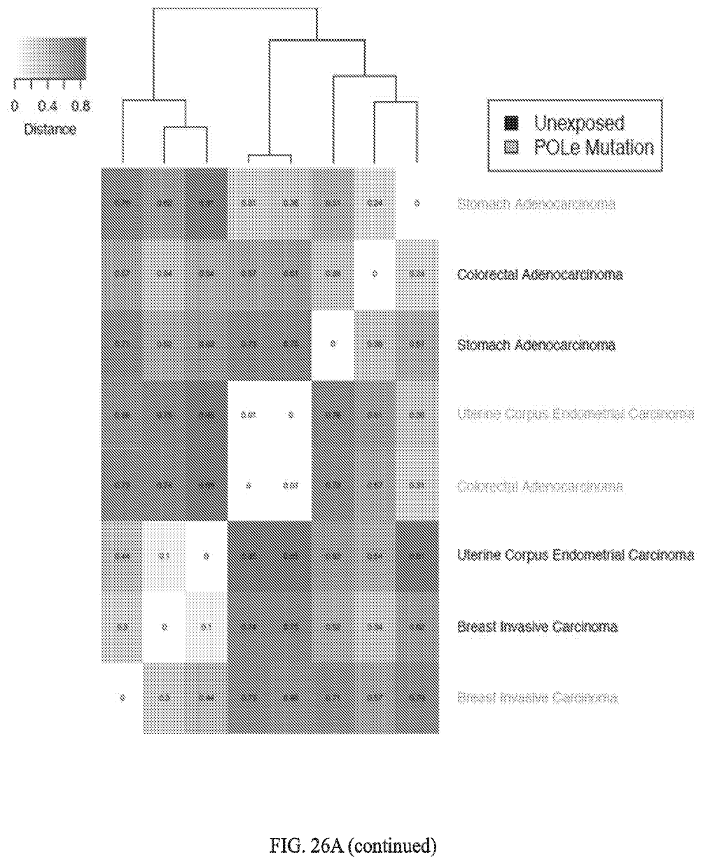

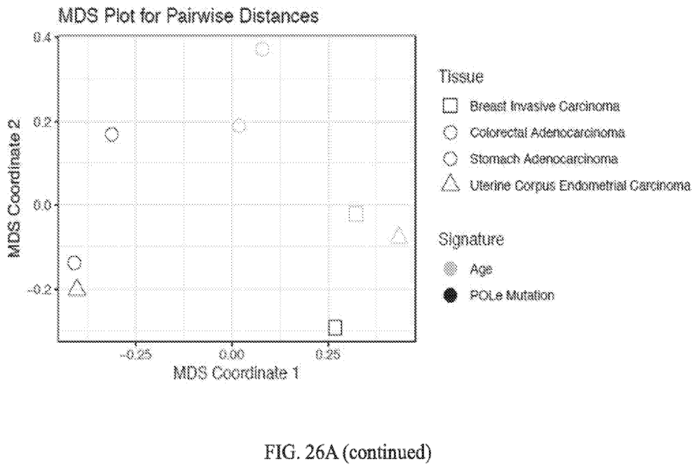

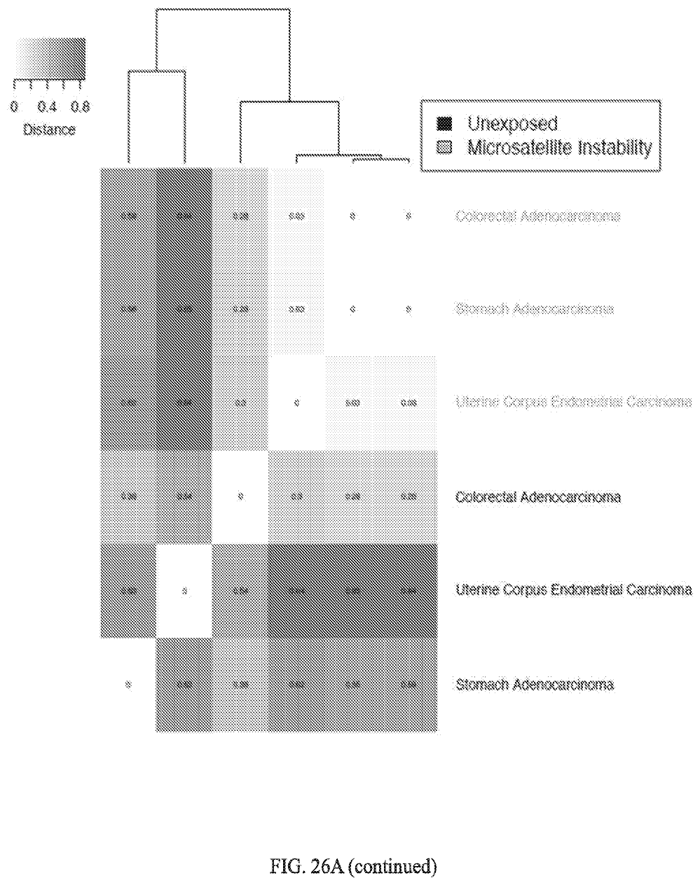

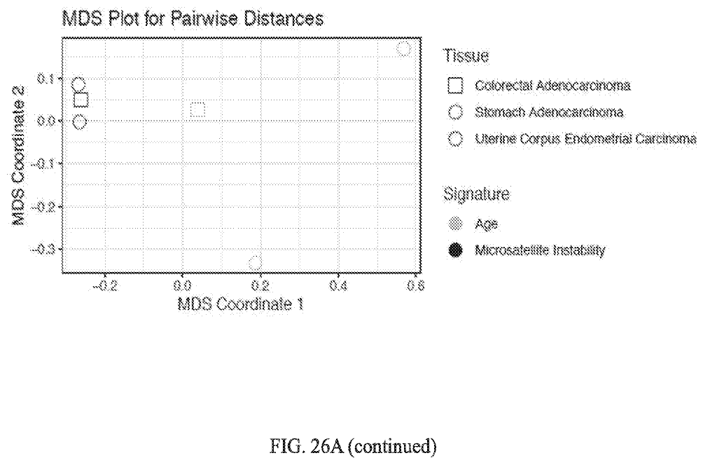

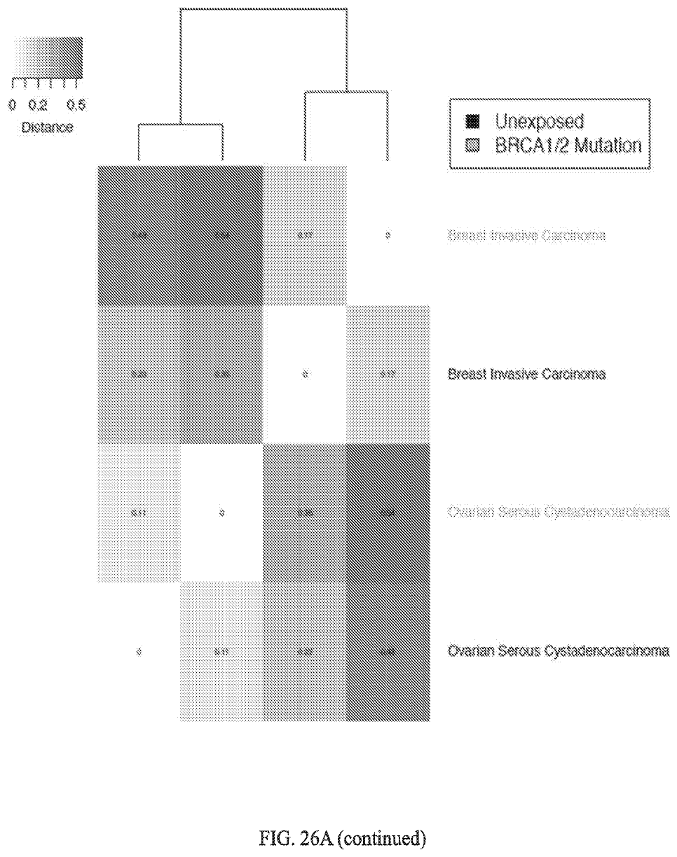

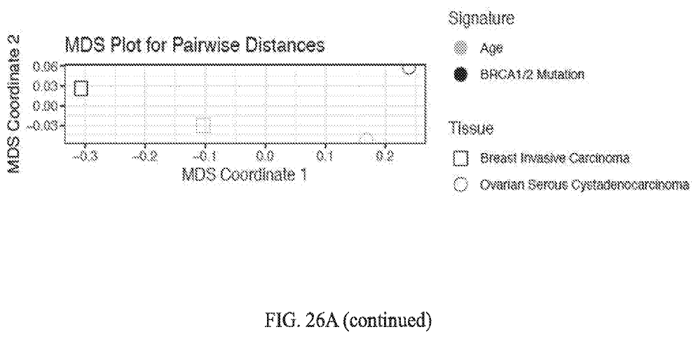

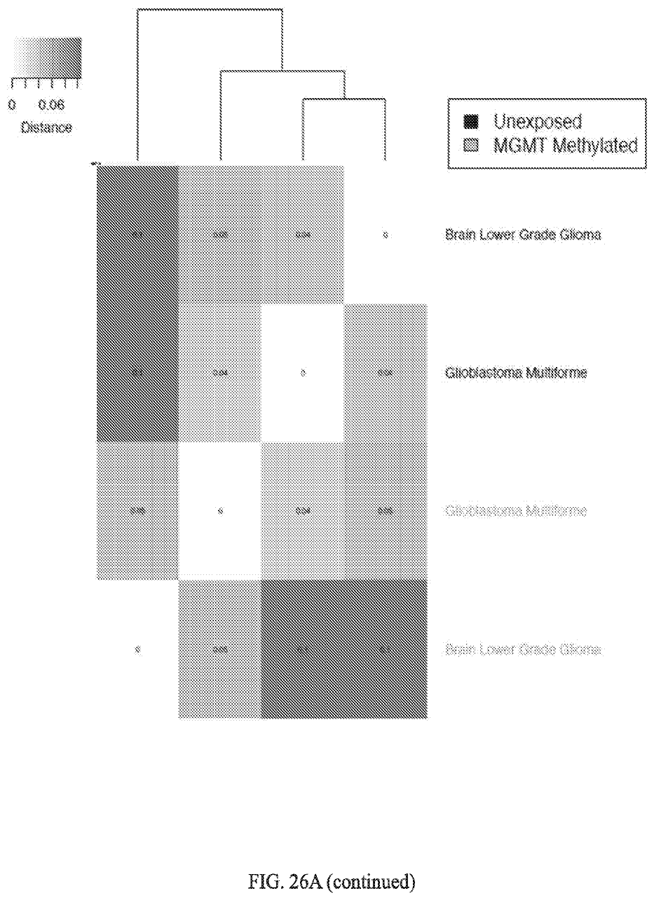

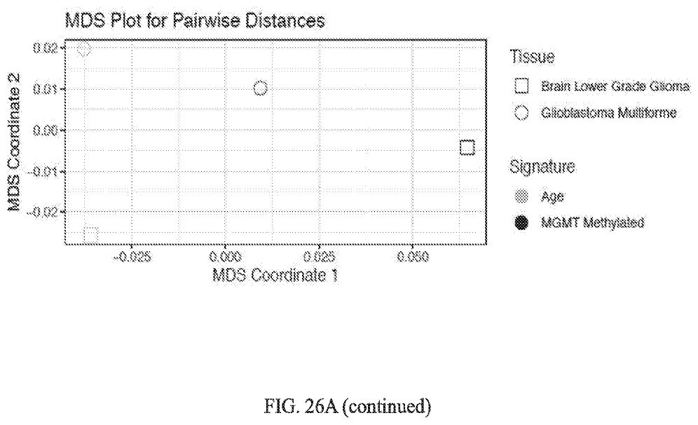

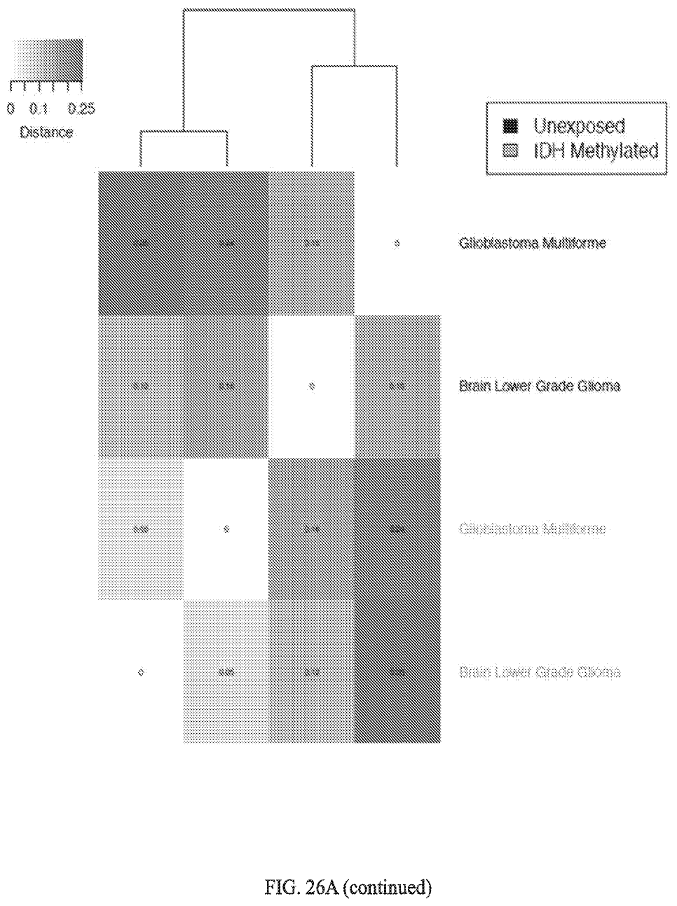

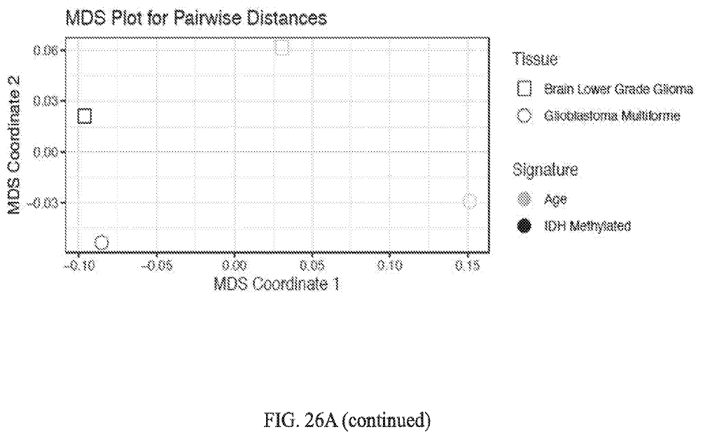

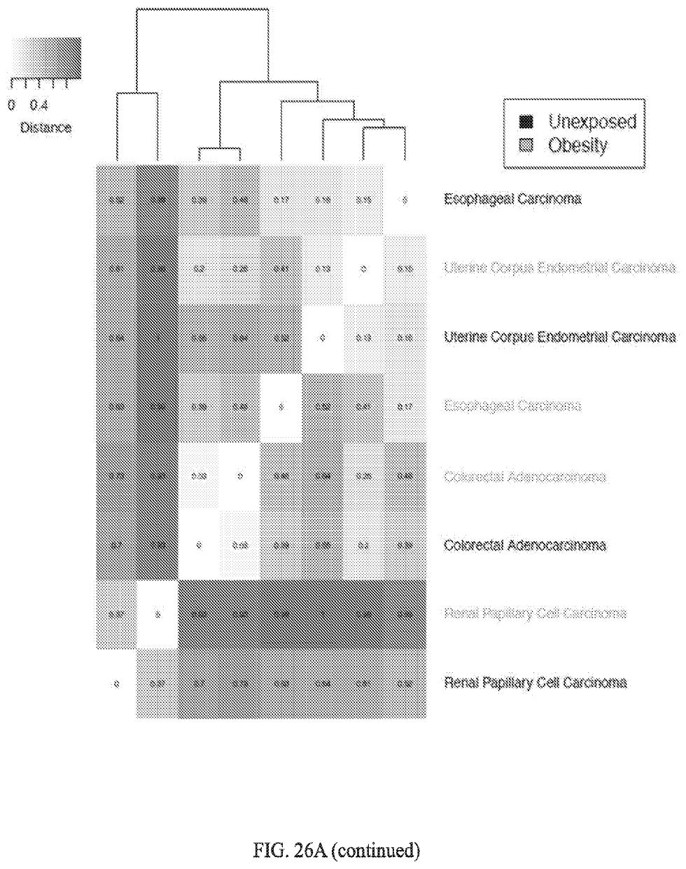

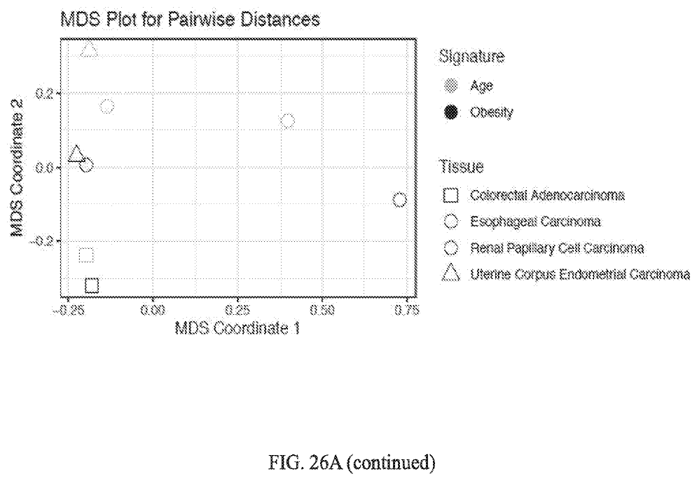

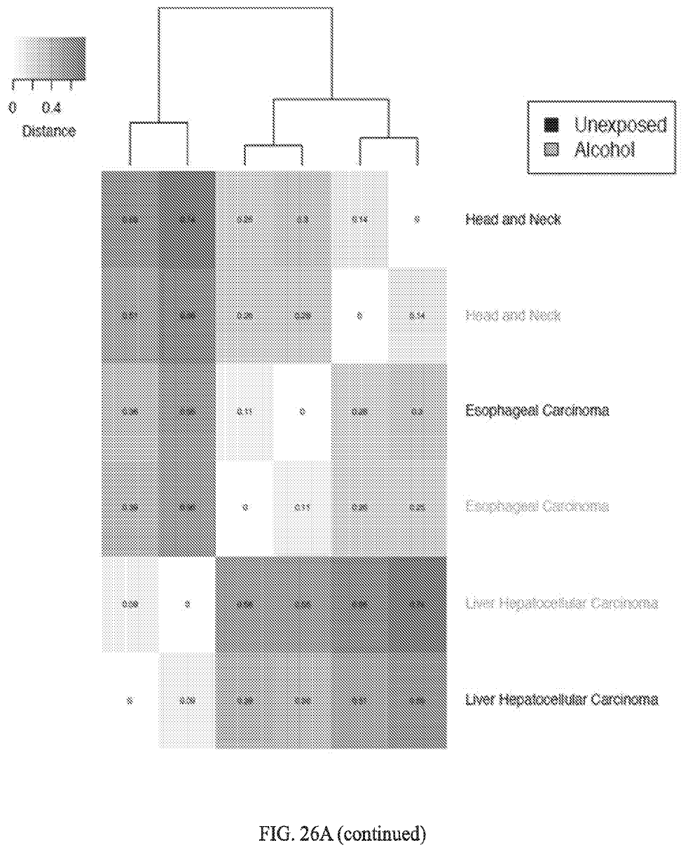

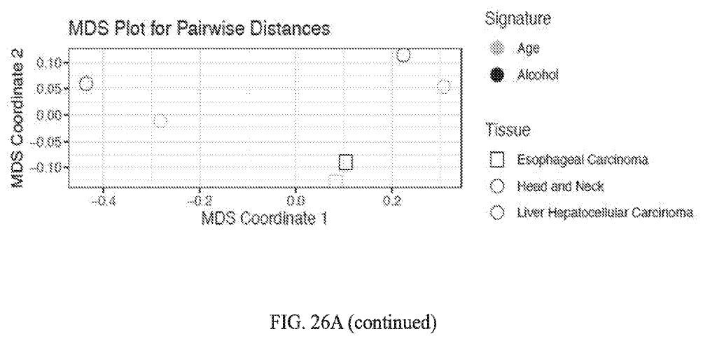

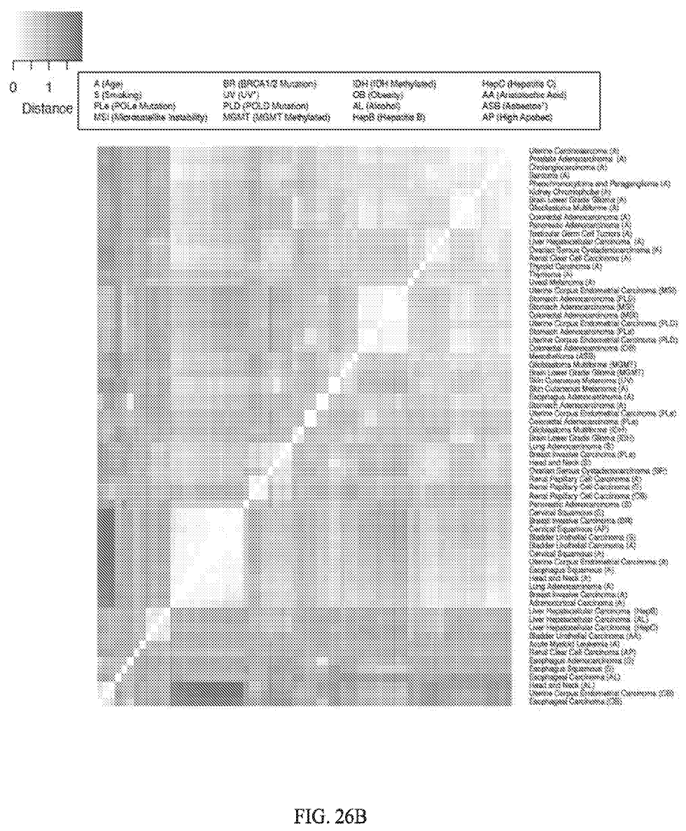

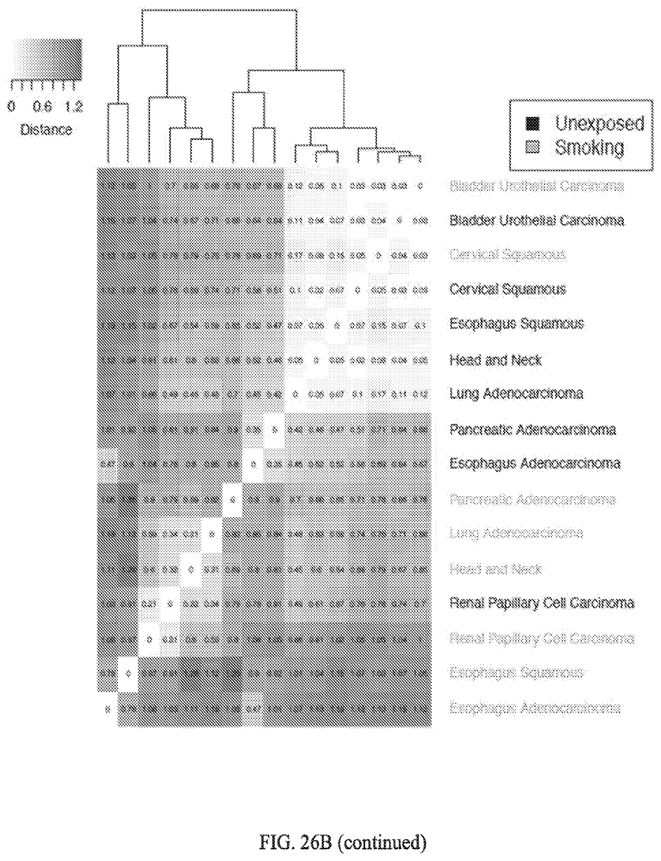

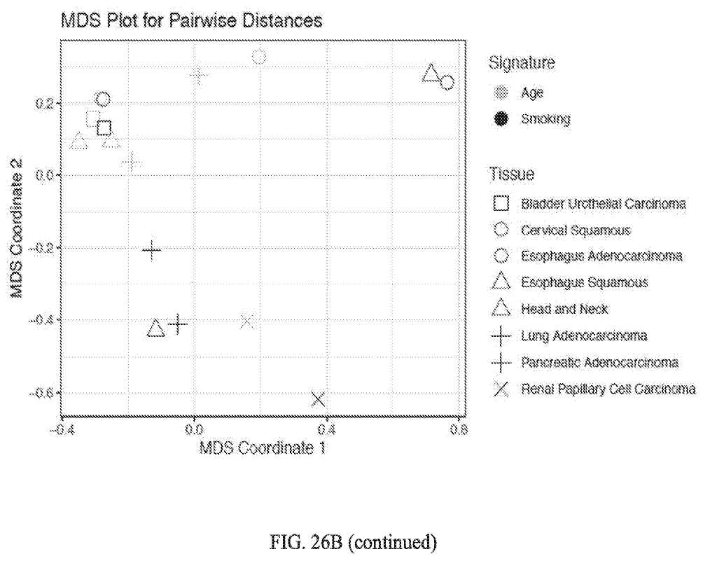

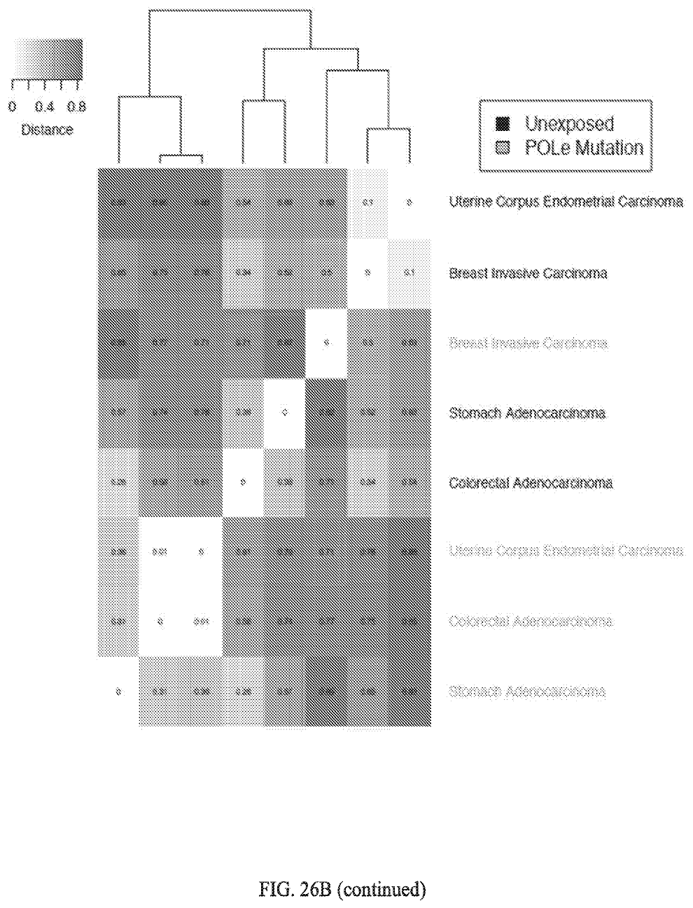

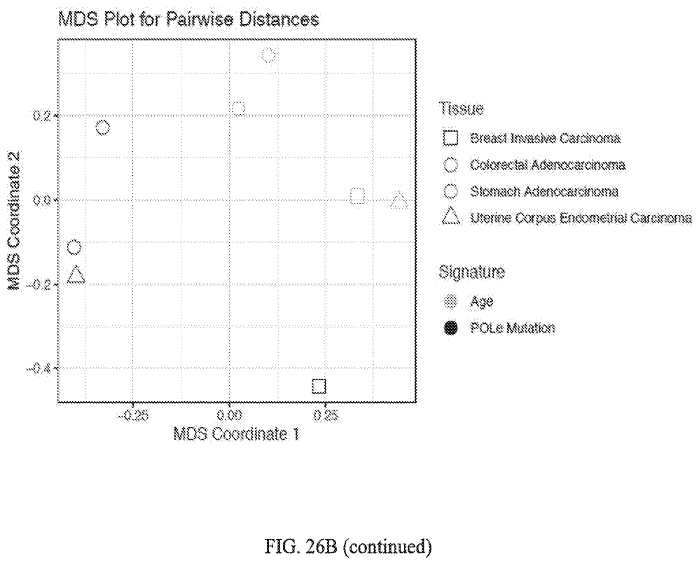

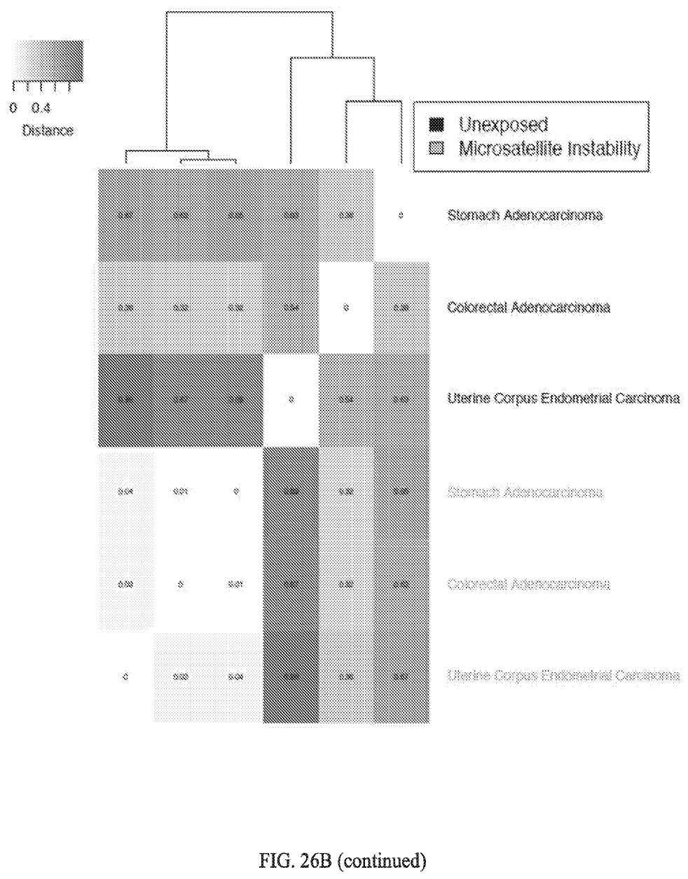

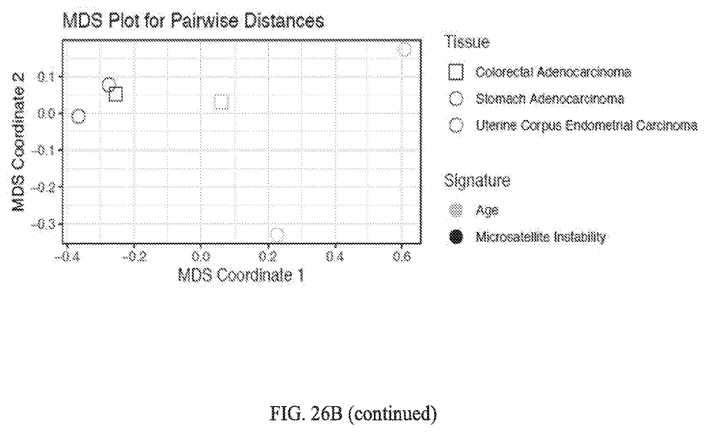

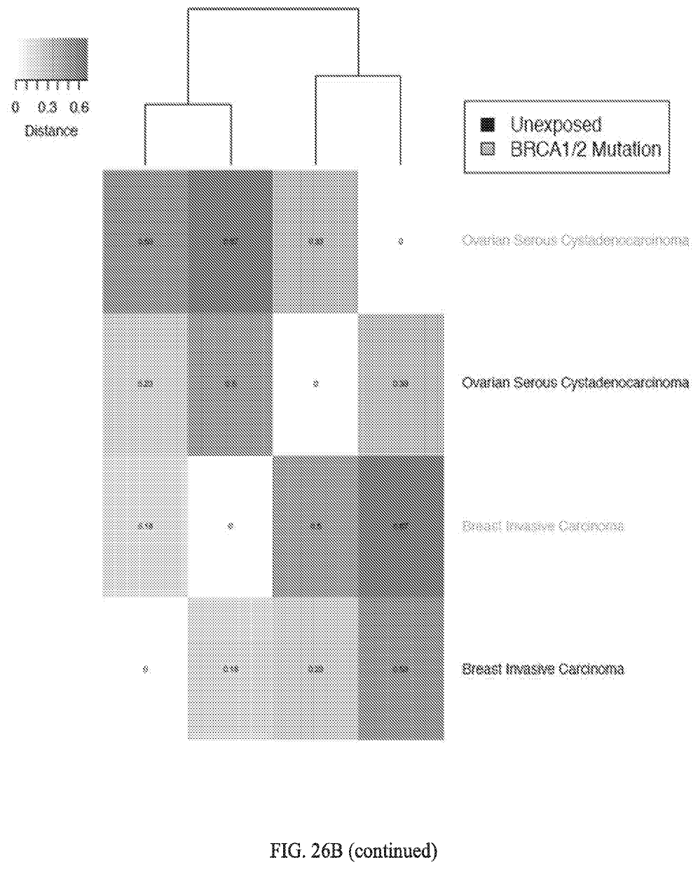

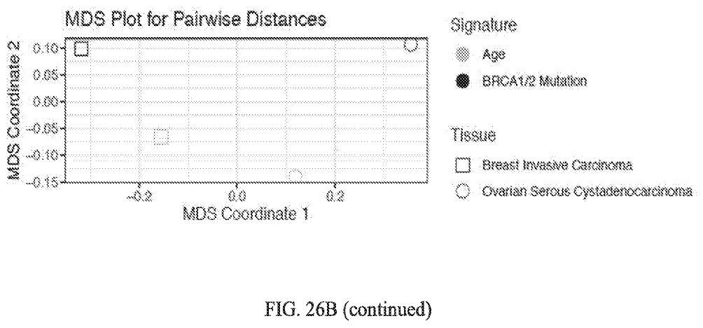

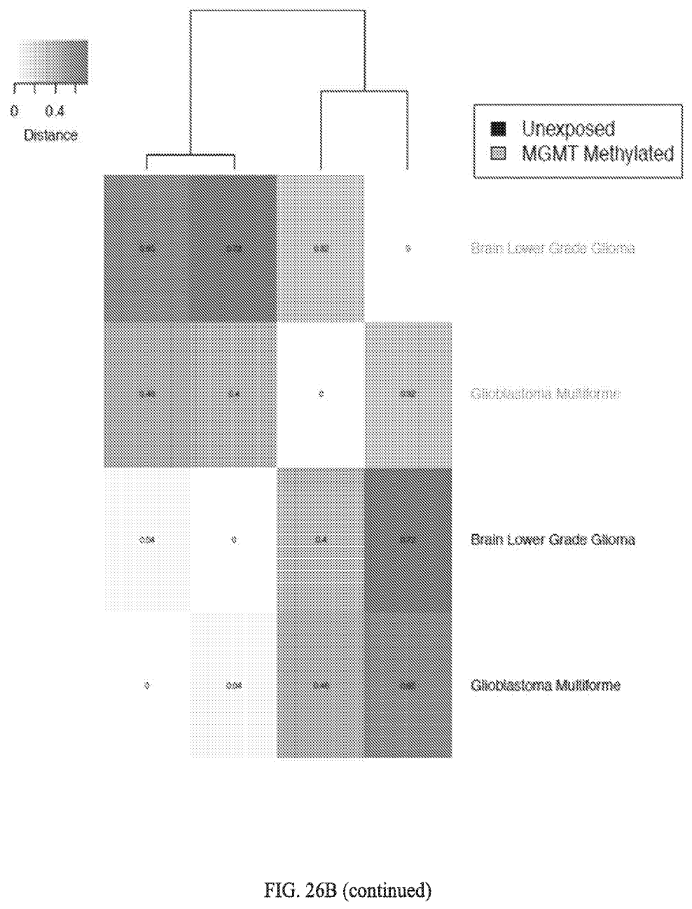

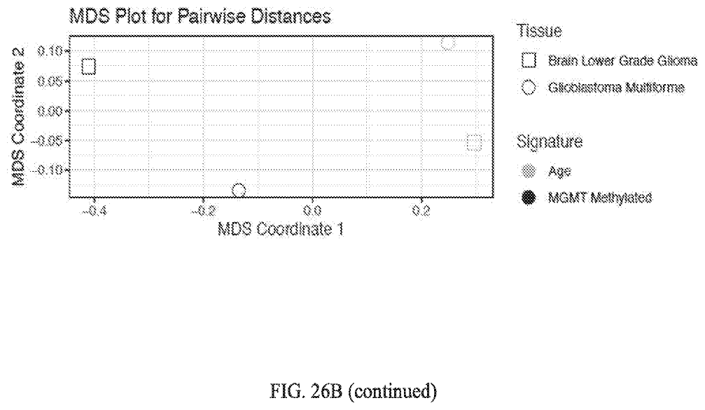

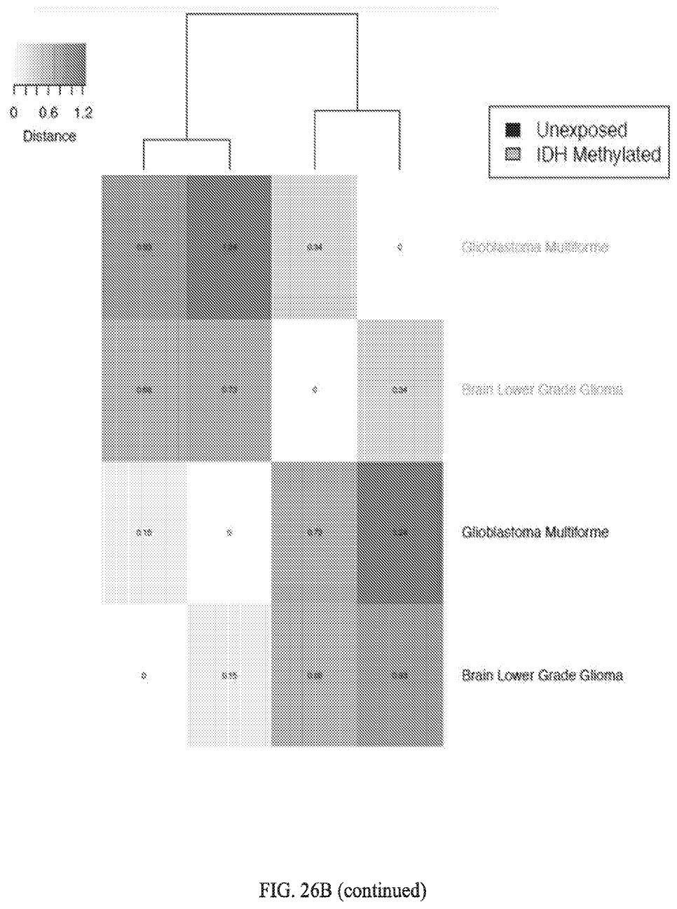

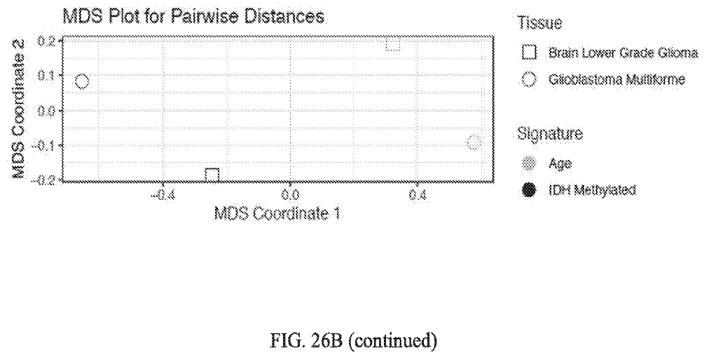

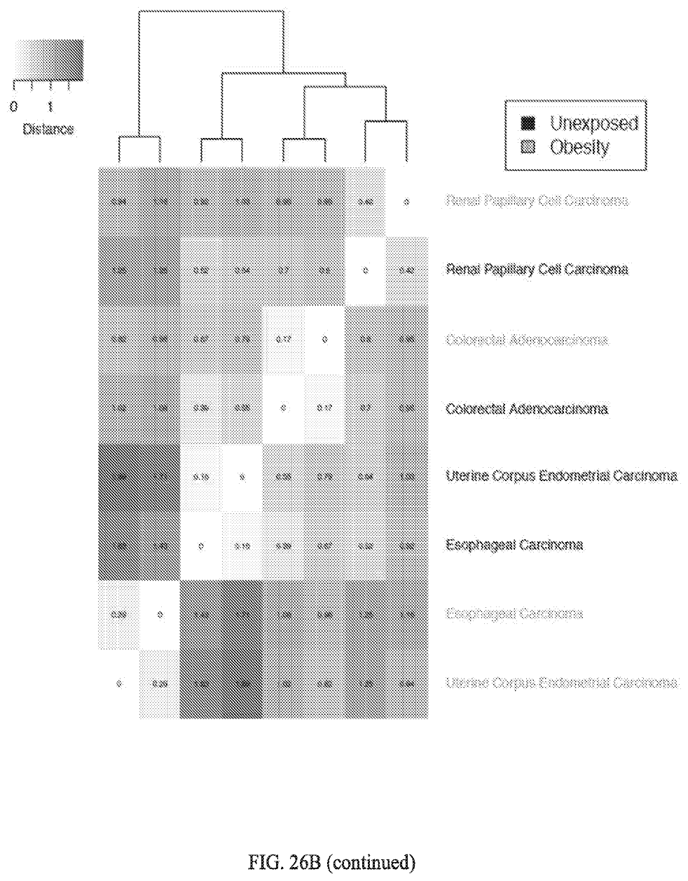

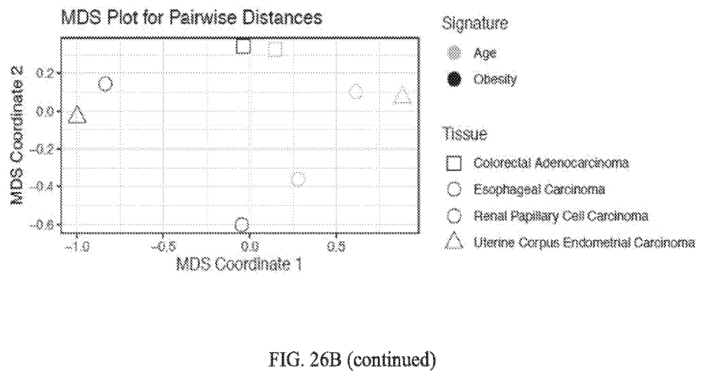

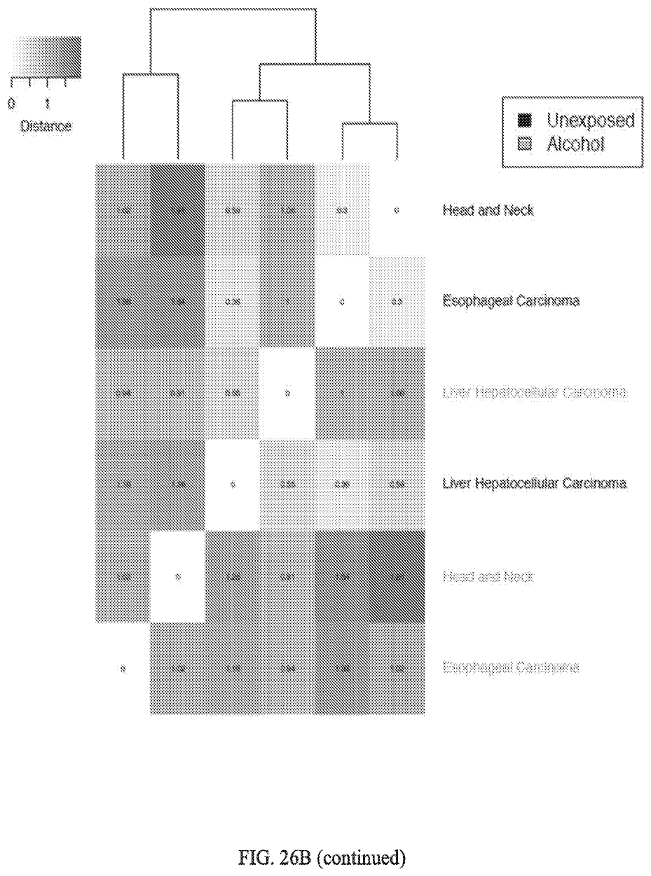

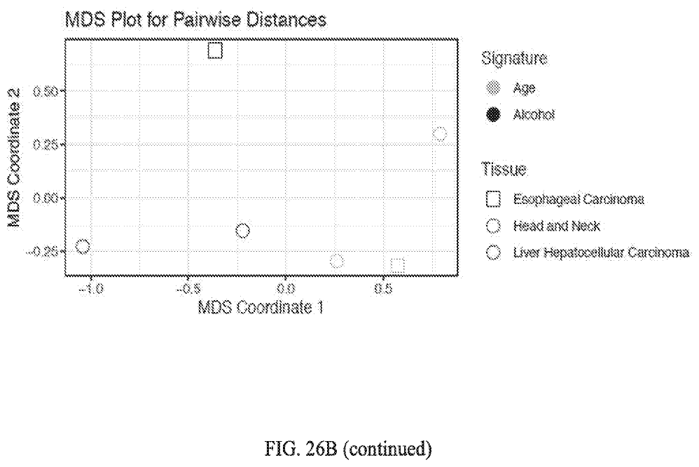

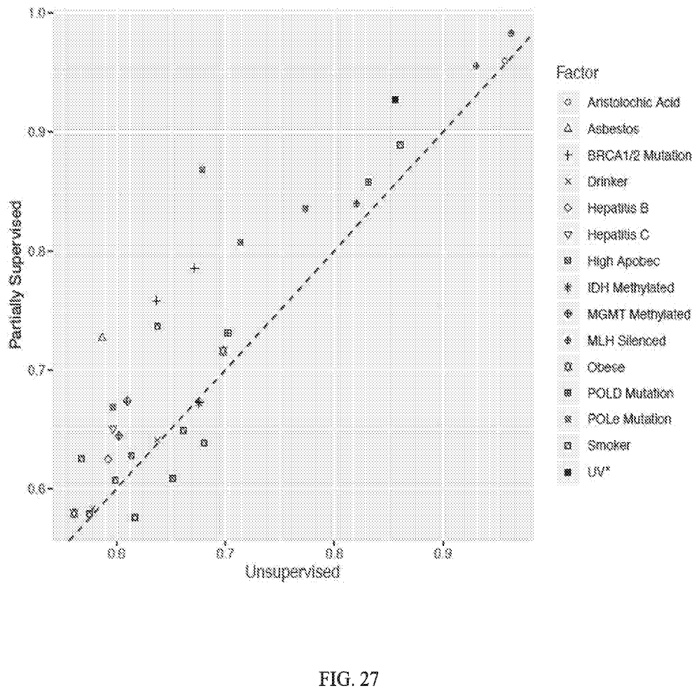

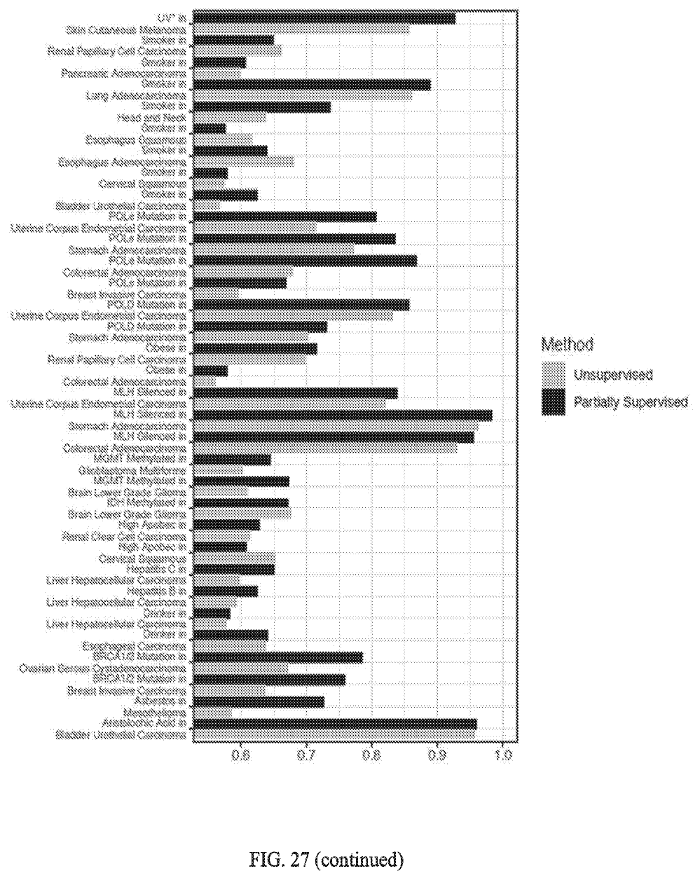

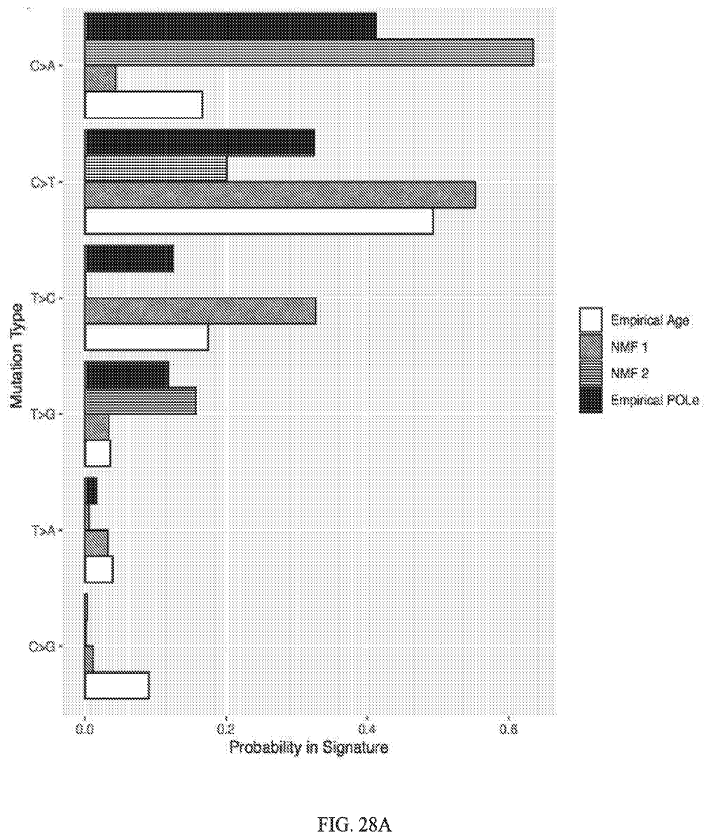

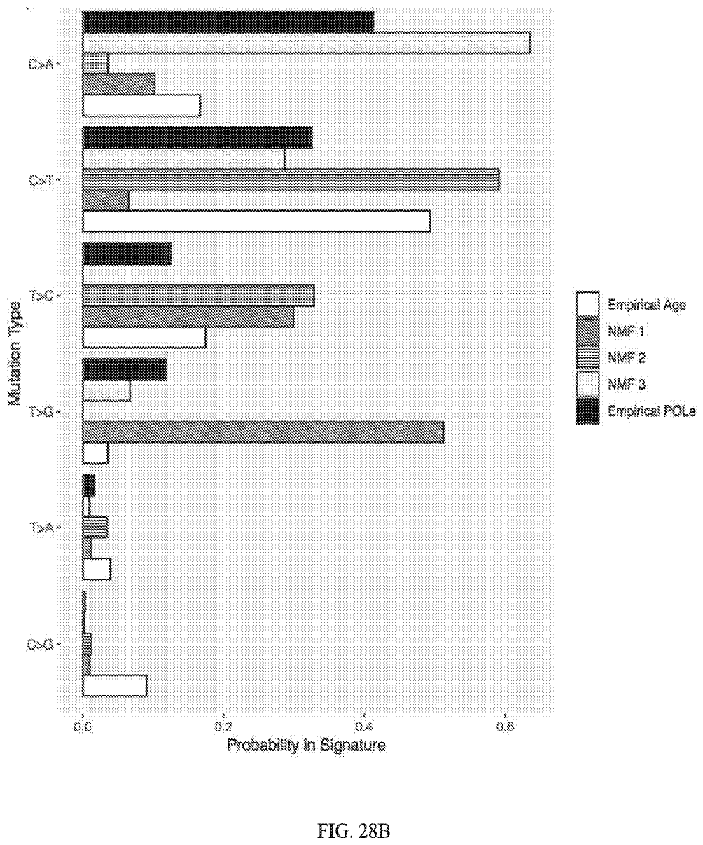

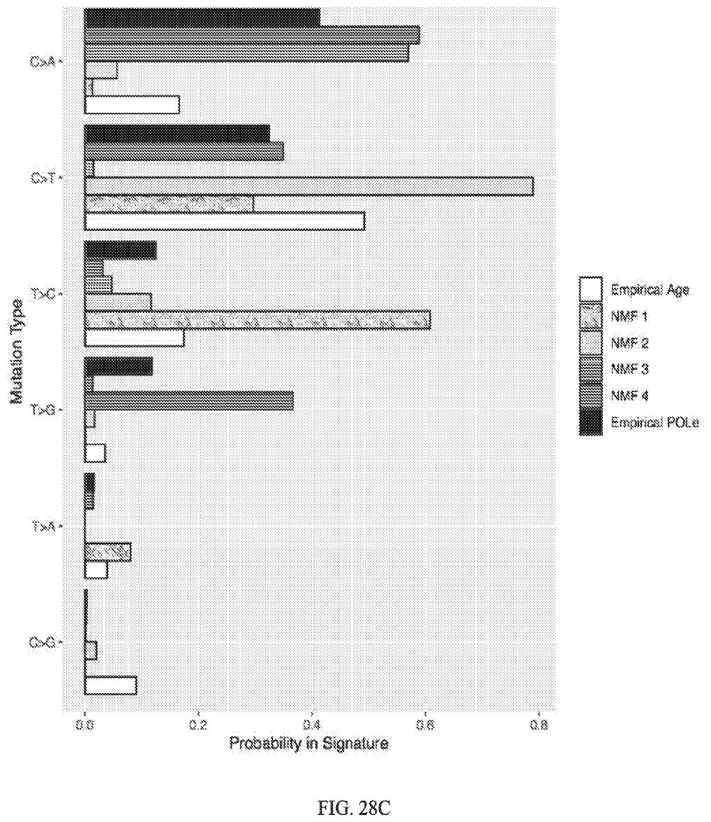

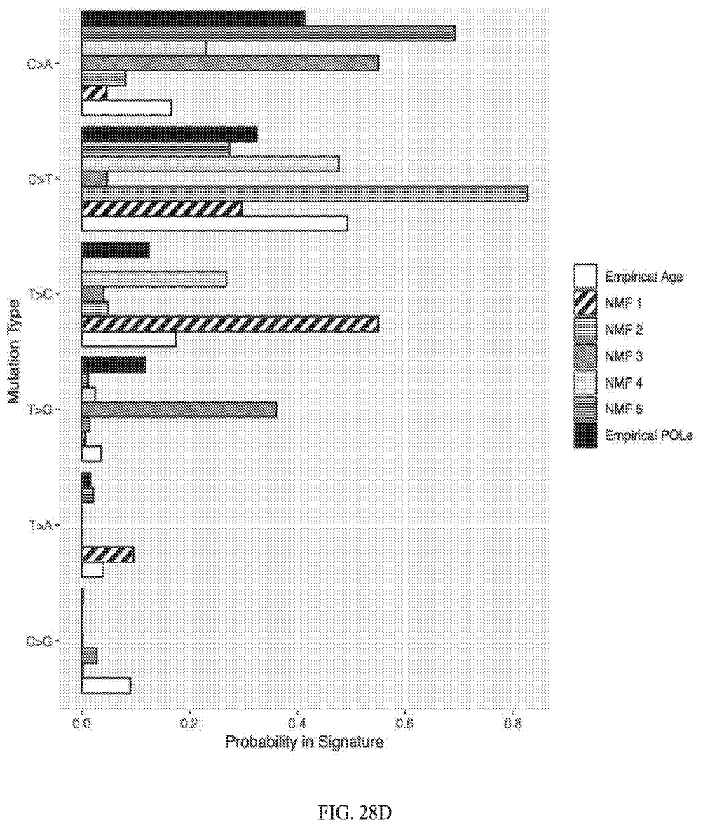

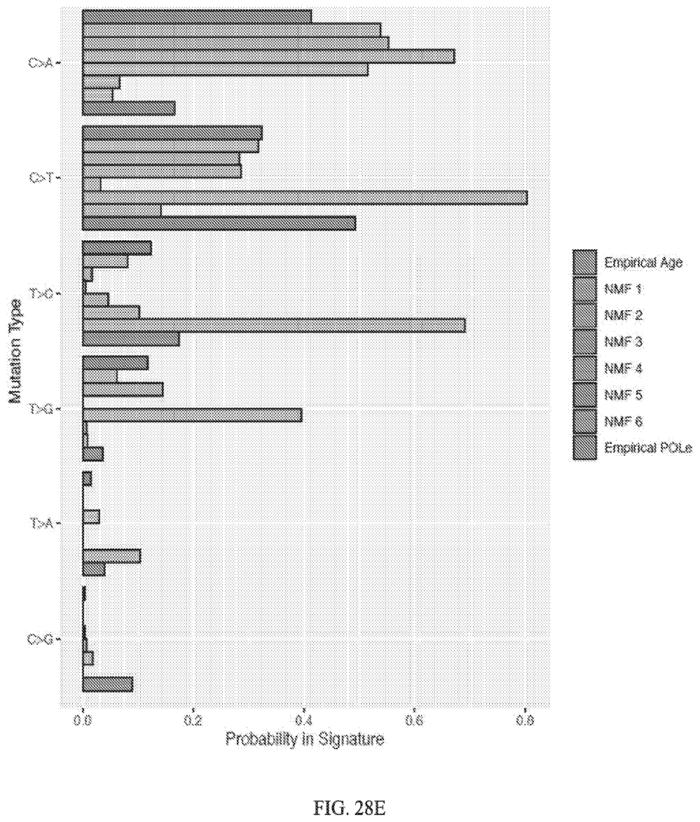

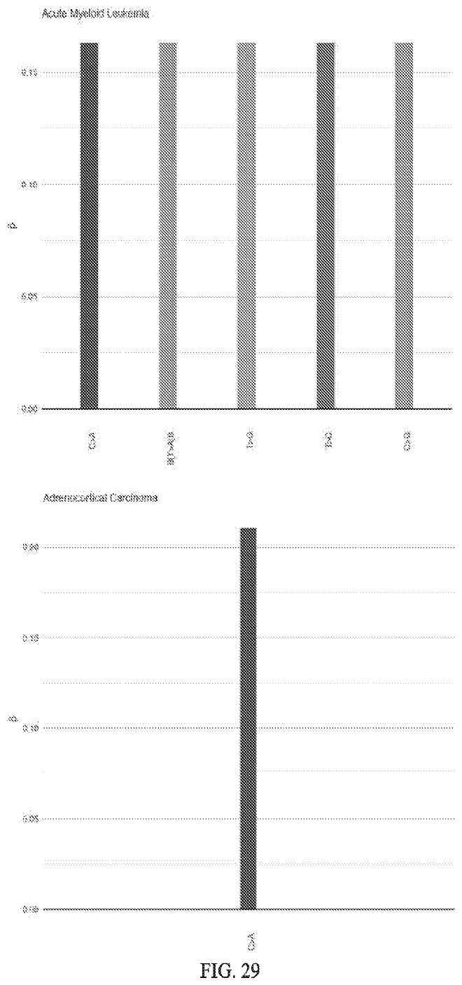

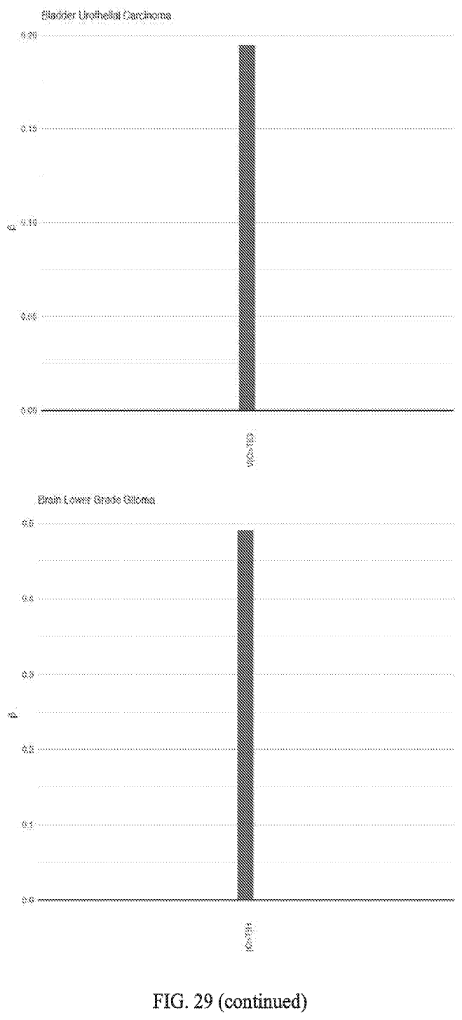

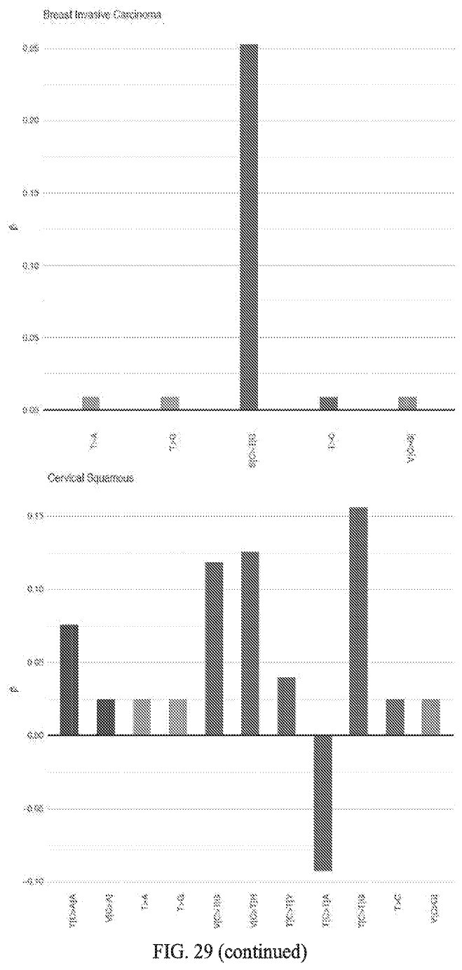

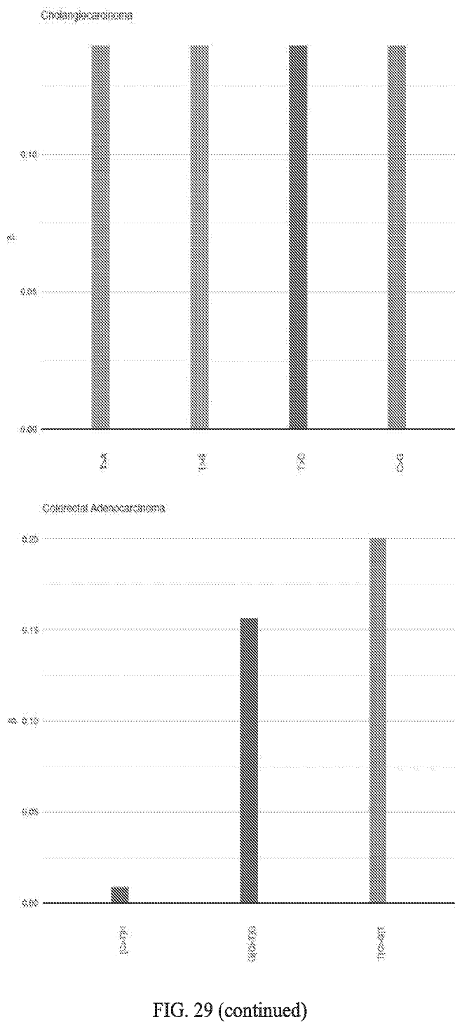

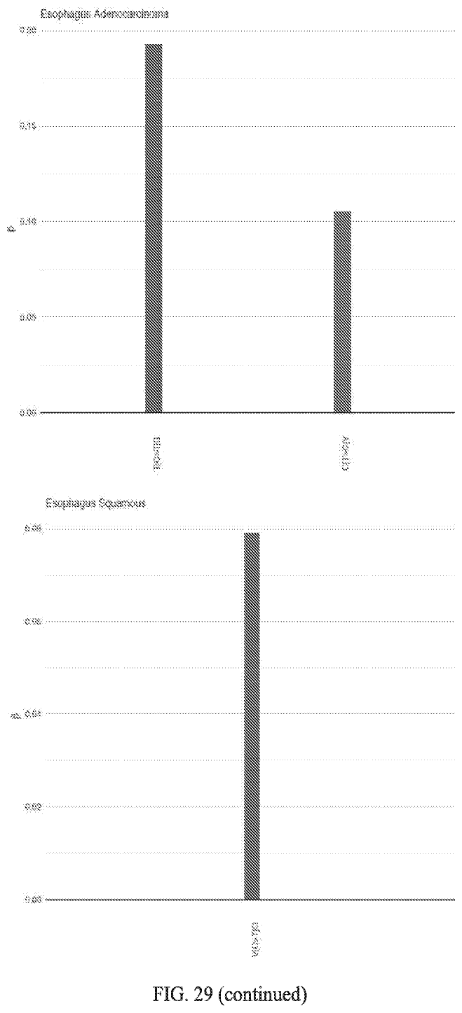

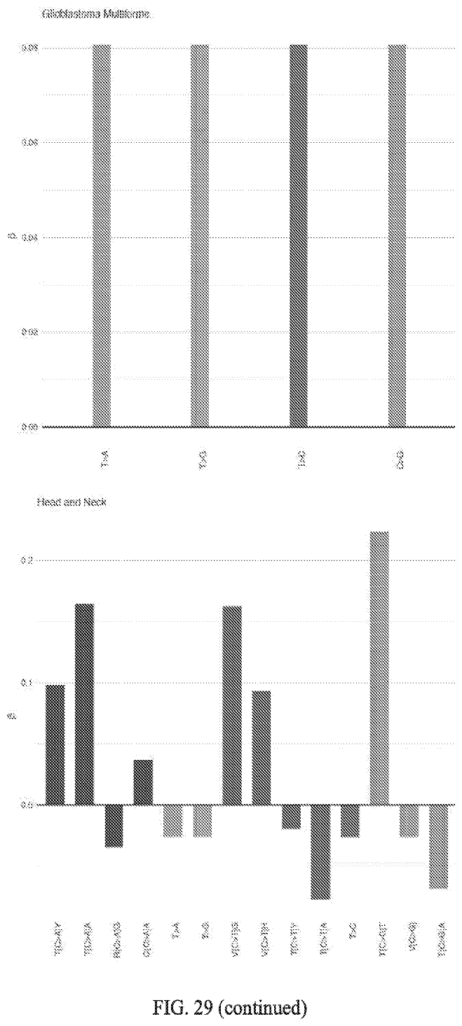

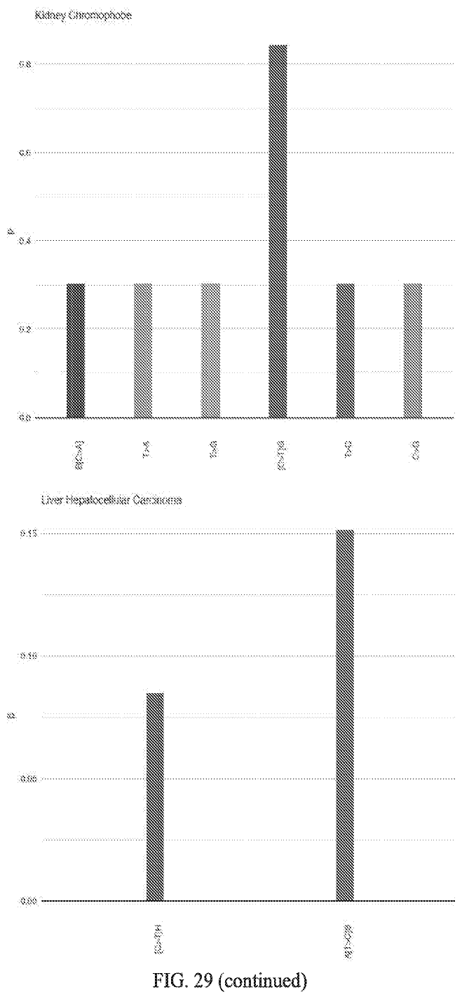

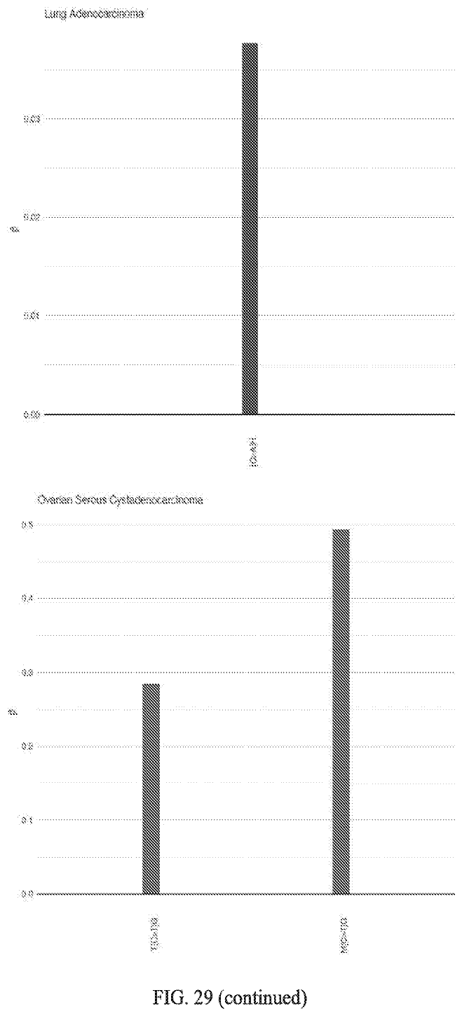

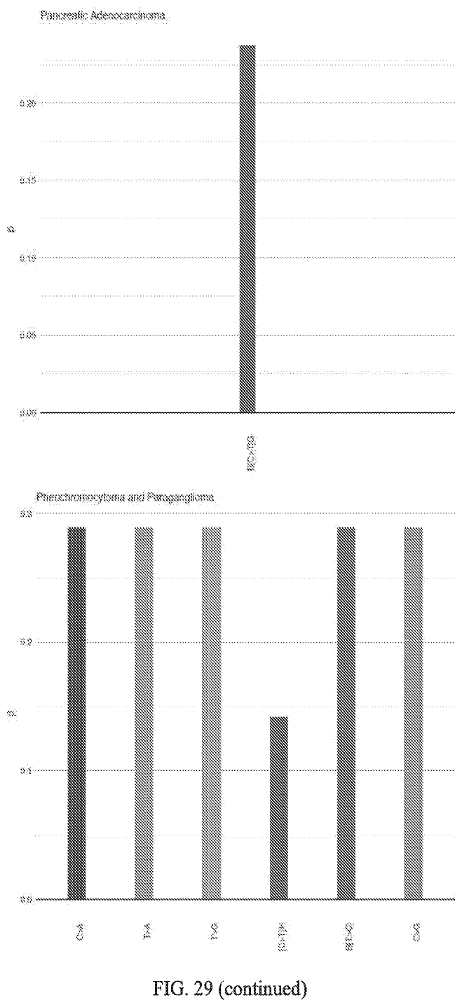

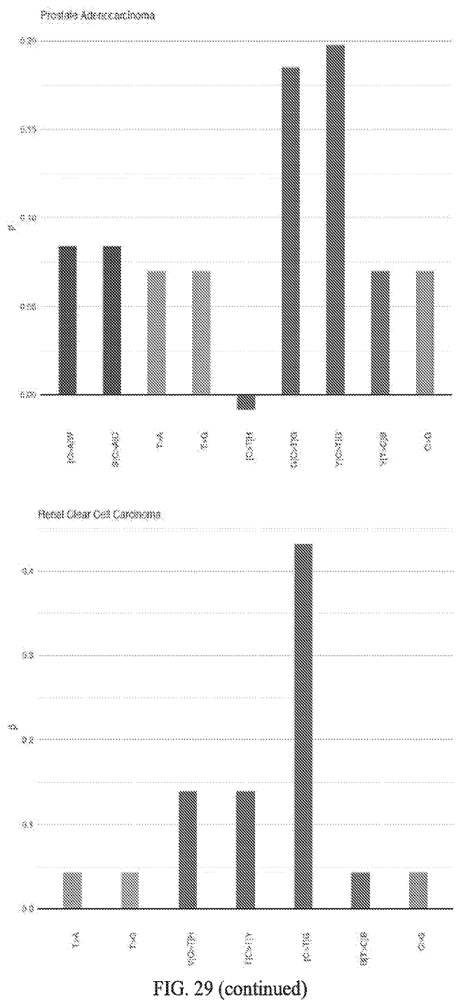

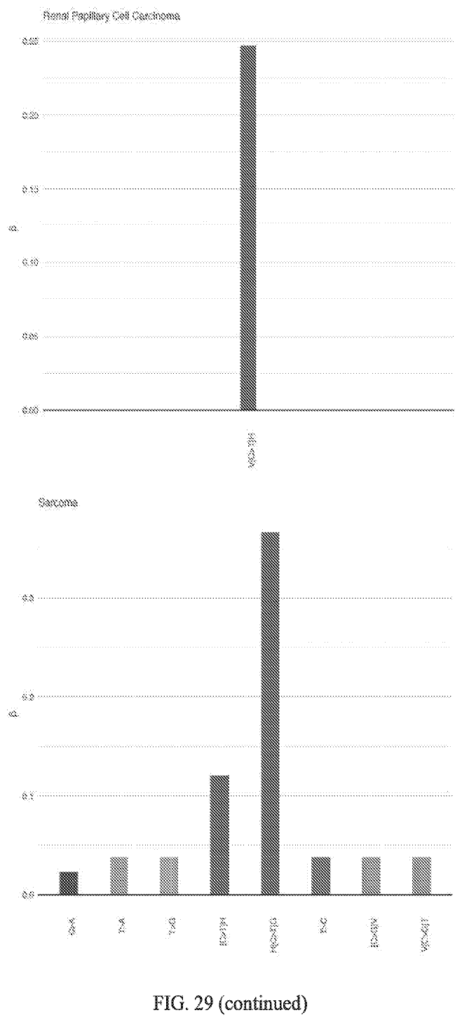

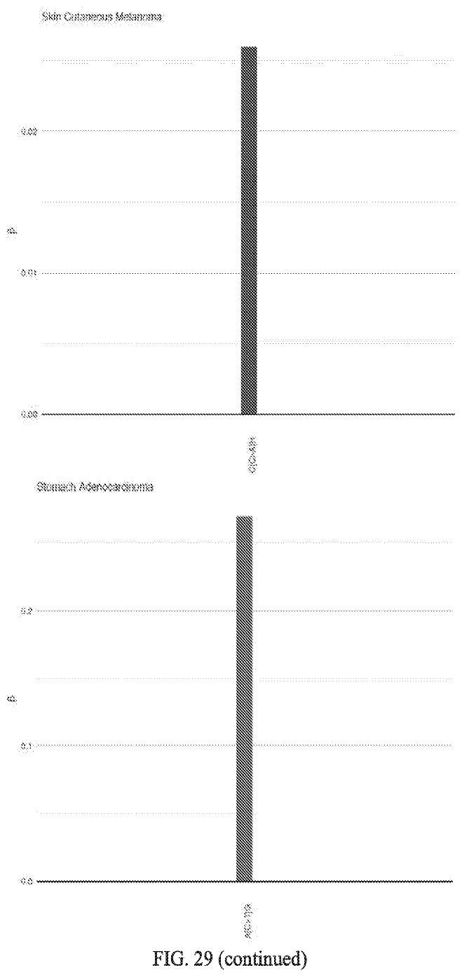

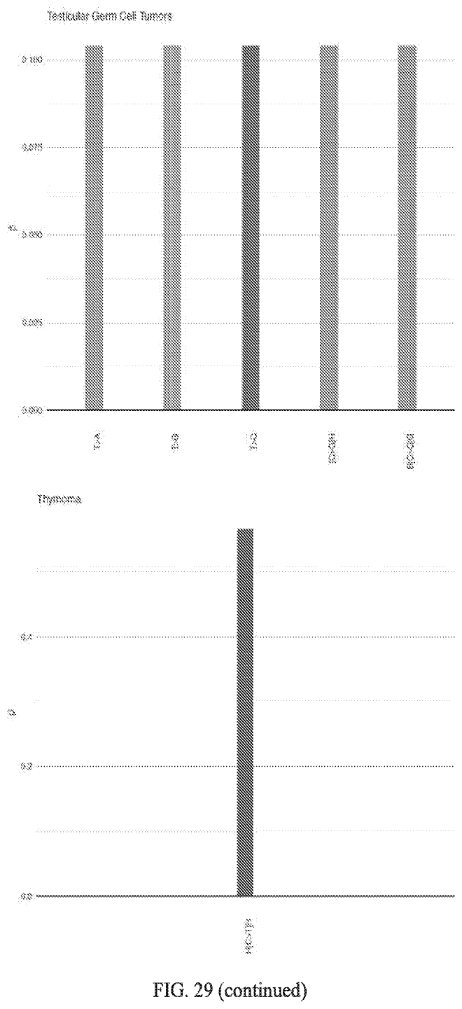

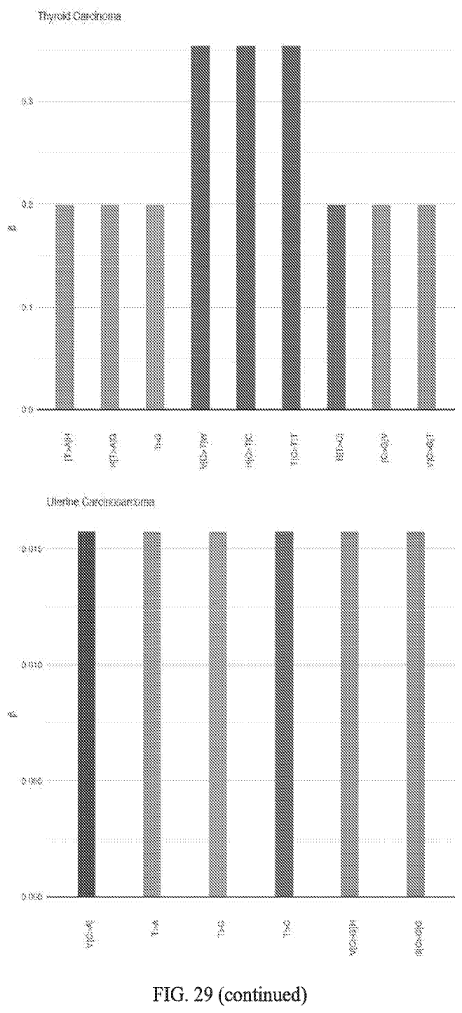

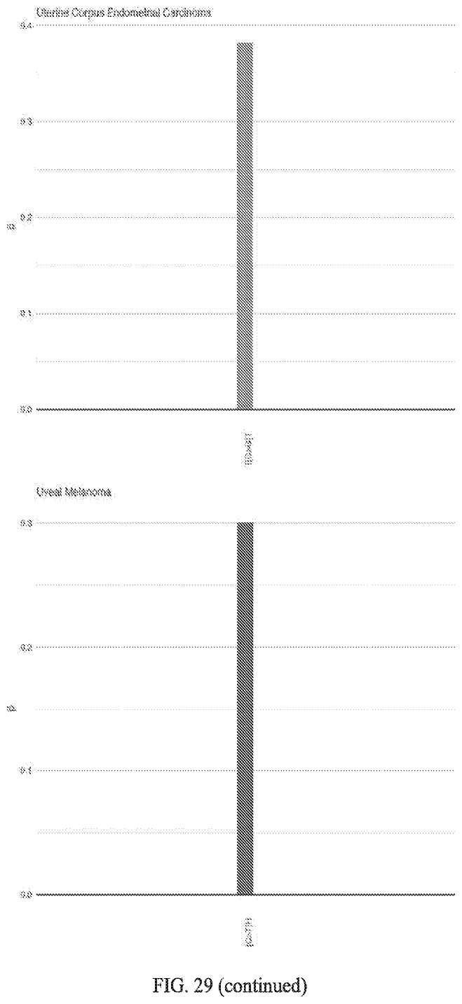

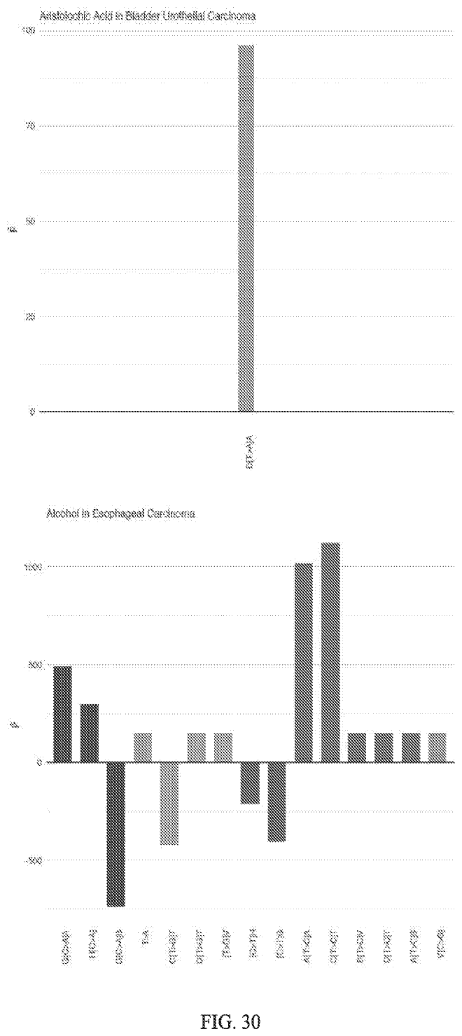

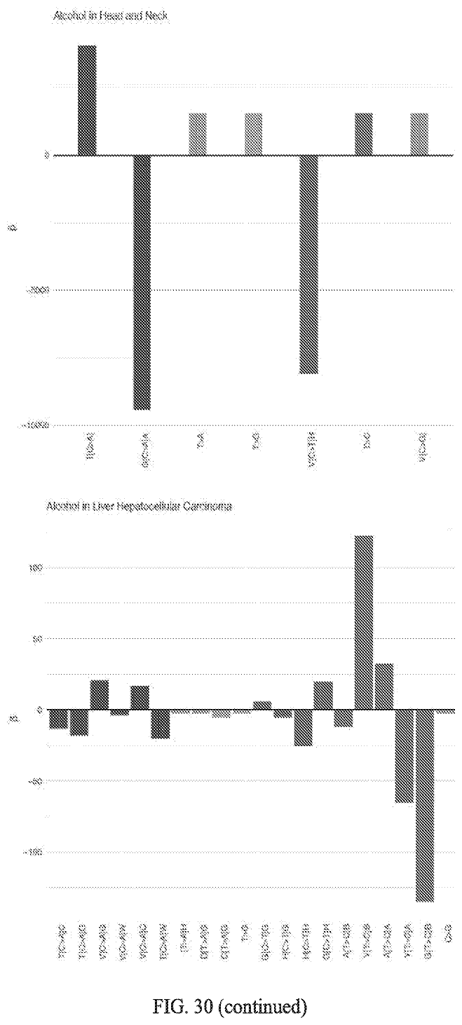

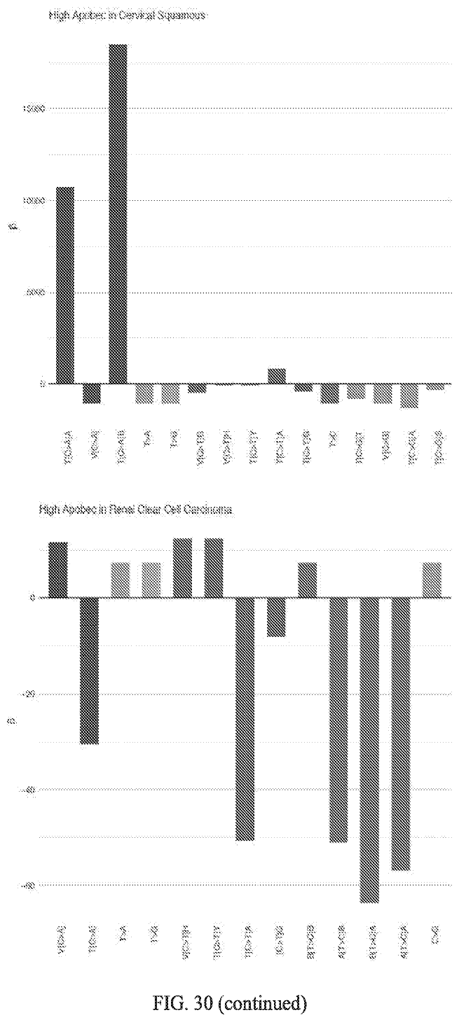

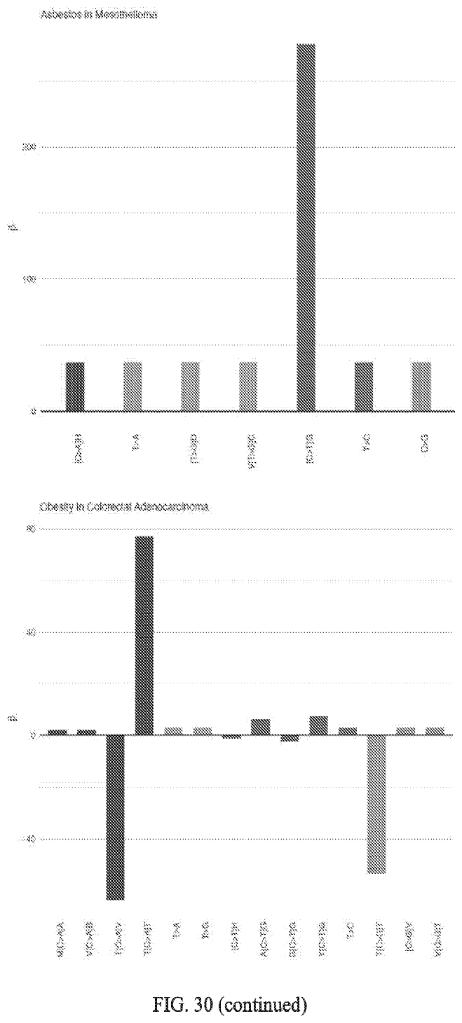

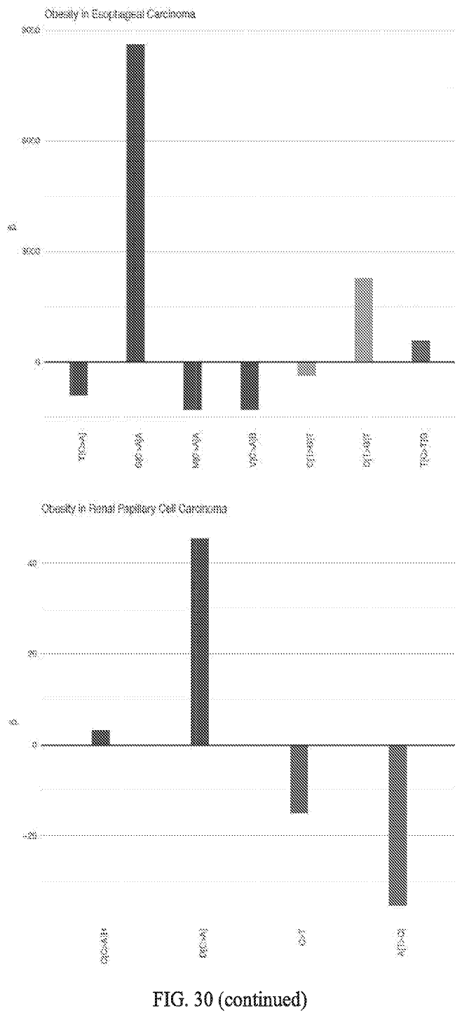

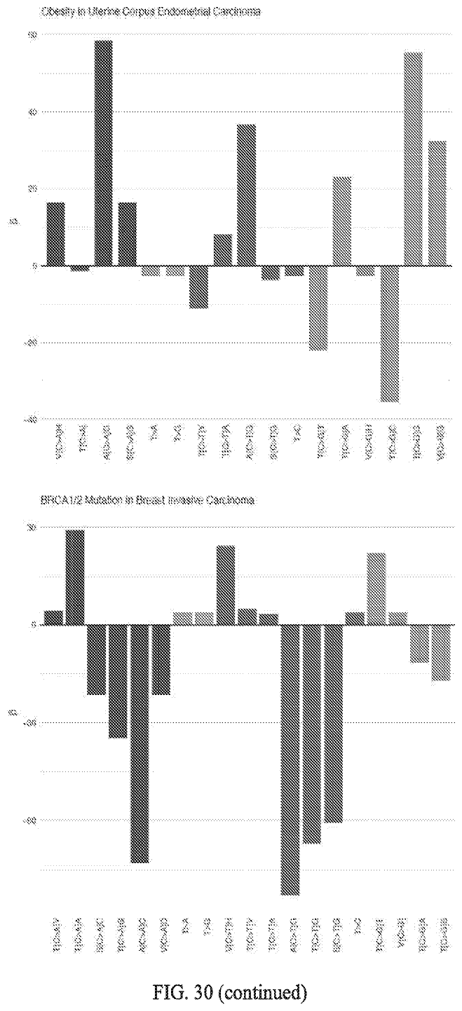

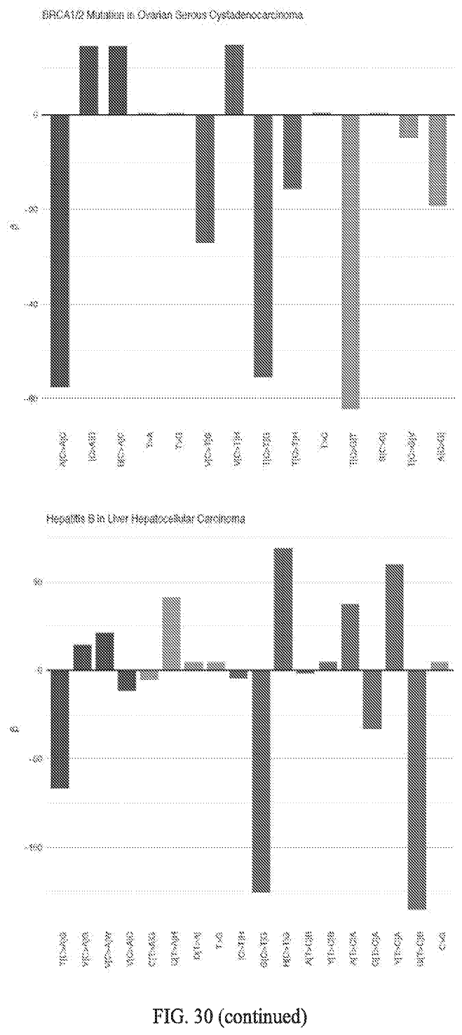

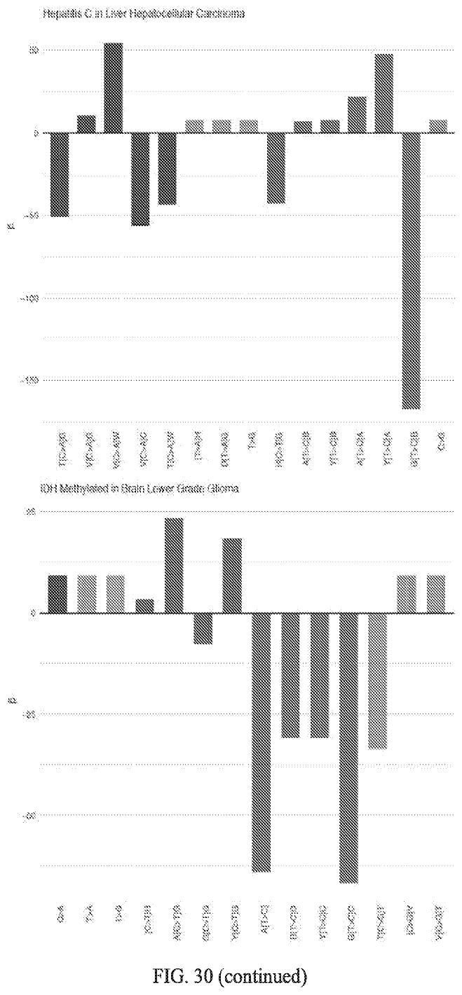

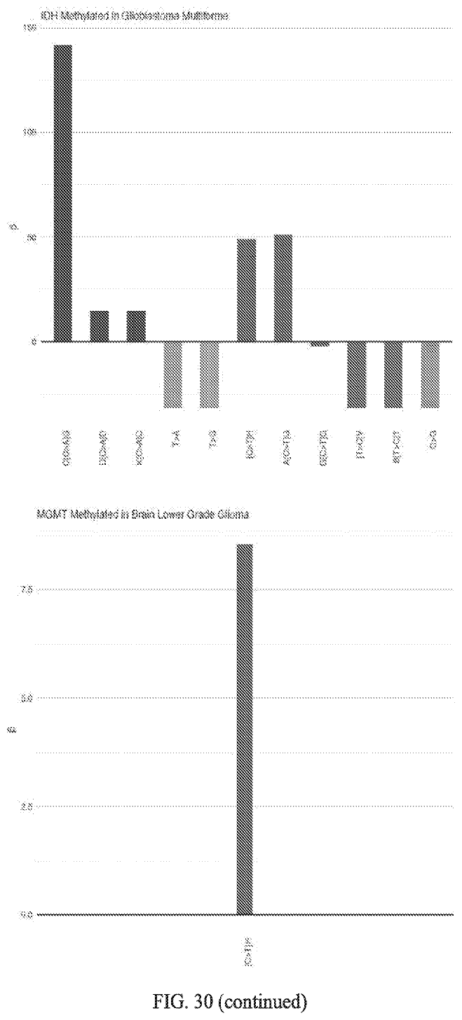

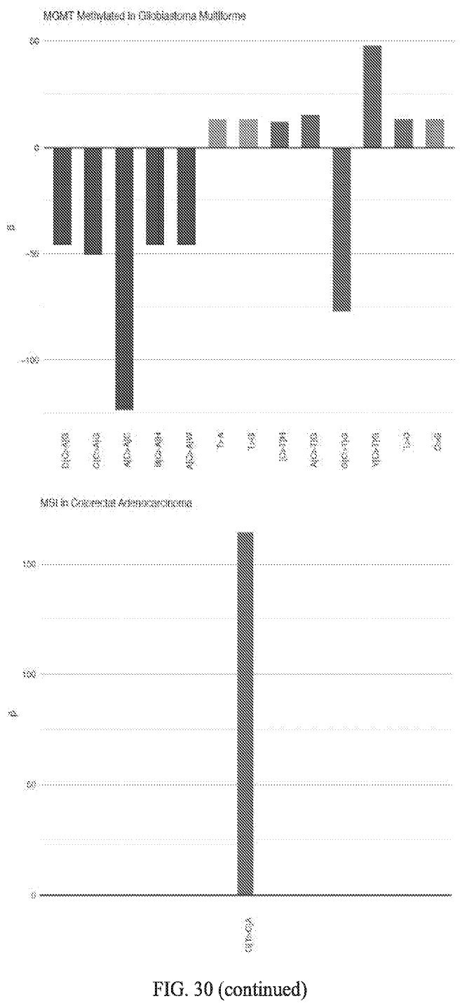

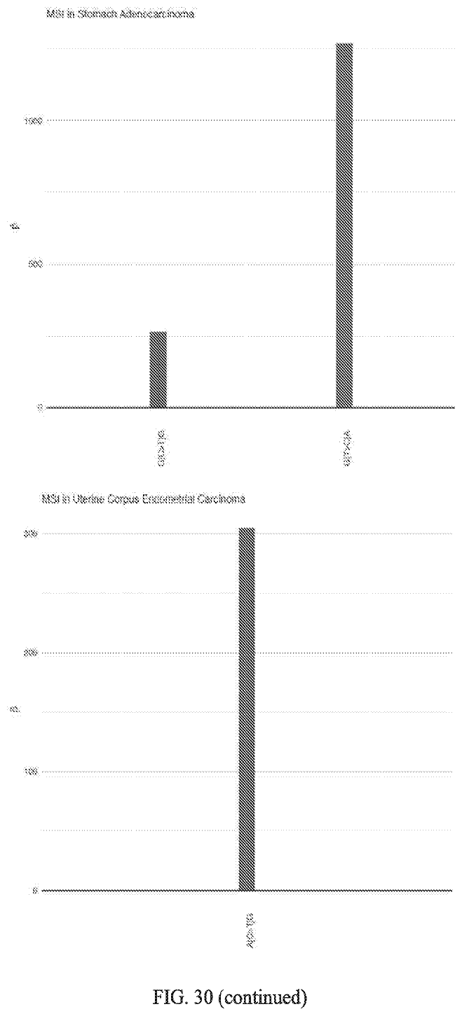

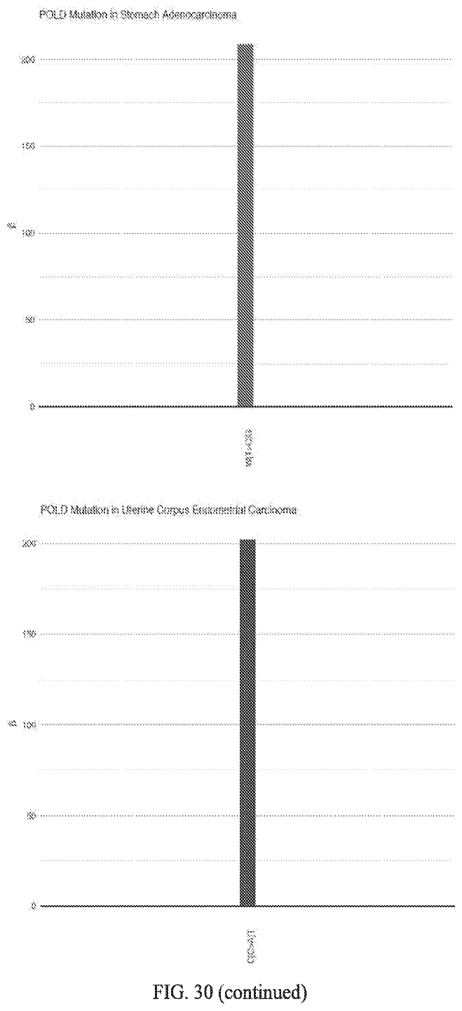

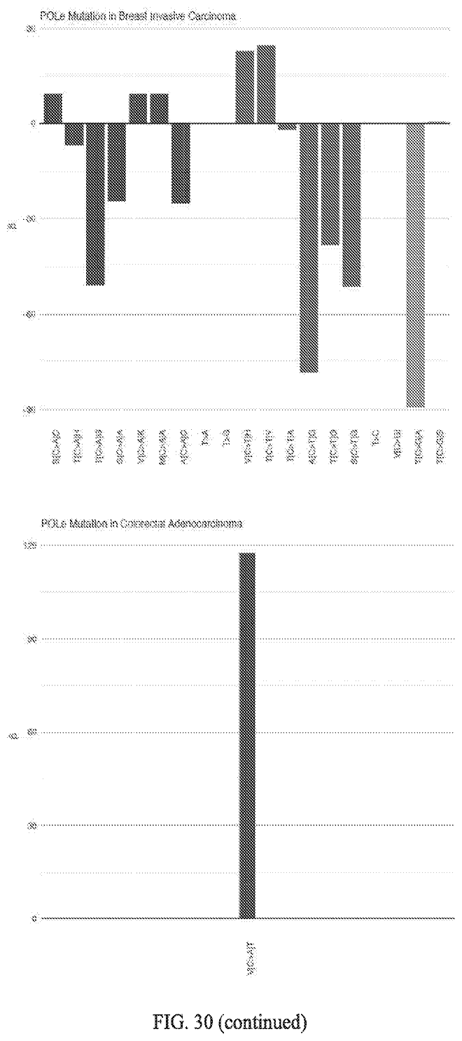

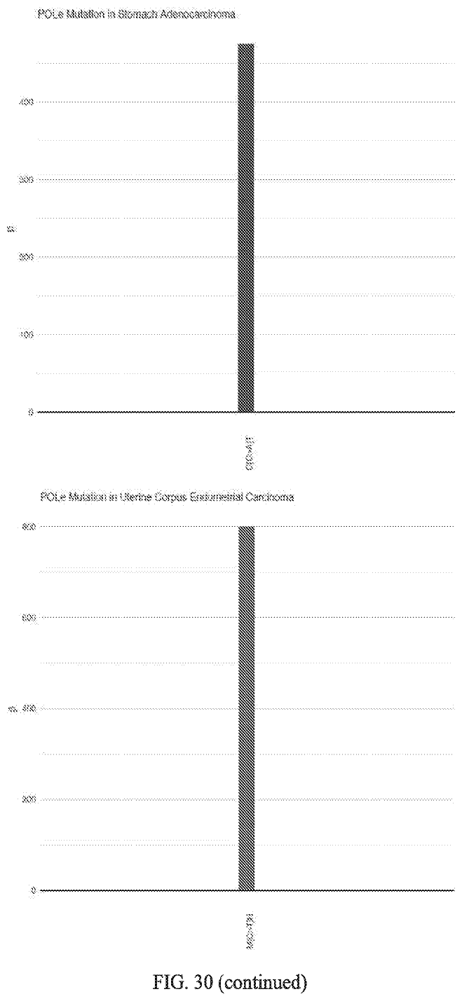

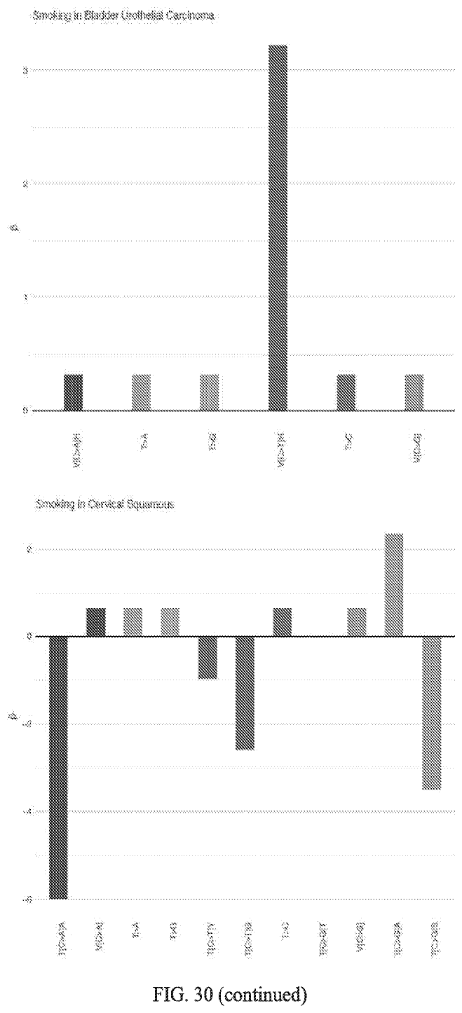

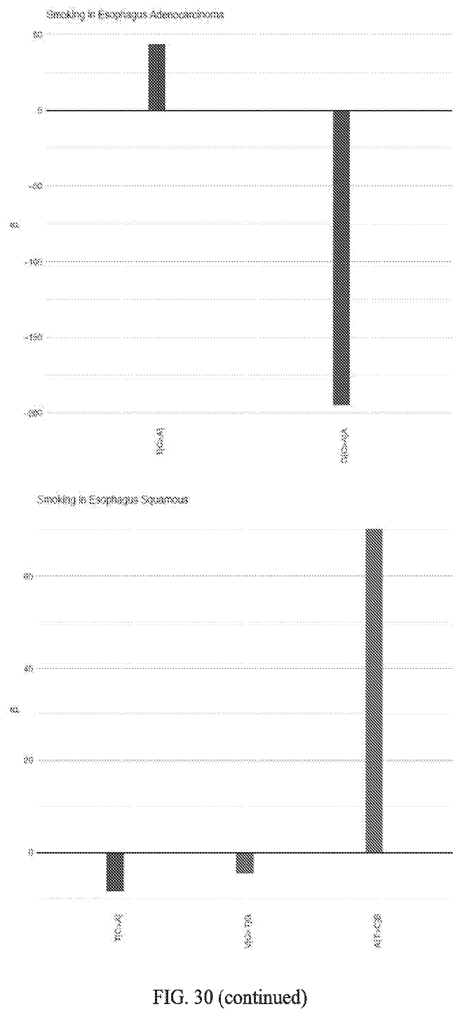

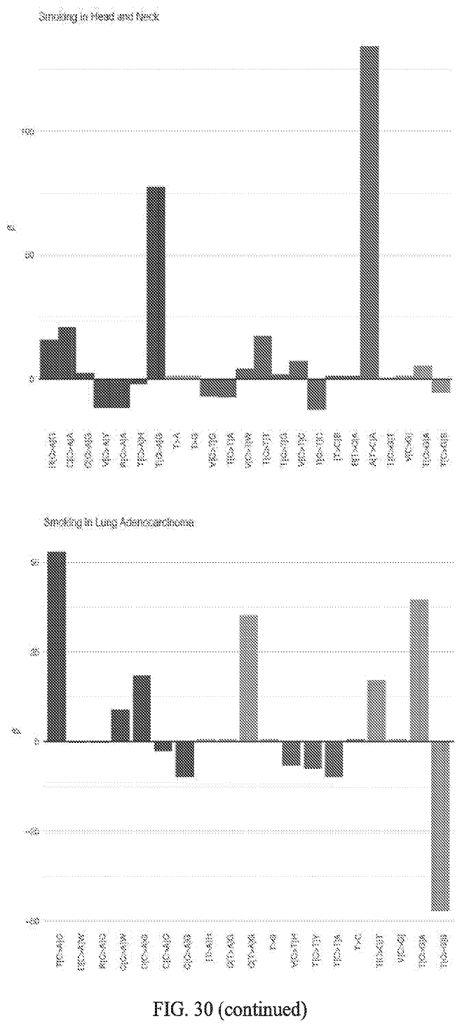

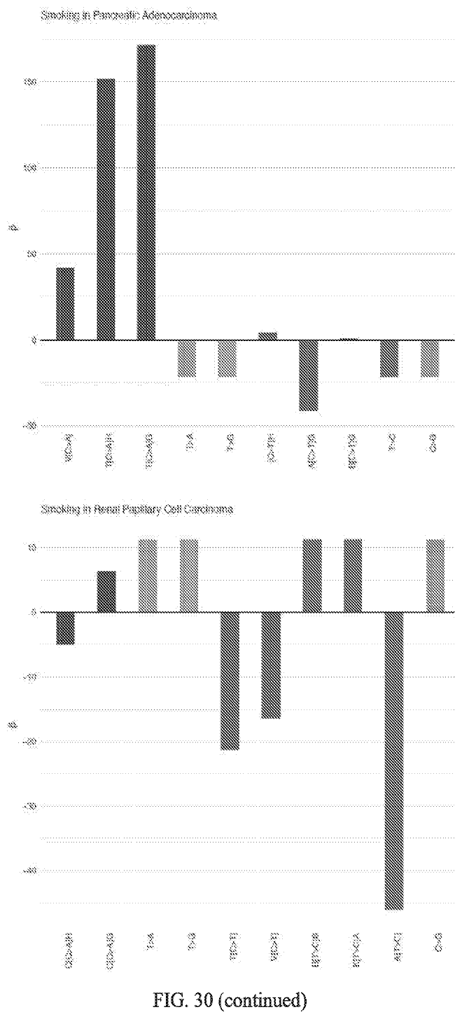

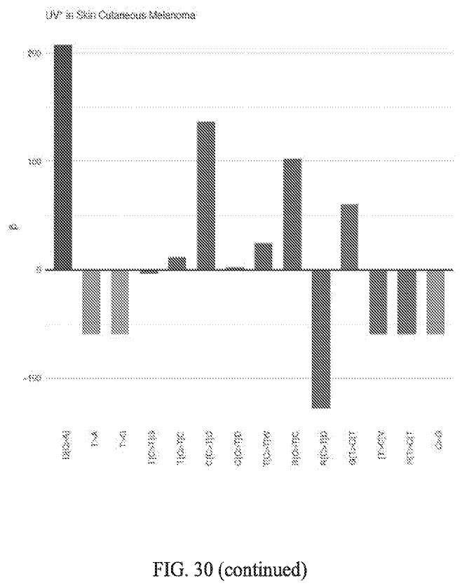

FIG. 7 show an example process 700 for detecting an etiological factor of a disease. The process 700 can be performed by, for example, computational systems and users that have access to the data described with respect to FIGS. 6A and 6B.

Training data is received 702.

Sets of features are generated from nucleotide mutations 704 until all groups of mutations are processed 706.

A machine learning model is trained 708 on the features.

Training of the machine learning model comprises organizing the features into a partition tree that includes layers of nodes, each node representing a particular type of mutation and each child of the node representing possible mutations that are a type of mutation in the particular node.

Training of the machine learning model further comprises pruning the partition tree by removing a pruned node and all other nodes that are children of the pruned node.

Training of the machine learning model comprises selecting some, but not all, of the nodes as candidate nodes to be used for candidate testing; and testing the candidate nodes to generate first-phase candidate nodes.

Training of the machine learning model further comprises generating second-phase candidates by for each particular first-phase candidate node, adjusting a value for each parent node that is also a first-phase candidate node, the adjustment being based on the particular first-phase candidate node; selecting, as a second-phase candidate, a first-phase candidate with a remaining value above a threshold value.

Classifiers are generated 710. The classifiers are configured to operate by receiving a new-genomic-data-object, the new-genomic-data-object specific to the subject having the disease and generate, from the new-genomic-data-object, a etiological-classification for the new-genomic-data-object, the etiological-classification.

Training of the machine learning model further comprises: generating final candidates by: combining second-phase candidates of training data that did have a particular tag with training data that did not have the particular tag.

A subject's genome is received 712 as a subject-genomic-data-object.

Etiologic factor(s) are detected 714 by providing the subject-genomic-data-object to the classifier.

FIG. 8 is a schematic diagram that shows an example of a computing system 800. The computing system 800 can be used for some or all of the operations described previously, according to some implementations. The computing system 800 includes a processor 810, a memory 820, a storage device 830, and an input/output device 840. Each of the processor 810, the memory 820, the storage device 830, and the input/output device 840 are interconnected using a system bus 850. The processor 810 is capable of processing instructions for execution within the computing system 800. In some implementations, the processor 810 is a single-threaded processor. In some implementations, the processor 810 is a multi-threaded processor. The processor 810 is capable of processing instructions stored in the memory 820 or on the storage device 830 to display graphical information for a user interface on the input/output device 840.

The memory 820 stores information within the computing system 800. In some implementations, the memory 820 is a computer-readable medium. In some implementations, the memory 820 is a volatile memory unit. In some implementations, the memory 820 is a non-volatile memory unit.

The storage device 830 is capable of providing mass storage for the computing system 800. In some implementations, the storage device 830 is a computer-readable medium. In various different implementations, the storage device 830 may be a floppy disk device, a hard disk device, an optical disk device, or a tape device.

The input/output device 840 provides input/output operations for the computing system 800. In some implementations, the input/output device 840 includes a keyboard and/or pointing device. In some implementations, the input/output device 840 includes a display unit for displaying graphical user interfaces.

Some features described can be implemented in digital electronic circuitry, or in computer hardware, firmware, software, or in combinations of them. The apparatus can be implemented in a computer program product tangibly embodied in an information carrier, e.g., in a machine-readable storage device, for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM (erasable programmable read-only memory), EEPROM (electrically erasable programmable read-only memory), and flash memory devices; magnetic disks such as internal hard disks and removable disks; magneto-optical disks; and CD-ROM (compact disc read-only memory) and DVD-ROM (digital versatile disc read-only memory) disks. The processor and the memory can be supplemented by, or incorporated in, ASICs (application-specific integrated circuits).

To provide for interaction with a user, some features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

FIG. 9 is a schematic diagram that shows an example of a computing device and a mobile computing device.

FIG. 9 shows an example of a computing device 900 and an example of a mobile computing device that can be used to implement the techniques described here. The computing device 900 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 900 includes a processor 902, a memory 904, a storage device 906, a high-speed interface 908 connecting to the memory 904 and multiple high-speed expansion ports 910, and a low-speed interface 912 connecting to a low-speed expansion port 914 and the storage device 906. Each of the processor 902, the memory 904, the storage device 906, the high-speed interface 908, the high-speed expansion ports 910, and the low-speed interface 912, are interconnected using various busses, and can be mounted on a common motherboard or in other manners as appropriate. The processor 902 can process instructions for execution within the computing device 900, including instructions stored in the memory 904 or on the storage device 906 to display graphical information for a GUI on an external input/output device, such as a display 916 coupled to the high-speed interface 908. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 904 stores information within the computing device 900. In some implementations, the memory 904 is a volatile memory unit or units. In some implementations, the memory 904 is a non-volatile memory unit or units. The memory 904 can also be another form of computer-readable medium, such as a magnetic or optical disk.

The storage device 906 is capable of providing mass storage for the computing device 900. In some implementations, the storage device 906 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 904, the storage device 906, or memory on the processor 902.

The high-speed interface 908 manages bandwidth-intensive operations for the computing device 900, while the low-speed interface 912 manages lower bandwidth-intensive operations. Such allocation of functions is exemplary only. In some implementations, the high-speed interface 908 is coupled to the memory 904, the display 916 (e.g., through a graphics processor or accelerator), and to the high-speed expansion ports 910, which can accept various expansion cards (not shown). In the implementation, the low-speed interface 912 is coupled to the storage device 906 and the low-speed expansion port 914. The low-speed expansion port 914, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 900 can be implemented in a number of different forms, as shown in the figure. For example, it can be implemented as a standard server 920, or multiple times in a group of such servers. In addition, it can be implemented in a personal computer such as a laptop computer 922. It can also be implemented as part of a rack server system 924. Alternatively, components from the computing device 900 can be combined with other components in a mobile device (not shown), such as a mobile computing device 950. Each of such devices can contain one or more of the computing device 900 and the mobile computing device 950, and an entire system can be made up of multiple computing devices communicating with each other.

The mobile computing device 950 includes a processor 952, a memory 964, an input/output device such as a display 954, a communication interface 966, and a transceiver 968, among other components. The mobile computing device 950 can also be provided with a storage device, such as a micro-drive or other device, to provide additional storage. Each of the processor 952, the memory 964, the display 954, the communication interface 966, and the transceiver 968, are interconnected using various buses, and several of the components can be mounted on a common motherboard or in other manners as appropriate.

The processor 952 can execute instructions within the mobile computing device 950, including instructions stored in the memory 964. The processor 952 can be implemented as a chipset of chips that include separate and multiple analog and digital processors. The processor 952 can provide, for example, for coordination of the other components of the mobile computing device 950, such as control of user interfaces, applications run by the mobile computing device 950, and wireless communication by the mobile computing device 950.

The processor 952 can communicate with a user through a control interface 958 and a display interface 956 coupled to the display 954. The display 954 can be, for example, a TFT (Thin-Film-Transistor Liquid Crystal Display) display or an OLED (Organic Light Emitting Diode) display, or other appropriate display technology. The display interface 956 can comprise appropriate circuitry for driving the display 954 to present graphical and other information to a user. The control interface 958 can receive commands from a user and convert them for submission to the processor 952. In addition, an external interface 962 can provide communication with the processor 952, so as to enable near area communication of the mobile computing device 950 with other devices. The external interface 962 can provide, for example, for wired communication in some implementations, or for wireless communication in other implementations, and multiple interfaces can also be used.

The memory 964 stores information within the mobile computing device 950. The memory 964 can be implemented as one or more of a computer-readable medium or media, a volatile memory unit or units, or a non-volatile memory unit or units. An expansion memory 974 can also be provided and connected to the mobile computing device 950 through an expansion interface 972, which can include, for example, a SIMM (Single In Line Memory Module) card interface. The expansion memory 974 can provide extra storage space for the mobile computing device 950, or can also store applications or other information for the mobile computing device 950. Specifically, the expansion memory 974 can include instructions to carry out or supplement the processes described above, and can include secure information also. Thus, for example, the expansion memory 974 can be provide as a security module for the mobile computing device 950, and can be programmed with instructions that permit secure use of the mobile computing device 950. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 964, the expansion memory 974, or memory on the processor 952. In some implementations, the computer program product can be received in a propagated signal, for example, over the transceiver 968 or the external interface 962.

The mobile computing device 950 can communicate wirelessly through the communication interface 966, which can include digital signal processing circuitry where necessary. The communication interface 966 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 968 using a radio-frequency. In addition, short-range communication can occur, such as using a Bluetooth, WiFi, or other such transceiver (not shown). In addition, a GPS (Global Positioning System) receiver module 970 can provide additional navigation- and location-related wireless data to the mobile computing device 950, which can be used as appropriate by applications running on the mobile computing device 950.

The mobile computing device 950 can also communicate audibly using an audio codec 960, which can receive spoken information from a user and convert it to usable digital information. The audio codec 960 can likewise generate audible sound for a user, such as through a speaker, e.g., in a handset of the mobile computing device 950. Such sound can include sound from voice telephone calls, can include recorded sound (e.g., voice messages, music files, etc.) and can also include sound generated by applications operating on the mobile computing device 950.

The mobile computing device 950 can be implemented in a number of different forms, as shown in the figure. For example, it can be implemented as a cellular telephone 980. It can also be implemented as part of a smart-phone 982, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

FIG. 10 shows age signatures. For each indicated cancer type all selected features of its age signature are listed (IUPAC notations: B=not A, D=not C, H=not G, V=not T, W=A or T, S=C or G, M=A or C, K=G or T, R=A or G, Y=C or T). Black rectangles indicate the average frequency of a certain feature, out of the total number of somatic mutations, and compared to its expected frequency (white rectangles), as estimated by deconstructSigs.

FIG. 11 shows tissue recognition. Boxplots depicts the distribution of the prediction accuracies, as measured by AUC, obtained by LDA when classifying the indicated cancer type against each of the other types.

FIGS. 12A-12C show environmental and inherited factors' signatures. A) For each indicated cancer type and each indicated E or H factor, all selected features of its signature are listed (IUPAC notations: B=not A, D=not C, H=not G, V=not T, W=A or T, S=C or G, M=A or C, K=G or T, R=A or G, Y=C or T). Black rectangles indicate the average frequency of a certain feature, out of the total number of somatic mutations, and compared to its expected frequency (white rectangles), as estimated by deconstructSigs. B) Heat maps and multidimensional scaling (MDS) plots of the distances among signatures of the same environmental or inherited factor across cancer types. C) Heat map of the distances among all the supervised signatures obtained.

FIG. 13 shows comparisons of prediction accuracies. Comparison of the apparent prediction accuracies (in terms of AUC) are reported for all signatures of age, environmental, and inherited factors, for both the supervised and the unsupervised methodologies. Cross-validated accuracies (indicated as “CVed”) are reported for the supervised method only.

FIG. 14 shows partially supervised vs unsupervised methods' accuracies. Performance comparison in terms of AUC for the partially supervised method vs the unsupervised one.

FIG. 15 shows partially-supervised extension and the dimensionality issue with the unsupervised method. All selected features of the supervised and semi-supervised POL-ε signatures in UCEC-TCGA are listed and their frequencies compared (IUPAC notations: B=not A, D=not C, H=not G, V=not T, W=A or T, S=C or G, M=A or C, K=G or T, R=A or G, Y=C or T). Different plots are provided according to the different numbers of patterns (i.e. rank) unsupervised NMF was required to find: rank=1, 2, or 3. The larger the rank the greater the difference of the unsupervised signature from the correct supervised one.

FIG. 16 shows a flowchart of the supervised methodology for predictive mutational signatures. A schematic representation of the key steps contained in the supervised methodology. “ContextMatters” and “CombiningPartitions” are used to learn the candidate features. The final predictive features are then selected by learning the mutational differences between exposed and unexposed samples in the “PredictiveFeatures” step. These predictive features with their corresponding average rates derived during “Training” form the SuperSigs signature, which is then used to predict exposure to an etiological factor in the final “Prediction” step.

FIGS. 17A and 17B show supervised and unsupervised approaches to mutational signatures. A) The three possible scenarios in which the supervised and unsupervised approaches can be compared (black) and a summary of each comparison (red). B) Unsupervised versus random. The signature at the top of the figure is the unsupervised “aging” Signature 1 from Alexandrov et al. (Nature 500, 415-421 (2013)). The value of this signature once the “peak” at [C>T]G is removed was assessed, i.e. to evaluate how valuable is the rest of the distribution (colors not in bold) as found by the unsupervised method. The three signatures at the bottom of the figure are examples of randomly generated single peak signatures (one per color) based on sampling from a uniform distribution. Note that the peaks of these randomly generated signatures are not fixed values; they happen to carry by chance the highest weight of the distribution among a set of 30 signatures generated randomly.

FIGS. 18A-18D shows comparisons of prediction accuracies (AUCs) of unsupervised and supervised methodologies. Comparison of prediction accuracies (in terms of AUC) between supervised and unsupervised approaches for age (A), smoking (B), annotated etiological factors other than age found in Alexandrov et al. (Nature 500, 415-421 (2013)) (C), and all etiologic factors other than age (D. Each tissue is represented by a point, which depicts the prediction accuracies of the unsupervised approach (x-axis coordinate value) versus the supervised one (y-axis coordinate value). Apparent AUCs are reported in (A-C) and cross-validated in (D). The great majority of the points lie above the line, indicating the greater accuracy of the supervised approach.

FIGS. 19A-19C show SuperSigs in various tissue types. All predictive features of a signature are depicted (IUPAC notations: B=not A, D=not C, H=not G, V=not T, W=A or T, S=C or G, M=A or C, K=G or T, R=A or G, Y=C or T). The difference in the mean mutation count (for age) or in the mean rate (=mutation count/age, for all other exposures) between exposed and unexposed (old versus young for the age signature) is reported for each predictive feature. A) Examples of age signatures. FIG. 23 and Table 8 for the full list. B) Examples of environmental, DNA polymerization or repair, and other factors' signatures. FIG. 24 and Table 8 for the full list. C) Examples of smoking signatures in different tissues.

FIG. 20 shows the tissue dependence of mutational signatures. Heat map of the distances among mutational landscapes of different etiological factors for different tissues. Pearson's correlation was used to calculate the distance. The lower the distance the more similar the corresponding mutational landscapes are.

FIG. 21 shows mutational signatures of obesity in colon (COAD), esophageal (ESCA), kidney (KIRP), and uterine (UCEC) cancer patients. All features of a signature are depicted (IUPAC notations: B=not A, D=not C, H=not G, V=not T, W=A or T, S=C or G, M=A or C, K=G or T, R=A or G, Y=C or T). The difference in the mean mutation rate (mutation count/age) between exposed and unexposed is reported for each predictive feature present in the four mutational signatures for obesity.

FIGS. 22A-22F shows supervised feature engineering. Pictorial representation of the process used for determining the “candidate features”, by going “down and up the tree”, as described in Example 2. Bold line connecting two mutation types indicate statistical testing of significant differences between them.

FIG. 23 shows SuperSigs for age. For each indicated cancer type all selected features of its age signature are listed (IUPAC notations: B=not A, D=not C, H=not G, V=not T, W=A or T, S=C or G, M=A or C, K=G or T, R=A or G, Y=C or T). The difference in the mean mutation count between old and young is reported for each predictive feature.

FIG. 24 shows SuperSigs for environmental and inherited factors. For each indicated cancer type all selected features of a signature are listed (IUPAC notations: B=not A, D=not C, H=not G, V=not T, W=A or T, S=C or G, M=A or C, K=G or T, R=A or G, Y=C or T). The difference in the mean rate (=mutation count/age) between exposed and unexposed is reported for each predictive feature.

FIGS. 25A-25F show unsupervised, random, and supervised methods' comparisons. Comparison of the prediction accuracies (in terms of AUC) are reported for all signatures of age, environmental, and inherited factors, for the unsupervised, the randomly generated single peak signatures, and the supervised methodologies. Logistic Regression (Logit), Linear Discriminant Analysis (LDA), Non-negative Least Square Logit using the Betas (NNLS_Logit_betas), Non-negative Least Square Logit using the means (NNLS_Logit_means), Random Forest (RF), Unsupervised as in Alexandrov et al. (Nature 500, 415-421 (2013)) (Unsupervised), Best NMF, Matched NMF, Signature 1 as in Alexandrov et al. (Nature 500, 415-421 (2013)) (Signature1), and Single Peak (SinglePeak). All comparisons based on apparent AUC except for S4F. See the main text and the Method section for details.

FIGS. 26A-26B show the tissue dependence of the mutational signatures. Heatmaps (overall and for selected etiological factors) of the distance, in terms of correlation, between any two etiological factors' mutational landscapes. Distance not discounted for age (A) and discounted for age (B). The distance between any two mutational landscapes is given by 1—the Pearson's correlation between the two mutational landscapes.

FIG. 27 shows partially-supervised versus unsupervised methods. Performance comparison in terms of AUC for the partially supervised method and the unsupervised one.

FIGS. 28A-28E show model misspecification and the dimensionality issue with the unsupervised method. All selected features of the supervised and unsupervised POL-ε signatures in UCEC-TCGA are listed and their frequencies compared (IUPAC notations: B=not A, D=not C, H=not G, V=not T, W=A or T, S=C or G, M=A or C, K=G or T, R=A or G, Y=C or T). Different plots are provided according to the different numbers of patterns (i.e. rank) unsupervised NMF was required to find: A)-C) correspond to rank=1, 2, and 3, respectively. The larger the rank the greater the difference of the unsupervised signature from the correct supervised one.

FIG. 29 shows betas of SuperSigs for age. For each indicated cancer type all selected features of its age signature are listed (IUPAC notations: B=not A, D=not C, H=not G, V=not T, W=A or T, S=C or G, M=A or C, K=G or T, R=A or G, Y=C or T). The beta of each predictive feature in the logistic regression is reported.

FIG. 30 shows betas of SuperSigs for environmental and inherited factors. For each indicated cancer type all selected features of a signature are listed (IUPAC notations: B=not A, D=not C, H=not G, V=not T, W=A or T, S=C or G, M=A or C, K=G or T, R=A or G, Y=C or T). The beta of each predictive feature in the logistic regression is reported.

### DETAILED DESCRIPTION

The invention will be further described in the following examples, which do not limit the scope of the invention described in the claims.

### EXAMPLES

### Example 1: Supervised Mutational Signatures Predict Tissue-Specific Etiological Factors in Cancer

Determining the etiologic basis of the mutations that are responsible for cancer is one of the fundamental challenges in modern cancer research. Different mutational processes induce different types of DNA mutations, providing “mutational signatures” that have led to key insights into cancer etiology. The most widely used signatures for assessing genomic data are based on unsupervised patterns that are then retrospectively correlated with certain features of cancer. This Example shows that supervised, machine-learning techniques can identify signatures, called SuperSigs, which are more predictive than those currently available. Surprisingly, it was found that aging causes different SuperSigs in different tissues, and the same is true for environmental exposures. SuperSigs associated with obesity were discovered, the most important lifestyle factor contributing to cancer in Western populations.

After evaluating the performance of the current unsupervised signatures, a new supervised algorithm was developed to determine whether it would outperform previously described unsupervised signatures and used it to study patients in whom clinical as well as sequencing information was available. Several new signatures were discovered that were often more strongly predictive of specific etiologic factors than previously described unsupervised signatures.

### An Evaluation of the Current Unsupervised Mutational Signatures

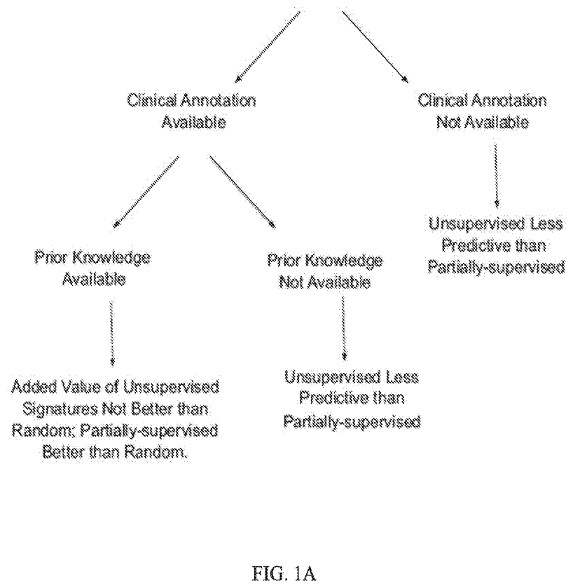

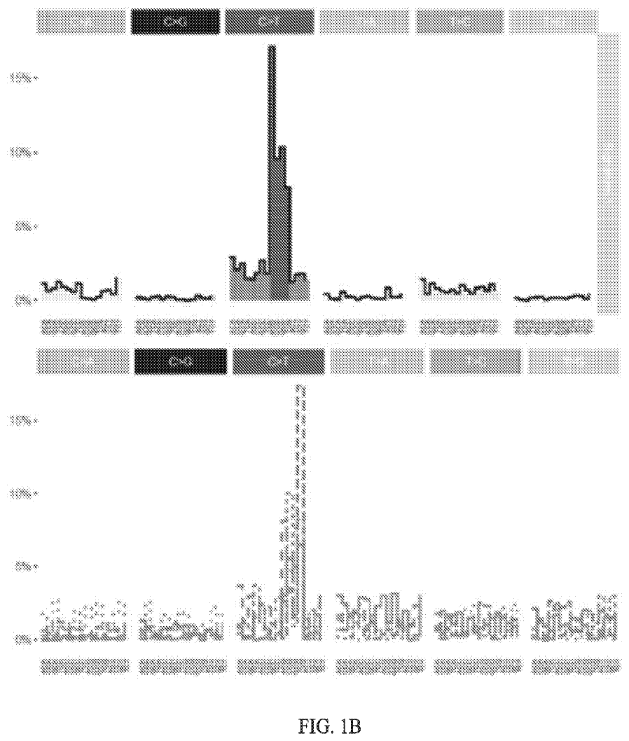

The value of a mutational signature can be assessed by either its prediction accuracy in classifying patients as exposed or not to the associated etiological factor, or by its correlation with exposure to that factor. Therefore, the statistical evaluation of a given mutational signature critically depends on the availability of clinical annotation data for the etiological factor associated to that signature. For example, in the absence of at least one set of patients for whom both sequencing data and smoking status information were available, it would be impossible to assess the value of a given mutational signature for smoking. When clinical annotation is available, it is also important to evaluate to what degree the given mutational signature improves upon some prior validated knowledge on the mutational effects of an etiological factor, if prior knowledge exists, e.g, the deamination at CpG dinucleotides with aging. The current unsupervised mutational signatures (see, e.g., Alexandrov et al., Nat Genet 47, 1402-1407 (2015); Alexandrov et al., Science 354, 618-622 (2016); Alexandrov et al., Nature 500, 415-421 (2013); and Alexandrov et al., Cell Rep 3, 246-259 (2013)) were evaluated in all of these three scenarios (FIG. 1A).

Consider first the case when clinical annotation is available and the main “peak” of a mutational signature, i.e. its most recurrent mutation, is already known before an unsupervised mutational signature is obtained. For example, prior validated knowledge indicated that aging induced [C>T]G mutations, and smoking C>A mutations. The added value of a mutational signature then depends on the extra information that that signature provides beyond the already known peak. This additional information is represented by the distribution of the weights on the other trinucleotides not previously described as significantly enriched by that etiological factor (FIG. 1B). Therefore, to statistically evaluate the added value provided by an unsupervised signature its performance was compared against fully random alternatives carrying no additional knowledge beyond the known peak, for both aging and smoking (FIG. 1B and STAR*Methods (see, e.g., cell.com/star-methods)). These prior knowledge signatures were termed as “randomly generated signatures” because they just add random noise around the already known peaks. This analysis shows that the unsupervised method has a lower accuracy than the randomly generated signature (AUC=0.81 versus 0.84), and a comparable correlation when classifying smoking status in lung adenocarcinoma (Table 1). Similarly, the unsupervised aging Signature 1 has a lower average accuracy than the randomly generated signature when classifying patients in young versus old (AUC=0.58 versus 0.65), as well as a lower correlation (0.14 versus 0.28) with the age of the patients (Table 1). A performance below or at par when compared against a randomly generated pattern implies that the unsupervised approach did not add any relevant information to the prior knowledge. Therefore, with the exception of the already known peak(s), the distributions of the unsupervised smoking and aging signatures across all 96 trinucleotides represent noise and carry no useful information. In contrast, the supervised approach largely increases the prediction accuracy (AUC=0.89 for smoking status and AUC=0.71 for age) and correlation (0.37 for smoking status and 0.38 for age) with respect to the randomly generated signatures (Table 1), implying that the supervised signatures add value—in term of both prediction accuracy or correlation—to the known mutational peaks. The following sections show that when prior knowledge is not available for both the cases where clinical annotation is available as well as when it is not, the supervised approach significantly outperforms the unsupervised one.

Moreover, aberrant results are obtained if the number of patterns selected during the unsupervised step is different from the true number of patterns present; the larger the difference between those two numbers the worse the results (see the “Partially-supervised Method Extension” section in the STAR★Methods for an example).

Supervised Method for Mutational Signatures with Low-Variance Features of Variable Length

Three key features differentiate the new approach to identify signatures from those previously published. First, the machine learning is supervised, i.e. it learns from the data by using the available annotation on clinical variables such as age, smoking status, and body mass index. After a supervised feature selection step, it then uses a supervised classification method—linear discriminant analysis (LDA)—to determine the mutational signatures. Besides classifying samples into exposed or not exposed, this second step provides a score for the evidence of a given exposure in each sample of the test set. This permits comparisons of the intensity of the exposure among different patients.

Second, a pre-determined base length, such as 3-base pairs, was not used as the fundamental unit of the mutational signatures. This provides greater flexibility because there is no reason to assume that all signatures are optimally described by the same base length units. In fact, even the same signature may be defined on units of variable base lengths. For example, a signature may be characterized by significantly elevated proportions of both C>A and A[C>T]G mutations, the former representing a single-base feature and the latter representing a 3-base feature of the signature.

Third, a probabilistic approach was employed to signature discovery. An important characteristic of any mutational process is its randomness. The effects of a mutational process on the genome are stochastic rather than deterministic, with certain mutation types being more probable (i.e. having higher frequencies) than others. Moreover, the mutational distribution caused by the same etiological factor varies greatly among exposed patients: a mutation type very frequent in some patients may not be common in others. From a biological point of view, it seems natural that each patient—and in fact each cell—may have her/his individualized signature characterizing a specific etiological factor. The signatures are therefore built only on selected features that are robust across the exposed population, i.e. features with relatively low variance, thereby increasing their predictive power.

SuperSigs Associated with Aging Vary with Tissue Type

It has long been known that certain types of mutations, such as C>T transitions resulting from cytosine deamination, accumulate with age. It was evaluated whether other mutational signatures of aging were present in cancers and whether they varied among tissue types. For this purpose, sequencing data from thirty types of cancers recorded in The Cancer Genome Atlas (TCGA) database were analyzed. To avoid confounding factors, this analysis was confined to patients without annotated cancer-associated environmental exposures and without known germline predispositions to cancer.

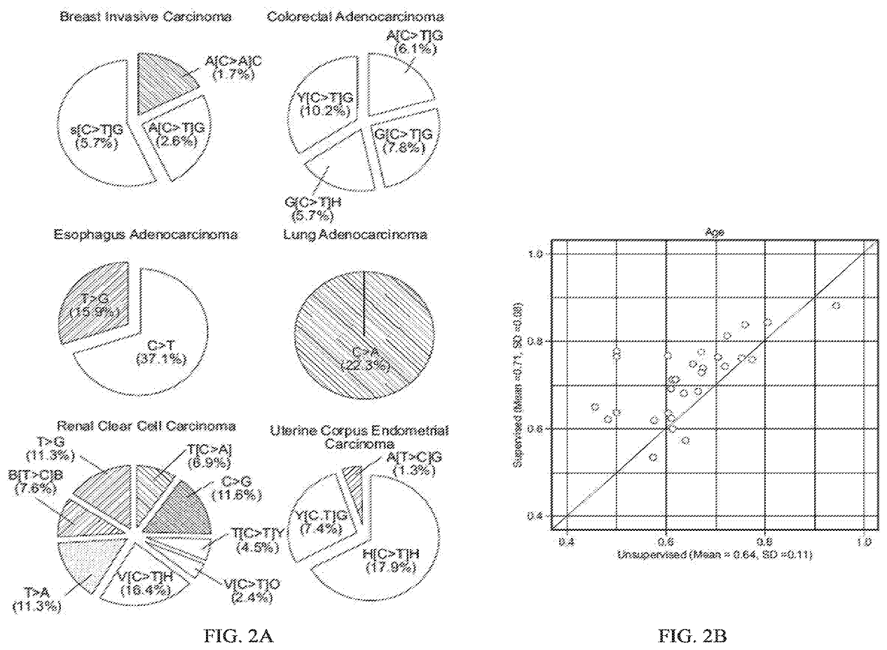

Signatures, which were termed “SuperSigs”, associated with aging in cancers of various types were discovered, examples of which are shown in FIG. 2A. C>T transitions are known to be associated with aging, and not surprisingly were found in large fractions in many of the aging signatures among various cancer types (FIG. 2A). However, others, such as C>A transversions in lung and kidney cancers, had not been described previously as age-associated mutations. Other SuperSigs associated with aging of specific tissues are described in FIG. 10. From this analysis, it is evident that the mutational processes associated with aging vary with cancer type. In fact, it was shown that any two cancer types can be distinguished with very high accuracy (˜90%) simply by their mutational landscape (FIG. 11).

It was then wondered whether patients were “young” or “old” (as measured by the lowest and highest tertile, respectively, of the age distribution) could be predicted from the SuperSigs in their cancers. As depicted in FIG. 2B, the average prediction's accuracy of the age SuperSigs—as measured by the AUC—was 0.71 (s.d.: 0.08) (see Table 2). These predictions, based on different aging processes in different tissues, were considerably more accurate than the average prediction accuracy (0.64; s.d.: 0.11) based on the two age-related signatures common to all tissues that were identified by unsupervised machine learning techniques (see FIG. 2B). The probability that SuperSig predictions were due to chance was 2.7×10⁻⁶ while the probability that unsupervised predictions were due to chance was only p=0.0012. The statistical significance of the predictions of SuperSigs was therefore a thousand fold higher than that of unsupervised signatures.

Supersigs Associated with Environmental and Other Factors Vary with Tissue Type

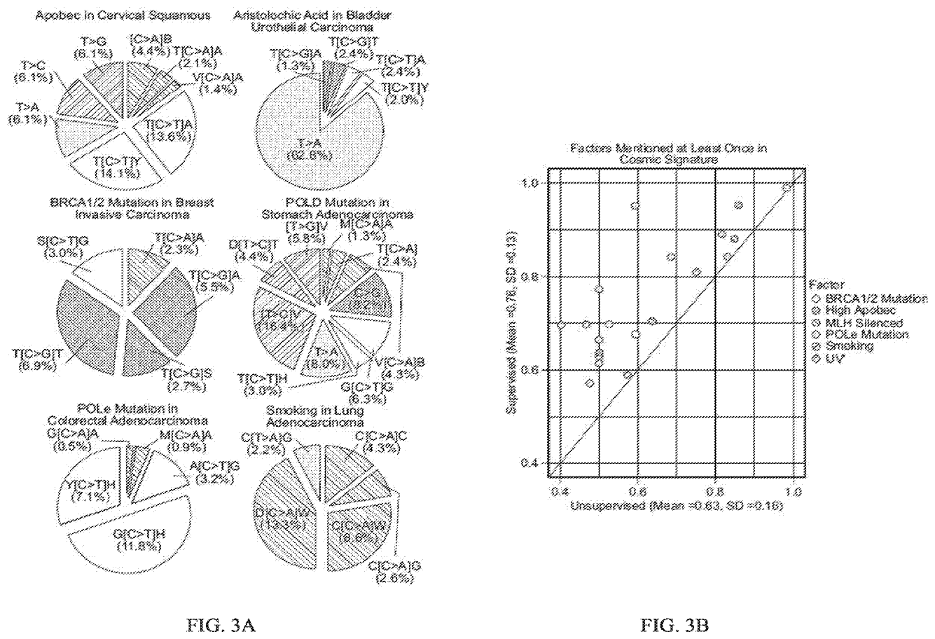

SuperSigs associated with specific environmental carcinogens were next identified. The analysis was performed after controlling for age and for other relevant covariates when available. SuperSigs for smoking, alcohol, hepatitis B and C virus infection (HBV, HCV), aristolochic acid (AA), and ultraviolet (UV) light were obtained (FIG. 3A and FIG. 12). It was also sought to identify mutational signatures associated with defective DNA polymerization or repair, controlling for age, for environmental exposures, and other relevant covariates. SuperSigs were thus obtained for mismatch repair deficiency, mutations in DNA polymerase delta or epsilon genes, mutations in the breast cancer susceptibility genes BRCA1 or BRCA2, methylation of the MGMT gene, and APOBEC (FIG. 3A and FIG. 12). Additional signatures were identified for cancers with low and high chromosome copy numbers and for IDH1 gene methylation (FIG. 12).

In addition to documenting that SuperSigs could be attributed to the factors noted above, whether an individual was exposed to the factor could be predicted simply from the SuperSigs in the individual's cancer genome sequencing data. For example, lung adenocarcinoma (LUAD) patients were able to be classified as smokers or non-smokers with 0.89 prediction accuracy. Similarly, patients with esophageal carcinomas (ESCA) were correctly classified as drinking alcohol more than once per week vs. less than once per week with 0.86 prediction accuracy (FIG. 3B). The average prediction accuracy of supervised signatures was 76% (s.d.: 0.12) (see Table 3). In contrast, the average prediction accuracy of the unsupervised signatures was considerably lower. When restricting the analysis to the same environmental and inherited factors, the method described herein provides an average 0.76 accuracy (s.d.: 0.13), versus an average 0.63 accuracy (s.d.: 0.16) in 20 comparisons. The probability that SuperSig predictions were due to chance was 2.8×10⁻⁷ while the probability that unsupervised predictions were due to chance was only p=0.006 (see FIG. 3B and Table 3). The statistical significance of the predictions of SuperSigs was therefore twenty thousand times higher than that of unsupervised signatures.

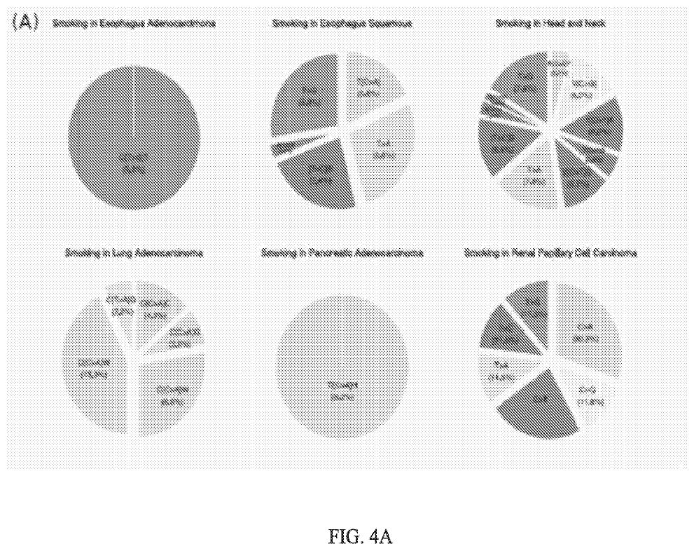

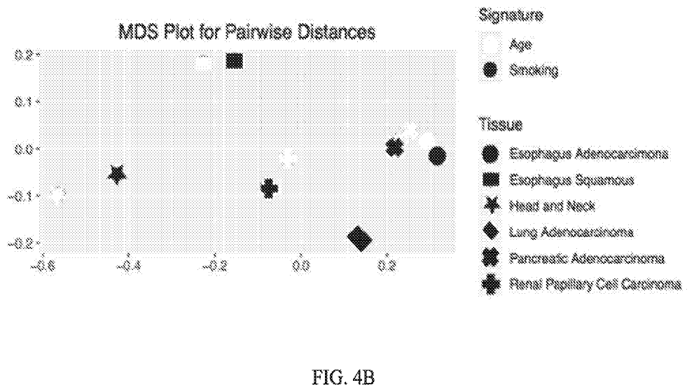

The SuperSigs associated with the same factors generally varied across tissues, just as they did with aging. For example, the SuperSigs associated with smoking were very different in lung, head and neck, pancreatic, and esophageal cancers (FIG. 4A). And the SuperSigs associated with BRCA gene mutations were considerably different between breast and ovarian cancers (FIG. 12). Only a few SuperSigs, such as the ones based on mismatch repair deficiency, did not vary much among tissue types (FIG. 12).

The tissue-specific SuperSigs associated with environmental factors were often similar to the aging signature of the same tissue (FIG. 12). For example, the smoking signatures were more similar to the aging signature of their respective tissues than to each other (FIG. 4B). These analyses then suggest that a major effect of environmental factors is often to simply increase the rate of cell division. Such increases would be linearly proportional to the increase in mutation rate and would not be associated with new signatures such as those caused by direct interaction of carcinogens with DNA. Increases in the rate of cell division are known to occur when tissues are damaged or inflamed (see, e.g., Cheah et al., Proc Natl Acad Sci USA 112, 4725-4730 (2015); and Walser et al., Proc Am Thorac Soc 5, 811-815 (2008)).

### SuperSigs for Obesity

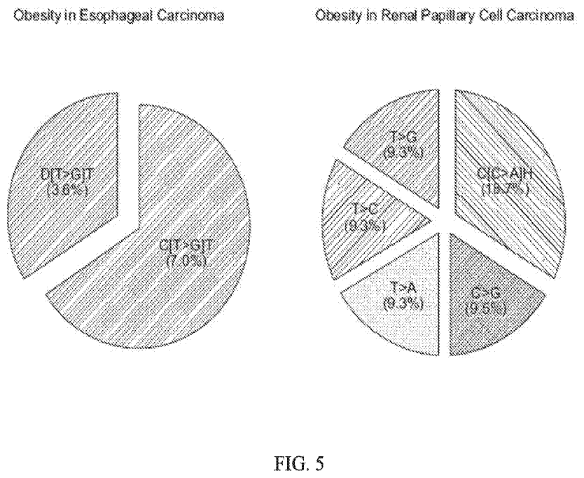

Obesity (as measured by a body mass index, BMI, greater than 30) has emerged as the major lifestyle factor contributing to cancer in general. How obesity contributes to cancer risk, however, is unknown. For example, obesity could lead to cancer by inducing mutations or by stimulating the growth of neoplastic cells that have already acquired mutations. If the former explanation were valid, there might be a mutational signature associated with obesity, but no such signature has been previously identified. Three cancer types associated with obesity in which adequate number of samples and body mass index data for a supervised machine learning approach were available: esophageal, uterine, and kidney cancer. SuperSigs were identify for obesity in two of these three cancer types (FIG. 5). And in cross-validation, which patients were obese was predicted simply by the SuperSigs in their cancers. The prediction accuracy was 0.77 in kidney cancer (kidney renal papillary cell carcinoma—KIRP), and 0.76 in esophageal cancer (ESCA) (FIG. 3B and Table 3). The obesity SuperSigs varied in the two cancer types, again emphasizing the tissue specificity of mutational signatures associated with the same risk factor.

The Proportion of Mutations Due to Aging

Finally, the supervised approach was applied to estimate the proportion of the overall mutational load that can be attributable to normal aging rather than to other mutational processes. When considering all 30 tissues, it was estimated that on average 66% (2.5% quantile: 0.13; median: 0.76; 97.5% quantile: 0.86) of the mutations can be attributable to the normal endogenous mutational processes associated with aging, that is normal DNA replication (Table 4). The proportion varied from 9% in endometrial cancer (UCEC-TCGA) patients with defects in the gene POL-ε to very high percentages like in patients with uveal melanoma (UM) where it was 85%. This estimated proportion is expected to be an overestimate, given the lack of full annotation for all environmental and inherited factors.

### Discussion

The results recorded above lead to several important conclusions. First, supervised machine learning led to new signatures for a variety of etiological factors. These new SuperSigs are better at predicting an exposure than the signatures derived from unsupervised learning.

A second observation is that the SuperSigs usually varied with tissue type. In the majority of previous studies of signatures, it has been assumed that a specific mutational process produces the same signature in all tissue types (see, e.g., Alexandrov et al., Nat Genet 47, 1402-1407 (2015); Alexandrov et al., Science 354, 618-622 (2016); Alexandrov et al., Nature 500, 415-421 (2013); and Alexandrov et al., Cell Rep 3, 246-259 (2013); see, e.g., Blokzijl et al., Nature 538, 260-264 (2016) and Hoang et al., Sci Transl Med 5, 197ra102 (2013) for exceptions). In contrast, the SuperSigs were usually tissue-specific. The fact that the same risk factor, such as alcohol, might give rise to different signatures in different tissues might be viewed as surprising given historical views of exogenous carcinogens such as UV light. However, recent studies have suggested that tissue-specific differences in chromatin organization might underlie the tissue specificity of mutations, at least during aging (Polak et al., Nature 518, 360-364 (2015)). Moreover, the tissue-specific nature of SuperSigs is consistent with the tissue specificity of cancer predisposition syndromes. For example, inherited mutations in the fundamental genes involved in DNA repair or recombination, such as BRCA2, might be expected to result in predispositions to cancers of all types, but they only increase cancer risk in a limited subset of tissues. These results show that the SuperSigs associated with BRCA2 indeed vary with tissue type. Clinical observations like these, together with the SuperSigs described here, support the idea that the nature of mutagenesis is highly dependent on tissue type, and often related to inflammation, suggesting important avenues for future research.

A total of 70 SuperSigs were defined but at most 2-3 of these SuperSigs appear to play a role in any single cancer. This stands in contrast to the widely used signatures discovered through unsupervised learning techniques. Even if only a subset of the unsupervised signatures are considered in the analysis of a given cancer type, there are multiple instances where each of these remaining unsupervised signatures is found in essentially every cancer patient. For example, signature 3, a signature for BRCA1 or 2 mutations, was found in virtually every breast cancer patient sequenced in TCGA (see Figure S32 in Alexandrov et al., Nature 500, 415-421 (2013)), whether the cancer had any relationship to the BRCA pathway or not. Similarly, signature 4, a signature for tobacco smoking, and signature 6, a signature associated with defective mismatch repair mechanisms (MMR), was found in virtually every liver cancer patient (see Figure S43 in Alexandrov et al., Nature 500, 415-421 (2013)), while MMR-deficiency is rare in liver cancers).

An important limitation of this method and of any other method is the quality of the clinical data currently available as well as the limited knowledge of the etiological factors patients are exposed to. There is currently much interest in performing genome-wide sequencing studies on very large numbers of cancer patients in whom clinical data are well-annotated. As such studies proceed, and as the knowledge of etiological fac tors advances, the power of the supervised learning approach described here will progressively increase. It is anticipated that this will lead to accurate estimates of the fraction of mutations attributable to each specific environmental, hereditary, and replicative factor. Conversely, in certain cohorts, this approach could lead to the detection of a sizable fraction of mutations that cannot be attributed to any known source, potentially leading to new insights into pathogenesis, and in particular, avoidable pathogenic agents. The supervised approach can be easily extended to a partially supervised one in order to deal with this situation.

A final conclusion relates to obesity. Obesity is now considered the primary environmental risk factor for cancers in general, and with its increasing incidence, the number of cancers impacted by it is huge (see, e.g., Giovannucci et al., Ann Intern Med 122, 327-334 (1995); Hruby et al., Am J Public Health 106, 1656-1662 (2016); and Song et al., Science 361, 1317-1318 (2018)). Yet the mechanisms underlying the effects of obesity on cancer risk are unknown. Numerous speculations about mechanism have been proposed, such as the effects of putative adipokines and a variety of other hormones or circulating metabolites on cell growth. The discovery of SuperSigs for obesity in some tissues indicates that at least in those tissues part of the risk from obesity may be attributed to mutagenesis. This observation thus leads to specific testable hypotheses that can advance the field. For example, what circulating molecules in obese patients increase the mutation rate, giving rise to the SuperSigs described here?

### Materials and Methods

### Methylation

The hypermethylation and hypomethylation were considered similarly but independently and the unit of analysis is a gene. For hypermethylation, genes that are not included in the PolyComb 27 dataset were filtered out. Also, genes with less than 3 or with more than 7 probes were filtered out for hypermethylation. Now, for each gene in each sample, the percentage of probes that are hypermethylated in the sample was calculate. Based on these percentages, an empirical frequency distribution was generate with the following binning: (0,0.1,0.3,0.5,0.7,0.9,1) with first bin including 0 and the last including 1. The number of genes in each one of the 6 bins was considered as one of the hypermethylation features, for a total of 6 features per patient. The Wilcoxon test was performed to test which features (i.e. bins) are significantly differentially methylated between the two groups of patients (exposed vs not exposed) and keep only the features with an FDR smaller than 0.01. The same process was applied for hypomethylation.

### Gene Expression

Gene expression was used in the standard log 2 scale which spans from 0 to 16. The genes with a median of less 3 or more than 13 among samples in each patient group (exposed vs not exposed) were filtered out. Only genes whose median difference between the two groups is at least 3 were kept. If no genes remain, the threshold was lowered from 3 to the maximum seen over all genes minus 0.5. Among the remaining genes, the significance of differential expression was calculate using the p-value from the Wilcoxon test and adjust it by Benjamini-Hochberg process and only the genes with at most an 0.01 FDR were kept. At most 10 genes were kept if more than 10 genes are significant, and the top 3 genes were kept if less than 3 genes are significant.

### Cross-Validation

10 times 5-fold CV was applied for Smoking in LUAD, Alcohol in LIHC, Smoking in PAAD, high BMI in UCEC, Smoking in KIRP, high BMI in KIRP, HepB in LIHC, HepC in LIHC with accuracy as the following:

| Exposure (Tissue) | AUC |
|---|---|
| SMOKING (LOAD) | 0.73 |
| ALCOHOL (LIHC) | 0.78 |
| SMOKING (PAAD) | 0.59 |
| BMI (UCEC) | 0.68 |
| SMOKING (MRP) | 0.46 |
| BMI (KIRP) | 0.47 |
| HepB (LIHC) | 0.59 |
| HepC (LIHC) | 0.65 |

### Data Preparation and Integration

Somatic exomic mutational data was downloaded from the TCGA Bioportal (portal.gdc.cancer.gov) and filtered out the mutations which have less than 5% Variant Allele Frequency (VAF). Out of the total thirty-three datasets available, large B-cell lymphoma (DLBC) was not included in the analysis because of the small number of samples available, while lung squamous cell carcinoma (LUSC) and mesothelioma (MESO) were excluded because of the extremely small number of patients unexposed to smoking and asbestos, respectively. For ovarian cancer (OV) and acute myeloid leukemia (LAML) whole genome sequencing data were used. The human genome reference build hg38 was used to determine the context (flanking bases) for each mutation. The clinical information was downloaded from the website Cbioportal (cbioportal.org). For calculating the background frequency of each trinucleotide on both the exome and the genome the R package, deconstructSigs was used. For the “Unsupervised Signature” method, the signatures were downloaded from the Cosmic Signature website (cancer.sanger.ac.uk/cosmic/signatures) and used the table cancer.sanger.ac.uk/signatures/matrix.png in order to determine which signatures were present in which tissue. The following method was used to assess the unsupervised signatures: to determine in a given patient the respective proportional contributions X of each mutational signature i=1, . . . , k, where a total of k signatures were present in that tissue, non-negative least square (FCNLS) was applied as in Alexandrov et al. (Nature 500, 415-421 (2013)) to

Yⱼ=Aⱼ₁X₁+Aⱼ₂X₂+ . . . +AⱼₖXₖ

i.e. Y=AX in matrix form, where Yⱼ is the total number of mutations of type j=1, . . . , 96, normalized so that ΣYⱼ=1 in that patient, and Aⱼᵢ is the relative frequency of mutation type j in the mutational signature i, across each one of the k signatures present in that tissue.

All analyses were performed using R version 3.5.2. LDA was performed using the function lda from the package MASS. Logistic regression was performed using glm from the STATS package. Non-negative matrix factorization (NMF) was performed using the function nmf with method “Lee” from the package NMF.

### Filtering of the Samples

To reduce the effect of confounding factors, a filtering scheme was applied as follows. In each tissue type, samples were divided into two main categories: 1) “unexposed”, meaning that based on the available clinical annotation, no known environmental factor was believed to have contributed to the development of the cancer (we treated NA environmental factors as unexposed), and 2) “exposed”. To mitigate the effects of other unknown factors in the unexposed group, any sample with a mutational load more than 3 times higher than the median number of mutations found among the unexposed samples was removed. Samples were also excluded if the total number of mutations was equal to zero on the exome, a probable indication of low neoplastic cell content. In general, samples with a mutation in POLE/POLE2/POLE3/POLE4 or POLD1/POLD2/POLD3/POLD4 genes were removed—except for when the signature for the specific effects of those mutations was the objective of the analysis. A tissue type was divided into subtypes whenever possible. Acute Myeloid Leukemia (AML) patients younger than 40 years old were not considered. Among the “exposed” samples, samples with known multi-factor exposures were excluded to minimize confounding factors and only evaluated samples with a single known exposure. For the age analysis, the unexposed samples were divided into three groups (younger, middle-aged, older), and eliminated the middle group before training the algorithm. When testing the algorithm, those two age groups were also considered.

### Comparison of Performance Between Unsupervised Signatures and Randomly Generated Signatures

To assess the value of the aging (#1) and smoking (#4) unsupervised signatures in Alexandrov et al. (Nature 500, 415-421 (2013)) beyond their main “peak”, i.e. C>A for smoking and [C>T]G for aging, since those peaks were already known. Thus, the value that the unsupervised signatures add to the previously known mutational peaks was evaluated. This essentially corresponds to evaluate if the part of the distribution of an unsupervised mutational signatures that is not the mutational “peak” adds any value to the peak, according to some measure of performance (prediction or correlation).

To do this, a “randomly generated smoking signature”, a signature for smoking in LUAD, was defined whose only property is a higher proportion of C>A mutations than the other mutation types and where, beside this “peak” at C>A, the proportion of all the other mutation types is assigned randomly. Similarly a “randomly generated aging signature”, a signature for aging, was defined whose only property is a higher proportion of [C>T]G mutations than the other mutation types and where, beside this “peak” at [C>T]G, the proportion of all the other mutation types is assigned randomly. This was done in two alternative ways: (i) generating the random signature using random samples or (ii) building a “randomly generated signature” from a uniform distribution. Specifically, for the smoking signature:
(i) To generate a randomly generated smoking signature by random samples, 30 samples out of all smokers and never-smokers were randomly sampled. the samples whose C>A portion is at least as high as 0.9 of the maximum proportion of C>A observed were filtered. Then, the “randomly generated smoking signature” is the one among the filtered sample with the minimum proportion of C>T substitutions. Non-negative linear regression was applied to calculate the effect of this signature.
(ii) To generate a randomly generated smoking signature by random distributions, the signature was generated in a two-step process. In step one, 30 probability distributions were generated over the six main mutation types (which lack suffix and prefix base) as follows. For each distribution, 6 numbers were generated from a uniform distribution and divide them by their sum. As in (i), only the samples whose C>A proportion is at least as high as 0.9 of the maximum proportion of C>A observed were kept. The “randomly generated smoking signature” using a random distribution is then the filtered sample with the minimum proportion of C>T substitutions. In step two, the obtained proportion of each of the six main mutation types were randomly broken down into the 16 fundamental mutations which form each of the six main mutations.

After obtaining these randomly generated signatures, the contribution of the random signature was calculated by applying non-negative linear regression. Thereafter, to evaluate the performance of the signature, the Area Under Curve obtained was calculated using the contribution (normalized by total number of mutations) of the randomly generated smoking signature to predict smoking status, as well as its Spearman correlation with the number of packs smoked by the person.

A similar process was applied to the age signature using the sequencing information of unexposed tissues only and it was compared with the performance of Signature 1 in Alexandrov et al. (Nature 500, 415-421 (2013)). The process was modified in three simple ways. It was assumed that the main types of mutations are: [C>T]G, [C>T]H, C>A, C>G, T>A, T>C, and T>G. Also, in the selection among the 30 signature candidates, only the samples whose [C>T]G proportion is at least as high as 0.9 of the maximum proportion of [C>T]G observed were kept. The randomly generated aging signature using random distribution is then the filtered sample with the maximum proportion of C>T substitutions. As usual, for age the contributions were not normalized by the total number of mutations.

### Supervised Feature Engineering

All six types of possible substitutions were considered, with or without the context bases flanking those substitutions, as potential features. These features have variable length and can be grouped into 3 categories. The first category, composed of single nucleotides, contains only the six types of possible substitutions, regardless of the bases before (prefix) or after (suffix): C>A, C>G, C>T, T>A, T>C, and T>G, where all substitutions are referred to by the pyrimidine of the mutated Watson-Crick base pair. The second category, composed of dinucleotides, includes 48 substitutions with a specific base as a prefix or as a suffix (e.g. A[C>T] and [C>T]G); there are 24 with a prefix and 24 with a suffix. The third category, composed of trinucleotides, includes 96 substitutions with both a prefix and a suffix (e.g. A[C>T]G or G[C>T]G). Finally, the total number of mutations, Tot, was considered as a feature. Hence, there was a list of 151 potential features (6+48+96+1). These features construct a partitioning tree. In other words, the total number of mutations found in a sample can be seen as the root of all mutation types, and it is partitioned into mutations of the first category as its children, i.e. substitutions with neither prefix or suffix (e.g. C>T). Each mutation in the second category is the child of one in the first category (e.g. [C>T]G and A[C>T] are both children of C>T) and each third-category mutation is the child of two parents of the second category (e.g. A[C>T]G is the child of both [C>T]G and A[C>T]). Importantly there is dependence among features found on the same path when moving along this tree from the root to the leaves. The way this dependence was dealt with is described in the next section.

If the number of training samples were below a threshold (60 unexposed samples or 15 exposed samples), or if the median total number of mutations was <20, only a subset of the 151 features was considered. This subset was composed of 6 features: the first category of mutations (single nucleotides) and the total number of mutations. The reason for this is that it was assumed that the signal/noise ratio would be too low to determine whether second category (dinucleotide) or third category (trinucleotides) context mattered.

For each feature, it is possible to consider its absolute count or its relative frequency (its absolute count divided by the total number of all mutation types). In a patient exposed only to “aging”, i.e. unexposed to any known environmental or inherited factor, the relative frequency of a mutation type is expected to remain constant irrespective of age—as dictated by the aging signature—while the absolute count is expected to increase with age. In contrast, in a patient exposed to an environmental or inherited factor, the relative frequency of a mutation type as well as the count may change with age. Thus, absolute counts were used for determining age signatures, while one analysis was performed using relative frequencies and another one using absolute counts for all other signatures. The results of these two separate analyses were often comparable, except in terms of prediction accuracy where absolute counts often have an advantage, as expected. Thus, the results were reported using relative frequencies to be conservative. To improve accuracy, a log transformation was applied to count features, which is a standard tool in these types of analyses.

Next, it was aimed to purge unrelated or low signal/noise mutation types out of the total 151 potential features. As mentioned, there is a hierarchy among the mutation types, with parents, children, grandchildren, etc. along the partitioning tree. In general, not all 151 potential features of this tree will have counts that are significantly different from what is expected by chance after controlling for their representation on the exome. For each tissue and for each exposure, it was started from the root of the tree and “went down the tree” to find features whose counts are significantly different from those expected. Specifically, the null hypothesis was that there is perfect dependence among the potential features found on the same path when moving along the tree from the root to the leaves. Unless proven otherwise, the count of a given feature could be explained by the count of any of its parent(s), or more precisely of any of its ancestors, after adjusting for its expected representation in the exome. As an example, the null hypothesis for the total number of observed C>T mutations was that this number would be equal to its expected value, which is given by the total number of mutations observed, Tot, adjusted for the normal frequency of the “C” nucleotide on the exome (vs the “T”s), and the fact that there are three equally probable mutation types (i.e. C>A, C>G, and C>T) under the null. Thus, since C (i.e. C:G) nucleotides have a frequency of 0.506 on the exome (0.409 on the genome), then the expected value of C>T mutations on the exome would be given by Tot*0.506*⅓, since it was assumed a priori that a C has the same probability to mutate to an A, a G, or a T. As another example, [C>T]G, which is the child of C>T and the grandchild of the total number of mutations, would be tested twice to see if it significantly exceeded its expected number based on the total number of mutations as well as the number of C>T. Thus, the expected value of [C>T]G mutations would be given by Tot*0.506*⅓*X, where X is the expected frequency of CG out of all C nucleotides in the exome, as estimated by deconstructSigs.

To test each hypothesis, a one-sided binomial test was applied at a 0.05 significance level with a Bonferroni correction for 151 tests to control for multiple testing. The binomial test was based on the sum of the total number of mutations observed for that potential feature across all training samples, and the probability of success was set equal to the frequency of that potential feature, as expected by its representation on the exome. If the null hypothesis was rejected, that potential feature was selected as a “first-phase” candidate feature for the next supervised selection step.

Once a temporary list of candidate features had been selected, this list was updated and pruned by “going up the tree” by testing parents that had children that had also been selected. Indeed, some parent mutations may have been selected only because their children had higher than expected frequencies. In other words, the parent was tested by removing the contribution of the selected child to see if the count/frequency of the leftover in that parent would still be significantly higher than expected by chance. If it were, then that parent remained in the list of first-phase candidate features but only after having subtracted the contribution of the first-phase candidate feature child. If not, the parent was eliminated as a feature in that particular analysis. The feature was named “remaining mutations”—when significant—containing the leftover of the total number of mutations. The list of features that remained after this second selection were termed “second-phase candidate features”.

For every factor other than age, the above feature-engineering step was applied separately to samples from patients that were respectively unexposed or exposed to the factor under consideration. It was then combined these two lists of second-phase candidate features by considering the new partition formed by all intersections and relative complements of the elements in the original two partitions, i.e. the two original sets of second-phase candidate features. This new partition is the smallest refinement of the two original partitions (see also Table 4). When completed, this process provided the final list of candidate features.

For aging signatures, the feature engineering steps described above were applied only to samples from patients who were unexposed to any known environmental or inherited factor. This is because the age signature is not expected to change with aging, but simply to increase in its intensity in terms of mutation counts. The resulting second-phase candidate features constituted its “candidate features” list.

### Supervised Feature Selection and Signatures

Once the list of candidate features was obtained, they were ranked using a bootstrap t-statistic with pooled variance for each class (young vs old, or unexposed vs exposed to an H or E factor) with 1000 iterations in the training set. For the analysis of absolute counts, features with negative median t-statistic were purged, in light of the biologically reasonable assumption that samples from older/exposed patients should not have a lower absolute count of a given mutation type than younger/unexposed patients. For the analysis of relative frequencies, features with negative median t-statistic were instead kept. The larger the absolute value of the t-statistic, the larger the evidence that the feature was affected by the tested variable (i.e., aging or some exposure). To stabilize the ranking of the features, first, second, and third category features were penalized by subtracting a penalty from the median t-statistics according to the following formula:

$$Penalty for feature i = \frac{{log}_{2} ( \frac{96}{\# of trinucleotides in feature i} )}{2 {log}_{2} ( 96 )}$$

This penalty function was chosen a priori, and not optimized in cross-validation. The penalty increases as features are further down the tree, with the largest penalty (0.5) being assigned to features of the third category, i.e. trinucleotides. features that had a t-statistics >3, or in cases where the signal was weak (i.e. when all candidate features had a t-statistics <3), all features with a t-statistic within 0.5 of the top feature, were then selected. Again these values were chosen a priori, and not optimized in cross-validation. The set of these selected features constitute what were defined as mutational signatures and were used in the next step for prediction. The mutational signatures for each factor (aging or exposure) are depicted in FIGS. 10 and 12.

### Prediction: LDA and Logistic Regression

The significance of the signatures can be assessed by their ability to distinguish between groups of patients, i.e. exposed vs unexposed, or younger vs older patients. Thus, after the feature selection step, two alternative classifiers—using two types of distribution families—were used to test the predictive accuracy of each mutational signature: linear discriminant analysis (LDA) and logistic regression (Logit). Both methods yielded very similar results, and the results of LDA are reported.

In LDA, a multivariate normal distribution is used to model the features' mutational frequencies of a group of patients, with a mean vector equal to the empirical mean vector and a covariance matrix for the dependencies among the features. In logistic regression, the maximum entropy distribution is instead used to model the features' mutational frequencies in a group of patients, where the constraint on the maximum entropy distribution is that the expected value of each feature is equal to that of its observed average. In information theory language, features modeled by a maximum entropy distribution have minimum information about each other. For both families of distributions, the log ratio test was then used.

In FIGS. 10 and 12, the signatures are represented by the average proportion of each selected feature among the samples of that phenotype. For age, the average proportion of each selected feature among all unexposed samples regardless of age status (i.e. young, middle-aged, old) was used. The information for the full distribution of each feature in each group of patients is instead provided in Table 6.

To compare the accuracy of the supervised and unsupervised methods, the area under the ROC curve (AUC) was selected. The results are presented in FIGS. 1B and 2B, and the values are reported in Tables 1 and 2. Ten times balanced 5-fold cross-validation were used to assess the robustness of the prediction accuracy. The cross-validated results are shown in FIG. 13. Note that no cross-validation was performed for the unsupervised method, and so the AUC for the unsupervised method in FIG. 13 is not cross-validated but apparent. A p-value was assigned to the average AUC for both supervised and unsupervised accuracies. Each AUC for a specific tissue, under the null, can be approximated by a normal distribution with mean 0.5 and with a standard deviation equivalent to that used to approximate the variance in the Wilcoxon-Mann-Whitney test, which is a function of just the sample sizes of two phenotypes. Moreover, since the average of many independent normal distribution is a normal distribution, the average of multiple AUCs can be approximated by a normal distribution with mean 0.5 and variance equal to the sum of the variances for each AUC divided by the square of the number of AUCs. Such combined variance for the 20 datasets compared was 0.0024. The final p-value can be calculated as the upper tail probability of the aforementioned combined normal.

If prediction accuracy were to be the only goal of the analysis, then other methods other than LDA and logistic regression, like for example Random Forest (RF), could be applied to achieve even higher accuracy (e.g. RF has an average 0.83 accuracy for the environmental and inherited factors' signatures, vs. 0.76 with LDA). At the same time, the results obtained with methods like RF are difficult to interpret in terms of the quantitative relationship among the selected features. However, there may be applications where accuracy is indeed the only goal.

### Projection of Mutational Signatures on a Common Refinement Partition

When comparing the signatures of two different exposures a problem is that lack of common features, or at least the lack of perfect overlap between the two sets of selected features contained in the signatures. For example, Exposure 1, may have as selected features [C>T]G, [C>T]H, and the remaining mutations, with proportions 15%, 5%, and 80% respectively, while Exposure 2 may have A[C>T], B[C>T], and the remaining mutations, with proportions 3%, 7%, and 90%. As mentioned, the combination of the two lists is provided by a new partition formed by all intersections and relative complements of the two original partitions, i.e. the two original sets of features. This new partition is the smallest refinement of the two original partitions. In the example, this refinement will contain the following features: A[C>T]G, B[C>T]G, A[C>T]H, B[C>T]H and the remaining of mutations (Table 5).

When “projecting” signatures of Exposure 1 and Exposure 2 onto the new partition uniform distribution of the number of mutations within each feature was assumed. In the example, probabilities were assigned to A[C>T]G, B[C>T]G, A[C>T]H, B[C>T]H, and the remaining mutations, i.e. every mutation except the 4 listed (Table 5). The proportion of a selected feature in a given signature represents the value assigned to that feature in that signature. By assuming a uniform distribution a signature can easily be projected onto any desired refinement partition. See Table 5 for a depiction of this assignment.

### Estimation of the Proportion of Mutations Due to Aging

To estimate the proportion of mutations due to aging in each specific sample, the median rate of mutations per year in the patient population of the corresponding cancer type and in the absence of any known environmental or inherited factor as first estimated. Then the frequency of each feature present in the cancer-specific supervised age signature was multiplied by that yearly mutation rate and by the patient's age of that specific sample. The number obtained by summing the above counts for each feature in the age signature is then divided by the total number of mutations observed in that sample. This resulting ratio, being forced to be not greater than 1, is the estimate for the proportion of somatic mutations attributable to age in that sample.

### Partially-Supervised Method Extension

One limitation of a supervised approach is that it cannot be applied to find signatures of factors for which no annotation is currently available. It may indeed be desirable to have a method that is able to discover patterns of exposures, even when they are unknown. This limitation, however, can be overcome by using the supervised step, already described, and following it with an unsupervised one. That is, all exposures with available annotations can be taken advantage of to discover their supervised signatures. After learning those signatures, the effects of those supervised signatures can be “subtracted” from the mutational load of the patients exposed to those annotated factors. An unsupervised analysis, such as non-negative matrix factorization (NMF), can then be performed on the leftover, to investigate the presence of further mutational patterns.

This Example provides an example of how the supervised learning of a mutational signature (specifically the aging signature in this example) can be used to improve the performance of an unsupervised approach by discounting the effects of that supervised signature on the test data (this methodology is referred to herein as “partially supervised”).

To simplify matters, features were not engineered; rather, the 96 fundamental mutations as in Alexandrov et al. (Nature 500, 415-421 (2013)) were used. Only the datasets that show a higher average rate of mutation per year in the exposed samples than in the unexposed samples were used. This increase in the rate is required to conform to the premise of non-negativity and linearity in the NMF model. One half of the unexposed samples were use as the training set to learn the age signature (thus a supervised signature) and to estimate the mutation rate (number of mutations accumulated per year of age) so that the effect of age on the test set can be discounted. Next the test set was formed by bootstrapping over the left-out half of the unexposed samples and all exposed ones.

NMF (Lee et al., Nature 401, 788-791(1999)) with rank equal to 3 was applied to decompose the test set, thus obtaining two matrices: one containing the unsupervised signatures and a second one with the corresponding contributions of each of those signatures in each patient. These contributions have not been discounted for age yet. This is the standard unsupervised approach. However, in order to estimate the discounted contributions of a signature in each test sample, the effect of age of a patient on each unsupervised signature was now discounted, by multiplying the learned supervised age signature by the age of the patient, times the estimated mutation rate, and then projecting this vector onto the directions identified by NMF using Non-negative Linear Regression, and then subtracting these projected contributions of age from the contributions of the 3 unsupervised signatures obtained by NMF. To conform with premises of NMF, the negative discounted contributions were set to zero.

The direction whose contribution, divided by the total number of mutations, is the most associated (in terms of the highest AUC) to the exposure status using the known ground-truth, for both the unsupervised and the partially supervised methods, by using the not discounted and discounted contributions, respectively, was chosen. The area under the curve was then used to evaluate the association of the signature with the exposure status, where the contribution of each signature has been divided by the number of total mutations.

This whole process (from the random selection of half of the unexposed patients used to learn the age signature and so on) was repeated 50 times, and the average AUC over them was taken to account for the effect of randomness. This is what is depicted in FIG. 14, where the increase in performance of the partially supervised method with respect to the unsupervised is evident.

These discounted contributions are then averaged. This is what was defined as the partially supervised signature and their contributions. Finally, to obtain the “partially supervised signatures” Non-negative Linear Regression was used again but this time where the coefficients are known and the signatures are unknown. In other words, the decomposition M=SC was still used. Originally, M and S were known and C was wanted. Now, M and C are known and S is wanted. This way the contributions stay the same.

For another example, pretend no annotation for the presence of defects in the gene POL-ε among patients with endometrial cancer in the UCEC-TCGA dataset and no known POL-ε signature. Also assume a supervised aging signature for that tissue, as shown in FIG. 2A. Based on the age of each patient in the UCEC dataset the amount of the aging signature present in each patient for each mutational feature can be estimated and the corresponding mutational load can be subtracted. Specifically, the mean count of a given feature attributed to age (young, old) was subtracted and estimated from the training samples. If the feature becomes negative after this subtraction, that feature was set to zero. This yields a “left-over” non-negative matrix that can then be decomposed via the classic NMF. The normalized results for this decomposition are depicted in FIG. 15A. This figure shows the striking similarity of this unsupervised pattern with the known POL-ε supervised signature (compare FIG. 15A with FIG. 12). In particular, the high frequency of T[C>A]T mutations is easily detected in the signature by NMF. Thus, the partially-supervised approach is able to find signatures even for factors for which annotation is not available.

Though the example described above is informative about the power of the semi-supervised approach, at least when the signal is very strong as in the case of a POL-ε mutation, it also illustrates a critical weakness of unsupervised approaches in general. The POL-ε signature in FIG. 15A was obtained by “telling” NMF to search for one (i.e. rank=1) pattern. For two or three signatures, respectively, NMF would have returned the patterns depicted in FIG. 15B-C. FIG. 15B-C show that the POL-ε signature has been parsed into multiple patterns: the more patterns the more the optimum signature is spread across different claimed signatures. Therefore, the quality of the results of NMF strongly depend on the number of signatures NMF is required to extract. Unfortunately there is no fully satisfactory rule to determine a priori how many patterns should be found by NMF. This is a problem that all unsupervised approaches have because the researcher is blind to the actual number of different exposures that are present among the patients in the dataset during the discovery phase. In some cases, after the supervised step, the distribution of mutation types can be considered without using NMF at all. This distribution in the example noted above, obtained the pattern depicted in FIG. 15D, which is again strikingly similar to the known supervised POL-ε signature.

### Example 2: Supervised Mutational Signatures for Obesity and Other Tissue-Specific Etiological Factors in Cancer

Determining the etiologic basis of the mutations that are responsible for cancer is one of the fundamental challenges in modern cancer research. Different mutational processes induce different types of DNA mutations, providing “mutational signatures” that have led to key insights into cancer etiology. The most widely used signatures for assessing genomic data are based on unsupervised patterns that are then retrospectively correlated with certain features of cancer.

This Example shows that supervised machine-learning techniques can identify signatures, called SuperSigs, that are more predictive than those currently available. Surprisingly, it was found that aging causes different SuperSigs in different tissues, and the same is true for environmental exposures. SuperSigs associated with obesity, the most important lifestyle factor contributing to cancer in Western populations, were discovered.

As demonstrated herein, a supervised algorithm has been developed to determine new mutational signatures, termed “SuperSigs”. It was then demonstrated that these supervised signatures could outperform previously described unsupervised signatures in predicting the presence of various etiological factors in patients for whom both clinical and sequencing information was available.

Supervised Method for Mutational Signatures with Low-Variance Features of Variable Length (SuperSigs)

To obtain SuperSigs signatures, sequencing data from thirty types of cancers recorded in The Cancer Genome Atlas (TCGA) database were analyzed. Four key features distinguish the approach for identifying signatures.

1) A primary methodological step is to use supervised machine learning, i.e. learn the signatures from the data, by using the available annotation on clinical variables such as age, smoking status, and body mass index. By using this information explicitly, stronger associations can be identified and better predictions can be made.

2) A pre-determined base length, such as 3-base pairs, is not specified as a fundamental unit of the mutational signatures. This provides greater flexibility because there is no reason to assume that all signatures are optimally described by the same base length units. In fact, a single signature may be defined on units of variable base lengths, featuring, for example, significantly elevated proportions of both C>A (i.e. a single-base substitution from C to A) and A[C>T]G (i.e. a single-base substitution from C to T with flanking bases A and G) mutations.

3) A probabilistic approach to signature discovery was employed. An important characteristic of any mutational process is its randomness. The mutational distribution caused by the same etiological factor varies greatly among exposed patients: a mutation type very frequent in some patients may not be common in others. From a biological point of view, it seems natural that each patient—and in fact each cell—may have her/his individualized signature characterizing a specific etiological factor. The signatures are therefore built only on a subset of selected features that are robust across the exposed population, i.e. features with relatively low variance, thereby increasing their predictive power.

4) There is no assumption that a given mutational process must have the same mutational signature across tissues, contrary to the approach developed by Alexandrov et al. (Nature 500, 415-421 (2013)) where a given signature (e.g. signature 1) is the same across all tissues.

The method for deriving mutational signatures is based on several steps. First, a nested tree containing all potential features was constructed, with all mutations as the root, and all six single-base substitutions (C>A, C>G, C>T, T>A, T>C, and T>G) as the first level, followed by single-base substitutions with one flanking base as the second level, and by single-base substitutions with two flanking bases as the third level, and where the edges are placed between features which share mutations (FIG. 16). In principle, the method can be applied to a tree with height greater than 3, by adding additional flanking bases, but here for simplicity and for comparing with current methods, only three levels were considered.

After “pruning” the tree in order to keep only the features that have counts significantly different from their expected values, these remaining features are ranked based on their ability to classify a given exposure, i.e. to discriminate exposed patients from unexposed ones, as measured by the area under the receiver operating characteristic (ROC) curve (AUC). The set of n top features that provide the highest prediction performance in terms of AUC form the signature for a given exposure and are used for prediction (FIG. 16).

The value of a mutational signature can be assessed by its prediction accuracy (AUC) in classifying patients as exposed or not to the associated etiological factor, or by its correlation with exposure to that factor. Statistical evaluations were provided for both, relying on the availability of clinical annotation for the etiological factor associated to that signature (FIG. 17A).

Mutational Signatures Add to Prior Knowledge about Etiologic Factors

In addition to simple performance, it is also important to evaluate the degree to which a given mutational signature improves upon prior knowledge about the mutational effects of an etiological factor (FIG. 17A). For example, consider the case when clinical annotation is available and the main “peak” of a mutational signature, i.e. its most common mutation, is already known before the mutational signature is obtained. The peak may be a nucleotide, a dinucleotide, or a trinucleotide, depending on the specific mutational process. For example, prior validated knowledge indicated that aging induces [C>T]G mutations, and smoking induces C>A mutations. The added value of a mutational signature then depends on the extra information that that signature provides beyond the already-known peak. This additional information is represented by the “left-over” distribution obtained once the peak is removed, i.e. the distribution of the weights of the other trinucleotides not previously described as significantly enriched by that etiological factor.

To statistically evaluate the added value provided by the signatures of Alexandrov and colleagues, hereafter termed “unsupervised”, as well as of the SuperSigs, both of their performances were compared against random alternatives carrying no additional knowledge beyond the known peak, for both aging and smoking. These prior knowledge signatures were termed “random” because they just reflect random noise around the already known peak (FIG. 17B). Such random signatures are of course only meaningful when there is a peak that is already known and cannot be meaningfully constructed without prior knowledge.

Sequencing data for thirty tumor types were obtained from the TCGA Genomics Commons. After splitting each dataset randomly into training and test partitions, the method above was applied to derive signatures of aging and smoking in the training data, evaluating performance in the test data. The SuperSigs aging signatures were applied to classify patients in a binary fashion (i.e., young versus old) yielded a median AUC of 0.72, calculated over 30 tumor types, significantly outperforming the random aging signature (single peak; median AUC=0.65), which was built on the well-supported observation that over time, cytosines will consistently deaminate to thymine in the CpG context (FIG. 18A, FIG. 25, Table 9). When the signatures are used in a regression setting, to predict age as a continuous variable, the median correlation for SuperSig predictions was rho=0.37. The analysis on the same data yielded a median AUC=0.58, and rho=0.25, for the unsupervised aging Signature 1 (FIG. 18A, FIG. 25, Table 9). The combination of the “clock-wise” unsupervised Signatures 1 and 5 performed slightly better (median AUC=0.64), although it did not improve on the random signature (FIG. 25, Table 9). Unsupervised signatures for aging were not present in four of the tissues, while all tissues had aging SuperSigs.

The performance of these signatures was next evaluated with respect to smoking status across eight tissues known to be significantly affected by smoking. The SuperSigs added value to prior knowledge while the unsupervised signatures did not (median AUCs for smoking: SuperSigs=0.88, single peak=0.57, unsupervised=0.56) (FIG. 18B FIG. 25, and Table 9). The correlation with smoking packs of the SuperSigs was much higher than the one obtained using the unsupervised smoking signatures (0.55 versus 0.23, respectively). These results were confirmed with cross-validation, and even when forcing on the SuperSigs the same prediction method, non-negative least squares (NNLS) (FIG. 25 and Table 9).

These data do not indicate that unsupervised signatures for aging and smoking are meaningless. However, the data indicate that the unsupervised signatures do not add any information to prior knowledge of a peak at [C>T]G for aging and at C>A for smoking. Optimally, an algorithm based on genome-wide cancer genomic sequencing data should add information that was not available from prior studies, and SuperSigs indeed added such information that goes beyond the previously known mutational peaks (FIG. 17A).

### Other Comparisons Between Supervised and Unsupervised Signatures

Supervised signatures perform better than unsupervised ones when no prior knowledge about an etiologic factor is available (second scenario in FIG. 17A). For those factors (other than age) which could be evaluated by unsupervised methods, the median AUC of the unsupervised method was 0.77, while the median AUC for SuperSigs was 0.99 (FIG. 18C-18D, FIG. 25, and Table 9).

The method can predict whether an individual patient was “exposed” to a given etiologic factor simply from the SuperSigs in that patient's cancer genome sequencing data. For example, the cross-validated AUC was 0.95 when classifying patients with lung adenocarcinomas (LUAD) as smokers versus never-smokers. Similarly, the AUC was 1.0 when classifying patients with head and neck cancers (HNSCC) as drinking alcohol more than once per week vs. less than once per week

When clinical annotation is not available for an etiologic factor (FIG. 17A), the unsupervised method may appear to be the only viable approach. However, a “partially-supervised” extension of the method is provided and again it was shown that it is superior to the unsupervised approach (see the “Partially-supervised method extension” section in the Methods).

SuperSigs for Aging and Other Factors Vary with Tissue Type

It has long been known that certain types of mutations, such as C>T transitions resulting from cytosine deamination, accumulate with age. It was wondered whether other mutational signatures of aging were present in cancers and whether they varied among tissue types. To avoid confounding factors as much as possible, the analysis was confined to patients without known cancer-associated environmental exposures and without known germline predispositions to cancer.

SuperSigs associated with aging were thereby obtained for each cancer type analyzed, examples of which are shown in FIG. 19A (see, also, FIG. 23 and Table 8). Not surprisingly, C>T transitions were found to be present in large fractions in many cancer types. However, others, such as C>A transversions in leukemias and prostate cancers, T>C transitions in esophageal adenocarcinomas, C>G transversions in head and neck, and any mutations of the T pyrimidine in breast cancers and testicular tumors, had not been previously described as major age-associated mutations (FIG. 19A and FIG. 23).

It was next sought to identify tissue-specific SuperSigs associated with specific environmental carcinogens. The analysis was performed after controlling for age and for other relevant covariates. Tissue-specific SuperSigs were obtained for smoking, alcohol, hepatitis B and C virus infection (HBV, HCV), aristolochic acid (AA), asbestos, and ultraviolet (UV) light (FIG. 19B, FIG. 24, and Table 8). It was also sought to identify mutational signatures associated with defective DNA polymerization or repair, controlling for age, and other relevant covariates. Tissue-specific SuperSigs were obtained for mismatch repair deficiency, mutations in DNA polymerase delta or epsilon genes, mutations in the breast cancer susceptibility genes BRCA1 or BRCA2, methylation of the MGMT and IDH1 genes, and APOBEC (FIG. 19B, FIG. 23, and Table 8).

In several cases, the SuperSigs associated with the same mutational factors varied across tissues, just as they did with aging. For example, the SuperSigs associated with smoking were very different in bladder, esophageal, head and neck, and lung cancers (FIG. 19C). And the SuperSigs associated with BRCA gene mutations were considerably different between breast and ovarian cancers (FIG. 24). There were, however, SuperSigs that did not vary much among tissue types, e.g. those based on mismatch repair deficiency, and some of those associated with inherited factors (FIG. 24).

Note that tissue specific differences with respect to etiologic factors are not possible to discover with the unsupervised approach described by Alexandrov et al. (Nature 500, 415-421 (2013)) because the identity of a given signature across multiple tissues was a key theoretical assumption underpinning their approach.

The heatmap in FIG. 20 shows the “closeness”—as measured by their correlation—between the mutational landscapes of any two cohorts of patients across all cancer types, clustering the more similar ones with each other (FIG. 26A). The distances obtained by this alternative analysis indicate that the mutational landscapes produced by aging are spread all across the range, providing further evidence that the mutational processes associated with aging vary greatly with tissue type. This remained true even when subtracting the aging effect from the mutational landscape of the exposed cohort (FIG. 26B).

Moreover, in several cases, the tissue-specific mutational landscape associated with an environmental factor was similar to the aging mutational landscape of the same tissue (FIGS. 20 and 26A). For example, the mutational landscape in smokers was more similar to the aging one in the corresponding tissue than to the ones of smokers in other tissues (FIG. 26A). This again remained true for bladder, cervical, esophageal, and kidney cancers even when subtracting the aging effect from the mutational landscape of the exposed cohort (FIG. 26B).

These analyses then suggest that a major effect of environmental factors may simply be to increase the rate of cell division. Such increases would be linearly proportional to the increase in mutation rate and would not be associated with new signatures such as those caused by direct interaction of carcinogens with DNA. Increases in the rate of cell division are known to occur when tissues are damaged or inflamed.

### SuperSigs for Obesity

Obesity (as measured by a body mass index, BMI, greater than 30) has emerged as the major lifestyle factor contributing to cancer in general. How obesity contributes to cancer risk, however, is unknown. For example, obesity could lead to cancer by inducing mutations or by stimulating the growth of neoplastic cells that have already acquired mutations. If the former explanation were valid, there might be a mutational signature associated with obesity, but no such signature has been previously identified. Four cancer types associated with obesity in which adequate number of samples and body mass index data for a supervised machine learning approach were available: colon, esophageal, kidney, and uterine cancer. SuperSigs for obesity were identified in all of these cancer types (FIG. 21). And in cross-validation, the ability to predict which patients were obese simply by the SuperSigs in their cancers—as measured by the AUC—was 0.76 in colon cancer (COAD), 0.91 in esophageal cancer (ESCA), 0.89 in kidney cancer (kidney renal papillary cell carcinoma—KIRP), and 0.84 in uterine cancer (UCEC) (Table 9). The obesity SuperSigs varied among the four cancer types, again emphasizing the tissue specificity of mutational signatures associated with the same risk factor.

A common characteristic of these obesity signatures is that the rate of accumulation of certain mutation types increases under the effect of obesity while other mutation types decrease (FIG. 21). This provides an explanation for the observation that often the total number of somatic mutations found in cancers of obese patients is not significantly different from that of non-obese patients, when controlling for age. Often only the mutational spectrum is different. Obesity may then induce interaction effects among mutational processes that go beyond the usual additive effects.

### The Proportion of Mutations Due to Aging

Finally, the supervised approach was applied to estimate the proportion of the overall mutational load that can be attributable to normal aging rather than to other mutational processes. When considering all 30 tissues, it was estimated that on average 70% of the mutations can be attributable to the normal endogenous mutational processes associated with aging, that is normal DNA replication (Table 10). This estimate is consistent with what previously reported in Tomasetti et al. (Science 355, 1330-1334 (2017)). The proportion varied widely across tissues, for example it is 2% on average in endometrial cancers (UCEC) of patients with POLe mutations to 90% in pancreatic cancer (PAAD) patients who smoke. This estimated proportion is expected to be an overestimate given the lack of full annotation for all environmental and inherited factors.

### Methods

### Data Preparation and Integration

We downloaded somatic exomic mutational data from the TCGA Bioportal (portal.gdc.cancer.gov) and filtered out the mutations which have less than 5% Variant Allele Frequency (VAF). Out of the total thirty-three datasets available, large B-cell lymphoma (DLBC) was not included in the analysis because of the small number of samples available, while lung squamous cell carcinoma (LUSC) and mesothelioma (MESO) were excluded because of the extremely small number of patients unexposed to smoking and asbestos, respectively. For ovarian cancer (OV) and acute myeloid leukemia (LAML) whole genome sequencing data were used. The human genome reference build hg38 was used to determine the context (flanking bases) for each mutation. The clinical information was downloaded from the website Cbioportal (cbioportal.org). For calculating the background frequency of each trinucleotide on both the exome and the genome the R package, deconstructSigs was used. For the Unsupervised Signature method (Alexandrov et al. Nature 500, 415-421 (2013)), the signatures were downloaded from the Cosmic Signature website (cancer.sanger.ac.uk/cosmic/signatures) and used the table cancer.sanger.ac.uk/signatures/matrix.png in order to determine which signatures were present in which tissue.

All analyses were performed using R version 3.5.2. Logistic regression was performed using glm from the STATS package. LDA was performed using the function lda from the package MASS. Non-negative matrix factorization (NMF) was performed using the function nmf with method “Lee” from the package NMF.

### Filtering of the Samples

To reduce the effect of confounding factors, several filtering criteria were applied. In each tissue type, samples were divided into two categories: 1) “unexposed”, meaning that no exposure to a known environmental factor was recorded, according to the available clinical annotation, and 2) “exposed”. To mitigate the effects of other unknown factors in the unexposed group, any sample with a mutational load more than 3 times higher than the median number of mutations found among the unexposed samples was removed. Samples were excluded if the total number of mutations was equal to zero on the exome, a probable indication of low neoplastic cell content. Samples with microsatellite instability (MSI) or with a mutation in POLE/POLE2/POLE3/POLE4 or POLD1/POLD2/POLD3/POLD4 genes were removed—except for when the signature for the specific effects of those mutations was the objective of the analysis—because of the known large increase in the number of mutations they induce. A tissue type was divided into subtypes whenever possible. Acute Myeloid Leukemia (AML) patients younger than 40 years old were not considered. Among the “exposed” samples, samples with known multi-factor exposures to minimize confounding factors were excluded and only samples with a single known exposure were evaluated. Samples with unknown exposure were treated as unexposed.

### Measuring Mutations

Mutation counts are used to characterize mutational burden when considering predictors of aging. For all other exposures, mutation rates (i.e. counts/age) are used. In a patient exposed only to time, i.e. unexposed to any known environmental or inherited factor, the rate of a mutation type is expected to remain constant irrespective of age—as dictated by the aging signature—while the absolute count is expected to increase with age. In contrast, in a patient exposed to an environmental or inherited factor, the rate of a mutation type as well as the count may change with respect to the age signature.

### Supervised Methodology for Generating Signatures (SuperSigs)

Details for the method developed to obtain the supervised mutational signatures are provided in FIG. 16.

At its simplest, a mutational signature of exposure is nothing more than a set of substitutions that characteristically occur at different rates in exposed tissue than in unexposed tissue. In practice, though, a few considerations suggested by prior biological knowledge quickly turn a simple calculation into a complex engineering problem. Specifically, a key principle of the SuperSig approach is that signatures may not be optimally described by the same base length units. Accordingly, all single-base substitutions, with or without the flanking context bases, were consider as potential, signature features. In addition to 6 single base substitutions: C>A, C>G, C>T, T>A, T>C, and T>G, named according to the pyrimidine of the mutated Watson-Crick base pair, there are 48 dinucleotides, in which the substitution is paired with a specific base as a prefix or as a suffix but not both (e.g. A[C>T] or [C>T]G), as well as 96 trinucleotides (e.g. A[C>T]G), which include both flanking bases as context. Hence, there is a list of 151 potential features (6+48+96+1).

The resulting flexibility carries a price, however, as features are no longer independent. The simple substitution C>T spawns dinucleotide children, such as A[C>T], and trinucleotide grandchildren like A[C>T]G. Frequent, exposure-driven A[C>T] substitutions would increase the observed rates of both the C>T parent and the trinucleotide children, making it difficult to assign ownership to the correct generation. The section ContextMatters describes an approach to solving this problem, while the section CombiningPartitions describes how candidate signature features are combined to create a final signature.

### Supervised Feature Engineering (ContextMatters)

The mutational family tree. The set of features described above thus form a family tree, in which the observed mutational rate (or count, when learning the mutational signatures of aging) for each substitution is propagated down the tree to children and grandchildren (FIG. 22). For completeness, the tree is augmented with a single root, Total Mutations, parent to all 6 simple substitutions, describing the overall mutation rate (or count, for aging). Such a tree can represent the mutations found in a single sample, or summarize results observed across a set of samples. In practice, two trees were built for each combination of exposure and tissue, to capture mutation rates separately in exposed and unexposed individuals, and combine them later.
Feature selection. Features of interest are selected in each tree by a two-phase process, first working down the tree from the root and then back up again. The very simple principle behind the first phase is that the mutation rate for each feature is to be compared to that expected by chance alone, to distinguish features that may be associated with exposure. As an unfortunate consequence of the family structure, however, the simplest implementation of this principle is biased toward the selection of late-generation features, where the propagation of individually insignificant deviations across 2 or 3 generations may add up to a significant cumulative difference. Thus, in practice each feature must pass a series of tests against a hierarchy of conditional null distributions defined by accounting for the observed mutation rates of each ancestor in turn. In consequence, unless proven otherwise, the mutational wealth of a given feature is explained by inheritance from its ancestors. This leads to the second phase of the process, where one works back up the tree, reevaluating all parent-child pairs selected in the first phase to make sure that one has not over-corrected, and erroneously attributed later generation wealth to earlier generations. Mathematical details are provided below.
Phase 1) Going down the tree. The hierarchy of conditional nulls is perhaps best described by example. If chance alone is at work, the expected number of C>T mutations would be Total_Mutation_Count*Normal_Frequency_of_C*⅓, the last factor accounting for three, equally likely substitutions for C. The C>T substitution would be selected as a candidate feature if the observed number of C>T mutations were significantly greater than the expected value, according to a one-sided binomial test. Moving down a generation, [C>T]A, as the child of the C>T substitution, and the grandchild of the total number of mutations (Total Mutations), would be tested twice to see if it significantly exceeded its expected number based on the total number of mutations as well as the number of C>T. The expected value of [C>T]A mutations would be given by Total_Mutation_Count*Normal_Frequency_of_C*⅓ *X, where X is the expected frequency of CA (i.e. C followed by an A) out of all C nucleotides in the exome, as estimated by deconstructSigs (FIG. 22).

The binomial test was based on an estimate of the sum of the number of mutations observed for that potential feature across all training samples, and the probability of success was set equal to the frequency of that potential feature, as expected by its representation on the exome. Specifically, the estimate of the sum of the number of mutations observed for that potential feature across all training samples was calculated by a bootstrap (100 times) for the sum of the pseudo count of that feature, of which the median was taken. The start for the pseudo count of the Total Mutations is set at 1000. For any other feature, the pseudo count starts from the proportion of that feature with respect to the exome, multiplied by 1000. Rounding was applied to the outcome.

All results were considered significant at a p-value of 0.05, subject to Bonferroni correction for 150 tests, as Total Mutations is not tested against. If the null hypothesis was rejected, that potential feature as a “first-phase” candidate feature was selected for the next supervised selection step. First-phase candidate features are colored in grey in FIG. 22.
Phase 2) Going back up. Once a list of first-phase candidate features had been thus selected, this list was pruned resulting in a smaller set of second-phase candidate features (FIG. 22). This was done by “going up the tree”, that is, by re-evaluating the significance of first-phase candidate features that are parents of first-phase candidate features. Indeed, some parent features may have been selected only because their children had higher than expected frequencies. The parent was tested by removing the contributions in terms of number of mutations present among the selected children to see if the count of the leftover in that parent would still be significantly higher than expected by chance. If it were, then that parent remained in the list as a second-phase candidate feature. And, for each sample, its mutation count is updated by removing the mutations of the second-phase candidate feature children. Instead, if not significant, the parent was eliminated as a feature in that particular analysis. The feature containing the leftover of the Total Mutations was named “remaining mutations” and was kept it as a second-phase candidate feature, to protect from discarding important correlations that may not be tested by the algorithm.
Combining partitions. For every factor other than age, the above feature-engineering (ContextMatters) step was applied separately to samples from patients that were respectively unexposed or exposed to the factor under consideration. These two lists of second-phase candidate features, which are both partitions, were then combined by considering all intersections and relative complements of the elements in the two original partitions, to form the minimal refinement of the two (see Table 7 for an example), and define this final list as the list of candidate features.

When combining two partitions, features may be overlapping. In that case the respective counts need to be distributed among the features of the refinement partition. Those counts were project as follows. For example, Partition 1, may consist of [C>T]G, [C>T]H, and the remaining mutations, with proportions 15%, 5%, and 80% respectively, while Partition 2 may consist of A[C>T], B[C>T], and the remaining mutations, with proportions 3%, 7%, and 90%, respectively. In the example, this refinement will contain the following features: A[C>T]G, B[C>T]G, A[C>T]H, B[C>T]H and the remaining of mutations (Table 7). When “projecting” counts of features in Partition 1 or Partition 2 onto a feature present in the refinement partition, the counts were split according to the expected frequencies observed on the exome (see Table 7, e.g. #ACG/#CG is the expected frequency of ACGs out of all CGs).

For aging signatures, the feature engineering steps described above were applied only to samples from patients who were unexposed to any known environmental or inherited factor. Therefore, this step of combining partitions was skipped, because there is only one partition, i.e. its second-phase candidate features, which automatically provided its “candidate features” list.

### Supervised Feature Selection (PredictiveFeatures)

Each feature was ranked according to its ability to discriminate exposed samples from unexposed, based on the rates for that feature (or counts, as appropriate for the exposure). Discriminatory performance was measured by the area under the receiver operating characteristic (ROC) curve (AUC). As above, rather than calculating the AUC directly, it was estimated robustly by taking the median over 1000 bootstrapped samples. Features for which the median AUC ≤0.5 on a balanced dataset are discarded.

Among all these features, the n top-ranked features that provided the highest AUC in an inner loop of 5 iterations of 5-fold cross-validation using a multivariate, logistic regression classifier (LR) were selected. These n features were defined as the predictive features for a given exposure.

For the age analysis, the unexposed samples were divided into three groups of equal size (younger, middle-aged, older), based on the quantiles of the age distribution, and discarded the middle group before training the algorithm.

### Signature Representation (Signatures)

The set of n predictive features selected above form the supervised signature (SuperSig). Two values are associated to each one of these predictive features: 1) the difference in mean counts (age) or rates (all other exposures) between the exposed and unexposed cohorts, and 2) the beta (β) coefficient for that feature as estimated by logistic regression. Both vectors yield critical information.

The difference in means for each feature, which is the only constraint used by logistic regression in maximizing entropy over the dataset, provide a natural measure of the difference in counts or rates for that feature induced by a given exposure. These values were report in the figures such as in FIGS. 23 and 24.

The beta coefficients of the features in a logistic regression have also an intuitive interpretation, since the logarithm of the odds of being in the exposed class C versus the unexposed one, given the mutational data (counts or rates), is given by

$$log \frac{p ( C = exposed \vert X = x )}{p ( C = unexposed \vert X = x )} = {β}^{T} x .$$

Therefore, e^{β} of a feature is the factor by which the odds of being in the exposed class increase for every extra unit increase in that feature, when all other features are kept constant. The β coefficients of the mutational signatures for each factor (aging or exposure) can be found in Table 8 and are depicted in FIGS. 29 and 30.

### Prediction Via Logistic Regression (Prediction)

Logistic Regression (LR) was used to test the predictive accuracy of each set of features representing a mutational signature as measured by AUC. the performance of Linear Discriminant Analysis (LDA) and Random Forest (RF), when applied to both feature selection and prediction was reported (Table 9). In both LR and LDA models the mean vectors equal the empirical mean vector. In addition, LDA also accounts for the dependencies among the features. All methods yielded relatively comparable results in cross-validation.

### Training

For the age analysis, the unexposed samples were again divided into three groups (younger, middle-aged, older) and discarded the middle group before training the algorithm. For all other exposures, unexposed and exposed formed the two groups except for ultraviolet light (UV) and asbestos, for which samples with respectively the lowest 10% and 33% of the Total Mutations count were used for the unexposed group, and all the other samples for the exposed one.

Training was performed using the counts the predictive features for age and the rates (=count/age) of the predictive features for all other exposures, over the two labeled groups, via 5 iterations of 5-fold cross-validation using LR.

### Testing

The same quantities, counts for age and rates for all other factors, are used for testing. Again, for age, the middle-aged group was excluded from the test set.

### Comparison of Performance Between Unsupervised, SuperSigs, and Randomly Generated Peak Signatures

When prior literature has established a strong relationship between an exposure and a particular mutational feature, i.e. [C>T]G for aging and C>A for smoking, it was evaluated whether any new candidate signatures actually improve on these central, peak feature. Specifically, the value of the aging (Signature #1) and smoking unsupervised signatures were assessed in Mucci et al. (JAMA 315, 68-76 (2016)), Stadler et al. (J Clin Oncol 28, 4255-4267 (2010)), Stewart et al. (“Cancer Etiology.” In: World Cancer Report 2014 (eds Stewart B W, Wild C P). IARC (2014)), and Tomasetti (Science 364, 938-939 (2019)), as well as of the SuperSigs, beyond the main “peaks” already known from prior knowledge, i.e. [C>T]G for aging and C>A for smoking. This essentially corresponds to evaluate if the part of the distribution of an unsupervised or supervised mutational signature that is not the mutational “peak” adds any value, according to some measure of performance (prediction or correlation).

To do this, a signature was generate for smoking, whose property is a higher proportion of C>A mutations than the other mutation types and where, beside this “peak” at C>A, the proportion of all the other mutation types is assigned randomly. Similarly a signature was generate for aging, whose property is a higher proportion of [C>T]G mutations than the other mutation types and where, beside this “peak” at [C>T]G, the proportion of all the other mutation types is assigned randomly. This was done by building “randomly generated single peak signatures”, or “single peak signatures” for brevity.

More precisely, for the smoking signature, this randomly generated smoking peak signature was created in a two-step process. In step one, 30 (since in Cosmic v.2 there are about 30 signatures) probability distributions were generated over the six main mutation types (which lack suffix and prefix base). Each distribution was created by sampling 6 numbers from a uniform distribution and by dividing them by their sum. The “smoking single peak signature” was then the distribution among them with the highest proportion of C>A substitutions. In step two, the obtained proportion of each of the six main mutation types was randomly broken down into the 16 fundamental trinucleotide mutations (16 for C>A, 16 for C>T, and so on).

A similar process was applied to the derivation of the randomly generated peak age signatures. The difference is that it was assumed the main types of mutations are now seven: [C>T]G, [C>T]H, C>A, C>G, T>A, T>C, and T>G, due to the fact that [C>T]G is needed as one of the features, since that is the peak obtained from prior-knowledge. Among the 30 signature candidates, the “aging single peak signature” is then the distribution with the maximum proportion of [C>T]G substitutions.

### Comparison of Alexandrov et al. (Nature 500, 415-421 (2013)), Randomly Generated Peak Signatures, and SuperSigs

In order to compare the prediction accuracy (AUC) of all three sets of signatures (Alexandrov et al., single peak, and SuperSigs), the same prediction methodology was applied that was previously used in Alexandrov et al. to determine the contribution of each signature in each patient: non-negative least squares (NNLS).

More specifically, to determine in a given patient the respective proportional contributions (used as a score) X of each mutational signature i=1, . . . , k, where a total of k signatures are present in that tissue, NNLS is applied to

Yᵢ=Aᵢ₁X₁+Aᵢ₂X₂+ . . . +AᵢₖXₖ

i.e. Y=AX in matrix form, where Y is the total number of mutations of type i, and Aᵢⱼ is the relative frequency (for Alexandrov et al. and single peak signatures) or the difference in mean count (SuperSigs for age) or rate (SuperSigs for all other etiological factors) of mutation type i in the mutational signature j, across each one of the k signatures present in that tissue.

The performance of the various methodologies is presented in FIG. 18, FIG. 25, and Table 9.

For Alexandrov et al. their Signature 1 was used for predicting age in one comparison, and the combination of the “clock-wise” unsupervised Signatures 1 and 5 as determined in Alexandrov et al., (Nat Genet 47, 1402-1407 (2015)) was used in the other comparison. The specific combination of signatures used for Alexandrov et al. in predicting smoking status was instead determined by the specific combinations provided for each tissue in Alexandrov et al. (Science 354, 618-622 (2016)).

### Comparison of Cross-Validated NMF Versus SuperSigs

Given that it was not possible to cross-validate directly the unsupervised method of Alexandrov et al. (Nature 500, 415-421 (2013)) the core methodology used in Alexandrov et al., which is non-negative matrix factorization (NMF), it was chosen to use and approximate their method in two alternative ways in order to perform cross-validation: 1) “BestNMF” and 2) “MatchedNMF”.

For both approaches, NMF was applied to the profile of the count mutations of the training samples, i.e. a matrix whose 96 rows represent mutation types and columns represent training samples. The rank parameter, r, of the NMF algorithm was set equal to what shown in Cosmic signature v2 (cancer.sanger.ac.uk/cosmic/signatures v2) for the tissue of interest. This parameter was hardwired to help the unsupervised method to limit model misspecification.

After obtaining the r signatures from NMF, two alternative methods were used to select among them the signature of a specific age or environmental factor: 1) for BestNMF, the signature whose contributions had the highest AUC in classifying exposure to the environmental factor on the training set were chosen; 2) for MatchedNMF, each of the identified signatures from the training set was paired to exactly one of those listed in Cosmic v2 for this specific tissue. This pairing process was obtained by maximizing the sum of the cosine similarity for each pair.

Then, on the test set, an NNLS algorithm was used to estimate the contribution of each signature on the test set.

The performance of the various methodologies is presented in FIG. 18, FIG. 25, and Table 9.

### Partially-Supervised Method Extension

One limitation of a supervised approach is that it cannot be applied to find signatures of factors for which no annotation is currently available. It may indeed be desirable to have a method that is able to discover patterns of exposures, even when they are unknown. This limitation, however, can be overcome by using the supervised step, already described, and following it with an unsupervised one. That is, one can first take advantage of all exposures with available annotations to discover their supervised signatures. After learning those signatures, the effects of those supervised signatures can be “subtracted” from the mutational load of the patients exposed to those annotated factors. An unsupervised analysis, such as non-negative matrix factorization (NMF), can then be performed on the leftover, to investigate the presence of further mutational patterns.

An example is provided here of how the supervised learning of a mutational signature (specifically the aging signature in this example) can be used to improve the performance of an unsupervised approach by discounting the effects of that supervised signature on the test data. This methodology is referred to hereafter to as “partially supervised”.

To simplify matters, features were not engineered; rather, the 96 fundamental mutations as in Alexandrov et al. (Nature 500, 415-421 (2013)) were used. Only the datasets that show a higher average rate of mutation per year in the exposed samples than in the unexposed samples were used. This increase in the rate is required to conform to the premise of non-negativity and linearity in the NMF model. One half of the unexposed samples were use as the training set to learn the rate of each feature of the age signature (thus a supervised signature) so that the effect of age (i.e. controlling for age) on the test set can be discounted. Next the test set was formed by bootstrapping over the left-out half of the unexposed samples and all exposed ones.

NMF with rank equal to 3 was applied to decompose the test set, Y, thus obtaining two matrices, A and X: one containing the unsupervised signatures (A) and a second one with the corresponding contributions of each of those signatures in each patient (X). These contributions have not been discounted for age yet. This is the standard unsupervised approach. However, in order to estimate the discounted contributions of a signature in each test sample, the effect of age of a patient on each unsupervised signature was discounted by multiplying the learned supervised age signature by the age of the patient, times the estimated mutation rate, and then projecting this vector onto the directions identified by NMF using NNLS, and then subtracting these projected contributions of age from the contributions of the 3 unsupervised signatures obtained by NMF. To conform to the premises of NMF, the negative discounted contributions was set to zero.

The direction whose contribution, divided by the total number of mutations, is the most associated (in terms of the highest AUC) to the exposure status using the known ground-truth, was chosen for both the unsupervised and the partially supervised methods, by using the not discounted and discounted contributions, respectively. To obtain the “partially supervised signatures” non-negative linear regression was used again but this time where the contributions (X) are known and the signatures (A) are unknown. In other words, the decomposition is still Y=AX, but now, Y and X are known and A is estimated.

The AUC was used to evaluate the association of the signature with the exposure status, for both the unsupervised and partially supervised approach, where the contribution of each signature has been divided by the number of total mutations. this whole process (from the random selection of half of the unexposed patients used to learn the age signature and so on) was repeated 50 times and the average AUC over them was taken to account for the effect of randomness. This is what is depicted in FIG. 27, where the increase in performance of the partially supervised method with respect to the unsupervised is evident.

In this partially supervised extension, NMF was used to easily compare with the unsupervised approach by Alexandrov et al. (Nature 500, 415-421 (2013)). However, other methodologies (e.g. a classifier based on EM) may provide even better performance.

### The Effect of Model Misspecification on the Unsupervised Signatures

If there was no annotation for the presence of defects in the gene POL-ε among patients with endometrial cancer in the UCEC-TCGA dataset and the POL-ε signature was not known, the normalized results for an NMF decomposition are depicted in FIG. 28A. This figure shows the striking similarity of this unsupervised pattern with the known POL-ε supervised signature (compare FIG. 24 with FIG. 28A). In particular, the high frequency of T[C>A]T mutations is easily detected in the signature by NMF. Thus, the unsupervised approach is able to find the signature even for factors for which annotation is not available, at least when the signal is very strong as in the case of a POL-ε mutation. The POL-ε signature in FIG. 28A was obtained by “telling” NMF to search for one (i.e. rank=1) pattern. If instead two, three, or four signatures were used, respectively, NMF would have returned the patterns depicted in FIG. 28B-28D. FIG. 28B-28D show that the POL-ε signature has been parsed into multiple patterns: the more patterns the more the optimum signature is spread across different claimed signatures. Therefore, the quality of the results of NMF strongly depend on the number of signatures NMF is required to extract. Unfortunately there is no fully satisfactory rule to determine a priori how many patterns should be found by NMF. This is a problem that all unsupervised approaches have because the researcher is blind to the actual number of different exposures that are present among the patients in the dataset during the discovery phase. In some cases, the distribution of mutation types can be considered without using NMF at all. If this distribution had been considered in the example noted above, the pattern depicted in FIG. 28E, which is again strikingly similar to the known supervised POL-ε signature would have been obtained.

### Estimation of the Proportion of Mutations Due to Aging

Each predictive feature of the SuperSigs can be represented by its rate. For age, the “rate” of feature i, rᵢ^{a}, is defined as the mean of the ratio:

$${r}_{i}^{a} = \frac{mean ( count of feature i )}{mean ( age )}$$

in unexposed patients. This rate estimates the number of mutations of that particular feature accumulating per year and attributable to age. To estimate the proportion of mutations due to aging in each specific sample ria of each feature i present in the SuperSig age signature was multiplied by the patient's age of that specific sample. The number obtained by summing the above counts for each feature in the age SuperSig is then divided by the total number of mutations observed in that sample. This resulting ratio, being forced to be not greater than 1, is the estimate for the proportion of somatic mutations attributable to age in that sample (see Table 10).

### Distances Among Mutational Landscapes of Different Exposures in Tissues

The mutational landscape of an exposure in a tissue was defined as the 96-long vector (96 trinucleotide mutations) where each entry is given by the average count of that mutation type in the cohort of the samples with that exposure divided by the average age in that cohort. The mutational landscape of aging is obtained in the same way using the cohort of samples without any known exposure (“unexposed”). Then, the distance between any two mutational landscapes is given by 1—the Pearson's correlation between the two mutational landscapes (see FIG. 20 and FIG. 26A). For the results in FIG. 26B the effect of age has been removed from the mutational landscape of all exposures but age, by subtracting the mutational landscape of age from the relevant exposed tissue. Replacing the distance based on correlation with one based on cosine similarity yields equivalent results.

Robustness Analysis with Respect to Mislabeling

To assess the robustness of the methodology with respect to the quality of the clinical annotation, the labels were switch from unexposed to exposed (or vice versa) for 5%, 10%, 20%, and 25% of the samples in the training set. For example, non-smokers would be mislabeled as smokers and vice versa. Then the supervised method is rerun, including feature engineering and selection, on the training set to obtain new signatures. These new signatures are then used for prediction in the test set, where the original labels were used as the ground truth. The performance is reported in Table 11. AUCs at the different mislabeling percentages were compare and it was found that the method still outperforms the unsupervised method up to a mislabeling proportion of 20%, reaching a comparable prediction performance at a mislabeling proportion of 25%.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

TABLE 1
Performance of SMOKING SIGNATURE
in LUNG ADENOCARCINOMA (LUAD)
1A - Uniformly generated random signatures (SMOKING in LUAD).

| AUC | | |
|---|---|---|
| UnsupSignature_mean | RandSampSignature_mean | SupSigPred_mean |
| 0.8147645 | 0.8367098 | 0.8919025 |
| Cor | | |
| UnsupSignature_mean | RandSampSignature_mean | SupSigPred_mean |
| 0.3439773 | 0.31946 | 0.366653 |

1B -random patient and classification (SMOKING in LUAD).

| | | |
|---|---|---|
| AUC | | |
| UnsupSignature_mean | RandSampSignature_mean | SupSigPred_mean |
| 0.8147645 | 0.8677438 | 0.8919025 |
| Cor | | |
| UnsupSignature_mean | RandSampSignature_mean | SupSigPred_mean |
| 0.3439773 | 0.3674401 | 0.366653 |

AGING SIGNATURE
2A-Uniformly generated random signatures (AGE)

| | | | |
|---|---|---|---|
| AUC:\n | | | |
| UnsupSignature1_mean | RandSampSignature_mean | SupSig_mean | DataSet |
| 0.5850694 | 0.6122049 | 0.6814236 | LAML |
| 0.6543367 | 0.6470536 | 0.7283163 | BLCA |
| 0.4707602 | 0.5449415 | 0.6666667 | LUAD |
| 0.7925926 | 0.8457778 | 0.8888889 | LGG |
| 0.6711587 | 0.7561378 | 0.7677133 | HNSCC |
| 0.5511123 | 0.7873782 | 0.8283898 | KIRC |
| 0.494302 | 0.7344587 | 0.7720798 | KIRP |
| 0.7093426 | 0.7958478 | 0.8546713 | KICH |
| 0.5492611 | 0.6596182 | 0.7487685 | LIHC |
| 0.6654412 | 0.6684477 | 0.6776961 | STAD |
| 0.5181487 | 0.6567964 | 0.7573615 | THCA |
| 0.29 | 0.47395 | 0.635 | UVM |
| 0.4830458 | 0.510934 | 0.6213563 | SKCM |
| 0.4713043 | 0.7182435 | 0.7878261 | ACC |
| 0.5532544 | 0.5940237 | 0.7662722 | CHOL |
| 0.6123016 | 0.648254 | 0.7141534 | GBM |
| 0.6040386 | 0.6817252 | 0.7539508 | CESC |
| 0.6098001 | 0.6400819 | 0.6547853 | COAD |
| 0.5233844 | 0.6680825 | 0.7842262 | PCPG |
| 0.6546053 | 0.658125 | 0.6003289 | PAAD |
| 0.5604516 | 0.6594787 | 0.689957 | PRAD |
| 0.5754986 | 0.5568519 | 0.6196581 | ESCSQ |
| 0.5734072 | 0.5685873 | 0.5457064 | ESCAD |
| 0.503125 | 0.68275 | 0.7052083 | UCEC |
| 0.6339869 | 0.590719 | 0.6372549 | UCS |
| 0.5551903 | 0.5924395 | 0.6276069 | BRCA |
| 0.692682 | 0.7927037 | 0.8287671 | SARC |
| 0.4328947 | 0.5556579 | 0.6042763 | TGCT |
| 0.5959806 | 0.6647678 | 0.7456687 | THYM |
| 0.6717922 | 0.5853287 | 0.7317073 | OV |
| Average: | | | |
| UnsupSignature1_mean | RandSampSignature_mean | SupSig_mean | |
| 0.5752756 | 0.6517122 | 0.7141895 | |
| Cor:\n | | | |
| UnsupSignature1_mean | RandSampSignature_mean | SupSig_mean | DataSet |
| 0.15673581 | 0.242041446 | 0.37820376 | LAML |
| 0.17371309 | 0.312139507 | 0.40008508 | BLCA |
| −0.07785031 | 0.006174119 | 0.23124601 | LUAD |
| 0.69622278 | 0.709864075 | 0.69862024 | LGG |
| 0.32588324 | 0.455935885 | 0.46203003 | HNSCC |
| 0.12387476 | 0.540741299 | 0.61039364 | KIRC |
| 0.0239592 | 0.399364113 | 0.43363672 | KIRP |
| 0.22939023 | 0.433804604 | 0.59493797 | KICH |
| 0.13699334 | 0.4356601 | 0.56349156 | LIHC |
| 0.27340753 | 0.354976799 | 0.35554616 | STAD |
| 0.062674 | 0.281414093 | 0.4114341 | THCA |
| −0.22763268 | 0.050825278 | 0.21500083 | UVM |
| 0.02400207 | −0.068028289 | 0.15468474 | SKCM |
| −0.16832296 | 0.311428028 | 0.40058681 | ACC |
| 0.06748897 | 0.234537152 | 0.52501383 | CHOL |
| 0.19367388 | 0.270784349 | 0.38630892 | GBM |
| 0.18065944 | 0.301743835 | 0.44360507 | CESC |
| 0.18848569 | 0.198866557 | 0.22118282 | COAD |
| 0.02557715 | 0.277985749 | 0.48825009 | PCPG |
| 0.28622692 | 0.227528732 | 0.15366795 | PAAD |
| 0.0699365 | 0.265560674 | 0.33195903 | PRAD |
| 0.09420754 | 0.083641642 | 0.24253521 | ESCSQ |
| 0.16197186 | 0.163907937 | 0.02555954 | ESCAD |
| 0.02113093 | 0.256499193 | 0.31423399 | UCEC |
| 0.2991348 | 0.218306278 | 0.34222433 | UCS |
| 0.13427725 | 0.194365912 | 0.22306646 | BRCA |
| 0.31643963 | 0.516019163 | 0.58739081 | SARC |
| −0.14232707 | 0.095853784 | 0.19748519 | TGCT |
| 0.19534576 | 0.374501084 | 0.51395133 | THYM |
| 0.25602365 | 0.10764116 | 0.36454989 | OV |
| Average: | | | |
| UnsupSignature1_mean | RandSampSignature_mean | SupSig_mean | |
| 0.1367101 | 0.2751361 | 0.3756961 | |

2B-random patient and classification (AGE)

| | | |
|---|---|---|
| AUC:\n | | |
| UnsupSignature1_mean | RandSampSignature_mean | SupSig_mean |
| 0.5850694 | 0.6415538 | 0.6814236 |
| 0.6543367 | 0.6088903 | 0.7283163 |
| 0.4707602 | 0.5751901 | 0.6666667 |
| 0.7925926 | 0.8802222 | 0.8888889 |
| 0.6711587 | 0.7287677 | 0.7677133 |
| 0.5511123 | 0.7611427 | 0.8283898 |
| 0.494302 | 0.7249003 | 0.7720798 |
| 0.7093426 | 0.7655363 | 0.8546713 |
| 0.5492611 | 0.6817734 | 0.7487685 |
| 0.6654412 | 0.6445221 | 0.6776961 |
| 0.5181487 | 0.6406057 | 0.7573615 |
| 0.29 | 0.4825875 | 0.635 |
| 0.4830458 | 0.513843 | 0.6213563 |
| 0.4713043 | 0.6926435 | 0.7878261 |
| 0.5532544 | 0.5798817 | 0.7662722 |
| 0.6123016 | 0.6398307 | 0.7141534 |
| 0.6040386 | 0.603356 | 0.7539508 |
| 0.6098001 | 0.6358309 | 0.6547853 |
| 0.5233844 | 0.6341305 | 0.7842262 |
| 0.6546053 | 0.6427961 | 0.6003289 |
| 0.5604516 | 0.6574951 | 0.689957 |
| 0.5754986 | 0.5108547 | 0.6196581 |
| 0.5734072 | 0.5488643 | 0.5457064 |
| 0.503125 | 0.6640799 | 0.7052083 |
| 0.6339869 | 0.5750654 | 0.6372549 |
| 0.5551903 | 0.5605937 | 0.6276069 |
| 0.692682 | 0.7845431 | 0.8287671 |
| 0.4328947 | 0.5590822 | 0.6042763 |
| 0.5959806 | 0.6819473 | 0.7456687 |
| 0.6717922 | 0.5373118 | 0.7317073 |
| Average: | | |
| UnsupSignature1_mean | RandSampSignature_mean | SupSig_mean |
| 0.5752756 | 0.6385947 | 0.7141895 |
| Cor:\n | | |
| UnsupSignature1_mean | RandSampSignature_mean | SupSig_mean |
| 0.15673581 | 0.27481973 | 0.37820376 |
| 0.17371309 | 0.250947 | 0.40008508 |
| −0.07785031 | 0.08682547 | 0.23124601 |
| 0.69622278 | 0.74057178 | 0.69862024 |
| 0.32588324 | 0.42562388 | 0.46203003 |
| 0.12387476 | 0.51373879 | 0.61039364 |
| 0.0239592 | 0.35434735 | 0.43363672 |
| 0.22939023 | 0.41299052 | 0.59493797 |
| 0.13699334 | 0.45837707 | 0.56349156 |
| 0.27340753 | 0.3369232 | 0.35554616 |
| 0.062674 | 0.22998396 | 0.4114341 |
| −0.22763268 | 0.0847141 | 0.21500083 |
| 0.02400207 | −0.04594026 | 0.15468474 |
| −0.16832296 | 0.31160424 | 0.40058681 |
| 0.06748897 | 0.26212126 | 0.52501383 |
| 0.19367388 | 0.25801023 | 0.38630892 |
| 0.18065944 | 0.15288311 | 0.44360507 |
| 0.18848569 | 0.18700577 | 0.22118282 |
| 0.02557715 | 0.22999744 | 0.48825009 |
| 0.28622692 | 0.1806183 | 0.15366795 |
| 0.0699365 | 0.26843498 | 0.33195903 |
| 0.09420754 | −0.0111336 | 0.24253521 |
| 0.16197186 | 0.04742665 | 0.02555954 |
| 0.02113093 | 0.22636112 | 0.31423399 |
| 0.2991348 | 0.19311108 | 0.34222433 |
| 0.13427725 | 0.13943674 | 0.22306646 |
| 0.31643963 | 0.50981152 | 0.58739081 |
| −0.14232707 | 0.10971681 | 0.19748519 |
| 0.19534576 | 0.40444446 | 0.51395133 |
| 0.25602365 | 0.03353961 | 0.36454989 |
| Average: | | |
| UnsupSignatur1e_mean | RandSampSignature_mean | SupSig_mean |
| 0.1367101 | 0.2542437 | 0.3756961 |

TABLE 2
Accuracy of age predictions. For each indicated cancer type the accuracies (AUC) of the supervised and unsupervised age signatures
are listed. For the supervised method, the accuracies are provided when using linear discriminant analysis (LDA), which is the methodology
reported in the main text, as well as for logistic regression (Logit), and random forest (RF). Both apparent and cross-validated
accuracies are reported for the supervised method. Only apparent accuracies are reported for the unsupervised method.

| | LDA | Logit | RF | Unsupervised | | | |
|---|---|---|---|---|---|---|---|
| | (Apparent) | (Apparent) | (Apparent) | (Apparent) | LDA | Logit | RF |
| Acute Myeloid Leukemia | 0.681423611 | 0.681423611 | 0.681423611 | 0.635416667 | 0.647675 | 0.648475 | 0.634275 |
| Stomach Adenocarcinoma | 0.68504902 | 0.685457516 | 0.759599673 | 0.665441176 | 0.615594949 | 0.619837374 | 0.618877778 |
| Thyroid Carcinoma | 0.75760447 | 0.757361516 | 0.788678328 | 0.774514091 | 0.746412972 | 0.746577176 | 0.769633415 |
| Uveal Melanoma | 0.635 | 0.635 | 0.635 | 0.5 | 0.635 | 0.635 | 0.60125 |
| Skin Cutaneous Melanoma | 0.621356336 | 0.621356336 | 0.621356336 | 0.483045806 | 0.587561728 | 0.588117284 | 0.597775849 |
| Adrenocortical Carcinoma | 0.777391304 | 0.777391304 | 0.847826087 | 0.5 | 0.7344 | 0.7318 | 0.7339 |
| Cholangiocarcinoma | 0.766272189 | 0.766272189 | 0.766272189 | 0.5 | 0.808611111 | 0.808611111 | 0.808611111 |
| Glioblastoma Multiforme | 0.712566138 | 0.711772487 | 0.766269841 | 0.612301587 | 0.653504274 | 0.653034188 | 0.665630342 |
| Cervical Squamous | 0.765364355 | 0.766681299 | 0.800373134 | 0.60403863 | 0.745243026 | 0.746131808 | 0.753912269 |
| Colorectal Adenocarcinoma | 0.624549328 | 0.624303507 | 0.759873812 | 0.609800066 | 0.576861087 | 0.577385872 | 0.613307411 |
| Pheochromocytoma and Paraganglioma | 0.762117347 | 0.760416667 | 0.816539116 | 0.753401361 | 0.685445679 | 0.686712346 | 0.691245679 |
| Bladder Urothelial Carcinoma | 0.744472789 | 0.74744898 | 0.80994898 | 0.654336735 | 0.68652963 | 0.687151852 | 0.696859259 |
| Pancreatic Adenocarcinoma | 0.573684211 | 0.573684211 | 0.573684211 | 0.638596491 | 0.61 | 0.61 | 0.501944444 |
| Prostate Adenocarcinoma | 0.690989247 | 0.691763441 | 0.717505376 | 0.608924731 | 0.647806452 | 0.647956989 | 0.669430108 |
| Esophagus Squamous | 0.61965812 | 0.61965812 | 0.61965812 | 0.575498575 | 0.526355556 | 0.527022222 | 0.519066667 |
| Esophagus Adenocarcimona | 0.542936288 | 0.534626039 | 0.83933518 | 0.573407202 | 0.499791667 | 0.497986111 | 0.512291667 |
| Uterine Corpus Endometrial Carcinoma | 0.710763889 | 0.711458333 | 0.778472222 | 0.618055556 | 0.63 | 0.628303571 | 0.644508929 |
| Uterine Carcinosarcoma | 0.630718954 | 0.637254902 | 0.923202614 | 0.5 | 0.471527778 | 0.471527778 | 0.423194444 |
| Breast Invasive Carcinoma | 0.636137622 | 0.635878402 | 0.648403441 | 0.60466596 | 0.588929492 | 0.588811648 | 0.575815133 |
| Sarcoma | 0.841204037 | 0.842645999 | 0.843096611 | 0.805875991 | 0.819305952 | 0.822454762 | 0.780882143 |
| Testicular Germ Cell Tumors | 0.600986842 | 0.599013158 | 0.699342105 | 0.613157895 | 0.56453125 | 0.564888393 | 0.525870536 |
| Thymoma | 0.742896743 | 0.742896743 | 0.831947332 | 0.718641719 | 0.733893495 | 0.733893495 | 0.749767219 |
| Lung Adenocarcinoma | 0.649691358 | 0.649691358 | 0.649691358 | 0.456790123 | 0.661597222 | 0.661597222 | 0.633263889 |
| Ovarian Serous Cystadenocarcinoma | 0.727995758 | 0.727995758 | 0.742311771 | 0.671792153 | 0.701035494 | 0.700510802 | 0.685050926 |
| Brain Lower Grade Glioma | 0.881481481 | 0.881481481 | 0.988888889 | 0.944444444 | 0.858888889 | 0.850555556 | 0.836944444 |
| Head and Neck | 0.775493193 | 0.775215338 | 0.82689636 | 0.671158655 | 0.728533411 | 0.725940171 | 0.733221154 |
| Renal Clear Cell Carcinoma | 0.809586864 | 0.811970339 | 0.839247881 | 0.724311441 | 0.755495338 | 0.758576146 | 0.761598193 |
| Renal Papillary Cell Carcinoma | 0.766381766 | 0.763532764 | 0.848290598 | 0.705128205 | 0.739588889 | 0.738233333 | 0.750944444 |
| Kidney Chromophobe | 0.837370242 | 0.837370242 | 0.932525952 | 0.761245675 | 0.698541667 | 0.700208333 | 0.710486111 |
| Liver Hepatocellular Carcinoma | 0.742610837 | 0.738916256 | 0.8091133 | 0.674876847 | 0.713288889 | 0.715511111 | 0.669644444 |
| Average | 0.710458478 | 0.710331277 | 0.772159148 | 0.638628926 | 0.66906503 | 0.669093722 | 0.662306767 |
| sd | 0.083901635 | 0.084468338 | 0.09927693 | 0.108117414 | 0.092756911 | 0.092371863 | 0.100432269 |

TABLE 3
Accuracy of environmental and inherited signatures' predictions. For each indicated cancer type,
and each environmental or inherited factor, the accuracies (AUC) of the supervised and unsupervised
age signatures are listed. For the supervised method, the accuracies are provided when using linear
discriminant analysis (LDA), which is the methodology reported in the main text, as well as for logistic
regression (Logit), and random forest (RF). Both apparent and cross-validated accuracies are reported
for the supervised method. Only apparent accuracies are reported for the unsupervised method.

| | LDA | Logit | RF | Unsupervised |
|---|---|---|---|---|
| | (Apparent) | (Apparent) | (Apparent) | (Apparent) |
| Smoking in Bladder Urothelial Carcinoma | 0.588814836 | 0.588814836 | 0.588814836 | 0.572935381 |
| Smoking in Lung Adenocarcinoma | 0.889866346 | 0.889396471 | 0.924872089 | 0.81476454 |
| Smoking in Head and Neck | 0.809148902 | 0.81128876 | 0.848514212 | 0.749899063 |
| Smoking in Renal Papillary Cell Carcinoma | 0.571428571 | 0.568452381 | 0.857142857 | 0.474702381 |
| Smoking in Pancreatic Adenocarcinoma | 0.613851992 | 0.613851992 | 0.613851992 | 0.5 |
| Smoking in Esophagus Squamous | 0.696811971 | 0.696811971 | 0.809043591 | 0.466818478 |
| Smoking in Esophagus Adenocarcimona | 0.664596273 | 0.664596273 | 0.664596273 | 0.5 |
| Smoking in Cervical Squamous | 0.628324057 | 0.628942486 | 0.734693878 | 0.5 |
| POLe Mutation in Uterine Corpus Endometrial Carcinoma | 0.841563786 | 0.838918283 | 0.93547913 | 0.684009406 |
| POLe Mutation in Stomach Adenocarcinoma | 0.771875 | 0.808854167 | 0.98203125 | 0.5 |
| POLe Mutation in Colorectal Adenocarcinoma | 0.952059659 | 0.952059659 | 0.992365057 | 0.592595881 |
| POLe Mutation in Breast Invasive Carcinoma | 0.695358466 | 0.71072129 | 0.862115929 | 0.401394639 |
| MLH Silenced in Uterine Corpus Endometrial Carcinoma | 0.879536102 | 0.878413767 | 0.950991395 | 0.846988403 |
| MLH Silenced in Stomach Adenocarcinoma | 0.98855906 | 0.987322202 | 0.999690785 | 0.979901051 |
| MLH Silenced in Colorectal Adenocarcinoma | 0.842105263 | 0.842105263 | 0.842105263 | 0.828947368 |
| BRCA1/2 Mutation in Breast Invasive Carcinoma | 0.697691198 | 0.727527375 | 0.832887701 | 0.52576182 |
| BRCA1/2 Mutation in Ovarian Serous Cystadenocarcinoma | 0.675438596 | 0.683829138 | 0.842486651 | 0.59382151 |
| UV* in Skin Cutaneous Melanoma | 0.953036122 | 0.953084739 | 0.975618649 | 0.857309543 |
| POLD Mutation in Uterine Corpus Endometrial Carcinoma | 0.863425926 | 0.872685185 | 0.903935185 | NA |
| High Copy Number in Uterine Corpus Endometrial Carcinoma | 0.792768959 | 0.791299236 | 0.854350382 | NA |
| Low Copy Number in Uterine Corpus Endometrial Carcinoma | 0.758487654 | 0.759259259 | 0.790509259 | NA |
| POLD Mutation in Stomach Adenocarcinoma | 0.895138889 | 0.94375 | 0.985763889 | NA |
| MGMT Methylated in Glioblastoma Multiforme | 0.690338052 | 0.690492767 | 0.726386633 | NA |
| MGMT Methylated in Brain Lower Grade Glioma | 0.630681818 | 0.630681818 | 0.630681818 | NA |
| IDH Methylated in Brain Lower Grade Glioma | 0.779395026 | 0.788155762 | 0.851998758 | NA |
| IDH Methylated in Glioblastoma Multiforme | 0.896995708 | 0.907457082 | 0.959629828 | NA |
| Obesity in Uterine Corpus Endometrial Carcinoma | 0.658166458 | 0.657853567 | 0.746088861 | NA |
| Obesity in Renal Papillary Cell Carcinoma | 0.766935484 | 0.771774194 | 0.878225806 | NA |
| Obesity in Esophageal Carcinoma | 0.756157635 | 0.756157635 | 0.83682266 | NA |
| Alcohol in Head and Neck | 0.589861751 | 0.592165899 | 0.900921659 | NA |
| Alcohol in Esophageal Carcinoma | 0.861111111 | 0.861111111 | 0.861111111 | NA |
| Alcohol in Liver Hepatocellular Carcinoma | 0.701274105 | 0.701274105 | 0.781680441 | NA |
| Hepatitis B in Liver Hepatocellular Carcinoma | 0.663409091 | 0.664015152 | 0.708409091 | NA |
| Hepatitis C in Liver Hepatocellular Carcinoma | 0.673570381 | 0.673570381 | 0.673570381 | NA |
| Aristolochic Acid in Bladder Urothelial Carcinoma | 0.964705882 | 0.993188854 | 0.995975232 | NA |
| Asbestos in Mesothelioma | 0.669886364 | 0.669886364 | 0.669886364 | NA |
| High Apobec in Cervical Squamous | 0.703770739 | 0.704977376 | 0.762745098 | 0.636802413 |
| High Apobec in Renal Clear Cell Carcinoma | 0.636921965 | 0.633550096 | 0.735789981 | 0.5 |
| Average | 0.755607084 | 0.760744655 | 0.829257473 | |
| sd | 0.117814605 | 0.121214905 | 0.116436847 | |
| Restricted Ave | 0.755607084 | 0.788914901 | 0.868324883 | 0.626332594 |
| Restricted sd | 0.128901122 | 0.131466466 | 0.105102027 | 0.164845749 |

| | LDA | Logit | RF |
|---|---|---|---|
| Smoking in Bladder Urothelial Carcinoma | 0.557573529 | 0.557851148 | 0.557458393 |
| Smoking in Lung Adenocarcinoma | 0.894646862 | 0.88696893 | 0.89651135 |
| Smoking in Head and Neck | 0.795417977 | 0.7878943 | 0.814029442 |
| Smoking in Renal Papillary Cell Carcinoma | 0.424652778 | 0.422222222 | 0.533541667 |
| Smoking in Pancreatic Adenocarcinoma | 0.553156177 | 0.541947552 | 0.502162005 |
| Smoking in Esophagus Squamous | 0.544778788 | 0.546518182 | 0.544133333 |
| Smoking in Esophagus Adenocarcimona | 0.565357143 | 0.563357143 | 0.574642857 |
| Smoking in Cervical Squamous | 0.534178655 | 0.53475117 | 0.506345906 |
| POLe Mutation in Uterine Corpus Endometrial Carcinoma | 0.814955065 | 0.814501634 | 0.857831393 |
| POLe Mutation in Stomach Adenocarcinoma | 0.715208333 | 0.726666667 | 0.7609375 |
| POLe Mutation in Colorectal Adenocarcinoma | 0.948669349 | 0.946999665 | 0.947061201 |
| POLe Mutation in Breast Invasive Carcinoma | 0.456331868 | 0.497740818 | 0.407857523 |
| MLH Silenced in Uterine Corpus Endometrial Carcinoma | 0.827759104 | 0.825385154 | 0.866177346 |
| MLH Silenced in Stomach Adenocarcinoma | 0.973744086 | 0.961480645 | 0.954312366 |
| MLH Silenced in Colorectal Adenocarcinoma | 0.839821429 | 0.836071429 | 0.819017857 |
| BRCA1/2 Mutation in Breast Invasive Carcinoma | 0.663334947 | 0.687003863 | 0.739967914 |
| BRCA1/2 Mutation in Ovarian Serous Cystadenocarcinoma | 0.504970238 | 0.521656746 | 0.669470899 |
| UV* in Skin Cutaneous Melanoma | 0.950858869 | 0.94615402 | 0.946741041 |
| POLD Mutation in Uterine Corpus Endometrial Carcinoma | 0.817493873 | 0.823848039 | 0.789669118 |
| High Copy Number in Uterine Corpus Endometrial Carcinoma | 0.710176675 | 0.717525531 | 0.674297386 |
| Low Copy Number in Uterine Corpus Endometrial Carcinoma | 0.622032664 | 0.616974689 | 0.615602847 |
| POLD Mutation in Stomach Adenocarcinoma | 0.81125 | 0.8875 | 0.8621875 |
| MGMT Methylated in Glioblastoma Multiforme | 0.680092477 | 0.679306097 | 0.67200656 |
| MGMT Methylated in Brain Lower Grade Glioma | 0.626100289 | 0.62034632 | 0.620779221 |
| IDH Methylated in Brain Lower Grade Glioma | 0.746118205 | 0.746215391 | 0.749801786 |
| IDH Methylated in Glioblastoma Multiforme | 0.871896392 | 0.855846438 | 0.869148936 |
| Obesity in Uterine Corpus Endometrial Carcinoma | 0.587741651 | 0.593281853 | 0.625733252 |
| Obesity in Renal Papillary Cell Carcinoma | 0.709077381 | 0.722470238 | 0.680446429 |
| Obesity in Esophageal Carcinoma | 0.652244444 | 0.648977778 | 0.6879 |
| Alcohol in Head and Neck | 0.429206349 | 0.424761905 | 0.472698413 |
| Alcohol in Esophageal Carcinoma | 0.859444444 | 0.859444444 | 0.838611111 |
| Alcohol in Liver Hepatocellular Carcinoma | 0.546237521 | 0.54450334 | 0.521022229 |
| Hepatitis B in Liver Hepatocellular Carcinoma | 0.538041394 | 0.538041394 | 0.520651416 |
| Hepatitis C in Liver Hepatocellular Carcinoma | 0.603453159 | 0.606623094 | 0.579004046 |
| Aristolochic Acid in Bladder Urothelial Carcinoma | 0.956764706 | 0.952058824 | 0.944558824 |
| Asbestos in Mesothelioma | 0.579104046 | 0.590367935 | 0.573089105 |
| High Apobec in Cervical Squamous | 0.608699301 | 0.606993007 | 0.59034965 |
| High Apobec in Renal Clear Cell Carcinoma | 0.433681933 | 0.431947479 | 0.437242017 |
| Average | 0.683007161 | 0.68611066 | 0.690078943 |
| sd | 0.162230383 | 0.161069197 | 0.160274687 |
| Restricted Ave | 0.720414279 | 0.723029451 | 0.742964092 |
| Restricted sd | 0.192133939 | 0.184101188 | 0.17917222 |

TABLE 4
Proportion of mutational load due to normal aging. For each indicated cancer type, and in the presence, or absence (“unexposed”), of
an indicated environmental or inherited factor, the distribution (2.5%, 50%, 97.5% percentiles) of the proportion of the overall mutational
load that can be attributable to normal aging is provided. This proportion was estimated by using the median (50% percentile) of the mutation
rate (year) in the patient population of the corresponding cancer type and in the absence of any known environmental or inherited factor.

| | | 50% | 50% | Age Signature | Exposure |
|---|---|---|---|---|---|
| | 50% | [Lower 2.5%] | [Upper 97.5%] | Sample Size | Sample Size |
| POLe Mutation in Colorectal Adenocarcinoma | 0.09130784 | 0.008593716 | 0.493325055 | 352 | 16 |
| POLe Mutation in Uterine Corpus Endometrial Carcinoma | 0.11501158 | 0.004084892 | 0.890404266 | 81 | 42 |
| MLH Silenced in Colorectal Adenocarcinoma | 0.1330663 | 0.051146202 | 0.166014906 | 352 | 6 |
| POLD Mutation in Uterine Corpus Endometrial Carcinoma | 0.16125052 | 0.022684635 | 0.449984749 | 81 | 16 |
| MLH Silenced in Stomach Adenocarcinoma | 0.17857501 | 0.088698113 | 0.548709321 | 159 | 20 |
| MLH Silenced in Uterine Corpus Endometrial Carcinoma | 0.2013287 | 0.055652337 | 0.405024373 | 81 | 33 |
| Aristolochic Acid in Bladder Urothelial Carcinoma | 0.20412619 | 0.019204306 | 0.501113544 | 147 | 19 |
| POLe Mutation in Stomach Adenocarcinoma | 0.20913625 | 0.02874153 | 0.54025735 | 159 | 11 |
| UV* in Skin Cutaneous Melanoma | 0.26207542 | 0.050518365 | 0.736029163 | 126 | 300 |
| Smoking in Lung Adenocarcinoma | 0.29201744 | 0.028888631 | 1 | 57 | 303 |
| POLD Mutation in Stomach Adenocarcinoma | 0.29683639 | 0.058874162 | 0.888871868 | 159 | 9 |
| BRCA1/2 Mutation in Breast Invasive Carcinoma | 0.34024335 | 0.038405257 | 0.953919764 | 691 | 34 |
| POLe Mutation in Breast Invasive Carcinoma | 0.51189936 | 0.058894951 | 0.960836739 | 691 | 13 |
| BRCA1/2 Mutation in Ovarian Serous Cystadenocarcinoma | 0.56514814 | 0.200365053 | 0.985214973 | 137 | 19 |
| Obesity in Renal Papillary Cell Carcinoma | 0.60351128 | 0.081675089 | 1 | 84 | 31 |
| Unexposed Uterine Corpus Endometrial Carcinoma | 0.61864617 | 0.078799616 | 0.993602244 | 81 | 81 |
| Obesity in Uterine Corpus Endometrial Carcinoma | 0.65294542 | 0.077162002 | 0.997575441 | 81 | 188 |
| Smoking in Head and Neck | 0.65386773 | 0.146782141 | 1 | 183 | 258 |
| Smoking in Bladder Urothelial Carcinoma | 0.66845217 | 0.179168704 | 1 | 147 | 203 |
| Smoking in Cervical Squamous | 0.69163208 | 0.150778816 | 1 | 217 | 49 |
| Smoking in Renal Papillary Cell Carcinoma | 0.69267146 | 0.183816836 | 0.989101002 | 84 | 16 |
| Hepatitis C in Liver Hepatocellular Carcinoma | 0.7089051 | 0.200272479 | 0.971000573 | 88 | 31 |
| Hepatitis B in Liver Hepatocellular Carcinoma | 0.70971703 | 0.314117123 | 0.96200605 | 88 | 75 |
| Unexposed Acute Myeloid Leukemia | 0.71883088 | 0.312710997 | 1 | 71 | 71 |
| Unexposed Adrenocortical Carcinoma | 0.72468891 | 0.302497437 | 1 | 74 | 74 |
| Alcohol in Liver Hepatocellular Carcinoma | 0.73055638 | 0.230861702 | 0.995407235 | 88 | 66 |
| Unexposed Breast Invasive Carcinoma | 0.73266481 | 0.296743614 | 1 | 691 | 691 |
| MGMT Methylated in Glioblastoma Multiforme | 0.73283329 | 0.42930326 | 0.957292268 | 190 | 93 |
| Obesity in Colorectal Adenocarcinoma | 0.73436876 | 0.126123803 | 0.984830154 | 352 | 76 |
| Unexposed Head and Neck | 0.73764707 | 0.332251235 | 1 | 183 | 183 |
| MGMT Methylated in Brain Lower Grade Glioma | 0.74002377 | 0.435900165 | 1 | 55 | 33 |
| Unexposed Bladder Urothelial Carcinoma | 0.75007554 | 0.289298114 | 1 | 147 | 147 |
| Unexposed Stomach Adenocarcinoma | 0.75576683 | 0.335033793 | 1 | 159 | 159 |
| High Copy Number in Uterine Corpus Endometrial Carcinoma | 0.75716038 | 0.17590482 | 0.999998988 | 81 | 42 |
| High Apobec in Renal Clear Cell Carcinoma | 0.75852961 | 0.592223166 | 0.954320931 | 197 | 24 |
| Low Copy Number in Uterine Corpus Endometrial Carcinoma | 0.76056856 | 0.144767093 | 0.997229605 | 81 | 64 |
| Unexposed Cervical Squamous | 0.76127952 | 0.262601223 | 1 | 217 | 217 |
| Unexposed Skin Cutaneous Melanoma | 0.76531505 | 0.265555713 | 1 | 126 | 125 |
| Unexposed Prostate Adenocarcinoma | 0.76731153 | 0.437957703 | 1 | 465 | 465 |
| Smoking in Pancreatic Adenocarcinoma | 0.76839297 | 0.2851577 | 1 | 58 | 51 |
| Unexposed Thyroid Carcinoma | 0.76922446 | 0.28683257 | 1 | 448 | 448 |
| High Apobec in Cervical Squamous | 0.77150187 | 0.244019916 | 1 | 217 | 65 |
| IDH Methylated in Glioblastoma Multiforme | 0.7716081 | 0.412633376 | 1 | 190 | 233 |
| IDH Methylated in Brain Lower Grade Glioma | 0.77351073 | 0.347753813 | 1 | 55 | 79 |
| Unexposed Glioblastoma Multiforme | 0.78675716 | 0.405031799 | 1 | 190 | 190 |
| Unexposed Pheochromocytoma and Paraganglioma | 0.78865758 | 0.433398438 | 1 | 149 | 149 |
| Unexposed Thymoma | 0.79127129 | 0.400768751 | 1 | 117 | 117 |
| Unexposed Lung Adenocarcinoma | 0.7913192 | 0.345454992 | 1 | 57 | 56 |
| Unexposed Testicular Germ Cell Tumors | 0.79219171 | 0.32826087 | 1 | 125 | 125 |
| Unexposed Ovarian Serous Cystadenocarcinoma | 0.79794653 | 0.333057089 | 1 | 137 | 137 |
| Unexposed Colorectal Adenocarcinoma | 0.80203204 | 0.403890838 | 1 | 352 | 352 |
| Unexposed Sarcoma | 0.80221285 | 0.395180941 | 1 | 233 | 233 |
| Unexposed Renal Clear Cell Carcinoma | 0.80709951 | 0.553521172 | 0.993008679 | 197 | 197 |
| Unexposed Liver Hepatocellular Carcinoma | 0.80758764 | 0.415139533 | 1 | 88 | 88 |
| Unexposed Renal Papillary Cell Carcinoma | 0.80776323 | 0.316966259 | 1 | 84 | 84 |
| Unexposed Uterine Carcinosarcoma | 0.82430449 | 0.483720959 | 1 | 54 | 54 |
| Smoking in Esophagus Squamous | 0.82995336 | 0.351044216 | 1 | 80 | 53 |
| Unexposed Kidney Chromophobe | 0.83259487 | 0.548801541 | 1 | 53 | 53 |
| Unexposed Esophagus Squamous | 0.83546228 | 0.414632783 | 1 | 80 | 80 |
| Unexposed Pancreatic Adenocarcinoma | 0.84160385 | 0.356986514 | 1 | 58 | 56 |
| Smoking in Esophagus Adenocarcimona | 0.84274508 | 0.449237327 | 1 | 58 | 35 |
| Unexposed Brain Lower Grade Glioma | 0.84510054 | 0.470757586 | 1 | 55 | 55 |
| Unexposed Esophagus Adenocarcimona | 0.84920996 | 0.471004785 | 1 | 58 | 58 |
| Unexposed Uveal Melanoma | 0.85149229 | 0.448106061 | 1 | 61 | 61 |
| Unexposed Cholangiocarcinoma | 0.86821106 | 0.59523065 | 1 | 43 | 43 |
| Alcohol in Head and Neck | 0.89189667 | 0.533406804 | 1 | 183 | 14 |
| Average | 0.6580552 | 0.274652365 | 0.929016352 | 166.4090909 | 113.166667 |
| Median | 0.7564636 | 0.299620526 | 1 | 125.5 | 65.5 |
| Lower 2.5% | 0.12629578 | 0.015225335 | 0.433124608 | 53.625 | 10.25 |
| Upper 97.5% | 0.85776183 | 0.56803442 | 1 | 691 | 454.375 |

TABLE 5
An example of projecting probabilities on a refinement partition: Exposure 1 signature ([C > T]G,
[C > T]H, Remaining) = (15%, 5%, 80%) and Exposure 2 signature (A[C > T], B[C > T],
Remaining) = (3%, 7%, 90%). H means “not G” and B means “not A”. The symbol ‘#’ before a k-
nucleotide represents the average count of that k-nucleotide on the genomic/exomic dataset where
the signature (Exposure 1 or Exposure 2) was extracted from.

| | | | Projected | | | | Projected |
|---|---|---|---|---|---|---|---|
| | | | signature | | | | signature |
| Exposure 1 | Proportion | | on | Exposure 2 | Proportion | | on |
| signature | of feature | Refinement | refinement | signature | of feature | Refinement | refinement |
| (features) | in signature | partition | partition | (features) | in signature | partition | partition |
| [C > T]G | 15% | A[C > T]G | $15 \% \frac{\# ACG}{\# CG}$ | A[C > T] | 3% | A[C > T]G | $3 \% \frac{\# ACG}{\# AC}$ |
| | | B[C > T]G | $15 \% \frac{\# BCG}{\# CG}$ | | | A[C > T]H | $3 \% \frac{\# BCG}{\# AC}$ |
| [C > T]H | 5% | A[C > T]H | $5 \% \frac{\# ACH}{\# CH}$ | B[C > T] | 7% | B[C > T]G | $7 \% \frac{\# ACH}{\# BC}$ |
| | | B[C > T]H | $5 \% \frac{\# BCH}{\# CH}$ | | | B[C > T]H | $7 \% \frac{\# BCH}{\# BC}$ |
| Remaining | 80% | Remaining | 80% | Remaining | 90% | Remaining | 90% |

TABLE 6
Signatures, their features, and their features' frequencies. For each indicated cancer
type, and each indicated environmental, inherited, or age factor, the selected features
of the corresponding signature, with their observed and expected frequencies, are provided.

| | | | | |
|---|---|---|---|---|
| V1 | V2 | V3 | V4 | V5 |

Age in Acute Myeloid Leukemia
Signature

| Mutation Type | C > A | |
|---|---|---|
| Frequency of Mutation | 0.16 | [±0.18] |

| | |
|---|---|
| Expected of Mutation | 0.14 |

Age in Bladder Urothelial Carcinoma
Signature

| Mutation Type | (ACG)[C > T]G | | (ACG)[C > A] | | (ACG)[C > T](ACT) | | (ACG)[C > G] | |
|---|---|---|---|---|---|---|---|---|
| Frequency of Mutation | 0.046 | [±0.042] | 0.056 | [±0.028] | 0.049 | [±0.024] | 0.056 | [±0.028] |

| | | | | |
|---|---|---|---|---|
| Expected of Mutation | 0.015 | 0.13 | 0.11 | 0.13 |

Age in Lung Adenocarcinoma
Signature

| Mutation Type | C > A | |
|---|---|---|
| Frequency of Mutation | 0.22 | [±0.13] |

| | |
|---|---|
| Expected of Mutation | 0.17 |

Age in Brain Lower Grade Glioma
Signature

| Mutation Type | C > T | | C > A | | C > G | | T > A | |
|---|---|---|---|---|---|---|---|---|
| Frequency of Mutation | 0.47 | [±0.17] | 0.11 | [±0.034] | 0.11 | [±0.034] | 0.11 | [±0.034] |

| | | | | |
|---|---|---|---|---|
| Expected of Mutation | 0.17 | 0.17 | 0.17 | 0.16 |

Age in Head and Neck
Signature

| Mutation Type | (AG)[C > A] | | (ACG)[C > T](CT) | | (ACG)[C > G] | | T > A | |
|---|---|---|---|---|---|---|---|---|
| Frequency of Mutation | 0.039 | [±0.015] | 0.037 | [±0.014] | 0.064 | [±0.024] | 0.083 | [±0.032] |

| | | | | |
|---|---|---|---|---|
| Expected of Mutation | 0.076 | 0.073 | 0.13 | 0.16 |

Age in Renal Clear Cell Carcinoma
Signature

| Mutation Type | (ACG)[C > T](ACT) | | C > G | | T > A | | T > G | |
|---|---|---|---|---|---|---|---|---|
| Frequency of Mutation | 0.16 | [±0.056] | 0.12 | [±0.024] | 0.11 | [±0.024] | 0.11 | [±0.024] |

| | | | | |
|---|---|---|---|---|
| Expected of Mutation | 0.11 | 0.17 | 0.16 | 0.16 |

Age in Renal Papillary Cell Carcinoma
Signature

| Mutation Type | (ACG)[C > T](ACT) | | (ATG)[C > A] | | C > G | | T > A | |
|---|---|---|---|---|---|---|---|---|
| Frequency of Mutation | 0.15 | [±0.073] | 0.088 | [±0.022] | 0.13 | [±0.032] | 0.12 | [±0.031] |

| | | | | |
|---|---|---|---|---|
| Expected of Mutation | 0.11 | 0.12 | 0.17 | 0.16 |

Age in Kidney Chromophobe
Signature

| Mutation Type | C > T | | C > G | | T > A | | T > G | |
|---|---|---|---|---|---|---|---|---|
| Frequency of Mutation | 0.36 | [±0.14] | 0.11 | [±0.045] | 0.11 | [±0.044] | 0.11 | [±0.044] |

| | | | | |
|---|---|---|---|---|
| Expected of Mutation | 0.17 | 0.17 | 0.16 | 0.16 |

Age in Liver Hepatocellular Carcinoma
Signature

| Mutation Type | (ACT)[C > T](ACT) | | A[T > C](CTG) | |
|---|---|---|---|---|
| Frequency of Mutation | 0.18 | [±0.052] | 0.046 | [±0.031] |

| | | |
|---|---|---|
| Expected of Mutation | 0.11 | 0.028 |

Age in Stomach Adenocarcinoma
Signature

| Mutation Type | (ACG)[C > T](ACT) | | (ACG)[C > A](CTG) | | (AC)[C > A]A | | C > G | |
|---|---|---|---|---|---|---|---|---|
| Frequency of Mutation | 0.15 | [±0.056] | 0.052 | [±0.011] | 0.016 | [±0.0034] | 0.1 | [±0.022] |

| | | | | |
|---|---|---|---|---|
| Expected of Mutation | 0.11 | 0.087 | 0.027 | 0.17 |

Age in Thyroid Carcinoma
Signature

| Mutation Type | C > G | | T > A | | T > G | | T > C | |
|---|---|---|---|---|---|---|---|---|
| Frequency of Mutation | 0.11 | [±0.047] | 0.11 | [±0.046] | 0.11 | [±0.046] | 0.11 | [±0.046] |

| | | | | |
|---|---|---|---|---|
| Expected of Mutation | 0.17 | 0.16 | 0.16 | 0.16 |

Age in Uveal Melanoma
Signature

| Mutation Type | C > T | |
|---|---|---|
| Frequency of Mutation | 0.35 | [±0.13] |

| | |
|---|---|
| Expected of Mutation | 0.17 |

Age in Skin Cutaneous Melanoma
Signature

| Mutation Type | C[C > A](ACT) | |
|---|---|---|
| Frequency of Mutation | 0.068 | [±0.11] |

| | |
|---|---|
| Expected of Mutation | 0.044 |

Age in Adrenocortical Carcinoma
Signature

| Mutation Type | C > A | | (ACG)[C > T](ACT) | |
|---|---|---|---|---|
| Frequency of Mutation | 0.21 | [±0.12] | 0.15 | [±0.081] |

| | | |
|---|---|---|
| Expected of Mutation | 0.17 | 0.11 |

Age in Cholangiocarcinoma
Signature

| Mutation Type | C > G | | T > A | | T > G | | T > C | |
|---|---|---|---|---|---|---|---|---|
| Frequency of Mutation | 0.094 | [±0.03] | 0.092 | [±0.029] | 0.092 | [±0.029] | 0.092 | [±0.029] |

| | | | | |
|---|---|---|---|---|
| Expected of Mutation | 0.17 | 0.16 | 0.16 | 0.16 |

Age in Glioblastoma Multiforme
Signature

| Mutation Type | (ATG)[C > A](ATG) | | (TG)[C > A]C | | C > G | | T > A | |
|---|---|---|---|---|---|---|---|---|
| Frequency of Mutation | 0.051 | [±0.012] | 0.016 | [±0.0039] | 0.1 | [±0.025] | 0.1 | [±0.024] |

| | | | | |
|---|---|---|---|---|
| Expected of Mutation | 0.083 | 0.026 | 0.17 | 0.16 |

Age in Cervical Squamous
Signature

| Mutation Type | (ACG)[C > A] | | (ACG)[C > T](ACT) | | (ACG)[C > G] | | T > A | |
|---|---|---|---|---|---|---|---|---|
| Frequency of Mutation | 0.056 | [±0.025] | 0.05 | [±0.022] | 0.056 | [±0.025] | 0.074 | [±0.032] |

| | | | | |
|---|---|---|---|---|
| Expected of Mutation | 0.13 | 0.11 | 0.13 | 0.16 |

Age in Colorectal Adenocarcinoma
Signature

| Mutation Type | G[C > T]G | | A[C > T]G | | (CT)[C > T]G | | G[C > T](ACT) | |
|---|---|---|---|---|---|---|---|---|
| Frequency of Mutation | 0.078 | [±0.051] | 0.061 | [±0.037] | 0.1 | [±0.046] | 0.057 | [±0.032] |

| | | | | |
|---|---|---|---|---|
| Expected of Mutation | 0.005 | 0.0036 | 0.0095 | 0.037 |

Age in Pheochromocytoma and Paraganglioma
Signature

| Mutation Type | C > A | | C > G | | T > A | | T > G | |
|---|---|---|---|---|---|---|---|---|
| Frequency of Mutation | 0.11 | [±0.038] | 0.11 | [±0.038] | 0.11 | [±0.038] | 0.11 | [±0.038] |

| | | | | |
|---|---|---|---|---|
| Expected of Mutation | 0.17 | 0.17 | 0.16 | 0.16 |

Age in Pancreatic Adenocarcinoma
Signature

| Mutation Type | C > T | |
|---|---|---|
| Frequency of Mutation | 0.48 | [±0.16] |

| | |
|---|---|
| Expected of Mutation | 0.17 |

Age in Prostate Adenocarcinoma
Signature

| Mutation Type | (ACG)[C > A](ACT) | | T[C > A](AT) | | C > G | | T > A | |
|---|---|---|---|---|---|---|---|---|
| Frequency of Mutation | 0.076 | [±0.019] | 0.018 | [±0.0044] | 0.12 | [±0.028] | 0.11 | [±0.028] |

| | | | | |
|---|---|---|---|---|
| Expected of Mutation | 0.11 | 0.026 | 0.17 | 0.16 |

Age in Esophagus Squamous
Signature

| Mutation Type | (ACG)[C > T]G | |
|---|---|---|
| Frequency of Mutation | 0.063 | [±0.038] |

| | |
|---|---|
| Expected of Mutation | 0.015 |

Age in Esophagus Adenocarcimona
Signature

| Mutation Type | C > T | | T > G | |
|---|---|---|---|---|
| Frequency of Mutation | 0.37 | [±0.095] | 0.16 | [±0.096] |

| | | |
|---|---|---|
| Expected of Mutation | 0.17 | 0.16 |

Age in Uterine Corpus Endometrial Carcinoma
Signature

| Mutation Type | (CT)[C > T]G | | (ACT)[C > T](ACT) | | A[T > C]G | |
|---|---|---|---|---|---|---|
| Frequency of Mutation | 0.074 | [±0.05] | 0.18 | [±0.075] | 0.013 | [±0.016] |

| | | | |
|---|---|---|---|
| Expected of Mutation | 0.0095 | 0.11 | 0.011 |

Age in Uterine Carcinosarcoma
Signature

| Mutation Type | C > G | | T > A | | T > G | | T > C | |
|---|---|---|---|---|---|---|---|---|
| Frequency of Mutation | 0.1 | [±0.027] | 0.1 | [±0.027] | 0.1 | [±0.027] | 0.1 | [±0.027] |

| | | | | |
|---|---|---|---|---|
| Expected of Mutation | 0.17 | 0.16 | 0.16 | 0.16 |

Age in Breast Invasive Carcinoma
Signature

| Mutation Type | (CG)[C > T]G | | A[C > A]C | | A[C > T]G | |
|---|---|---|---|---|---|---|
| Frequency of Mutation | 0.057 | [±0.049] | 0.017 | [±0.025] | 0.026 | [±0.031] |

| | | | |
|---|---|---|---|
| Expected of Mutation | 0.011 | 0.0098 | 0.0036 |

Age in Sarcoma
Signature

| Mutation Type | [C > T](ACT) | | (ACT)[C > T]G | | [C > G](ACG) | | (ACG)[C > G]T | |
|---|---|---|---|---|---|---|---|---|
| Frequency of Mutation | 0.26 | [±0.1] | 0.064 | [±0.05] | 0.071 | [±0.018] | 0.022 | [±0.0055] |

| | | | | |
|---|---|---|---|---|
| Expected of Mutation | 0.15 | 0.013 | 0.12 | 0.036 |

Age in Testicular Germ Cell Tumors
Signature

| Mutation Type | C > G | | T > A | | T > G | | T > C | |
|---|---|---|---|---|---|---|---|---|
| Frequency of Mutation | 0.1 | [±0.041] | 0.1 | [±0.04] | 0.1 | [±0.04] | 0.1 | [±0.04] |

| | | | | |
|---|---|---|---|---|
| Expected of Mutation | 0.17 | 0.16 | 0.16 | 0.16 |

Age in Thymoma
Signature

| Mutation Type | C > T | | C > G | | T > A | | T > G | |
|---|---|---|---|---|---|---|---|---|
| Frequency of Mutation | 0.3 | [±0.15] | 0.11 | [±0.042] | 0.11 | [±0.041] | 0.11 | [±0.041] |

| | | | | |
|---|---|---|---|---|
| Expected of Mutation | 0.17 | 0.17 | 0.16 | 0.16 |

Age in Ovarian Serous Cystadenocarcinoma
Signature

| Mutation Type | (ACT)[C > T]G | | (ACT)[C > A]G | |
|---|---|---|---|---|
| Frequency of Mutation | 0.037 | [±0.034] | 0.016 | [±0.015] |

| | | |
|---|---|---|
| Expected of Mutation | 0.005 | 0.005 |

Smoking in Bladder Urothelial Carcinoma
Signature

| Mutation Type | (ACG)[C > T]G | |
|---|---|---|
| Unexposed Mutation Freq. | 0.053 | [±0.049] |

| | |
|---|---|
| Exposed Mutation Freq. | 0.042 [±0.038] |

Smoking in Lung Adenocarcinoma
Signature

| Mutation Type | (ATG)[C > A](AT) | | C[C > A]C | | C[C > A](AT) | | C[T > A]G | |
|---|---|---|---|---|---|---|---|---|
| Unexposed Mutation Freq. | 0.071 | [±0.047] | 0.015 | [±0.023] | 0.043 | [±0.058] | 0.0075 | [±0.012] |
| Exposed Mutation Freq. | 0.13 | [±0.043] | 0.043 | [±0.024] | 0.086 | [±0.043] | 0.022 | [±0.014] |

Smoking in Head and Neck
Signature

| Mutation Type | A[T > C]A | | (ACG)[C > T]G | | (AG)[C > A](CT) | | (ACG)[C > G] | |
|---|---|---|---|---|---|---|---|---|
| Unexposed Mutation Freq. | 0.0053 | [±0.011] | 0.095 | [±0.054] | 0.016 | [±0.0069] | 0.046 | [±0.02] |
| Exposed Mutation Freq. | 0.015 | [±0.016] | 0.057 | [±0.044] | 0.021 | [±0.0073] | 0.06 | [±0.021] |

Smoking in Renal Papillary Cell Carcinoma
Signature

| Mutation Type | C > T | | C > A | | C > G | | T > A | |
|---|---|---|---|---|---|---|---|---|
| Unexposed Mutation Freq. | 0.26 | [±0.1] | 0.24 | [±0.17] | 0.13 | [±0.032] | 0.12 | [±0.031] |
| Exposed Mutation Freq. | 0.23 | [±0.11] | 0.3 | [±0.25] | 0.12 | [±0.05] | 0.12 | [±0.049] |

Smoking in Pancreatic Adenocarcinoma
Signature

| Mutation Type | T[C > A](ACT) | |
|---|---|---|
| Unexposed Mutation Freq. | 0.042 | [±0.038] |
| Exposed Mutation Freq. | 0.062 | [±0.05] |

Smoking in Esophagus Squamous
Signature

| Mutation Type | T[C > A] | | T > A | | T > G | | [T > C](CTG) | |
|---|---|---|---|---|---|---|---|---|
| Unexposed Mutation Freq. | 0.075 | [±0.029] | 0.076 | [±0.024] | 0.076 | [±0.024] | 0.064 | [±0.02] |
| Exposed Mutation Freq. | 0.058 | [±0.024] | 0.088 | [±0.028] | 0.088 | [±0.028] | 0.074 | [±0.023] |

Smoking in Esophagus Adenocarcimona
Signature

| Mutation Type | C[T > G]T | |
|---|---|---|
| Unexposed Mutation Freq. | 0.06 | [±0.054] |
| Exposed Mutation Freq. | 0.092 | [±0.063] |

Smoking in Cervical Squamous
Signature

| Mutation Type | T[C > T]G | | T[C > T]A | | T[C > G]A | |
|---|---|---|---|---|---|---|
| Unexposed Mutation Freq. | 0.048 | [±0.03] | 0.12 | [±0.07] | 0.068 | [±0.056] |
| Exposed Mutation Freq. | 0.04 | [±0.024] | 0.14 | [±0.076] | 0.076 | [±0.057] |

POLe Mutation in Uterine Corpus Endometrial Carcinoma
Signature

| Mutation Type | (AG)[C > A](ACG) | | (ACG)[C > G] | | T[C > G](CG) | | T > A | |
|---|---|---|---|---|---|---|---|---|
| Unexposed Mutation Freq. | 0.027 | [±0.011] | 0.062 | [±0.026] | 0.0083 | [±0.0035] | 0.081 | [±0.034] |
| Exposed Mutation Freq. | 0.016 | [±0.01] | 0.036 | [±0.024] | 0.0048 | [±0.0032] | 0.047 | [±0.032] |

POLe Mutation in Stomach Adenocarcinoma
Signature

| Mutation Type | T[C > T](ACT) | | G[C > T]G | | (ACG)[C > T](ACT) | | C > G | |
|---|---|---|---|---|---|---|---|---|
| Unexposed Mutation Freq. | 0.081 | [±0.047] | 0.048 | [±0.032] | 0.15 | [±0.056] | 0.074 | [±0.027] |
| Exposed Mutation Freq. | 0.036 | [±0.023] | 0.097 | [±0.048] | 0.2 | [±0.055] | 0.051 | [±0.043] |

POLe Mutation in Colorectal Adenocarcinoma
Signature

| Mutation Type | (CT)[C > T](ACT) | | G[C > T](ACT) | | (AC)[C > A]A | | G[C > A]A | |
|---|---|---|---|---|---|---|---|---|
| Unexposed Mutation Freq. | 0.12 | [±0.056] | 0.057 | [±0.032] | 0.029 | [±0.022] | 0.024 | [±0.021] |
| Exposed Mutation Freq. | 0.071 | [±0.031] | 0.12 | [±0.063] | 0.0089 | [±0.0065] | 0.0049 | [±0.0083] |

POLe Mutation in Breast Invasive Carcinoma
Signature

| Mutation Type | A[C > T]G | | T[C > G]A | | (ACG)[C > A](ATG) | | (CG)[C > A]C | |
|---|---|---|---|---|---|---|---|---|
| Unexposed Mutation Freq. | 0.026 | [±0.031] | 0.023 | [±0.033] | 0.093 | [±0.065] | 0.028 | [±0.019] |
| Exposed Mutation Freq. | 0.012 | [±0.023] | 0.03 | [±0.032] | 0.13 | [±0.1] | 0.037 | [±0.031] |

MLH Silenced in Uterine Corpus Endometrial Carcinoma
Signature

| Mutation Type | C[T > C]G | | G[C > T](ACT) | | C[C > A]T | | G[T > C]G | |
|---|---|---|---|---|---|---|---|---|
| Unexposed Mutation Freq. | 0.015 | [±0.019] | 0.089 | [±0.065] | 0.023 | [±0.027] | 0.0097 | [±0.014] |
| Exposed Mutation Freq. | 0.042 | [±0.018] | 0.18 | [±0.054] | 0.053 | [±0.017] | 0.022 | [±0.012] |

MLH Silenced in Stomach Adenocarcinoma
Signature

| Mutation Type | C[C > A]T | | G[C > T](ACT) | | (ACT)[T > C]G | | (AG)[C > A](CTG) | |
|---|---|---|---|---|---|---|---|---|
| Unexposed Mutation Freq· | 0.012 | [±0.02] | 0.054 | [±0.036] | 0.029 | [±0.023] | 0.028 | [±0.008] |
| Exposed Mutation Freq. | 0.056 | [±0.014] | 0.14 | [±0.033] | 0.077 | [±0.021] | 0.015 | [±0.0038] |

MLH Silenced in Colorectal Adenocarcinoma
Signature

| Mutation Type | T > C | |
|---|---|---|
| Unexposed Mutation Freq. | 0.11 | [±0.095] |
| Exposed Mutation Freq. | 0.22 | [±0.062] |

BRCA1/2 Mutation in Breast Invasive Carcinoma
Signature

| Mutation Type | T[C > G]T | | T[C > G]A | | T[C > G](CG) | | (CG)[C > T]G | |
|---|---|---|---|---|---|---|---|---|
| Unexposed Mutation Freq. | 0.03 | [±0.037] | 0.023 | [±0.032] | 0.018 | [±0.026] | 0.057 | [±0.049] |
| Exposed Mutation Freq. | 0.069 | [±0.069] | 0.055 | [±0.06] | 0.027 | [±0.024] | 0.03 | [±0.028] |

BRCA1/2 Mutation in Ovarian Serous Cystadenocarcinoma
Signature

| Mutation Type | G[C > A](AT) | | C[T > A]G | | G[C > T](ACT) | | (ACT)[C > A]C | |
|---|---|---|---|---|---|---|---|---|
| Unexposed Mutation Freq. | 0.029 | [±0.024] | 0.018 | [±0.018] | 0.068 | [±0.089] | 0.054 | [±0.037] |
| Exposed Mutation Freq. | 0.035 | [±0.01] | 0.024 | [±0.019] | 0.046 | [±0.02] | 0.073 | [±0.069] |

UV* in Skin Cutaneous Melanoma
Signature

| Mutation Type | (ATG)[C > A] | | C > G | | T > A | | T > G | |
|---|---|---|---|---|---|---|---|---|
| Unexposed Mutation Freq. | 0.063 | [±0.029] | 0.09 | [±0.041] | 0.088 | [±0.04] | 0.088 | [±0.04] |
| Exposed Mutation Freq. | 0.019 | [±0.0058] | 0.026 | [±0.0082] | 0.026 | [±0.008] | 0.026 | [±0.008] |

POLD Mutation in Uterine Corpus Endometrial Carcinoma
Signature

| Mutation Type | (CT)[T > C]G | | G[C > T](ACT) | | C[C > A]T | | G[T > C]G | |
|---|---|---|---|---|---|---|---|---|
| Unexposed Mutation Freq. | 0.024 | [±0.025] | 0.089 | [±0.065] | 0.023 | [±0.027] | 0.0097 | [±0.014] |
| Exposed Mutation Freq. | 0.055 | [±0.028] | 0.17 | [±0.068] | 0.061 | [±0.044] | 0.023 | [±0.02] |

High Copy Number in Uterine Corpus Endometrial Carcinoma
Signature

| Mutation Type | G[C > T](ACT) | | C[C > T]G | | (AG)[C > A] | | (ACG)[C > G] | |
|---|---|---|---|---|---|---|---|---|
| Unexposed Mutation Freq. | 0.089 | [±0.065] | 0.038 | [±0.032] | 0.037 | [±0.015] | 0.062 | [±0.024] |
| Exposed Mutation Freq. | 0.043 | [±0.038] | 0.018 | [±0.019] | 0.047 | [±0.017] | 0.078 | [±0.028] |

Low Copy Number in Uterine Corpus Endometrial Carcinoma
Signature

| Mutation Type | (ATG)[C > A] | | (ACG)[C > G] | | T[C > G](CG) | | T > A | |
|---|---|---|---|---|---|---|---|---|
| Unexposed Mutation Freq. | 0.063 | [±0.024] | 0.066 | [±0.026] | 0.0088 | [±0.0034] | 0.086 | [±0.034] |
| Exposed Mutation Freq. | 0.08 | [±0.022] | 0.085 | [±0.024] | 0.011 | [±0.0031] | 0.11 | [±0.031] |

POLD Mutation in Stomach Adenocarcinoma
Signature

| Mutation Type | T[C > T](ACT) | | T[C > A] | | [T > C](ACG) | | (ATG)[T > C]T | |
|---|---|---|---|---|---|---|---|---|
| Unexposed Mutation Freq. | 0.081 | [±0.047] | 0.057 | [±0.042] | 0.097 | [±0.035] | 0.026 | [±0.0096] |
| Exposed Mutation Freq. | 0.03 | [±0.012] | 0.024 | [±0.019] | 0.16 | [±0.062] | 0.044 | [±0.017] |

MGMT Methylated in Glioblastoma Multiforme
Signature

| Mutation Type | (CT)[C > T](ACT) | | (ATG)[C > A](ATG) | | (TG)[C > A]C | | C > G | |
|---|---|---|---|---|---|---|---|---|
| Unexposed Mutation Freq. | 0.12 | [±0.064] | 0.051 | [±0.011] | 0.016 | [±0.0034] | 0.1 | [±0.022] |
| Exposed Mutation Freq. | 0.17 | [±0.076] | 0.046 | [±0.0093] | 0.015 | [±0.0029] | 0.094 | [±0.019] |

MGMT Methylated in Brain Lower Grade Glioma
Signature

| Mutation Type | [C > T](ACT) | |
|---|---|---|
| Unexposed Mutation Freq. | 0.26 | [±0.14] |
| Exposed Mutation Freq. | 0.33 | [±0.16] |

IDH Methylated in Brain Lower Grade Glioma
Signature

| Mutation Type | A[C > T]G | | (CT)[C > T]G | | G[T > C]C | | A[T > C](ATG) | |
|---|---|---|---|---|---|---|---|---|
| Unexposed Mutation Freq. | 0.033 | [±0.04] | 0.057 | [±0.052] | 0.023 | [±0.033] | 0.054 | [±0.05] |
| Exposed Mutation Freq. | 0.064 | [±0.054] | 0.082 | [±0.052] | 0.0079 | [±0.015] | 0.035 | [±0.029] |

IDH Methylated in Glioblastoma Multiforme
Signature

| Mutation Type | T > C | | (CT)[C > T]G | | G[C > T]G | | A[C > T]G | |
|---|---|---|---|---|---|---|---|---|
| Unexposed Mutation Freq. | 0.23 | [±0.069] | 0.035 | [±0.04] | 0.039 | [±0.041] | 0.036 | [±0.034] |
| Exposed Mutation Freq. | 0.14 | [±0.061] | 0.074 | [±0.047] | 0.069 | [±0.043] | 0.071 | [±0.048] |

Obesity in Uterine Corpus Endometrial Carcinoma
Signature

| Mutation Type | A[C > T]G | | G[C > T]G | |
|---|---|---|---|---|
| Unexposed Mutation Freq. | 0.032 | [±0.034] | 0.047 | [±0.042] |
| Exposed Mutation Freq. | 0.048 | [±0.037] | 0.062 | [±0.043] |

Obesity in Renal Papillary Cell Carcinoma
Signature

| Mutation Type | C[C > A](ACT) | | C > G | | T > A | | T > G | |
|---|---|---|---|---|---|---|---|---|
| Unexposed Mutation Freq. | 0.06 | [±0.063] | 0.14 | [±0.029] | 0.13 | [±0.028] | 0.13 | [±0.028] |
| Exposed Mutation Freq. | 0.19 | [±0.17] | 0.095 | [±0.051] | 0.093 | [±0.05] | 0.093 | [±0.05] |

Obesity in Esophageal Carcinoma
Signature

| Mutation Type | (ATG)[T > G]T | | C[T > G]T | |
|---|---|---|---|---|
| Unexposed Mutation Freq. | 0.018 | [±0.028] | 0.031 | [±0.062] |
| Exposed Mutation Freq. | 0.036 | [±0.025] | 0.07 | [±0.059] |

Obesity in Colorectal Adenocarcinoma
Signature

| Mutation Type | (CT)[C > T]G | | G[T > C]A | | A[C > T]G | | T[C > A]A | |
|---|---|---|---|---|---|---|---|---|
| Unexposed Mutation Freq. | 0.1 | [±0.041] | 0.0054 | [±0.0078] | 0.055 | [±0.028] | 0.02 | [±0.016] |
| Exposed Mutation Freq. | 0.11 | [±0.04] | 0.0078 | [±0.011] | 0.06 | [±0.028] | 0.018 | [±0.013] |

Alcohol in Head and Neck
Signature

| Mutation Type | C > G | | T > A | | T > G | | T > C | |
|---|---|---|---|---|---|---|---|---|
| Unexposed Mutation Freq. | 0.19 | [±0.12] | 0.059 | [±0.032] | 0.059 | [±0.032] | 0.059 | [±0.032] |
| Exposed Mutation Freq. | 0.16 | [±0.13] | 0.066 | [±0.034] | 0.066 | [±0.034] | 0.066 | [±0.034] |

Alcohol in Esophageal Carcinoma
Signature

| Mutation Type | C > T | |
|---|---|---|
| Unexposed Mutation Freq. | 0.44 | [±0.078] |
| Exposed Mutation Freq. | 0.34 | [±0.051] |

Alcohol in Liver Hepatocellular Carcinoma
Signature

| Mutation Type | (AC)[C > A]G | | A[T > C]A | | (ACT)[C > T](ACT) | | (AC)[C > A](AT) | |
|---|---|---|---|---|---|---|---|---|
| Unexposed Mutation Freq. | 0.012 | [±0.014] | 0.013 | [±0.015] | 0.18 | [±0.052] | 0.052 | [±0.032] |
| Exposed Mutation Freq. | 0.018 | [±0.016] | 0.018 | [±0.014] | 0.16 | [±0.052] | 0.059 | [±0.024] |

Hepatitis B in Liver Hepatocellular Carcinoma
Signature

| Mutation Type | G[T > C](CTG) | | A[T > C]A | |
|---|---|---|---|---|
| Unexposed Mutation Freq. | 0.038 | [±0.026] | 0.013 | [±0.015] |
| Exposed Mutation Freq. | 0.029 | [±0.02] | 0.02 | [±0.02] |

Hepatitis C in Liver Hepatocellular Carcinoma
Signature

| Mutation Type | G[C > T](ACT) | |
|---|---|---|
| Unexposed Mutation Freq. | 0.069 | [±0.035] |
| Exposed Mutation Freq. | 0.05 | [±0.025] |

Aristolochic Acid in Bladder Urothelial Carcinoma
Signature

| Mutation Type | T > A | | T[C > T](CT) | | T[C > T]A | | T[C > G]A | |
|---|---|---|---|---|---|---|---|---|
| Unexposed Mutation Freq. | 0.039 | [±0.029] | 0.11 | [±0.052] | 0.13 | [±0.066] | 0.076 | [±0.049] |
| Exposed Mutation Freq. | 0.63 | [±0.22] | 0.028 | [±0.03] | 0.028 | [±0.045] | 0.018 | [±0.023] |

Asbestos in Mesothelioma
Signature

| Mutation Type | [C > A]G | |
|---|---|---|
| Unexposed Mutation Freq. | 0.13 | [±0.17] |
| Exposed Mutation Freq. | 0.051 | [±0.043] |

High Apobec in Cervical Squamous
Signature

| Mutation Type | [C > A](CTG) | | (ACG)[C > A]A | | T > A | | T > G | |
|---|---|---|---|---|---|---|---|---|
| Unexposed Mutation Freq. | 0.057 | [±0.025] | 0.019 | [±0.0083] | 0.08 | [±0.035] | 0.08 | [±0.035] |
| Exposed Mutation Freq. | 0.044 | [±0.022] | 0.014 | [±0.0072] | 0.061 | [±0.031] | 0.061 | [±0.031] |

High Apobec in Renal Clear Cell Carcinoma
Signature

| Mutation Type | A[T > C]A | | A[T > C](CTG) | | T[C > T]A | |
|---|---|---|---|---|---|---|
| Unexposed Mutation Freq. | 0.0087 | [±0.013] | 0.034 | [±0.025] | 0.028 | [±0.022] |
| Exposed Mutation Freq. | 0.013 | [±0.015] | 0.043 | [±0.029] | 0.034 | [±0.025] |

| | | | | |
|---|---|---|---|---|
| V1 | V6 | V7 | V8 | V9 |

Age in Acute Myeloid Leukemia
Signature

Mutation Type
Frequency of Mutation
Expected of Mutation

Age in Bladder Urothelial Carcinoma
Signature

| Mutation Type | T > A | | T > G | | T > C | | T[C > A]A | |
|---|---|---|---|---|---|---|---|---|
| Frequency of Mutation | 0.073 | [±0.036] | 0.073 | [±0.036] | 0.073 | [±0.036] | 0.025 | [±0.027] |

| | | | | |
|---|---|---|---|---|
| Expected of Mutation | 0.16 | 0.16 | 0.16 | 0.012 |

Age in Lung Adenocarcinoma
Signature

Mutation Type
Frequency of Mutation
Expected of Mutation

Age in Brain Lower Grade Glioma
Signature

| Mutation Type | T > G | | T > C | |
|---|---|---|---|---|
| Frequency of Mutation | 0.11 | [±0.034] | 0.11 | [±0.034] |

| | | |
|---|---|---|
| Expected of Mutation | 0.16 | 0.16 |

Age in Head and Neck
Signature

| Mutation Type | T > G | | T > C | | (ACG)[C > T]A | | T[C > G]T | |
|---|---|---|---|---|---|---|---|---|
| Frequency of Mutation | 0.083 | [±0.032] | 0.083 | [±0.032] | 0.05 | [±0.029] | 0.056 | [±0.046] |

| | | | | |
|---|---|---|---|---|
| Expected of Mutation | 0.16 | 0.16 | 0.039 | 0.014 |

Age in Renal Clear Cell Carcinoma
Signature

| Mutation Type | (CTG)[T > C](CTG) | | (ACG)[C > T]G | | T[C > A] | | T[C > T](CT) | |
|---|---|---|---|---|---|---|---|---|
| Frequency of Mutation | 0.076 | [±0.016] | 0.034 | [±0.036] | 0.069 | [±0.041] | 0.043 | [±0.027] |

| | | | | |
|---|---|---|---|---|
| Expected of Mutation | 0.11 | 0.015 | 0.043 | 0.027 |

Age in Renal Papillary Cell Carcinoma
Signature

| Mutation Type | T > G | | (CTG)[T > C](CTG) | | (TG)[T > C]A | | A[T > C](CTG) | |
|---|---|---|---|---|---|---|---|---|
| Frequency of Mutation | 0.12 | [±0.031] | 0.082 | [±0.021] | 0.01 | [±0.0025] | 0.039 | [±0.034] |

| | | | | |
|---|---|---|---|---|
| Expected of Mutation | 0.16 | 0.11 | 0.014 | 0.028 |

Age in Kidney Chromophobe
Signature

| Mutation Type | T > C | | C > A | |
|---|---|---|---|---|
| Frequency of Mutation | 0.11 | [±0.044] | 0.21 | [±0.14] |

| | | |
|---|---|---|
| Expected of Mutation | 0.16 | 0.17 |

Age in Liver Hepatocellular Carcinoma
Signature

Mutation Type
Frequency of Mutation
Expected of Mutation

Age in Stomach Adenocarcinoma
Signature

| Mutation Type | T > A | | [T > G](ACG) | | [T > C](ACG) | | (ATG)[T > C]T | |
|---|---|---|---|---|---|---|---|---|
| Frequency of Mutation | 0.099 | [±0.021] | 0.072 | [±0.015] | 0.072 | [±0.015] | 0.019 | [±0.0041] |

| | | | | |
|---|---|---|---|---|
| Expected of Mutation | 0.16 | 0.12 | 0.12 | 0.032 |

Age in Thyroid Carcinoma
Signature

| Mutation Type | C > T | | C > A | |
|---|---|---|---|---|
| Frequency of Mutation | 0.34 | [±0.18] | 0.22 | [±0.18] |

| | | |
|---|---|---|
| Expected of Mutation | 0.17 | 0.17 |

Age in Uveal Melanoma
Signature

Mutation Type
Frequency of Mutation
Expected of Mutation

Age in Skin Cutaneous Melanoma
Signature

Mutation Type
Frequency of Mutation
Expected of Mutation

Age in Adrenocortical Carcinoma
Signature

Mutation Type
Frequency of Mutation
Expected of Mutation

Age in Cholangiocarcinoma
Signature

Mutation Type
Frequency of Mutation
Expected of Mutation

Age in Glioblastoma Multiforme
Signature

| Mutation Type | T > G | | T > C | | (CT)[C > T]G | | (CT)[C > T](ACT) | |
|---|---|---|---|---|---|---|---|---|
| Frequency of Mutation | 0.1 | [±0.024] | 0.1 | [±0.024] | 0.074 | [±0.052] | 0.13 | [±0.067] |

| | | | | |
|---|---|---|---|---|
| Expected of Mutation | 0.16 | 0.16 | 0.0095 | 0.083 |

Age in Cervical Squamous
Signature

| Mutation Type | T > G | | T > C | | (ACG)[C > T]G | | T[C > T]G | |
|---|---|---|---|---|---|---|---|---|
| Frequency of Mutation | 0.074 | [±0.032] | 0.074 | [±0.032] | 0.098 | [±0.06] | 0.047 | [±0.03] |

| | | | | |
|---|---|---|---|---|
| Expected of Mutation | 0.16 | 0.16 | 0.015 | 0.0035 |

Age in Colorectal Adenocarcinoma
Signature

Mutation Type
Frequency of Mutation
Expected of Mutation

Age in Pheochromocytoma and Paraganglioma
Signature

| Mutation Type | T > C | | C > T | |
|---|---|---|---|---|
| Frequency of Mutation | 0.21 | [±0.13] | 0.36 | [±0.17] |

| | | |
|---|---|---|
| Expected of Mutation | 0.16 | 0.17 |

Age in Pancreatic Adenocarcinoma
Signature

Mutation Type
Frequency of Mutation
Expected of Mutation

Age in Prostate Adenocarcinoma
Signature

| Mutation Type | T > G | | [T > C](CTG) | | (CT)[T > C]A | | (CT)[C > T]G | |
|---|---|---|---|---|---|---|---|---|
| Frequency of Mutation | 0.11 | [±0.028] | 0.095 | [±0.023] | 0.0088 | [±0.0021] | 0.056 | [±0.056] |

| | | | | |
|---|---|---|---|---|
| Expected of Mutation | 0.16 | 0.14 | 0.013 | 0.0095 |

Age in Esophagus Squamous
Signature

Mutation Type
Frequency of Mutation
Expected of Mutation

Age in Esophagus Adenocarcimona
Signature

Mutation Type
Frequency of Mutation
Expected of Mutation

Age in Uterine Corpus Endometrial Carcinoma
Signature

Mutation Type
Frequency of Mutation
Expected of Mutation

Age in Uterine Carcinosarcoma
Signature

| Mutation Type | C > T | | C > A | |
|---|---|---|---|---|
| Frequency of Mutation | 0.39 | [±0.11] | 0.2 | [±0.066] |

| | | |
|---|---|---|
| Expected of Mutation | 0.17 | 0.17 |

Age in Breast Invasive Carcinoma
Signature

Mutation Type
Frequency of Mutation
Expected of Mutation

Age in Sarcoma
Signature

| Mutation Type | T > A | | T > G | | T > C | | C > A | |
|---|---|---|---|---|---|---|---|---|
| Frequency of Mutation | 0.099 | [±0.025] | 0.099 | [±0.025] | 0.099 | [±0.025] | 0.23 | [±0.092] |

| | | | | |
|---|---|---|---|---|
| Expected of Mutation | 0.16 | 0.16 | 0.16 | 0.17 |

Age in Testicular Germ Cell Tumors
Signature

| Mutation Type | C > T | |
|---|---|---|
| Frequency of Mutation | 0.37 | [±0.14] |

| | |
|---|---|
| Expected of Mutation | 0.17 |

Age in Thymoma
Signature

| Mutation Type | T > C | |
|---|---|---|
| Frequency of Mutation | 0.11 | [±0.041] |

| | |
|---|---|
| Expected of Mutation | 0.16 |

Age in Ovarian Serous Cystadenocarcinoma
Signature

Mutation Type
Frequency of Mutation
Expected of Mutation

Smoking in Bladder Urothelial Carcinoma
Signature

Mutation Type
Unexposed Mutation Freq.
Exposed Mutation Freq.

Smoking in Lung Adenocarcinoma
Signature

| Mutation Type | C[C > A]G | |
|---|---|---|
| Unexposed Mutation Freq. | 0.017 | [±0.047] |
| Exposed Mutation Freq. | 0.026 | [±0.027] |

Smoking in Head and Neck
Signature

| Mutation Type | T > A | | T > G | | [T > C](CTG) | | (CTG)[T > C]A | |
|---|---|---|---|---|---|---|---|---|
| Unexposed Mutation Freq. | 0.06 | [±0.026] | 0.06 | [±0.026] | 0.051 | [±0.022] | 0.007 | [±0.003] |
| Exposed Mutation Freq. | 0.078 | [±0.027] | 0.078 | [±0.027] | 0.066 | [±0.023] | 0.009 | [±0.0031] |

Smoking in Renal Papillary Cell Carcinoma
Signature

| Mutation Type | T > G | | T > C | |
|---|---|---|---|---|
| Unexposed Mutation Freq. | 0.12 | [±0.031] | 0.12 | [±0.031] |
| Exposed Mutation Freq. | 0.12 | [±0.049] | 0.12 | [±0.049] |

Smoking in Pancreatic Adenocarcinoma
Signature

Mutation Type
Unexposed Mutation Freq.
Exposed Mutation Freq.

Smoking in Esophagus Squamous
Signature

| Mutation Type | (CTG)[T > C]A | |
|---|---|---|
| Unexposed Mutation Freq. | 0.0088 | [±0.0028] |
| Exposed Mutation Freq. | 0.01 | [±0.0032] |

Smoking in Esophagus Adenocarcimona
Signature

Mutation Type
Unexposed Mutation Freq.
Exposed Mutation Freq.

Smoking in Cervical Squamous
Signature

Mutation Type
Unexposed Mutation Freq.
Exposed Mutation Freq.

POLe Mutation in Uterine Corpus Endometrial Carcinoma
Signature

| Mutation Type | [T > G](ACG) | | (ACT) [T > C] (ACT) | | T[T > G]T | | (ACG)[T > G]T | |
|---|---|---|---|---|---|---|---|---|
| Unexposed Mutation Freq. | 0.059 | [±0.025] | 0.045 | [±0.019] | 0.0043 | [±0.0095] | 0.01 | [±0.011] |
| Exposed Mutation Freq. | 0.034 | [±0.023] | 0.026 | [±0.018] | 0.036 | [±0.035] | 0.026 | [±0.017] |

POLe Mutation in Stomach Adenocarcinoma
Signature

| Mutation Type | T > A | | [T > G](ACG) | | [T > C](ACG) | | (ATG)[T > C]T | |
|---|---|---|---|---|---|---|---|---|
| Unexposed Mutation Freq. | 0.072 | [±0.027] | 0.052 | [±0.019] | 0.097 | [±0.035] | 0.026 | [±0.0096] |
| Exposed Mutation Freq. | 0.049 | [±0.042] | 0.036 | [±0.03] | 0.14 | [±0.061] | 0.037 | [±0.016] |

POLe Mutation in Colorectal Adenocarcinoma
Signature

| Mutation Type | A[C > T]G | |
|---|---|---|
| Unexposed Mutation Freq. | 0.061 | [±0.037] |
| Exposed Mutation Freq. | 0.032 | [±0.028] |

POLe Mutation in Breast Invasive Carcinoma
Signature

| Mutation Type | (ACG)[C > G] | | T > A | | T > G | | T > C | |
|---|---|---|---|---|---|---|---|---|
| Unexposed Mutation Freq. | 0.07 | [±0.027] | 0.091 | [±0.035] | 0.091 | [±0.035] | 0.091 | [±0.035] |
| Exposed Mutation Freq. | 0.062 | [±0.026] | 0.081 | [±0.034] | 0.081 | [±0.034] | 0.081 | [±0.034] |

MLH Silenced in Uterine Corpus Endometrial Carcinoma
Signature

| Mutation Type | (ATG)[C > A] | | (ACG)[C > G] | | T[C > G](CG) | | T > A | |
|---|---|---|---|---|---|---|---|---|
| Unexposed Mutation Freq. | 0.063 | [±0.024] | 0.066 | [±0.026] | 0.0088 | [±0.0034] | 0.086 | [±0.034] |
| Exposed Mutation Freq. | 0.038 | [±0.015] | 0.041 | [±0.016] | 0.0054 | [±0.0021] | 0.053 | [±0.021] |

MLH Silenced in Stomach Adenocarcinoma
Signature

| Mutation Type | A[C > A]A | | C > G | | T > A | | [T > G](ACG) | |
|---|---|---|---|---|---|---|---|---|
| Unexposed Mutation Freq· | 0.006 | [±0.0017] | 0.089 | [±0.026] | 0.087 | [±0.025] | 0.063 | [±0.018] |
| Exposed Mutation Freq. | 0.0032 | [±0.0008] | 0.047 | [±0.012] | 0.046 | [±0.012] | 0.033 | [±0.0086] |

MLH Silenced in Colorectal Adenocarcinoma
Signature

Mutation Type
Unexposed Mutation Freq.
Exposed Mutation Freq.

BRCA1/2 Mutation in Breast Invasive Carcinoma
Signature

| Mutation Type | T[C > A]A | |
|---|---|---|
| Unexposed Mutation Freq. | 0.017 | [±0.025] |
| Exposed Mutation Freq. | 0.023 | [±0.021] |

BRCA1/2 Mutation in Ovarian Serous Cystadenocarcinoma
Signature

| Mutation Type | (ACT)[C > T]G | |
|---|---|---|
| Unexposed Mutation Freq. | 0.037 | [±0.035] |
| Exposed Mutation Freq. | 0.025 | [±0.017] |

UV* in Skin Cutaneous Melanoma
Signature

| Mutation Type | [T > C](ACG) | | (ACT)[T > C]T | | T[C > T]C | | (AG)[C > T]G | |
|---|---|---|---|---|---|---|---|---|
| Unexposed Mutation Freq. | 0.064 | [±0.029] | 0.02 | [±0.009] | 0.087 | [±0.086] | 0.046 | [±0.047] |
| Exposed Mutation Freq. | 0.019 | [±0.0058] | 0.0058 | [±0.0018] | 0.22 | [±0.084] | 0.0034 | [±0.004] |

POLD Mutation in Uterine Corpus Endometrial Carcinoma
Signature

| Mutation Type | A[T > C]G | |
|---|---|---|
| Unexposed Mutation Freq. | 0.013 | [±0.016] |
| Exposed Mutation Freq. | 0.027 | [±0.013] |

High Copy Number in Uterine Corpus Endometrial Carcinoma
Signature

| Mutation Type | T > A | | T > G | | (ACT)[T > C](ACT) | | G[T > C](CT) | |
|---|---|---|---|---|---|---|---|---|
| Unexposed Mutation Freq. | 0.081 | [±0.031] | 0.081 | [±0.031] | 0.045 | [±0.017] | 0.0079 | [±0.0031] |
| Exposed Mutation Freq. | 0.1 | [±0.036] | 0.1 | [±0.036] | 0.057 | [±0.02] | 0.01 | [±0.0036] |

Low Copy Number in Uterine Corpus Endometrial Carcinoma
Signature

| Mutation Type | T > G | | (ACT)[T > C](CT) | | (CT)[T > C]G | | G[T > C]A | |
|---|---|---|---|---|---|---|---|---|
| Unexposed Mutation Freq. | 0.086 | [±0.034] | 0.038 | [±0.015] | 0.024 | [±0.025] | 0.012 | [±0.014] |
| Exposed Mutation Freq. | 0.11 | [±0.031] | 0.049 | [±0.014] | 0.012 | [±0.015] | 0.0082 | [±0.014] |

POLD Mutation in Stomach Adenocarcinoma
Signature

| Mutation Type | (ACG)[C > A](CTG | | (AC)[C > A]A | | C > G | | T > A | |
|---|---|---|---|---|---|---|---|---|
| Unexposed Mutation Freq. | 0.047 | [±0.015] | 0.014 | [±0.0046] | 0.092 | [±0.029] | 0.089 | [±0.029] |
| Exposed Mutation Freq. | 0.043 | [±0.019] | 0.013 | [±0.0056] | 0.082 | [±0.036] | 0.08 | [±0.035] |

MGMT Methylated in Glioblastoma Multiforme
Signature

| Mutation Type | T > A | | T > G | | T > C | |
|---|---|---|---|---|---|---|
| Unexposed Mutation Freq. | 0.1 | [±0.022] | 0.1 | [±0.022] | 0.1 | [±0.022] |
| Exposed Mutation Freq. | 0.092 | [±0.018] | 0.092 | [±0.018] | 0.092 | [±0.018] |

MGMT Methylated in Brain Lower Grade Glioma
Signature

Mutation Type
Unexposed Mutation Freq.
Exposed Mutation Freq.

IDH Methylated in Brain Lower Grade Glioma
Signature

| Mutation Type | (CT)[T > C]C | |
|---|---|---|
| Unexposed Mutation Freq. | 0.034 | [±0.038] |
| Exposed Mutation Freq. | 0.018 | [±0.023] |

IDH Methylated in Glioblastoma Multiforme
Signature

| Mutation Type | A[C > T](ACT) | |
|---|---|---|
| Unexposed Mutation Freq. | 0.029 | [±0.019] |
| Exposed Mutation Freq. | 0.053 | [±0.039] |

Obesity in Uterine Corpus Endometrial Carcinoma
Signature

Mutation Type
Unexposed Mutation Freq.
Exposed Mutation Freq.

Obesity in Renal Papillary Cell Carcinoma
Signature

| Mutation Type | T > C | |
|---|---|---|
| Unexposed Mutation Freq. | 0.13 | [±0.028] |
| Exposed Mutation Freq. | 0.093 | [±0.05] |

Obesity in Esophageal Carcinoma
Signature

Mutation Type
Unexposed Mutation Freq.
Exposed Mutation Freq.

Obesity in Colorectal Adenocarcinoma
Signature

| Mutation Type | G[C > T]G | |
|---|---|---|
| Unexposed Mutation Freq. | 0.074 | [±0.036] |
| Exposed Mutation Freq. | 0.076 | [±0.036] |

Alcohol in Head and Neck
Signature

| Mutation Type | C > T | | C > A | |
|---|---|---|---|---|
| Unexposed Mutation Freq. | 0.45 | [±0.073] | 0.18 | [±0.072] |
| Exposed Mutation Freq. | 0.42 | [±0.15] | 0.21 | [±0.15] |

Alcohol in Esophageal Carcinoma
Signature

Mutation Type
Unexposed Mutation Freq.
Exposed Mutation Freq.

Alcohol in Liver Hepatocellular Carcinoma
Signature

Mutation Type
Unexposed Mutation Freq.
Exposed Mutation Freq.

Hepatitis B in Liver Hepatocellular Carcinoma
Signature

Mutation Type
Unexposed Mutation Freq.
Exposed Mutation Freq.

Hepatitis C in Liver Hepatocellular Carcinoma
Signature

Mutation Type
Unexposed Mutation Freq.
Exposed Mutation Freq.

Aristolochic Acid in Bladder Urothelial Carcinoma
Signature

| Mutation Type | T[C > G]T | |
|---|---|---|
| Unexposed Mutation Freq. | 0.1 | [±0.062] |
| Exposed Mutation Freq. | 0.024 | [±0.03] |

Asbestos in Mesothelioma
Signature

Mutation Type
Unexposed Mutation Freq.
Exposed Mutation Freq.

High Apobec in Cervical Squamous
Signature

| Mutation Type | T > C | | T[C > T](CT) | | T[C > T]A | | T[C > A]A | |
|---|---|---|---|---|---|---|---|---|
| Unexposed Mutation Freq. | 0.08 | [±0.035] | 0.11 | [±0.053] | 0.1 | [±0.076] | 0.014 | [±0.014] |
| Exposed Mutation Freq. | 0.061 | [±0.031] | 0.14 | [±0.063] | 0.14 | [±0.063] | 0.021 | [±0.017] |

High Apobec in Renal Clear Cell Carcinoma
Signature

Mutation Type
Unexposed Mutation Freq.
Exposed Mutation Freq.

| V1 | V10 | | V11 | | V12 | |
|---|---|---|---|---|---|---|
| Age in Acute Myeloid Leukemia | | | | | | |
| Signature | | | | | | |
| Mutation Type | | | | | | |
| Frequency of Mutation | | | | | | |
| Expected of Mutation | | | | | | |
| Age in Bladder Urothelial Carcinoma | | | | | | |
| Signature | | | | | | |
| Mutation Type | | | | | | |
| Frequency of Mutation | | | | | | |
| Expected of Mutation | | | | | | |
| Age in Lung Adenocarcinoma | | | | | | |
| Signature | | | | | | |
| Mutation Type | | | | | | |
| Frequency of Mutation | | | | | | |
| Expected of Mutation | | | | | | |
| Age in Brain Lower Grade Glioma | | | | | | |
| Signature | | | | | | |
| Mutation Type | | | | | | |
| Frequency of Mutation | | | | | | |
| Expected of Mutation | | | | | | |
| Age in Head and Neck | | | | | | |
| Signature | | | | | | |
| Mutation Type | T[C > T](CT) | | T[C > G]A | | | |
| Frequency of Mutation | 0.089 | [±0.051] | 0.049 | [±0.045] | | |
| Expected of Mutation | 0.027 | | 0.012 | | | |
| Age in Renal Clear Cell Carcinoma | | | | | | |
| Signature | | | | | | |
| Mutation Type | | | | | | |
| Frequency of Mutation | | | | | | |
| Expected of Mutation | | | | | | |
| Age in Renal Papillary Cell Carcinoma | | | | | | |
| Signature | | | | | | |
| Mutation Type | | | | | | |
| Frequency of Mutation | | | | | | |
| Expected of Mutation | | | | | | |
| Age in Kidney Chromophobe | | | | | | |
| Signature | | | | | | |
| Mutation Type | | | | | | |
| Frequency of Mutation | | | | | | |
| Expected of Mutation | | | | | | |
| Age in Liver Hepatocellular Carcinoma | | | | | | |
| Signature | | | | | | |
| Mutation Type | | | | | | |
| Frequency of Mutation | | | | | | |
| Expected of Mutation | | | | | | |
| Age in Stomach Adenocarcinoma | | | | | | |
| Signature | | | | | | |
| Mutation Type | A[C > T]G | | G[C > T]G | | T[C > A] | |
| Frequency of Mutation | 0.035 | [±0.026] | 0.048 | [±0.032] | 0.056 | [±0.042] |
| Expected of Mutation | 0.0036 | | 0.005 | | 0.043 | |
| Age in Thyroid Carcinoma | | | | | | |
| Signature | | | | | | |
| Mutation Type | | | | | | |
| Frequency of Mutation | | | | | | |
| Expected of Mutation | | | | | | |
| Age in Uveal Melanoma | | | | | | |
| Signature | | | | | | |
| Mutation Type | | | | | | |
| Frequency of Mutation | | | | | | |
| Expected of Mutation | | | | | | |
| Age in Skin Cutaneous Melanoma | | | | | | |
| Signature | | | | | | |
| Mutation Type | | | | | | |
| Frequency of Mutation | | | | | | |
| Expected of Mutation | | | | | | |
| Age in Adrenocortical Carcinoma | | | | | | |
| Signature | | | | | | |
| Mutation Type | | | | | | |
| Frequency of Mutation | | | | | | |
| Expected of Mutation | | | | | | |
| Age in Cholangiocarcinoma | | | | | | |
| Signature | | | | | | |
| Mutation Type | | | | | | |
| Frequency of Mutation | | | | | | |
| Expected of Mutation | | | | | | |
| Age in Glioblastoma Multiforme | | | | | | |
| Signature | | | | | | |
| Mutation Type | | | | | | |
| Frequency of Mutation | | | | | | |
| Expected of Mutation | | | | | | |
| Age in Cervical Squamous | | | | | | |
| Signature | | | | | | |
| Mutation Type | | | | | | |
| Frequency of Mutation | | | | | | |
| Expected of Mutation | | | | | | |
| Age in Colorectal Adenocarcinoma | | | | | | |
| Signature | | | | | | |
| Mutation Type | | | | | | |
| Frequency of Mutation | | | | | | |
| Expected of Mutation | | | | | | |
| Age in Pheochromocytoma and Paraganglioma | | | | | | |
| Signature | | | | | | |
| Mutation Type | | | | | | |
| Frequency of Mutation | | | | | | |
| Expected of Mutation | | | | | | |
| Age in Pancreatic Adenocarcinoma | | | | | | |
| Signature | | | | | | |
| Mutation Type | | | | | | |
| Frequency of Mutation | | | | | | |
| Expected of Mutation | | | | | | |
| Age in Prostate Adenocarcinoma | | | | | | |
| Signature | | | | | | |
| Mutation Type | G[C > T](ACT) | | G[C > T]G | | | |
| Frequency of Mutation | 0.073 | [±0.075] | 0.048 | [±0.053] | | |
| Expected of Mutation | 0.037 | | 0.005 | | | |
| Age in Esophagus Squamous | | | | | | |
| Signature | | | | | | |
| Mutation Type | | | | | | |
| Frequency of Mutation | | | | | | |
| Expected of Mutation | | | | | | |
| Age in Esophagus Adenocarcimona | | | | | | |
| Signature | | | | | | |
| Mutation Type | | | | | | |
| Frequency of Mutation | | | | | | |
| Expected of Mutation | | | | | | |
| Age in Uterine Corpus Endometrial Carcinoma | | | | | | |
| Signature | | | | | | |
| Mutation Type | | | | | | |
| Frequency of Mutation | | | | | | |
| Expected of Mutation | | | | | | |
| Age in Uterine Carcinosarcoma | | | | | | |
| Signature | | | | | | |
| Mutation Type | | | | | | |
| Frequency of Mutation | | | | | | |
| Expected of Mutation | | | | | | |
| Age in Breast Invasive Carcinoma | | | | | | |
| Signature | | | | | | |
| Mutation Type | | | | | | |
| Frequency of Mutation | | | | | | |
| Expected of Mutation | | | | | | |
| Age in Sarcoma | | | | | | |
| Signature | | | | | | |
| Mutation Type | T[C > G]T | | | | | |
| Frequency of Mutation | 0.021 | [±0.025] | | | | |
| Expected of Mutation | 0.014 | | | | | |
| Age in Testicular Germ Cell Tumors | | | | | | |
| Signature | | | | | | |
| Mutation Type | | | | | | |
| Frequency of Mutation | | | | | | |
| Expected of Mutation | | | | | | |
| Age in Thymoma | | | | | | |
| Signature | | | | | | |
| Mutation Type | | | | | | |
| Frequency of Mutation | | | | | | |
| Expected of Mutation | | | | | | |
| Age in Ovarian Serous Cystadenocarcinoma | | | | | | |
| Signature | | | | | | |
| Mutation Type | | | | | | |
| Frequency of Mutation | | | | | | |
| Expected of Mutation | | | | | | |
| Smoking in Bladder Urothelial Carcinoma | | | | | | |
| Signature | | | | | | |
| Mutation Type | | | | | | |
| Unexposed Mutation Freq. | | | | | | |
| Exposed Mutation Freq. | | | | | | |
| Smoking in Lung Adenocarcinoma | | | | | | |
| Signature | | | | | | |
| Mutation Type | | | | | | |
| Unexposed Mutation Freq. | | | | | | |
| Exposed Mutation Freq. | | | | | | |
| Smoking in Head and Neck | | | | | | |
| Signature | | | | | | |
| Mutation Type | T[C > T]A | | T[C > T]G | | | |
| Unexposed Mutation Freq. | 0.091 | [±0.061] | 0.037 | [±0.028] | | |
| Exposed Mutation Freq. | 0.06 | [±0.047] | 0.024 | [±0.022] | | |
| Smoking in Renal Papillary Cell Carcinoma | | | | | | |
| Signature | | | | | | |
| Mutation Type | | | | | | |
| Unexposed Mutation Freq. | | | | | | |
| Exposed Mutation Freq. | | | | | | |
| Smoking in Pancreatic Adenocarcinoma | | | | | | |
| Signature | | | | | | |
| Mutation Type | | | | | | |
| Unexposed Mutation Freq. | | | | | | |
| Exposed Mutation Freq. | | | | | | |
| Smoking in Esophagus Squamous | | | | | | |
| Signature | | | | | | |
| Mutation Type | | | | | | |
| Unexposed Mutation Freq. | | | | | | |
| Exposed Mutation Freq. | | | | | | |
| Smoking in Esophagus Adenocarcimona | | | | | | |
| Signature | | | | | | |
| Mutation Type | | | | | | |
| Unexposed Mutation Freq. | | | | | | |
| Exposed Mutation Freq. | | | | | | |
| Smoking in Cervical Squamous | | | | | | |
| Signature | | | | | | |
| Mutation Type | | | | | | |
| Unexposed Mutation Freq. | | | | | | |
| Exposed Mutation Freq. | | | | | | |
| POLe Mutation in Uterine Corpus Endometrial Carcinoma | | | | | | |
| Signature | | | | | | |
| Mutation Type | G[T > C]T | | T[C > A]T | | | |
| Unexposed Mutation Freq. | 0.0069 | [±0.01] | 0.018 | [±0.019] | | |
| Exposed Mutation Freq. | 0.011 | [±0.0067] | 0.17 | [±0.16] | | |
| POLe Mutation in Stomach Adenocarcinoma | | | | | | |
| Signature | | | | | | |
| Mutation Type | | | | | | |
| Unexposed Mutation Freq. | | | | | | |
| Exposed Mutation Freq. | | | | | | |
| POLe Mutation in Colorectal Adenocarcinoma | | | | | | |
| Signature | | | | | | |
| Mutation Type | | | | | | |
| Unexposed Mutation Freq. | | | | | | |
| Exposed Mutation Freq. | | | | | | |
| POLe Mutation in Breast Invasive Carcinoma | | | | | | |
| Signature | | | | | | |
| Mutation Type | T[C > A]G | | | | | |
| Unexposed Mutation Freq. | 0.0084 | [±0.018] | | | | |
| Exposed Mutation Freq. | 0.0072 | [±0.01] | | | | |
| MLH Silenced in Uterine Corpus Endometrial Carcinoma | | | | | | |
| Signature | | | | | | |
| Mutation Type | T > G | | (ACT) [T > C](CT) | | | |
| Unexposed Mutation Freq. | 0.086 | [±0.034] | 0.038 | [±0.015] | | |
| Exposed Mutation Freq. | 0.053 | [±0.021] | 0.023 | [±0.009] | | |
| MLH Silenced in Stomach Adenocarcinoma | | | | | | |
| Signature | | | | | | |
| Mutation Type | (AT)[T > C](CT) | | C[T > C]C | | G[T > C]G | |
| Unexposed Mutation Freq· | 0.024 | [±0.0069] | 0.0071 | [±0.002] | 0.0068 | [±0.0088] |
| Exposed Mutation Freq. | 0.013 | [±0.0033] | 0.0038 | [±0.00097] | 0.027 | [±0.01] |

| MLH Silenced in Colorectal Adenocarcinoma | | | | |
|---|---|---|---|---|
| Signature | | | | |
| Mutation Type | | | | |
| Unexposed Mutation Freq. | | | | |
| Exposed Mutation Freq. | | | | |
| BRCA1/2 Mutation in Breast Invasive Carcinoma | | | | |
| Signature | | | | |
| Mutation Type | | | | |
| Unexposed Mutation Freq. | | | | |
| Exposed Mutation Freq. | | | | |
| BRCA1/2 Mutation in Ovarian Serous Cystadenocarcinoma | | | | |
| Signature | | | | |
| Mutation Type | | | | |
| Unexposed Mutation Freq. | | | | |
| Exposed Mutation Freq. | | | | |
| UV* in Skin Cutaneous Melanoma | | | | |
| Signature | | | | |
| Mutation Type | T[C > T](AT) | | C[C > T]C | |
| Unexposed Mutation Freq. | 0.08 | [±0.071] | 0.041 | [±0.042] |
| Exposed Mutation Freq. | 0.16 | [±0.062] | 0.083 | [±0.031] |
| POLD Mutation in Uterine Corpus Endometrial Carcinoma | | | | |
| Signature | | | | |
| Mutation Type | | | | |
| Unexposed Mutation Freq. | | | | |
| Exposed Mutation Freq. | | | | |
| High Copy Number in Uterine Corpus Endometrial Carcinoma | | | | |
| Signature | | | | |
| Mutation Type | G[C > T]G | | T[C > G]T | |
| Unexposed Mutation Freq. | 0.058 | [±0.052] | 0.03 | [±0.049] |
| Exposed Mutation Freq. | 0.03 | [±0.031] | 0.044 | [±0.041] |
| Low Copy Number in Uterine Corpus Endometrial Carcinoma | | | | |
| Signature | | | | |
| Mutation Type | T[C > G]T | | | |
| Unexposed Mutation Freq. | 0.03 | [±0.049] | | |
| Exposed Mutation Freq. | 0.012 | [±0.018] | | |
| POLD Mutation in Stomach Adenocarcinoma | | | | |
| Signature | | | | |
| Mutation Type | [T > G](ACG) | | G[C > T]G | |
| Unexposed Mutation Freq. | 0.065 | [±0.021] | 0.048 | [±0.032] |
| Exposed Mutation Freq. | 0.058 | [±0.025] | 0.063 | [±0.042] |

MGMT Methylated in Glioblastoma Multiforme
Signature
Mutation Type
Unexposed Mutation Freq.
Exposed Mutation Freq.
MGMT Methylated in Brain Lower Grade Glioma
Signature
Mutation Type
Unexposed Mutation Freq.
Exposed Mutation Freq.
IDH Methylated in Brain Lower Grade Glioma
Signature
Mutation Type
Unexposed Mutation Freq.
Exposed Mutation Freq.
IDH Methylated in Glioblastoma Multiforme
Signature
Mutation Type
Unexposed Mutation Freq.
Exposed Mutation Freq.
Obesity in Uterine Corpus Endometrial Carcinoma
Signature
Mutation Type
Unexposed Mutation Freq.
Exposed Mutation Freq.
Obesity in Renal Papillary Cell Carcinoma
Signature
Mutation Type
Unexposed Mutation Freq.
Exposed Mutation Freq.
Obesity in Esophageal Carcinoma
Signature
Mutation Type
Unexposed Mutation Freq.
Exposed Mutation Freq.
Obesity in Colorectal Adenocarcinoma
Signature
Mutation Type
Unexposed Mutation Freq.
Exposed Mutation Freq.
Alcohol in Head and Neck
Signature
Mutation Type
Unexposed Mutation Freq.
Exposed Mutation Freq.
Alcohol in Esophageal Carcinoma
Signature
Mutation Type
Unexposed Mutation Freq.
Exposed Mutation Freq.
Alcohol in Liver Hepatocellular Carcinoma
Signature
Mutation Type
Unexposed Mutation Freq.
Exposed Mutation Freq.
Hepatitis B in Liver Hepatocellular Carcinoma
Signature
Mutation Type
Unexposed Mutation Freq.
Exposed Mutation Freq.
Hepatitis C in Liver Hepatocellular Carcinoma
Signature
Mutation Type
Unexposed Mutation Freq.
Exposed Mutation Freq.
Aristolochic Acid in Bladder Urothelial Carcinoma
Signature
Mutation Type
Unexposed Mutation Freq.
Exposed Mutation Freq.
Asbestos in Mesothelioma
Signature
Mutation Type
Unexposed Mutation Freq.
Exposed Mutation Freq.
High Apobec in Cervical Squamous
Signature
Mutation Type
Unexposed Mutation Freq.
Exposed Mutation Freq.
High Apobec in Renal Clear Cell Carcinoma
Signature
Mutation Type
Unexposed Mutation Freq.
Exposed Mutation Freq.

TABLE 7
An example of projecting counts on a refinement partition: Partition 1 ([C > T]G, [C > T]H,
Remaining) = (15, 5, 65) and Partition 2 (A[C > T], B[C > T], Remaining) = (6, 14, 180).
H means “not G” and B means “not A”. The symbol ‘#’ before a k-nucleotide represents
the average count of that k-nucleotide on the exomic data.

| | | | Projected | | | | Projected |
|---|---|---|---|---|---|---|---|
| | | | counts on | | | | counts on |
| Partition 1 | Counts of | Refinement | refinement | Partition 2 | Counts of | Refinement | refinement |
| (features) | feature | partition | partition | (features) | feature | partition | partition |
| [C > T]G | 15 | A[C > T]G | $15 \frac{\# ACG}{\# CG}$ | A[C > T] | 6 | A[C > T]G | $6 \frac{\# ACG}{\# AC}$ |
| | | B[C > T]G | $15 \frac{\# BCG}{\# CG}$ | | | A[C > T]H | $6 \frac{\# BCG}{\# AC}$ |
| [C > T]H | 5 | A[C > T]H | $5 \frac{\# ACH}{\# CH}$ | B[C > T] | 14 | B[C > T]G | $14 \frac{\# ACH}{\# BC}$ |
| | | B[C > T]H | $5 \frac{\# BCH}{\# CH}$ | | | B[C > T]H | $14 \frac{\# BCH}{\# BC}$ |
| Remaining | 65 | Remaining | 65 | Remaining | 180 | Remaining | 180 |

TABLE 8
SuperSigs and their predictive features. The set of n predictive features
forming the supervised signature (SuperSig) are listed for each tissue
type and for each etiological exposure. Two values are associated to
each one of these predictive features: 1) the difference in mean counts
(age) or rates (all other exposures) between the exposed and unexposed
cohorts, and 2) the beta (β) coefficient for that feature as estimated
by logistic regression. See also FIG. 29 and FIG. 30.

| tissue | factor | labels_iupac | differences | betas |
|---|---|---|---|---|
| LAML | AGE | C > A | 0.822795613 | 0.162825325 |
| LAML | AGE | C > G | 0.822795613 | 0.162825325 |
| LAML | AGE | T > G | 0.803554637 | 0.162825325 |
| LAML | AGE | T > C | 0.803554637 | 0.162825325 |
| LAML | AGE | B[T > A]B | 0.538966166 | 0.162825325 |
| BLCA | AGE | V[C > T]G | 1.900510204 | 0.194464563 |
| LUAD | AGE | [C > A]H | 4.166666667 | 0.037578637 |
| LGG | AGE | [C > T]H | 5.466666667 | 0.490777075 |
| HNSCC | AGE | V[C > T]H | 6.061128091 | 0.093120062 |
| HNSCC | AGE | T[C > G]T | 6.576271186 | 0.22278826 |
| HNSCC | AGE | T > A | 2.637071235 | −0.025812865 |
| HNSCC | AGE | T > G | 2.637071235 | −0.025812865 |
| HNSCC | AGE | T > C | 2.637071235 | −0.025812865 |
| HNSCC | AGE | V[C > G] | 2.015154731 | −0.025812865 |
| HNSCC | AGE | T[C > T]Y | 5.194776327 | −0.019183696 |
| HNSCC | AGE | T[C > G]A | 5.259238677 | −0.067864515 |
| HNSCC | AGE | V[C > T]G | 3.093081412 | 0.162570731 |
| HNSCC | AGE | T[C > T]A | 5.621561545 | −0.076415714 |
| HNSCC | AGE | T[C > A]Y | 2.080300083 | 0.097884319 |
| HNSCC | AGE | T[C > A]A | 1.290914143 | 0.163974225 |
| HNSCC | AGE | R[C > A]G | 0.797443734 | −0.033831838 |
| HNSCC | AGE | C[C > A]A | 1.827729925 | 0.037095464 |
| KIRC | AGE | V[C > T]H | 4.134710145 | 0.138886834 |
| KIRC | AGE | T[C > T]Y | 1.00326655 | 0.138886834 |
| KIRC | AGE | C > G | 2.748729888 | 0.043172537 |
| KIRC | AGE | T > A | 2.684451174 | 0.043172537 |
| KIRC | AGE | T > G | 2.684451174 | 0.043172537 |
| KIRC | AGE | B[T > C]B | 1.800535136 | 0.043172537 |
| KIRC | AGE | [C > T]G | 1.244173729 | 0.431453832 |
| KIRP | AGE | V[C > T]H | 3.846153846 | 0.246561028 |
| KICH | AGE | C > G | 1.206703306 | 0.302839698 |
| KICH | AGE | T > A | 1.178484696 | 0.302839698 |
| KICH | AGE | T > G | 1.178484696 | 0.302839698 |
| KICH | AGE | T > C | 1.178484696 | 0.302839698 |
| KICH | AGE | B[C > A] | 0.963724957 | 0.302839698 |
| KICH | AGE | [C > T]G | 1.352941176 | 0.841140755 |
| LIHC | AGE | [C > T]H | 7.589901478 | 0.084808564 |
| LIHC | AGE | A[T > C]B | 1.75 | 0.151243743 |
| STAD | AGE | A[C > T]G | 1.368665851 | 0.269779928 |
| THCA | AGE | T > G | 0.554257212 | 0.198826784 |
| THCA | AGE | [C > G]V | 0.398606306 | 0.198826784 |
| THCA | AGE | [T > A]H | 0.383379093 | 0.198826784 |
| THCA | AGE | B[T > C] | 0.436015185 | 0.198826784 |
| THCA | AGE | V[C > G]T | 0.122414023 | 0.198826784 |
| THCA | AGE | H[T > A]G | 0.132198929 | 0.198826784 |
| THCA | AGE | V[C > T]W | 0.763434582 | 0.353497863 |
| THCA | AGE | H[C > T]C | 0.373254923 | 0.353497863 |
| THCA | AGE | T[C > T]T | 0.140861515 | 0.353497863 |
| UVM | AGE | [C > T]H | 1.6 | 0.300385726 |
| SKCM | AGE | C[C > A]H | 5.43902439 | 0.025955808 |
| ACC | AGE | C > A | 4.554782609 | 0.21023825 |
| CHOL | AGE | C > G | 1.683370209 | 0.139449349 |
| CHOL | AGE | T > A | 1.644004802 | 0.139449349 |
| CHOL | AGE | T > G | 1.644004802 | 0.139449349 |
| CHOL | AGE | T > C | 1.644004802 | 0.139449349 |
| GBM | AGE | C > G | 1.322399462 | 0.080603833 |
| GBM | AGE | T > A | 1.291475312 | 0.080603833 |
| GBM | AGE | T > G | 1.291475312 | 0.080603833 |
| GBM | AGE | T > C | 1.291475312 | 0.080603833 |
| CESC | AGE | V[C > T]G | 4.407751938 | 0.117897168 |
| CESC | AGE | V[C > T]H | 5.721447028 | 0.125458162 |
| CESC | AGE | T > A | 2.453689625 | 0.024595564 |
| CESC | AGE | T > G | 2.453689625 | 0.024595564 |
| CESC | AGE | T > C | 2.453689625 | 0.024595564 |
| CESC | AGE | V[C > A] | 1.875021118 | 0.024595564 |
| CESC | AGE | V[C > G] | 1.875021118 | 0.024595564 |
| CESC | AGE | T[C > T]G | 1.733850129 | 0.155518279 |
| CESC | AGE | T[C > T]Y | 3.142118863 | 0.039707517 |
| CESC | AGE | T[C > A]A | 0.471834625 | 0.075396192 |
| CESC | AGE | T[C > T]A | 0.605167959 | −0.092812045 |
| COAD | AGE | [C > T]H | 4.580645161 | 0.008638578 |
| COAD | AGE | G[C > T]G | 1.983870968 | 0.156418976 |
| COAD | AGE | T[C > G]T | 0.919354839 | 0.200385424 |
| PCPG | AGE | C > A | 0.625936075 | 0.289038246 |
| PCPG | AGE | C > G | 0.625936075 | 0.289038246 |
| PCPG | AGE | T > A | 0.611298636 | 0.289038246 |
| PCPG | AGE | T > G | 0.611298636 | 0.289038246 |
| PCPG | AGE | B[T > C] | 0.480887722 | 0.289038246 |
| PCPG | AGE | [C > T]H | 0.968112245 | 0.141413166 |
| PAAD | AGE | B[C > T]G | 2.326315789 | 0.237162716 |
| PRAD | AGE | C > G | 0.629081754 | 0.069797531 |
| PRAD | AGE | T > A | 0.614370753 | 0.069797531 |
| PRAD | AGE | T > G | 0.614370753 | 0.069797531 |
| PRAD | AGE | Y[T > C]B | 0.308843406 | 0.069797531 |
| PRAD | AGE | [C > A]W | 0.886279003 | 0.084040029 |
| PRAD | AGE | S[C > A]C | 0.233075836 | 0.084040029 |
| PRAD | AGE | Y[C > T]G | 0.493548387 | 0.197472582 |
| PRAD | AGE | G[C > T]G | 0.376774194 | 0.184871784 |
| PRAD | AGE | [C > T]H | 0.848602151 | −0.008204745 |
| ESCSQ | AGE | V[C > T]G | 1.186609687 | 0.079008804 |
| ESCAD | AGE | T[C > T]G | 1.368421053 | 0.192565049 |
| ESCAD | AGE | C[T > C]V | 3.210526316 | 0.105254454 |
| UCEC | AGE | T[C > G]T | 13.18181818 | 0.381612256 |
| UCS | AGE | T > A | 0.537334252 | 0.015733182 |
| UCS | AGE | T > G | 0.537334252 | 0.015733182 |
| UCS | AGE | T > C | 0.537334252 | 0.015733182 |
| UCS | AGE | V[C > A] | 0.410611456 | 0.015733182 |
| UCS | AGE | V[C > G]H | 0.363086642 | 0.015733182 |
| UCS | AGE | S[C > G]G | 0.035867773 | 0.015733182 |
| BRCA | AGE | S[C > T]G | 0.75838341 | 0.252539991 |
| BRCA | AGE | T > A | 0.433218588 | 0.008548394 |
| BRCA | AGE | T > G | 0.433218588 | 0.008548394 |
| BRCA | AGE | T > C | 0.433218588 | 0.008548394 |
| BRCA | AGE | V[C > G] | 0.331050021 | 0.008548394 |
| SARC | AGE | [C > T]H | 4.632299928 | 0.120335384 |
| SARC | AGE | H[C > T]G | 1.868601298 | 0.366208681 |
| SARC | AGE | T > A | 1.852757841 | 0.037491439 |
| SARC | AGE | T > G | 1.852757841 | 0.037491439 |
| SARC | AGE | T > C | 1.852757841 | 0.037491439 |
| SARC | AGE | [C > G]V | 1.332451691 | 0.037491439 |
| SARC | AGE | V[C > G]T | 0.409202688 | 0.037491439 |
| SARC | AGE | C > A | 4.04109589 | 0.022435647 |
| TGCT | AGE | T > A | 0.344871746 | 0.103919744 |
| TGCT | AGE | T > G | 0.344871746 | 0.103919744 |
| TGCT | AGE | T > C | 0.344871746 | 0.103919744 |
| TGCT | AGE | [C > G]H | 0.315218527 | 0.103919744 |
| TGCT | AGE | B[C > G]G | 0.030429393 | 0.103919744 |
| THYM | AGE | H[C > T]H | 1.45045045 | 0.56621015 |
| OV | AGE | M[C > T]G | 1.290562036 | 0.492588817 |
| OV | AGE | T[C > T]G | 0.488335101 | 0.284023339 |
| BLCA | SMOKING | V[C > T]H | 0.002381002 | 3.218808358 |
| BLCA | SMOKING | T > A | 0.001462352 | 0.313589376 |
| BLCA | SMOKING | T > G | 0.001462352 | 0.313589376 |
| BLCA | SMOKING | T > C | 0.001462352 | 0.313589376 |
| BLCA | SMOKING | V[C > G] | 0.001117477 | 0.313589376 |
| BLCA | SMOKING | V[C > A]H | 9.88E−04 | 0.313589376 |
| LUAD | SMOKING | T[C > A]C | 0.003854193 | 52.91541827 |
| LUAD | SMOKING | D[C > A]W | 0.01361221 | −0.326868334 |
| LUAD | SMOKING | R[C > A]C | 0.004374657 | −0.326868334 |
| LUAD | SMOKING | C[C > A]W | 0.008827107 | 8.930427814 |
| LUAD | SMOKING | D[C > A]G | 0.00408822 | 18.49649523 |
| LUAD | SMOKING | T > G | 0.00516727 | 0.665906625 |
| LUAD | SMOKING | T > C | 0.00516727 | 0.665906625 |
| LUAD | SMOKING | V[C > G] | 0.003948642 | 0.665906625 |
| LUAD | SMOKING | [T > A]H | 0.003574195 | 0.665906625 |
| LUAD | SMOKING | D[T > A]G | 0.001022643 | 0.665906625 |
| LUAD | SMOKING | C[C > A]C | 0.004709159 | −2.554418014 |
| LUAD | SMOKING | C[C > A]G | 0.002313972 | −9.824806718 |
| LUAD | SMOKING | C[T > A]G | 0.002252288 | 35.14196571 |
| LUAD | SMOKING | V[C > T]H | 0.007112641 | −6.625101666 |
| LUAD | SMOKING | T[C > T]Y | 0.002726902 | −7.376837429 |
| LUAD | SMOKING | T[C > G]T | 0.001586473 | 17.27566939 |
| LUAD | SMOKING | T[C > G]A | 0.001439334 | 39.45533447 |
| LUAD | SMOKING | T[C > G]S | 0.001082839 | −47.22619691 |
| LUAD | SMOKING | T[C > T]A | 0.002069133 | −9.945495987 |
| HNSCC | SMOKING | T > A | 0.002073398 | 1.02433393 |
| HNSCC | SMOKING | T > G | 0.002073398 | 1.02433393 |
| HNSCC | SMOKING | V[C > G] | 0.001584416 | 1.02433393 |
| HNSCC | SMOKING | [T > C]B | 0.001747832 | 1.02433393 |
| HNSCC | SMOKING | B[T > C]A | 2.40E−04 | 1.02433393 |
| HNSCC | SMOKING | V[C > T]W | 0.003120564 | 3.973083073 |
| HNSCC | SMOKING | A[T > C]A | 5.25E−04 | 134.3183787 |
| HNSCC | SMOKING | V[C > T]C | 0.002403398 | 7.261316973 |
| HNSCC | SMOKING | V[C > A]Y | 0.002728714 | −11.76979669 |
| HNSCC | SMOKING | R[C > A]A | 8.77E−04 | −11.76979669 |
| HNSCC | SMOKING | T[C > A]H | 0.001908147 | −1.998342138 |
| HNSCC | SMOKING | C[C > A]G | 9.82E−04 | 2.400889557 |
| HNSCC | SMOKING | C[C > A]A | 0.002317201 | 20.63776673 |
| HNSCC | SMOKING | T[C > A]G | 4.88E−04 | 77.53030476 |
| HNSCC | SMOKING | R[C > A]G | 6.50E−04 | 15.66562047 |
| HNSCC | SMOKING | T[C > T]C | 0.001915636 | −12.4763584 |
| HNSCC | SMOKING | T[C > G]S | 2.90E−04 | −5.661739188 |
| HNSCC | SMOKING | T[C > G]T | 4.72E−04 | 0.349613357 |
| HNSCC | SMOKING | T[C > T]T | 8.78E−04 | 17.10716576 |
| HNSCC | SMOKING | V[C > T]G | −2.97E−04 | −7.235989699 |
| HNSCC | SMOKING | T[C > G]A | 4.53E−04 | 5.030776898 |
| HNSCC | SMOKING | T[C > T]G | 7.33E−05 | 1.850408158 |
| HNSCC | SMOKING | T[C > T]A | 1.36E−04 | −7.288456274 |
| KIRP | SMOKING | C[C > A]G | 0.003853441 | 6.347594314 |
| KIRP | SMOKING | C[C > A]H | 0.002732937 | −4.97892693 |
| KIRP | SMOKING | T[C > T] | −3.98E−04 | −21.2145163 |
| KIRP | SMOKING | V[C > T] | −2.52E−04 | −16.31469673 |
| KIRP | SMOKING | C > G | 5.99E−04 | 11.23858751 |
| KIRP | SMOKING | T > A | 5.85E−04 | 11.23858751 |
| KIRP | SMOKING | T > G | 5.85E−04 | 11.23858751 |
| KIRP | SMOKING | B[T > C]B | 3.92E−04 | 11.23858751 |
| KIRP | SMOKING | K[T > C]A | 4.82E−05 | 11.23858751 |
| KIRP | SMOKING | A[T > C] | −1.42E−04 | −45.96461196 |
| PAAD | SMOKING | T[C > A]G | 3.33E−04 | 171.1752347 |
| PAAD | SMOKING | T[C > A]H | 3.36E−04 | 151.7872601 |
| PAAD | SMOKING | V[C > A] | 5.46E−04 | 41.65527202 |
| PAAD | SMOKING | B[C > T]G | 3.74E−04 | 0.902949564 |
| PAAD | SMOKING | [C > T]H | 2.98E−04 | 4.164344561 |
| PAAD | SMOKING | A[C > T]G | −7.97E−05 | −41.28524529 |
| PAAD | SMOKING | C > G | −9.94E−05 | −21.65424411 |
| PAAD | SMOKING | T > A | −9.71E−05 | −21.65424411 |
| PAAD | SMOKING | T > G | −9.71E−05 | −21.65424411 |
| PAAD | SMOKING | T > C | −9.71E−05 | −21.65424411 |
| ESCSQ | SMOKING | A[T > C]B | 7.30E−04 | 70.14606632 |
| ESCSQ | SMOKING | V[C > T]G | −2.46E−04 | −4.517359062 |
| ESCSQ | SMOKING | T[C > A] | −5.26E−04 | −8.429838481 |
| ESCAD | SMOKING | G[C > A]A | −6.49E−04 | 195.0510129 |
| ESCAD | SMOKING | T[C > A] | 0.001207777 | 43.47516368 |
| CESC | SMOKING | T[C > T]G | −7.37E−04 | −2.579095464 |
| CESC | SMOKING | T[C > G]S | −6.35E−04 | −3.493886644 |
| CESC | SMOKING | T[C > G]T | −0.001471515 | −0.014289306 |
| CESC | SMOKING | T[C > T]Y | −0.002020054 | −0.960046865 |
| CESC | SMOKING | T[C > A]A | −2.98E−04 | −6.000430319 |
| CESC | SMOKING | T > A | 5.05E−04 | 0.650171295 |
| CESC | SMOKING | T > G | 5.05E−04 | 0.650171295 |
| CESC | SMOKING | T > C | 5.05E−04 | 0.650171295 |
| CESC | SMOKING | V[C > A] | 3.86E−04 | 0.650171295 |
| CESC | SMOKING | V[C > G] | 3.86E−04 | 0.650171295 |
| CESC | SMOKING | T[C > G]A | −9.59E−04 | 2.340591295 |
| UCEC | POLE | M[C > T]H | 0.067068344 | 798.743625 |
| STAD | POLE | C[C > A]T | 0.009284301 | 474.4760036 |
| COAD | POLE | V[C > A]T | 0.04740875 | 117.4375505 |
| BRCA | POLE | T[C > A]G | 3.40E−04 | −50.81890522 |
| BRCA | POLE | V[C > T]H | 0.004284715 | 22.66073605 |
| BRCA | POLE | T[C > T]Y | 0.002347474 | 24.48532606 |
| BRCA | POLE | V[C > A]K | 0.005740796 | 9.180529304 |
| BRCA | POLE | M[C > A]A | 0.002992879 | 9.180529304 |
| BRCA | POLE | S[C > A]C | 0.002992197 | 9.180529304 |
| BRCA | POLE | G[C > A]A | 0.001296408 | −24.31284003 |
| BRCA | POLE | A[C > A]C | 0.001002733 | −24.97505595 |
| BRCA | POLE | T[C > T]A | 6.72E−04 | −1.873444344 |
| BRCA | POLE | T > A | 0.0017973 | 0.260345272 |
| BRCA | POLE | T > G | 0.0017973 | 0.260345272 |
| BRCA | POLE | T > C | 0.0017973 | 0.260345272 |
| BRCA | POLE | V[C > G] | 0.001373431 | 0.260345272 |
| BRCA | POLE | T[C > A]H | 0.004163989 | −6.67574091 |
| BRCA | POLE | T[C > G]S | 2.48E−05 | 0.542776738 |
| BRCA | POLE | S[C > T]G | −7.46E−05 | −51.10144997 |
| BRCA | POLE | T[C > G]A | −2.04E−04 | −89.05355767 |
| BRCA | POLE | T[C > T]G | 5.58E−05 | −38.08301014 |
| BRCA | POLE | A[C > T]G | −1.23E−04 | −78.35476346 |
| UCEC | MSI | A[C > T]G | 0.006110755 | 304.3731618 |
| STAD | MSI | G[C > T]G | 0.013940773 | 264.0907263 |
| STAD | MSI | G[T > C]A | 0.003746603 | 1266.741125 |
| COAD | MSI | G[T > C]A | 0.004884243 | 164.0922269 |
| BRCA | BRCA | V[C > T]H | 0.00735949 | 24.27948756 |
| BRCA | BRCA | T[C > A]Y | 0.002758222 | 4.209571688 |
| BRCA | BRCA | T[C > A]G | 3.28E−04 | −34.71199559 |
| BRCA | BRCA | T[C > T]Y | 0.010931784 | 4.859995715 |
| BRCA | BRCA | T[C > G]T | 0.009785423 | 22.11525128 |
| BRCA | BRCA | T[C > A]A | 0.002478387 | 29.0851841 |
| BRCA | BRCA | T[C > G]S | 0.003160096 | −16.95722531 |
| BRCA | BRCA | T[C > G]A | 0.008200517 | −11.5889167 |
| BRCA | BRCA | T[C > T]A | 0.013914981 | 3.321087834 |
| BRCA | BRCA | T > A | 0.003452888 | 3.874758344 |
| BRCA | BRCA | T > G | 0.003452888 | 3.874758344 |
| BRCA | BRCA | T > C | 0.003452888 | 3.874758344 |
| BRCA | BRCA | V[C > G] | 0.002638573 | 3.874758344 |
| BRCA | BRCA | T[C > T]G | 0.001187005 | −66.93549821 |
| BRCA | BRCA | A[C > A]C | 7.33E−05 | −72.94011143 |
| BRCA | BRCA | V[C > A]D | 0.001598481 | −21.47371329 |
| BRCA | BRCA | S[C > A]C | 4.74E−04 | −21.47371329 |
| BRCA | BRCA | S[C > T]G | 2.34E−04 | −60.74323487 |
| BRCA | BRCA | A[C > T]G | −2.34E−05 | −82.88142549 |
| OV | BRCA | [C > A]D | 0.003530292 | 14.61561575 |
| OV | BRCA | B[C > A]C | 0.001178124 | 14.61561575 |
| OV | BRCA | V[C > T]H | 0.003280396 | 14.84144831 |
| OV | BRCA | T > A | 0.002581313 | 0.438321984 |
| OV | BRCA | T > G | 0.002581313 | 0.438321984 |
| OV | BRCA | T > C | 0.002581313 | 0.438321984 |
| OV | BRCA | S[C > G] | 0.001440335 | 0.438321984 |
| OV | BRCA | T[C > T]H | 8.37E−04 | −15.70658949 |
| OV | BRCA | A[C > A]C | 1.19E−04 | −57.62102607 |
| OV | BRCA | T[C > G]V | 2.05E−04 | −4.705588576 |
| OV | BRCA | A[C > G] | 2.37E−04 | −19.16113359 |
| OV | BRCA | V[C > T]G | 2.40E−05 | −27.11396147 |
| OV | BRCA | T[C > G]T | −6.40E−05 | −62.27078706 |
| OV | BRCA | T[C > T]G | −6.23E−05 | −55.39999924 |
| SKCM | UV* | C[C > T]C | 0.023544357 | 135.9078586 |
| SKCM | UV* | C[C > T]D | 0.037453644 | 2.076205489 |
| SKCM | UV* | T[C > T]C | 0.062499374 | 11.82128401 |
| SKCM | UV* | T[C > T]W | 0.044790859 | 24.4359456 |
| SKCM | UV* | T[C > T]G | 0.011150999 | −2.894308453 |
| SKCM | UV* | R[C > T]C | 0.012056509 | 102.2053362 |
| SKCM | UV* | D[C > A] | 0.007565221 | 207.0149609 |
| SKCM | UV* | G[T > C]T | 0.002292982 | 59.83010431 |
| SKCM | UV* | C > G | 0.00452755 | −59.5125581 |
| SKCM | UV* | T > A | 0.004421674 | −59.5125581 |
| SKCM | UV* | T > G | 0.004421674 | −59.5125581 |
| SKCM | UV* | [T > C]V | 0.00320509 | −59.5125581 |
| SKCM | UV* | H[T > C]T | 0.001001909 | −59.5125581 |
| SKCM | UV* | R[C > T]D | 0.005162876 | −127.2424776 |
| UCEC | POLD | C[C > A]T | 0.016237897 | 201.9390541 |
| STAD | POLD | W[T > C]G | 0.008022029 | 208.6075187 |
| GBM | MGMT | [C > T]H | 0.001304782 | 12.06580785 |
| GBM | MGMT | C[C > A]G | −1.61E−04 | −50.36582587 |
| GBM | MGMT | Y[C > T]G | 4.81E−04 | 47.76794828 |
| GBM | MGMT | B[C > A]H | −6.22E−04 | −45.57999266 |
| GBM | MGMT | D[C > A]G | −6.28E−05 | −45.57999266 |
| GBM | MGMT | A[C > A]W | −1.06E−04 | −45.57999266 |
| GBM | MGMT | C > G | 1.11E−04 | 13.12444027 |
| GBM | MGMT | T > A | 1.09E−04 | 13.12444027 |
| GBM | MGMT | T > G | 1.09E−04 | 13.12444027 |
| GBM | MGMT | T > C | 1.09E−04 | 13.12444027 |
| GBM | MGMT | A[C > T]G | 2.13E−04 | 15.28834636 |
| GBM | MGMT | G[C > T]G | −1.19E−04 | −77.06516588 |
| GBM | MGMT | A[C > A]C | −1.36E−04 | −123.225394 |
| LGG | MGMT | [C > T]H | 0.001277172 | 8.539100585 |
| LGG | IDH | A[T > C] | −8.08E−04 | −63.92144832 |
| LGG | IDH | Y[C > T]G | 2.41E−04 | 18.3020845 |
| LGG | IDH | B[T > C]D | −9.00E−04 | −30.7167126 |
| LGG | IDH | Y[T > C]C | −2.48E−04 | −30.7167126 |
| LGG | IDH | A[C > T]G | 2.50E−04 | 23.32648387 |
| LGG | IDH | T[C > G]T | −2.46E−04 | −33.35253461 |
| LGG | IDH | G[T > C]C | −3.61E−04 | −66.49194457 |
| LGG | IDH | G[C > T]G | −1.50E−04 | −7.566752823 |
| LGG | IDH | [C > T]H | −3.03E−05 | 3.358138418 |
| LGG | IDH | C > A | −3.59E−05 | 9.244828124 |
| LGG | IDH | T > A | −3.51E−05 | 9.244828124 |
| LGG | IDH | T > G | −3.51E−05 | 9.244828124 |
| LGG | IDH | [C > G]V | −2.52E−05 | 9.244828124 |
| LGG | IDH | V[C > G]T | −7.75E−06 | 9.244828124 |
| GBM | IDH | C > G | −0.002663938 | −31.37863023 |
| GBM | IDH | T > A | −0.002601642 | −31.37863023 |
| GBM | IDH | T > G | −0.002601642 | −31.37863023 |
| GBM | IDH | [T > C]V | −0.001885824 | −31.37863023 |
| GBM | IDH | B[T > C]T | −5.68E−04 | −31.37863023 |
| GBM | IDH | A[C > T]G | 3.11E−04 | 51.12029655 |
| GBM | IDH | C[C > A]G | 1.80E−04 | 141.5044627 |
| GBM | IDH | [C > T]H | −8.13E−04 | 49.01528791 |
| GBM | IDH | G[C > T]G | 4.72E−05 | −1.886969344 |
| GBM | IDH | D[C > A]D | −0.001181546 | 14.6006604 |
| GBM | IDH | K[C > A]C | −3.72E−04 | 14.6006604 |
| UCEC | BMI | A[C > A]G | 6.88E−05 | 58.28972554 |
| UCEC | BMI | A[C > T]G | 2.85E−04 | 36.61826159 |
| UCEC | BMI | V[C > A]H | 9.09E−04 | 16.41637459 |
| UCEC | BMI | S[C > A]G | 8.98E−05 | 16.41637459 |
| UCEC | BMI | T[C > G]T | −6.61E−04 | −21.82224382 |
| UCEC | BMI | T[C > T]A | −4.19E−04 | 8.020171099 |
| UCEC | BMI | T[C > T]Y | −1.75E−04 | −11.05733131 |
| UCEC | BMI | T[C > G]C | −2.27E−04 | −35.37247584 |
| UCEC | BMI | T[C > G]G | −8.90E−06 | 55.22643596 |
| UCEC | BMI | T[C > G]A | −4.10E−04 | 22.80912455 |
| UCEC | BMI | T[C > A] | −9.31E−05 | −1.351500458 |
| UCEC | BMI | T > A | 9.34E−05 | −2.529748051 |
| UCEC | BMI | T > G | 9.34E−05 | −2.529748051 |
| UCEC | BMI | T > C | 9.34E−05 | −2.529748051 |
| UCEC | BMI | V[C > G]H | 6.31E−05 | −2.529748051 |
| UCEC | BMI | S[C > T]G | 3.49E−04 | −3.621242718 |
| UCEC | BMI | V[C > G]G | 1.65E−04 | 32.40432229 |
| KIRP | BMI | D[C > A] | 0.00260618 | 45.37250889 |
| KIRP | BMI | C[C > A]H | 0.017485328 | 3.08944698 |
| KIRP | BMI | A[T > C] | −2.19E−04 | −35.43303612 |
| KIRP | BMI | C > T | −8.91E−04 | −14.9871381 |
| ESCA | BMI | T[C > A] | −0.002378027 | −898.8449791 |
| ESCA | BMI | G[C > A]A | 2.41E−04 | 8617.561311 |
| ESCA | BMI | V[C > A]B | −0.002554718 | −1288.258786 |
| ESCA | BMI | M[C > A]A | −7.77E−04 | −1288.258786 |
| ESCA | BMI | C[T > G]T | 0.00111906 | −368.8776757 |
| ESCA | BMI | T[C > T]G | −4.25E−04 | 582.285238 |
| ESCA | BMI | D[T > G]T | 7.73E−04 | 2270.053081 |
| COAD | BMI | T[C > A]V | −1.60E−04 | −63.41852595 |
| COAD | BMI | T[C > G]T | −2.19E−04 | −53.1562501 |
| COAD | BMI | Y[C > T]G | 8.91E−04 | 7.375355964 |
| COAD | BMI | A[C > T]G | 5.53E−04 | 6.15758336 |
| COAD | BMI | V[C > A]B | 6.43E−04 | 2.046889675 |
| COAD | BMI | M[C > A]A | 1.96E−04 | 2.046889675 |
| COAD | BMI | G[C > T]G | 9.34E−04 | −2.082165624 |
| COAD | BMI | T[C > A]T | 2.71E−04 | 76.95313357 |
| COAD | BMI | [C > T]H | 0.001694774 | −0.915341631 |
| COAD | BMI | T > A | 4.87E−04 | 2.923946154 |
| COAD | BMI | T > G | 4.87E−04 | 2.923946154 |
| COAD | BMI | T > C | 4.87E−04 | 2.923946154 |
| COAD | BMI | [C > G]V | 3.50E−04 | 2.923946154 |
| COAD | BMI | V[C > G]T | 1.08E−04 | 2.923946154 |
| HNSCC | ALCOHOL | V[C > T]H | −0.002568167 | −8081.534896 |
| HNSCC | ALCOHOL | T[C > A] | −6.66E−04 | 4083.240397 |
| HNSCC | ALCOHOL | G[C > A]A | −2.97E−04 | 9431.646697 |
| HNSCC | ALCOHOL | T > A | −4.36E−04 | 1565.530351 |
| HNSCC | ALCOHOL | T > G | −4.36E−04 | 1565.530351 |
| HNSCC | ALCOHOL | T > C | −4.36E−04 | 1565.530351 |
| HNSCC | ALCOHOL | V[C > G] | −3.34E−04 | 1565.530351 |
| ESCA | ALCOHOL | H[C > A] | 0.002928588 | 296.602829 |
| ESCA | ALCOHOL | C[T > C]T | 0.001064218 | 1120.339803 |
| ESCA | ALCOHOL | C[T > G]T | 0.00113103 | −418.5600537 |
| ESCA | ALCOHOL | A[T > C]A | 5.27E−04 | 1016.681368 |
| ESCA | ALCOHOL | [C > T]H | −0.001175418 | −211.5001355 |
| ESCA | ALCOHOL | T > A | 7.58E−04 | 148.0218794 |
| ESCA | ALCOHOL | V[C > G] | 5.79E−04 | 148.0218794 |
| ESCA | ALCOHOL | [T > G]V | 5.49E−04 | 148.0218794 |
| ESCA | ALCOHOL | B[T > C]V | 4.31E−04 | 148.0218794 |
| ESCA | ALCOHOL | D[T > G]T | 1.48E−04 | 148.0218794 |
| ESCA | ALCOHOL | D[T > C]T | 1.48E−04 | 148.0218794 |
| ESCA | ALCOHOL | A[T > C]S | 8.75E−05 | 148.0218794 |
| ESCA | ALCOHOL | G[C > A]A | 3.59E−04 | 491.5609162 |
| ESCA | ALCOHOL | [C > T]G | −3.49E−04 | −402.6213358 |
| ESCA | ALCOHOL | G[C > A]B | −2.62E−04 | −736.7119568 |
| LIHC | ALCOHOL | Y[T > C]B | 0.001154162 | 122.3544839 |
| LIHC | ALCOHOL | A[T > C]A | 2.97E−04 | 32.3339868 |
| LIHC | ALCOHOL | V[C > A]G | 2.26E−04 | 20.65305035 |
| LIHC | ALCOHOL | V[C > A]W | 6.04E−04 | −3.506725046 |
| LIHC | ALCOHOL | G[C > T]H | 3.97E−04 | 19.67204081 |
| LIHC | ALCOHOL | V[C > A]C | 4.61E−04 | 16.8571888 |
| LIHC | ALCOHOL | T[C > A]G | 7.92E−06 | −17.63184771 |
| LIHC | ALCOHOL | H[C > T]G | 1.48E−05 | −5.190689269 |
| LIHC | ALCOHOL | Y[T > C]A | 2.71E−05 | −65.31448915 |
| LIHC | ALCOHOL | G[T > C]B | −2.10E−04 | −134.7244679 |
| LIHC | ALCOHOL | A[T > C]B | 2.05E−04 | −11.74956725 |
| LIHC | ALCOHOL | C > G | 4.19E−04 | −2.562044945 |
| LIHC | ALCOHOL | T > G | 4.09E−04 | −2.562044945 |
| LIHC | ALCOHOL | [T > A]H | 2.83E−04 | −2.562044945 |
| LIHC | ALCOHOL | D[T > A]G | 8.09E−05 | −2.562044945 |
| LIHC | ALCOHOL | T[C > A]C | 4.73E−05 | −12.92256308 |
| LIHC | ALCOHOL | C[T > A]G | 8.30E−04 | −5.037804972 |
| LIHC | ALCOHOL | G[C > T]G | −1.78E−04 | 5.570329712 |
| LIHC | ALCOHOL | T[C > A]W | −1.10E−06 | −19.65534781 |
| LIHC | ALCOHOL | H[C > T]H | −5.06E−04 | −25.11362966 |
| LIHC | HepB | C[T > A]H | 8.18E−04 | 40.91834356 |
| LIHC | HepB | G[T > C]B | −4.58E−04 | −134.919894 |
| LIHC | HepB | V[C > A]W | 9.85E−04 | 21.08251879 |
| LIHC | HepB | C > G | 8.19E−04 | 4.453855831 |
| LIHC | HepB | T > G | 8.00E−04 | 4.453855831 |
| LIHC | HepB | D[T > A] | 5.56E−04 | 4.453855831 |
| LIHC | HepB | Y[T > C]B | 4.02E−04 | 4.453855831 |
| LIHC | HepB | A[T > C]A | 3.32E−04 | 37.41333501 |
| LIHC | HepB | H[C > T]G | 5.44E−04 | 68.73517462 |
| LIHC | HepB | A[T > C]B | 5.66E−04 | −1.53938766 |
| LIHC | HepB | Y[T > C]A | 1.97E−04 | 59.65436897 |
| LIHC | HepB | V[C > A]G | 3.25E−04 | 14.29565561 |
| LIHC | HepB | V[C > A]C | 6.89E−04 | −11.65622571 |
| LIHC | HepB | [C > T]H | 0.001248643 | −4.616550159 |
| LIHC | HepB | T[C > A]G | 3.19E−05 | −66.64624483 |
| LIHC | HepB | C[T > A]G | 4.91E−04 | −5.416037112 |
| LIHC | HepB | G[T > C]A | 4.04E−05 | −33.39524467 |
| LIHC | HepB | G[C > T]G | −1.10E−04 | −125.3982196 |
| LIHC | HepC | V[C > A]W | 0.001034676 | 54.12897988 |
| LIHC | HepC | A[T > C]A | 2.52E−04 | 21.60512912 |
| LIHC | HepC | Y[T > C]A | 1.45E−04 | 47.49231923 |
| LIHC | HepC | V[C > A]G | 3.41E−04 | 10.04155523 |
| LIHC | HepC | C > G | 5.52E−04 | 7.407823371 |
| LIHC | HepC | T > G | 5.40E−04 | 7.407823371 |
| LIHC | HepC | [T > A]H | 3.73E−04 | 7.407823371 |
| LIHC | HepC | Y[T > C]B | 2.71E−04 | 7.407823371 |
| LIHC | HepC | D[T > A]G | 1.07E−04 | 7.407823371 |
| LIHC | HepC | G[T > C]B | −4.79E−04 | −167.0528828 |
| LIHC | HepC | A[T > C]B | 2.80E−04 | 6.591656833 |
| LIHC | HepC | H[C > T]G | −2.59E−04 | −42.30992472 |
| LIHC | HepC | V[C > A]C | 2.64E−05 | −55.74523417 |
| LIHC | HepC | T[C > A]G | 3.59E−05 | −50.41533656 |
| LIHC | HepC | T[C > A]W | 1.49E−04 | −43.11592378 |
| BLCA | AAcid | D[T > A]A | 0.074829627 | 95.95218692 |
| MESO | Asb* | [C > T]G | 5.92E−04 | 277.311545 |
| MESO | Asb* | C > G | 5.94E−04 | 36.5744555 |
| MESO | Asb* | T > A | 5.80E−04 | 36.5744555 |
| MESO | Asb* | T > C | 5.80E−04 | 36.5744555 |
| MESO | Asb* | [C > A]H | 5.30E−04 | 36.5744555 |
| MESO | Asb* | [T > G]D | 4.30E−04 | 36.5744555 |
| MESO | Asb* | V[T > G]C | 1.06E−04 | 36.5744555 |
| CESC | APOBEC | T[C > A]B | 0.001554717 | 18482.29688 |
| CESC | APOBEC | T[C > A]A | 0.001114974 | 10738.54636 |
| CESC | APOBEC | T[C > T]A | 0.002766779 | 806.7242792 |
| CESC | APOBEC | T[C > T]Y | 0.003188246 | −66.6071713 |
| CESC | APOBEC | T[C > G]A | 0.00213553 | −1295.420832 |
| CESC | APOBEC | T[C > T]G | 6.10E−04 | −398.230882 |
| CESC | APOBEC | T > A | −4.02E−04 | −1047.019501 |
| CESC | APOBEC | T > G | −4.02E−04 | −1047.019501 |
| CESC | APOBEC | T > C | −4.02E−04 | −1047.019501 |
| CESC | APOBEC | V[C > A] | −3.07E−04 | −1047.019501 |
| CESC | APOBEC | V[C > G] | −3.07E−04 | −1047.019501 |
| CESC | APOBEC | T[C > G]T | 0.002162507 | −799.1173319 |
| CESC | APOBEC | T[C > G]S | 0.001110844 | −282.349177 |
| CESC | APOBEC | V[C > T]G | −2.64E−04 | −458.6957236 |
| CESC | APOBEC | V[C > T]H | 2.22E−05 | −67.27653778 |
| KIRC | APOBEC | V[C > T]H | 4.68E−04 | 12.3328489 |
| KIRC | APOBEC | T[C > T]Y | 1.14E−04 | 12.3328489 |
| KIRC | APOBEC | A[T > C]A | −5.56E−05 | −56.74988937 |
| KIRC | APOBEC | B[T > C]A | −1.75E−04 | −63.63282981 |
| KIRC | APOBEC | V[C > A] | 3.20E−04 | 11.55669328 |
| KIRC | APOBEC | A[T > C]B | −1.44E−04 | −50.96059574 |
| KIRC | APOBEC | C > G | 1.30E−04 | 7.36696687 |
| KIRC | APOBEC | T > A | 1.27E−04 | 7.36696687 |
| KIRC | APOBEC | T > G | 1.27E−04 | 7.36696687 |
| KIRC | APOBEC | B[T > C]B | 8.53E−05 | 7.36696687 |
| KIRC | APOBEC | [C > T]G | −1.37E−04 | −8.096188464 |
| KIRC | APOBEC | T[C > T]A | −1.13E−04 | −50.5505232 |
| KIRC | APOBEC | T[C > A] | −1.17E−04 | −30.49965586 |

TABLE 9
Comparisons of prediction accuracy (AUC) and correlation across methods. The AUCs and correlations, both apparent and cross-validated, are reported for age and all other
etiological factors across all tissue types for each one of the mutational signature methodologies considered in this study: Logistic Regression (Logit), Linear Discriminant Analysis (LDA), Nonnegative
Least Square Logit using the Betas (NNLS_Logit_betas), Non-negative Least Square Logit using the means (NNLS_Logit_means), Random Forest (RF), Unsupervised as in Alexandrov
et al. (Unsupervised), Best_NMF, Matched_NMF, Signature 1 as in Alexandrov et al. (Signature1), and Single Peak (SinglePeak).
Age Apparent

| type | tissue | factor | Best_NMF | LDA | Logit | Matched_NMF | NNLS_Logit_betas | NNLS_Logit_means | RF | Signature1 | SinglePeak | Unsupervised |
|---|---|---|---|---|---|---|---|---|---|---|---|---|
| Apparent | ACC | AGE | 0.616521739 | 0.768695652 | 0.768695652 | NA | 0.768695652 | 0.768695652 | 0.782608696 | 0.471304348 | 0.74 | NA |
| Apparent | BLCA | AGE | 0.638605442 | 0.72130102 | 0.72130102 | 0.491709184 | 0.72130102 | 0.72130102 | 0.81079932 | 0.654336735 | 0.624787415 | 0.654336735 |
| Apparent | BRCA | AGE | 0.620643337 | 0.623848238 | 0.623753977 | 0.575162012 | 0.623753977 | 0.59191705 | 0.664899258 | 0.555190291 | 0.568080594 | 0.60466596 |
| Apparent | CESC | AGE | 0.698191214 | 0.764857881 | 0.824289406 | 0.698191214 | 0.824289406 | 0.720413437 | 0.789147287 | 0.56873385 | 0.614728682 | 0.56873385 |
| Apparent | CHOL | AGE | 0.526627219 | 0.766272189 | 0.766272189 | NA | 0.766272189 | 0.766272189 | 0.766272189 | 0.553254438 | 0.627218935 | NA |
| Apparent | COAD | AGE | 0.642299688 | 0.69640999 | 0.696930281 | 0.642299688 | 0.696930281 | 0.675078044 | 0.735431842 | 0.590530697 | 0.68405307 | 0.590530697 |
| Apparent | ESCAD | AGE | 0.620498615 | 0.670360111 | 0.670360111 | 0.501385042 | 0.670360111 | 0.63434903 | 0.717451524 | 0.573407202 | 0.516620499 | 0.573407202 |
| Apparent | ESCSQ | AGE | 0.595441595 | 0.61965812 | 0.61965812 | 0.595441595 | 0.61965812 | 0.61965812 | 0.646723647 | 0.575498575 | 0.487179487 | 0.575498575 |
| Apparent | GBM | AGE | 0.677777778 | 0.690608466 | 0.690608466 | 0.627777778 | 0.690608466 | 0.690608466 | 0.748015873 | 0.612301587 | 0.682671958 | 0.612301587 |
| Apparent | HNSCC | AGE | 0.72381217 | 0.8291192 | 0.830508475 | 0.614337316 | 0.830508475 | 0.741872742 | 0.835787719 | 0.671158655 | 0.745762712 | 0.671158655 |
| Apparent | KICH | AGE | 0.825259516 | 0.865051903 | 0.865051903 | 0.541522491 | 0.865051903 | 0.844290657 | 0.903114187 | 0.709342561 | 0.858131488 | 0.761245675 |
| Apparent | KIRC | AGE | 0.662870763 | 0.812235169 | 0.812235169 | 0.575476695 | 0.812235169 | 0.761917373 | 0.801112288 | 0.551112288 | 0.771716102 | 0.724311441 |
| Apparent | KIRP | AGE | 0.695156695 | 0.753561254 | 0.753561254 | 0.695156695 | 0.753561254 | 0.753561254 | 0.77991453 | 0.494301994 | 0.717948718 | 0.705128205 |
| Apparent | LAML | AGE | 0.706597222 | 0.683159722 | 0.683159722 | 0.706597222 | 0.683159722 | 0.683159722 | 0.689236111 | 0.585069444 | 0.615451389 | 0.635416667 |
| Apparent | LGG | AGE | 0.759259259 | 0.883333333 | 0.883333333 | 0.85 | 0.883333333 | 0.883333333 | 0.95 | 0.792592593 | 0.877777778 | 0.944444444 |
| Apparent | LIHC | AGE | 0.620689655 | 0.759236453 | 0.756773399 | 0.564655172 | 0.756773399 | 0.745689655 | 0.751847291 | 0.549261084 | 0.674261084 | 0.674876847 |
| Apparent | LUAD | AGE | 0.604938272 | 0.643518519 | 0.643518519 | 0.564814815 | 0.643518519 | 0.643518519 | 0.75154321 | 0.456790123 | 0.574074074 | 0.456790123 |
| Apparent | OV | AGE | 0.525980912 | 0.693796394 | 0.711293743 | 0.51378579 | 0.711293743 | 0.707051962 | 0.693796394 | 0.671792153 | 0.540031813 | 0.671792153 |
| Apparent | PAAD | AGE | 0.71754386 | 0.680701754 | 0.680701754 | 0.71754386 | 0.680701754 | 0.680701754 | 0.71754386 | 0.638596491 | 0.533333333 | 0.638596491 |
| Apparent | PCPG | AGE | 0.704294218 | 0.767857143 | 0.763605442 | 0.742772109 | 0.763605442 | 0.758503401 | 0.771896259 | 0.523384354 | 0.77827381 | 0.753401361 |
| Apparent | PRAD | AGE | 0.606924731 | 0.686903226 | 0.688795699 | 0.606924731 | 0.688795699 | 0.667462366 | 0.716258065 | 0.560451613 | 0.691784946 | 0.608924731 |
| Apparent | SARC | AGE | 0.749188897 | 0.829848594 | 0.832552271 | 0.798485941 | 0.832552271 | 0.781903389 | 0.828947368 | 0.692682048 | 0.793979813 | 0.805875991 |
| Apparent | SKCM | AGE | 0.628792385 | 0.621356336 | 0.621356336 | 0.628792385 | 0.621356336 | 0.621356336 | 0.700178465 | 0.483045806 | 0.533908388 | 0.483045806 |
| Apparent | STAD | AGE | 0.624235006 | 0.66119951 | 0.66119951 | 0.624235006 | 0.66119951 | 0.66119951 | 0.693574051 | 0.6000612 | 0.594614443 | 0.6000612 |
| Apparent | TGCT | AGE | 0.692763158 | 0.601644737 | 0.601644737 | 0.601973684 | 0.601644737 | 0.601644737 | 0.675986842 | 0.432894737 | 0.6 | 0.613157895 |
| Apparent | THCA | AGE | 0.664990282 | 0.777575316 | 0.777429543 | 0.664990282 | 0.777429543 | 0.774951409 | 0.81350826 | 0.518148688 | 0.745310982 | 0.774514091 |
| Apparent | THYM | AGE | 0.727650728 | 0.755024255 | 0.755024255 | 0.684684685 | 0.755024255 | 0.755024255 | 0.772002772 | 0.595980596 | 0.710672211 | 0.718641719 |
| Apparent | UCEC | AGE | 0.727272727 | 0.743801653 | 0.743801653 | 0.504132231 | 0.743801653 | 0.743801653 | 0.809917355 | 0.661157025 | 0.578512397 | 0.561983471 |
| Apparent | UCS | AGE | 0.598039216 | 0.62254902 | 0.62254902 | NA | 0.62254902 | 0.62254902 | 0.743464052 | 0.633986928 | 0.609477124 | NA |
| Apparent | UVM | AGE | 0.735 | 0.69375 | 0.69375 | NA | 0.69375 | 0.69375 | 0.70125 | 0.29 | 0.58625 | NA |
| Apparent | Median | AGE | 0.663930522 | 0.708855505 | 0.716297382 | 0.619286161 | 0.716297382 | 0.713732699 | 0.75169525 | 0.574452889 | 0.626003175 | 0.637006579 |
| Apparent | Subset median | AGE | 0.67138403 | 0.708855505 | 0.716297382 | 0.619286161 | 0.716297382 | 0.713732699 | 0.75169525 | 0.58028401 | 0.649524249 | 0.637006579 |
| Apparent | Overall median | AGE | 0.663930522 | 0.708855505 | 0.716297382 | 0.604449208 | 0.716297382 | 0.713732699 | 0.75169525 | 0.574452889 | 0.626003175 | 0.612729741 |

Other Exposures Apparent

| type | tissue | factor | Best_NMF | LDA | Logit | Matched_NMF | NNLS_Logit_betas | NNLS_Logit_means | RF | Signature1 | SinglePeak | Unsupervised |
|---|---|---|---|---|---|---|---|---|---|---|---|---|
| Apparent | BLCA | AAcid | 0.940557276 | 0.995665635 | 0.995665635 | 0.940557276 | 0.995665635 | 0.995665635 | 1 | NA | NA | 0.964396285 |
| Apparent | ESCA | ALCOHOL | 0.68287037 | 0.99537037 | 1 | NA | 0.805555556 | 0.782407407 | 0.967592593 | NA | NA | NA |
| Apparent | HNSCC | ALCOHOL | 0.589861751 | 0.99078341 | 1 | NA | 0.75 | 0.5 | 0.956221198 | NA | NA | NA |
| Apparent | LIHC | ALCOHOL | 0.604683196 | 0.945936639 | 0.968491736 | NA | 0.911157025 | 0.625516529 | 0.900740358 | NA | NA | NA |
| Apparent | CESC | APOBEC | 0.670889894 | 0.943891403 | 1 | 0.62745098 | 0.961538462 | 0.642835596 | 0.946153846 | NA | NA | 0.638612368 |
| Apparent | KIRC | APOBEC | 0.625963391 | 0.885356455 | 0.899566474 | NA | 0.899566474 | 0.65438343 | 0.98265896 | NA | NA | NA |
| Apparent | MESO | Asb* | 0.9375 | 0.9875 | 0.984090909 | NA | 0.984090909 | 0.922727273 | 1 | NA | NA | NA |
| Apparent | COAD | BMI | 0.601992699 | 0.842865835 | 0.87336477 | NA | 0.860282933 | 0.560769699 | 0.951057195 | NA | NA | NA |
| Apparent | ESCA | BMI | 0.684729064 | 0.966748768 | 1 | NA | 0.965517241 | 0.497536946 | 0.948891626 | NA | NA | NA |
| Apparent | KIRP | BMI | 0.74516129 | 0.947580645 | 0.939516129 | NA | 0.952822581 | 0.836290323 | 0.992741935 | NA | NA | NA |
| Apparent | UCEC | BMI | 0.614565708 | 0.836717428 | 0.866469261 | NA | 0.862803158 | 0.644247039 | 0.978355894 | NA | NA | NA |
| Apparent | BRCA | BRCA | 0.755708344 | 0.940411425 | 0.981511391 | 0.755708344 | 0.96933441 | 0.849965998 | 0.952078375 | NA | NA | 0.67027417 |
| Apparent | OV | BRCA | 0.812738368 | 0.941266209 | 0.961098398 | 0.663615561 | 0.961098398 | 0.793668955 | 0.845728452 | NA | NA | 0.809687262 |
| Apparent | LIHC | HepB | 0.589090909 | 0.926666667 | 0.956969697 | NA | 0.956969697 | 0.664393939 | 0.926742424 | NA | NA | NA |
| Apparent | LIHC | HepC | 0.654325513 | 0.92228739 | 0.958944282 | NA | 0.958944282 | 0.682917889 | 0.965175953 | NA | NA | NA |
| Apparent | GBM | IDH | 0.719957082 | 0.979613734 | 0.982296137 | NA | 0.93776824 | 0.5 | 0.987392704 | NA | NA | NA |
| Apparent | LGG | IDH | 0.785620667 | 0.917186907 | 0.938122995 | NA | 0.929569206 | 0.5 | 0.983082123 | NA | NA | NA |
| Apparent | GBM | MGMT | 0.660787499 | 0.920321807 | 0.940047962 | NA | 0.937881953 | 0.840179469 | 0.998530208 | NA | NA | NA |
| Apparent | LGG | MGMT | 0.695887446 | 0.748917749 | 0.748917749 | NA | 0.748917749 | 0.748917749 | 0.811417749 | NA | NA | NA |
| Apparent | COAD | MSI | 0.985375119 | 0.999810066 | 0.999050332 | 0.985375119 | 0.999050332 | 0.999050332 | 0.999335233 | NA | NA | 0.967046534 |
| Apparent | STAD | MSI | 0.956380208 | 0.999925606 | 1 | 0.998480903 | 1 | 1 | 1 | NA | NA | 0.999855324 |
| Apparent | UCEC | MSI | 0.941137566 | 0.999669312 | 0.999669312 | 0.975694444 | 0.999669312 | 0.999669312 | 0.999834656 | NA | NA | 1 |
| Apparent | STAD | POLD | 0.969017094 | 1 | 1 | NA | 1 | 1 | 1 | NA | NA | NA |
| Apparent | UCEC | POLD | 0.902777778 | 0.998015873 | 0.998015873 | NA | 0.998015873 | 0.998015873 | 1 | NA | NA | NA |
| Apparent | BRCA | POLE | 0.670679887 | 0.950900164 | 0.982760502 | 0.58858139 | 0.982760502 | 0.716093835 | 0.984397163 | NA | NA | 0.423294835 |
| Apparent | COAD | POLE | 0.926923077 | 1 | 1 | 0.649679487 | 1 | 1 | 1 | NA | NA | 0.72275641 |
| Apparent | STAD | POLE | 0.955409357 | 1 | 1 | NA | 1 | 1 | 1 | NA | NA | NA |
| Apparent | UCEC | POLE | 0.896825397 | 1 | 1 | 0.752380952 | 1 | 1 | 1 | NA | NA | 0.734126984 |
| Apparent | BLCA | SMOKING | 0.629527673 | 0.701477833 | 0.701709649 | 0.629527673 | 0.701709649 | 0.693480151 | 0.744537815 | NA | 0.640220226 | 0.683917705 |
| Apparent | CESC | SMOKING | 0.561678832 | 0.629927007 | 0.624543796 | NA | 0.580109489 | 0.582664234 | 0.795757299 | NA | 0.42810219 | NA |
| Apparent | ESCAD | SMOKING | 0.640372671 | 0.991304348 | 0.961490683 | NA | 0.889440994 | 0.891925466 | 0.995031056 | NA | 0.582608696 | NA |
| Apparent | ESCSQ | SMOKING | 0.586857515 | 0.815875081 | 0.828236825 | 0.394274561 | 0.821080026 | 0.575471698 | 0.841899805 | NA | 0.526350033 | 0.470071568 |
| Apparent | HNSCC | SMOKING | 0.75880168 | 0.871810401 | 0.913840439 | 0.67748708 | 0.909439599 | 0.779796512 | 0.942344961 | NA | 0.695332687 | 0.818213017 |
| Apparent | KIRP | SMOKING | 0.62797619 | 0.889136905 | 0.874255952 | 0.519345238 | 0.796130952 | 0.696428571 | 0.99702381 | NA | 0.608258929 | 0.625744048 |
| Apparent | LUAD | SMOKING | 0.872402631 | 0.91684347 | 0.953264969 | 0.883679649 | 0.953883781 | 0.907413956 | 0.955298208 | NA | 0.909809961 | 0.910619192 |
| Apparent | PAAD | SMOKING | 0.607210626 | 0.849778621 | 0.877292853 | NA | 0.878399747 | 0.656230234 | 0.977229602 | NA | 0.548545225 | NA |
| Apparent | SKCM | UV* | 0.939423404 | 0.978636364 | 1 | 0.921678254 | 1 | 0.969444444 | 0.994292929 | NA | NA | 0.949632943 |
| Apparent | Median | NA | 0.695887446 | 0.945936639 | 0.968491736 | 0.714934016 | 0.953883781 | 0.779796512 | 0.98265896 | NA | NA | NA |
| Apparent | Subset median | NA | 0.842570499 | 0.947395783 | 0.989213068 | 0.714934016 | 0.976047456 | 0.878689977 | 0.989345046 | NA | NA | 0.771907123 |
| Apparent | Subset smoking | SMOKING | 0.629527673 | 0.871810401 | 0.874255952 | 0.629527673 | 0.821080026 | 0.696428571 | 0.942344961 | NA | 0.640220226 | 0.683917705 |
| | median | | | | | | | | | | | |
| Apparent | Overall smoking | SMOKING | 0.628751932 | 0.860794511 | 0.875774403 | 0.509672619 | 0.849739887 | 0.694954361 | 0.948821585 | NA | 0.567304481 | 0.562872024 |
| | median | | | | | | | | | | | |

Age Cross-Validated

| type | tissue | factor | Best_NMF | LDA | Logit | Matched_NMF | NNLS_Logit_betas | NNLS_Logit_mea ns | RF | Signature1 | SinglePeak | Unsupervised |
|---|---|---|---|---|---|---|---|---|---|---|---|---|
| Cross-validated | ACC | AGE | 0.6148 | 0.7166 | 0.7166 | NA | 0.7166 | 0.7342 | 0.7226 | NA | NA | NA |
| Cross-validated | BLCA | AGE | 0.59362716 | 0.656577778 | 0.659160494 | 0.51942963 | 0.659160494 | 0.644859259 | 0.700548148 | NA | NA | NA |
| Cross-validated | BRCA | AGE | 0.603975808 | 0.601398544 | 0.601985496 | 0.569491516 | 0.602009292 | 0.576024192 | 0.636424181 | NA | NA | NA |
| Cross-validated | CESC | AGE | 0.664351852 | 0.700092593 | 0.735185185 | 0.676882716 | 0.735185185 | 0.685493827 | 0.659290123 | NA | NA | NA |
| Cross-validated | CHOL | AGE | 0.411111111 | 0.799444444 | 0.799444444 | NA | 0.799444444 | 0.801666667 | 0.739444444 | NA | NA | NA |
| Cross-validated | COAD | AGE | 0.619796187 | 0.627379191 | 0.62035092 | 0.64112426 | 0.62035092 | 0.635434747 | 0.657501644 | NA | NA | NA |
| Cross-validated | ESCAD | AGE | 0.456666667 | 0.529166667 | 0.490833333 | 0.55 | 0.489166667 | 0.528333333 | 0.482083333 | NA | NA | NA |
| Cross-validated | ESCSQ | AGE | 0.509666667 | 0.5368 | 0.528266667 | 0.495066667 | 0.5296 | 0.533555556 | 0.464155556 | NA | NA | NA |
| Cross-validated | GBM | AGE | 0.638205128 | 0.635918803 | 0.634102564 | 0.630363248 | 0.634102564 | 0.647435897 | 0.699369658 | NA | NA | NA |
| Cross-validated | HNSCC | AGE | 0.70961927 | 0.730356449 | 0.718275058 | 0.659480381 | 0.718275058 | 0.731015929 | 0.746230575 | NA | NA | NA |
| Cross-validated | KICH | AGE | 0.889166667 | 0.801388889 | 0.784444444 | 0.613611111 | 0.784444444 | 0.810833333 | 0.811944444 | NA | NA | NA |
| Cross-validated | KIRC | AGE | 0.655011655 | 0.778296426 | 0.777169775 | 0.615827506 | 0.777169775 | 0.753581974 | 0.730574981 | NA | NA | NA |
| Cross-validated | KIRP | AGE | 0.685422222 | 0.706822222 | 0.705488889 | 0.697422222 | 0.705488889 | 0.714822222 | 0.7182 | NA | NA | NA |
| Cross-validated | LAML | AGE | 0.5673 | 0.68765 | 0.68845 | 0.5593 | 0.68845 | 0.69005 | 0.6366 | NA | NA | NA |
| Cross-validated | LGG | AGE | 0.757777778 | 0.855555556 | 0.838333333 | 0.881111111 | 0.838333333 | 0.855 | 0.891111111 | NA | NA | NA |
| Cross-validated | LIHC | AGE | 0.607288889 | 0.741066667 | 0.725466667 | 0.658444444 | 0.7268 | 0.753955556 | 0.683711111 | NA | NA | NA |
| Cross-validated | LUAD | AGE | 0.454861111 | 0.461111111 | 0.464444444 | 0.539305556 | 0.468194444 | 0.475277778 | 0.464444444 | NA | NA | NA |
| Cross-validated | OV | AGE | 0.487487654 | 0.634941358 | 0.628691358 | 0.532768519 | 0.628691358 | 0.622524691 | 0.610774691 | NA | NA | NA |
| Cross-validated | PAAD | AGE | 0.603333333 | 0.692777778 | 0.692777778 | 0.672222222 | 0.692777778 | 0.697222222 | 0.666666667 | NA | NA | NA |
| Cross-validated | PC PG | AGE | 0.685195062 | 0.721311111 | 0.722044444 | 0.73722963 | 0.722044444 | 0.743333333 | 0.74968642 | NA | NA | NA |
| Cross-validated | PRAD | AGE | 0.593569892 | 0.64172043 | 0.644172043 | 0.596021505 | 0.644172043 | 0.661419355 | 0.646193548 | NA | NA | NA |
| Cross-validated | SARC | AGE | 0.732239683 | 0.808830952 | 0.802935714 | 0.801934921 | 0.802935714 | 0.777162698 | 0.769865079 | NA | NA | NA |
| Cross-validated | SKCM | AGE | 0.624131944 | 0.579517747 | 0.579239969 | 0.412391975 | 0.579239969 | 0.584864969 | 0.646246142 | NA | NA | NA |
| Cross-validated | STAD | AGE | 0.606577227 | 0.647120743 | 0.647223942 | 0.607072583 | 0.647162023 | 0.634908841 | 0.651799106 | NA | NA | NA |
| Cross-validated | TGCT | AGE | 0.659732143 | 0.549910714 | 0.554196429 | 0.607232143 | 0.552767857 | 0.551607143 | 0.54875 | NA | NA | NA |
| Cross-validated | THCA | AGE | 0.67701642 | 0.750474548 | 0.75440312 | 0.681228243 | 0.75440312 | 0.766828407 | 0.777423645 | NA | NA | NA |
| Cross-validated | THYM | AGE | 0.742971939 | 0.748016582 | 0.729980867 | 0.67375 | 0.729980867 | 0.717059949 | 0.767755102 | NA | NA | NA |
| Cross-validated | UCEC | AGE | 0.656666667 | 0.657777778 | 0.672777778 | 0.327222222 | 0.672777778 | 0.669444444 | 0.595555556 | NA | NA | NA |
| Cross-validated | UCS | AGE | 0.487777778 | 0.519722222 | 0.501388889 | NA | 0.501388889 | 0.5325 | 0.497638889 | NA | NA | NA |
| Cross-validated | UVM | AGE | 0.60125 | 0.65875 | 0.65875 | NA | 0.65875 | 0.65875 | 0.64 | NA | NA | NA |
| Cross-validated | Median | AGE | 0.617298093 | 0.6732 | 0.680613889 | 0.614719308 | 0.680613889 | 0.677469136 | 0.662978395 | NA | NA | NA |
| Cross-validated | Subset median | AGE | 0.631168536 | 0.672713889 | 0.680613889 | 0.614719308 | 0.680613889 | 0.677469136 | 0.662978395 | NA | NA | NA |
| Cross-validated | Overall median | AGE | 0.617298093 | 0.6732 | 0.680613889 | 0.607152363 | 0.680613889 | 0.677469136 | 0.662978395 | NA | NA | NA |

Other Exposures Cross-Validated

| type | tissue | factor | Best_NMF | LDA | Logit | Matched_NMF | NNLS_Logit_betas | NNLS_Logit_means | RF | Signature1 | SinglePeak | Unsupervised |
|---|---|---|---|---|---|---|---|---|---|---|---|---|
| Cross-validated | BLCA | AAcid | 0.907843137 | 0.982745098 | 0.964117647 | 0.920196078 | 0.982745098 | 0.982745098 | 0.968333333 | NA | NA | NA |
| Cross-validated | ESCA | ALCOHOL | 0.477222222 | 0.905555556 | 0.896388889 | NA | 0.815 | 0.548888889 | 0.78 | NA | NA | NA |
| Cross-validated | HNSCC | ALCOHOL | 0.530873016 | 0.907936508 | 0.905714286 | NA | 0.644920635 | 0.5 | 0.833174603 | NA | NA | NA |
| Cross-validated | LIHC | ALCOHOL | 0.600288731 | 0.82438124 | 0.825065 | NA | 0.819948026 | 0.612044818 | 0.815605114 | NA | NA | NA |
| Cross-validated | CESC | APOBEC | 0.594853147 | 0.896251748 | 0.942727273 | 0.638293706 | 0.95186014 | 0.64558042 | 0.935496503 | NA | NA | NA |
| Cross-validated | KIRC | APOBEC | 0.538339496 | 0.759668908 | 0.810732773 | NA | 0.771678992 | 0.655262185 | 0.92417479 | NA | NA | NA |
| Cross-validated | MESO | Asb* | 0.954 | 0.960375 | 0.9575 | NA | 0.96 | 0.937 | 0.994 | NA | NA | NA |
| Cross-validated | COAD | BMI | 0.534055672 | 0.753743137 | 0.758187115 | NA | 0.757384149 | 0.554754482 | 0.818753081 | NA | NA | NA |
| Cross-validated | ESCA | BMI | 0.620555556 | 0.949333333 | 0.914666667 | NA | 0.862777778 | 0.578444444 | 0.893422222 | NA | NA | NA |
| Cross-validated | KIRP | BMI | 0.697857143 | 0.853809524 | 0.891309524 | NA | 0.891845238 | 0.818392857 | 0.93047619 | NA | NA | NA |
| Cross-validated | UCEC | BMI | 0.6060087 | 0.786400641 | 0.835353938 | NA | 0.827253434 | 0.6007587 | 0.913224588 | NA | NA | NA |
| Cross-validated | BRCA | BRCA | 0.667683543 | 0.906588714 | 0.959947003 | 0.688692375 | 0.945120566 | 0.844943573 | 0.926600572 | NA | NA | NA |
| Cross-validated | OV | BRCA | 0.802962963 | 0.896468254 | 0.898474427 | 0.754902998 | 0.894115961 | 0.785171958 | 0.816869489 | NA | NA | NA |
| Cross-validated | LIHC | HepB | 0.503712418 | 0.857490196 | 0.862457516 | NA | 0.861678468 | 0.673891068 | 0.828732026 | NA | NA | NA |
| Cross-validated | LIHC | HepC | 0.562916278 | 0.81443822 | 0.803243075 | NA | 0.793432929 | 0.663709928 | 0.852637722 | NA | NA | NA |
| Cross-validated | GBM | IDH | 0.745726179 | 0.946876349 | 0.954147394 | NA | 0.860157262 | 0.5 | 0.94011255 | NA | NA | NA |
| Cross-validated | LGG | IDH | 0.788231288 | 0.890183821 | 0.921110669 | NA | 0.890146844 | 0.622012063 | 0.97561849 | NA | NA | NA |
| Cross-validated | GBM | MGMT | 0.662820322 | 0.881578793 | 0.899104861 | NA | 0.897116866 | 0.797577895 | 0.974355023 | NA | NA | NA |
| Cross-validated | LGG | MGMT | 0.715685426 | 0.747132035 | 0.747132035 | NA | 0.746829004 | 0.746699134 | 0.76757215 | NA | NA | NA |
| Cross-validated | COAD | MSI | 0.977880342 | 0.969606838 | 0.980871795 | 0.963196581 | 0.964478632 | 0.964478632 | 0.981162393 | NA | NA | NA |
| Cross-validated | STAD | MSI | 0.976055724 | 0.999702311 | 0.987958435 | 0.998455603 | 0.999689908 | 0.99956438 | 0.989515873 | NA | NA | NA |
| Cross-validated | UCEC | MSI | 0.939369748 | 0.993235294 | 0.963046218 | 0.976951155 | 0.994243697 | 0.994243697 | 0.987731092 | NA | NA | NA |
| Cross-validated | STAD | POLD | 0.95082073 | 0.926432749 | 0.912988506 | NA | 0.926666667 | 0.960439605 | 0.962807018 | NA | NA | NA |
| Cross-validated | UCEC | POLD | 0.88922619 | 0.966666667 | 0.948571429 | NA | 0.966666667 | 0.9625 | 0.957916667 | NA | NA | NA |
| Cross-validated | BRCA | POLE | 0.469724969 | 0.903721093 | 0.886692027 | 0.634795392 | 0.88392337 | 0.698014508 | 0.924340435 | NA | NA | NA |
| Cross-validated | COAD | POLE | 0.837521368 | 1 | 1 | 0.733504274 | 1 | 1 | 1 | NA | NA | NA |
| Cross-validated | STAD | POLE | 0.929585098 | 0.999655172 | 0.99 | NA | 0.999655172 | 0.999655172 | 0.99 | NA | NA | NA |
| Cross-validated | UCEC | POLE | 0.762397959 | 0.973877551 | 0.982857143 | 0.736938776 | 0.973877551 | 0.973877551 | 0.991428571 | NA | NA | NA |
| Cross-validated | BLCA | SMOKING | 0.651931851 | 0.836043042 | 0.837337159 | 0.663608321 | 0.830949785 | 0.694619799 | 0.812570301 | NA | NA | NA |
| Cross-validated | CESC | SMOKING | 0.541655093 | 0.541795635 | 0.534373347 | NA | 0.502739749 | 0.515274471 | 0.68760582 | NA | NA | NA |
| Cross-validated | ESCAD | SMOKING | 0.586714286 | 0.942 | 0.928142857 | NA | 0.832 | 0.743714286 | 0.895428571 | NA | NA | NA |
| Cross-validated | ESCSQ | SMOKING | 0.463909091 | 0.827709091 | 0.83689697 | 0.533684848 | 0.802418182 | 0.550454545 | 0.805424242 | NA | NA | NA |
| Cross-validated | HNSCC | SMOKING | 0.753517425 | 0.857825236 | 0.891917798 | 0.73403354 | 0.889929522 | 0.786812932 | 0.915038462 | NA | NA | NA |
| Cross-validated | KIRP | SMOKING | 0.573621324 | 0.853284314 | 0.867757353 | 0.523443627 | 0.816182598 | 0.665098039 | 0.945343137 | NA | NA | NA |
| Cross-validated | LUAD | SMOKING | 0.862842504 | 0.889390277 | 0.957405951 | 0.884045453 | 0.952749275 | 0.908656973 | 0.947780731 | NA | NA | NA |
| Cross-validated | PAAD | SMOKING | 0.56522028 | 0.707107226 | 0.780668998 | NA | 0.802482517 | 0.653162005 | 0.939846154 | NA | NA | NA |
| Cross-validated | SKCM | UV* | 0.931461899 | 0.975431222 | 0.998454949 | 0.888488009 | 0.998811566 | 0.988281106 | 0.982681222 | NA | NA | NA |
| Cross-validated | Median | NA | 0.667683543 | 0.896468254 | 0.905714286 | 0.735486158 | 0.889929522 | 0.743714286 | 0.93047619 | NA | NA | NA |
| Cross-validated | Subset median | NA | 0.782680461 | 0.905154903 | 0.958676477 | 0.735486158 | 0.952304707 | 0.876800273 | 0.946561934 | NA | NA | NA |
| Cross-validated | Subset smoking | SMOKING | 0.651931851 | 0.853284314 | 0.867757353 | 0.663608321 | 0.830949785 | 0.694619799 | 0.915038462 | NA | NA | NA |
| | median | | | | | | | | | | | |
| Cross-validated | Overall smoking | SMOKING | 0.580167805 | 0.844663678 | 0.852547256 | 0.528564238 | 0.823566191 | 0.679858919 | 0.905233516 | NA | NA | NA |
| | median | | | | | | | | | | | |

Correlations Apparent

| type | tissue | factor | Unsupervised | Best_NMF | LDA | Logit | Matched_NMF | NNLS_Logit_betas | NNLS_Logit_m | RF |
|---|---|---|---|---|---|---|---|---|---|---|
| Apparent | ACC | AGE | NA | 0.18001398 | 0.397504213 | 0.397504213 | NA | 0.397504213 | 0.397504213 | 0.421178083 |
| Apparent | BLCA | AGE | 0.173713086 | 0.276320421 | 0.351519108 | 0.351519108 | 0.106792658 | 0.351519108 | 0.351519108 | 0.525579224 |
| Apparent | BRCA | AGE | 0.214659352 | 0.217571231 | 0.229039729 | 0.229084627 | 0.126274523 | 0.229084627 | 0.174249119 | 0.3136537 |
| Apparent | CESC | AGE | 0.17716557 | 0.444304373 | 0.459867679 | 0.579359606 | 0.444304373 | 0.579359606 | 0.341442014 | 0.499264273 |
| Apparent | CHOL | AGE | NA | 0.201062182 | 0.525013832 | 0.525013832 | NA | 0.525013832 | 0.525013832 | 0.508482114 |
| Apparent | COAD | AGE | 0.168611506 | 0.169959448 | 0.256983562 | 0.258470099 | 0.169959448 | 0.258470099 | 0.248665882 | 0.328203537 |
| Apparent | ESCAD | AGE | 0.161971855 | 0.229746452 | 0.233480099 | 0.233480099 | 0.08210146 | 0.233480099 | 0.129198908 | 0.297154963 |
| Apparent | ESCSQ | AGE | 0.094207536 | 0.198388635 | 0.242535209 | 0.242535209 | 0.198388635 | 0.242535209 | 0.242535209 | 0.285180095 |
| Apparent | GBM | AGE | 0.193673875 | 0.339778695 | 0.342304837 | 0.342304837 | 0.215720878 | 0.342304837 | 0.342304837 | 0.453678834 |
| Apparent | HNSCC | AGE | 0.325883242 | 0.375464615 | 0.529249368 | 0.530416768 | 0.224064138 | 0.530416768 | 0.450130187 | 0.598317397 |
| Apparent | KICH | AGE | 0.492162054 | 0.417461786 | 0.572313778 | 0.572313778 | 0.092743631 | 0.572313778 | 0.606730616 | 0.633980734 |
| Apparent | KIRC | AGE | 0.462923717 | 0.36897178 | 0.586169231 | 0.582922865 | 0.133401378 | 0.582922865 | 0.547038575 | 0.584072396 |
| Apparent | KIRP | AGE | 0.318716325 | 0.293825793 | 0.427270039 | 0.427270039 | 0.293825793 | 0.427270039 | 0.427270039 | 0.473425325 |
| Apparent | LAML | AGE | 0.253906351 | 0.372785424 | 0.38237786 | 0.38237786 | 0.372785424 | 0.38237786 | 0.38237786 | 0.390936891 |
| Apparent | LGG | AGE | 0.807428883 | 0.474381435 | 0.626458484 | 0.626458484 | 0.618836353 | 0.626458484 | 0.626458484 | 0.771930382 |
| Apparent | LIHC | AGE | 0.301583456 | 0.312306025 | 0.560325052 | 0.55309254 | 0.185346766 | 0.55309254 | 0.55704934 | 0.566359187 |
| Apparent | LUAD | AGE | −0.122528392 | 0.12165694 | 0.158201043 | 0.158201043 | 0.036106987 | 0.158201043 | 0.158201043 | 0.36718498 |
| Apparent | OV | AGE | 0.256023646 | 0.001523397 | 0.313109099 | 0.326955939 | 0.021773355 | 0.326955939 | 0.319285634 | 0.313810694 |
| Apparent | PAAD | AGE | 0.27639139 | 0.426077034 | 0.243414759 | 0.243414759 | 0.426077034 | 0.243414759 | 0.243414759 | 0.324038473 |
| Apparent | PCPG | AGE | 0.421590185 | 0.436951542 | 0.458246273 | 0.451848189 | 0.435492739 | 0.451848189 | 0.444186287 | 0.464484809 |
| Apparent | PRAD | AGE | 0.241827838 | 0.202868944 | 0.32129503 | 0.320699157 | 0.202868944 | 0.320699157 | 0.329505238 | 0.378918108 |
| Apparent | SARC | AGE | 0.553493701 | 0.445706484 | 0.580717638 | 0.591213017 | 0.538970776 | 0.591213017 | 0.513581972 | 0.587256609 |
| Apparent | SKCM | AGE | 0.024002067 | 0.186239156 | 0.154684745 | 0.154684745 | 0.186239156 | 0.154684745 | 0.154684745 | 0.239785551 |
| Apparent | STAD | AGE | 0.242864715 | 0.28433389 | 0.339563766 | 0.339563766 | 0.28433389 | 0.339563766 | 0.339563766 | 0.378991664 |
| Apparent | TGCT | AGE | 0.200845234 | 0.362002791 | 0.207645103 | 0.207645103 | 0.169910888 | 0.207645103 | 0.207645103 | 0.307269672 |
| Apparent | THCA | AGE | 0.454175461 | 0.249166513 | 0.446863176 | 0.446517424 | 0.249166513 | 0.446517424 | 0.444463232 | 0.510615723 |
| Apparent | THYM | AGE | 0.471443394 | 0.430521766 | 0.519229636 | 0.519229636 | 0.453529914 | 0.519229636 | 0.519229636 | 0.52864063 |
| Apparent | UCEC | AGE | 0.328999645 | 0.406319494 | 0.420285909 | 0.420285909 | −0.034570569 | 0.420285909 | 0.420285909 | 0.462910606 |
| Apparent | UCS | AGE | NA | 0.090219104 | 0.224089459 | 0.224089459 | NA | 0.224089459 | 0.224089459 | 0.379897307 |
| Apparent | UVM | AGE | NA | 0.32751797 | 0.27542575 | 0.27542575 | NA | 0.27542575 | 0.27542575 | 0.286661335 |
| Apparent | BLCA | SMOKING | 0.231136478 | 0.009237775 | 0.285317238 | 0.285814218 | 0.028366293 | 0.061987393 | 0.089100166 | 0.34359043 |
| Apparent | CESC | SMOKING | NA | 0.107989813 | 0.18995346 | 0.174973678 | NA | 0.143237732 | 0.114483394 | 0.472287368 |
| Apparent | ESCAD | SMOKING | NA | 0.144977779 | 0.704442308 | 0.682047294 | NA | 0.588772306 | 0.243172295 | 0.709255814 |
| Apparent | ESCSQ | SMOKING | −0.014737175 | 0.144980264 | 0.439260252 | 0.424643107 | −0.22861357 | 0.378074782 | 0.154979916 | 0.439537334 |
| Apparent | HNSCC | SMOKING | 0.526860117 | 0.230890881 | 0.551732852 | 0.637840373 | 0.248483992 | 0.549063218 | 0.342137614 | 0.671912912 |
| Apparent | KIRP | SMOKING | 0.11735761 | 0.211595184 | 0.595575223 | 0.607955377 | 0 | 0.570058634 | 0.167776469 | 0.745869121 |
| Apparent | LUAD | SMOKING | 0.325144457 | 0.217056785 | 0.453787202 | 0.497495039 | −0.254420675 | 0.452379819 | 0.198542893 | 0.499914399 |
| Apparent | PAAD | SMOKING | NA | 0.068284722 | 0.608687572 | 0.649249901 | NA | 0.665108377 | −0.256809857 | 0.795209869 |
| Age median subset | | | 0.254964999 | 0.303065909 | 0.366948484 | 0.366948484 | 0.200628789 | 0.366948484 | 0.346911973 | 0.437428458 |
| Smoking median subset | | | 0.231136478 | 0.144979021 | 0.502760027 | 0.552725208 | 0 | 0.500721518 | 0.161378193 | 0.585913655 |

Correlations Cross-Validated

| type | tissue | factor | Unsupervised | Best_NMF | LDA | Logit | Matched_NMF | NNLS_Logit_betas | NNLS_Logit_m | RF |
|---|---|---|---|---|---|---|---|---|---|---|
| Cross-validated | ACC | AGE | NA | 0.213990051 | 0.326422885 | 0.331689522 | NA | 0.331689522 | 0.359671095 | 0.32508247 |
| Cross-validated | BLCA | AGE | NA | 0.217841911 | 0.280706763 | 0.287901211 | NA | 0.287901211 | 0.256477881 | 0.355618622 |
| Cross-validated | BRCA | AGE | NA | 0.204576946 | 0.191791856 | 0.192882688 | NA | 0.192904312 | 0.152514576 | 0.269219011 |
| Cross-validated | CESC | AGE | NA | 0.324493867 | 0.383107053 | 0.429729536 | NA | 0.429729536 | 0.316513752 | 0.302395947 |
| Cross-validated | CHOL | AGE | NA | −0.118647509 | 0.569909517 | 0.577909517 | NA | 0.577909517 | 0.552195232 | 0.475236152 |
| Cross-validated | COAD | AGE | NA | 0.145533248 | 0.156665089 | 0.147630763 | NA | 0.147630763 | 0.173107655 | 0.214960136 |
| Cross-validated | ESCAD | AGE | NA | −0.126968671 | 0.048665601 | −0.007067749 | NA | −0.009821932 | 0.039523586 | −0.03803266 |
| Cross-validated | ESCSQ | AGE | NA | −0.004107435 | 0.090220892 | 0.091248872 | NA | 0.092354099 | 0.076572685 | −0.03878108 |
| Cross-validated | GBM | AGE | NA | 0.271461833 | 0.262963075 | 0.258141921 | NA | 0.258141921 | 0.278739141 | 0.366629611 |
| Cross-validated | HNSCC | AGE | NA | 0.359627439 | 0.420517918 | 0.387902872 | NA | 0.387902872 | 0.432455417 | 0.472530335 |
| Cross-validated | KICH | AGE | NA | 0.565550679 | 0.479169332 | 0.447906774 | NA | 0.447906774 | 0.528564202 | 0.493725984 |
| Cross-validated | KIRC | AGE | NA | 0.339329989 | 0.524595077 | 0.519172902 | NA | 0.519172902 | 0.515658057 | 0.471803063 |
| Cross-validated | KIRP | AGE | NA | 0.304551735 | 0.369347489 | 0.366607734 | NA | 0.366607734 | 0.37105491 | 0.367897251 |
| Cross-validated | LAML | AGE | NA | 0.047813583 | 0.361063587 | 0.361279208 | NA | 0.361279208 | 0.36394947 | 0.2836549 |
| Cross-validated | LGG | AGE | NA | 0.39038318 | 0.576555642 | 0.515065868 | NA | 0.515065868 | 0.548039718 | 0.642875851 |
| Cross-validated | LIHC | AGE | NA | 0.292714529 | 0.55236605 | 0.5334195 | NA | 0.535546495 | 0.56753603 | 0.455608563 |
| Cross-validated | LUAD | AGE | NA | −0.13718159 | −0.152987595 | −0.143594013 | NA | −0.139278602 | −0.137314525 | −0.15611342 |
| Cross-validated | OV | AGE | NA | −0.023115709 | 0.209788508 | 0.198889129 | NA | 0.199024513 | 0.188326593 | 0.165074626 |
| Cross-validated | PAAD | AGE | NA | 0.191237904 | 0.271304548 | 0.266980048 | NA | 0.266980048 | 0.273837191 | 0.259538471 |
| Cross-validated | PCPG | AGE | NA | 0.369146696 | 0.36712326 | 0.37042699 | NA | 0.37042699 | 0.42449412 | 0.41449093 |
| Cross-validated | PRAD | AGE | NA | 0.155097229 | 0.250199782 | 0.25077874 | NA | 0.25077874 | 0.308174728 | 0.271818817 |
| Cross-validated | SARC | AGE | NA | 0.43153039 | 0.540257189 | 0.530371302 | NA | 0.530371302 | 0.484522667 | 0.494929553 |
| Cross-validated | SKCM | AGE | NA | 0.171972771 | 0.102862581 | 0.113271055 | NA | 0.113271055 | 0.119166414 | 0.171179996 |
| Cross-validated | STAD | AGE | NA | 0.21892295 | 0.319248611 | 0.321113647 | NA | 0.320891379 | 0.302202991 | 0.310365929 |
| Cross-validated | TGCT | AGE | NA | 0.300479154 | 0.124907292 | 0.127336965 | NA | 0.125514269 | 0.111603352 | 0.119898937 |
| Cross-validated | THCA | AGE | NA | 0.282342178 | 0.398826664 | 0.40591695 | NA | 0.40591695 | 0.43157614 | 0.4508313 |
| Cross-validated | THYM | AGE | NA | 0.444529332 | 0.492402851 | 0.460339987 | NA | 0.460339987 | 0.431637104 | 0.50358831 |
| Cross-validated | UCEC | AGE | NA | 0.277670548 | 0.259172958 | 0.33333666 | NA | 0.33333666 | 0.31733666 | 0.143814741 |
| Cross-validated | UCS | AGE | NA | −0.033127655 | 0.037154417 | −0.022028102 | NA | −0.022028102 | 0.068496186 | 0.01085272 |
| Cross-validated | UVM | AGE | NA | 0.126016821 | 0.212889284 | 0.212889284 | NA | 0.212889284 | 0.212889284 | 0.188065936 |
| Cross-validated | BLCA | SMOKING | NA | 0.096972165 | 0.454395502 | 0.455238833 | NA | 0.364790876 | 0.067754334 | 0.441650111 |
| Cross-validated | CESC | SMOKING | NA | 0.195318928 | 0.05291906 | 0.030295421 | NA | −0.016736684 | −0.027519145 | 0.286177104 |
| Cross-validated | ESCAD | SMOKING | NA | −0.033776498 | 0.66985837 | 0.649212483 | NA | 0.541835441 | 0.328981552 | 0.541690379 |
| Cross-validated | ESCSQ | SMOKING | NA | 0.192021966 | 0.494496164 | 0.423306039 | NA | 0.377501857 | 0.057732325 | 0.418778911 |
| Cross-validated | HNSCC | SMOKING | NA | −0.039380234 | 0.528928998 | 0.607637859 | NA | 0.510269397 | 0.325424547 | 0.643917893 |
| Cross-validated | KIRP | SMOKING | NA | −0.280857649 | 0.555626291 | 0.6089357 | NA | 0.609017617 | 0.137300264 | 0.690116105 |
| Cross-validated | LUAD | SMOKING | NA | −0.073180806 | 0.425653494 | 0.483880165 | NA | 0.393586738 | 0.193566026 | 0.490451266 |
| Cross-validated | PAAD | SMOKING | NA | 0.260985507 | 0.374974806 | 0.490543073 | NA | 0.551995497 | 0.033086262 | 0.73167609 |
| Age median subset | | | NA | 0.218382431 | 0.299977687 | 0.326401585 | NA | 0.326290451 | 0.31234424 | 0.306380938 |
| Smoking median subset | | | NA | 0.031597833 | 0.474445833 | 0.487211619 | NA | 0.451928067 | 0.102527299 | 0.516070822 |

The “Subset median” AUC is the median AUC calculated only over the tissues where Alexandrov et al. found an age signature.
To calculate the “Overall median” AUC, whenever Alexandrov et al. methodology was not able to detect the age signature in a tissue, and therefore its intensities were not provided (NA), a 0.5 AUC was assigned to that signature for that tissue for their methodology.
The “Subset median” AUC is the median AUC calculated only over the tissues where Alexandrov et al. found a signature for the given exposure.
The “Subset smoking median” was instead calculated by restricting the set of tissues to those where Alexandrov et al. detecetd smoking signatures.
To calculate the “Overall smoking median” AUC, whenever Alexandrov et al. methodology was not able to detect a smoking signature in a tissue, and therefore its intensities were not provided (NA), a 0.5 AUC was assigned for their methodology to the smoking signature for that tissue.

TABLE 10
Estimated contributions of the age signature in different tissue types. For each tissue type and for each
etiological factor the estimated mean and median contribution of that factor, out of the total number of
mutations present in that tissue, are reported together with the sample sizes (number of patients analyzed).

| | | Mean | Median |
|---|---|---|---|
| Tissue | Exposure | (Explained by Age) | (Explained by Age) |
| Uterine Corpus Endometrial Carcinoma | POLe Mutation | 0.045755922 | 0.023948199 |
| Colorectal Adenocarcinoma | POLe Mutation | 0.052761356 | 0.03684821 |
| Skin Cutaneous Melanoma | UV* | 0.105800021 | 0.081241722 |
| Uterine Corpus Endometrial Carcinoma | POLD Mutation | 0.112400896 | 0.118262467 |
| Stomach Adenocarcinoma | POLD Mutation | 0.116846045 | 0.09198678 |
| Stomach Adenocarcinoma | POLe Mutation | 0.122890331 | 0.096980256 |
| Uterine Corpus Endometrial Carcinoma | Microsatellite Instability | 0.139959289 | 0.125852051 |
| Colorectal Adenocarcinoma | Microsatellite Instability | 0.142197056 | 0.115702479 |
| Stomach Adenocarcinoma | Microsatellite Instability | 0.146206016 | 0.129836552 |
| Bladder Urothelial Carcinoma | Aristolochic Acid | 0.24013558 | 0.180882353 |
| Lung Adenocarcinoma | Smoking | 0.281117772 | 0.173853606 |
| Breast Invasive Carcinoma | BRCA1/2 Mutation | 0.34418737 | 0.248477617 |
| Head and Neck | Smoking | 0.516830074 | 0.504766773 |
| Mesothelioma | Asbestos* | 0.536384961 | 0.548318958 |
| Breast Invasive Carcinoma | POLe Mutation | 0.540860474 | 0.628826531 |
| Ovarian Serous Cystadenocarcinoma | BRCA1/2 Mutation | 0.555360933 | 0.505248619 |
| Cervical Squamous | Smoking | 0.640003082 | 0.719166667 |
| Cervical Squamous | High Apobec | 0.647027165 | 0.694075587 |
| Bladder Urothelial Carcinoma | Smoking | 0.664568082 | 0.718397997 |
| Renal Papillary Cell Carcinoma | Obesity | 0.667675247 | 0.763044201 |
| Head and Neck | Unexposed | 0.698318485 | 0.720680958 |
| Acute Myeloid Leukemia | Unexposed | 0.715471131 | 0.692307692 |
| Brain Lower Grade Glioma | MGMT Methylated | 0.716964067 | 0.714891362 |
| Renal Papillary Cell Carcinoma | Smoking | 0.720564429 | 0.787649925 |
| Cervical Squamous | Unexposed | 0.727532815 | 0.779781421 |
| Liver Hepatocellular Carcinoma | Hepatitis C | 0.730204239 | 0.765863169 |
| Liver Hepatocellular Carcinoma | Hepatitis B | 0.743337793 | 0.759640341 |
| Skin Cutaneous Melanoma | Unexposed | 0.74546021 | 0.748834978 |
| Uterine Corpus Endometrial Carcinoma | Unexposed | 0.747868514 | 0.874960636 |
| Liver Hepatocellular Carcinoma | Alcohol | 0.752404868 | 0.822341272 |
| Glioblastoma Multiforme | MGMT Methylated | 0.756618145 | 0.772791024 |
| Thyroid Carcinoma | Unexposed | 0.759585525 | 0.7875 |
| Breast Invasive Carcinoma | Unexposed | 0.763898284 | 0.841836735 |
| Bladder Urothelial Carcinoma | Unexposed | 0.775417488 | 0.905844156 |
| Renal Clear Cell Carcinoma | High Apobec | 0.78022672 | 0.771243895 |
| Adrenocortical Carcinoma | Unexposed | 0.781765033 | 0.879538939 |
| Prostate Adenocarcinoma | Unexposed | 0.782512287 | 0.795698925 |
| Kidney Chromophobe | Unexposed | 0.786042629 | 0.749433107 |
| Colorectal Adenocarcinoma | Obesity | 0.787309401 | 0.88578149 |
| Lung Adenocarcinoma | Unexposed | 0.788563582 | 0.87247755 |
| Esophagus Squamous | Smoking | 0.793385856 | 0.89899506 |
| Stomach Adenocarcinoma | Unexposed | 0.794451019 | 0.925126727 |
| Ovarian Serous Cystadenocarcinoma | Unexposed | 0.794763528 | 0.917156863 |
| Sarcoma | Unexposed | 0.803955569 | 0.849206349 |
| Thymoma | Unexposed | 0.806541749 | 0.855555556 |
| Pancreatic Adenocarcinoma | Smoking | 0.811928213 | 0.897142857 |
| Head and Neck | Alcohol | 0.818666553 | 0.876994681 |
| Esophageal Carcinoma | Alcohol | 0.820074891 | 0.842341734 |
| Esophagus Adenocarcinoma | Smoking | 0.82380059 | 0.844056318 |
| Pheochromocytoma and Paraganglioma | Unexposed | 0.825504094 | 0.869565217 |
| Pancreatic Adenocarcinoma | Unexposed | 0.827174344 | 0.879973475 |
| Esophagus Squamous | Unexposed | 0.827183106 | 0.953233284 |
| Colorectal Adenocarcinoma | Unexposed | 0.829086944 | 0.895517677 |
| Testicular Germ Cell Tumors | Unexposed | 0.829642612 | 0.89516129 |
| Liver Hepatocellular Carcinoma | Unexposed | 0.829914796 | 0.928167003 |
| Brain Lower Grade Glioma | IDH Methylated | 0.830532648 | 0.867948718 |
| Glioblastoma Multiforme | Unexposed | 0.830640972 | 0.897726719 |
| Renal Clear Cell Carcinoma | Unexposed | 0.83880815 | 0.873773417 |
| Uterine Corpus Endometrial Carcinoma | Obesity | 0.839465015 | 0.990582192 |
| Esophagus Adenocarcinoma | Unexposed | 0.844070855 | 0.935151515 |
| Esophageal Carcinoma | Obesity | 0.848752763 | 0.985785632 |
| Brain Lower Grade Glioma | Unexposed | 0.849959196 | 0.899068323 |
| Renal Papillary Cell Carcinoma | Unexposed | 0.850206311 | 0.914583333 |
| Uveal Melanoma | Unexposed | 0.853972571 | 0.895833333 |
| Cholangiocarcinoma | Unexposed | 0.85467562 | 0.854166667 |
| Uterine Carcinosarcoma | Unexposed | 0.859688079 | 0.910860838 |
| Glioblastoma Multiforme | IDH Methylated | 0.921260827 | 1 |
| | Mean | 0.658763286 | 0.698335671 |
| | Median | 0.775417488 | 0.822341272 |

TABLE 11
Comparisons of prediction accuracy (AUC) with different mislabeled proportions (5%, 10%, 20%, and 25% of samples mislabeled) in the training set. The AUCs, both apparent and cross-validated
(CV), are reported for age and all other etiological factors across all tissue types for each one of the mutational signature methodologies considered in this study: Logistic Regression (Logit), Linear Discriminant
Analysis (LDA), Non-negative Least Square Logit using the Betas (NNLS_Logit_betas), Non-negative Least Square Logit using the means (NNLS_Logit_means), Random Forest (RF), Unsupervised
as in Alexandrov et al. (Unsupervised), Best_NMF, Matched_NMF, Signature 1 as in Alexandrov et al. (Signature1), and Single Peak (SinglePeak).
Age Apparent (5%)

| type | tissue | factor | Best_NMF | LDA | Logit | Matched_NMF | NNLS_Logit_betas | NNLS_Logit_means | RF | Sig0.5ture1 | SinglePeak | Unsupervised |
|---|---|---|---|---|---|---|---|---|---|---|---|---|
| Apparent | ACC | AGE | 0.59826087 | 0.80347826 | 0.80173913 | NA | 0.80173913 | 0.789565217 | 0.768695652 | 0.471304348 | 0.74 | NA |
| Apparent | BLCA | AGE | 0.634566327 | 0.72810374 | 0.7272534 | 0.488095238 | 0.727253401 | 0.661777211 | 0.77912415 | 0.654336735 | 0.62478741 | 0.654336735 |
| Apparent | BRCA | AGE | 0.62109108 | 0.62351832 | 0.62349476 | 0.581182986 | 0.623494757 | 0.59191705 | 0.672734771 | 0.555190291 | 0.56808059 | 0.60466596 |
| Apparent | CESC | AGE | 0.721447028 | 0.77674419 | 0.83617571 | 0.721447028 | 0.836175711 | 0.719896641 | 0.756072351 | 0.56873385 | 0.61472868 | 0.56873385 |
| Apparent | CHOL | AGE | 0.49112426 | 0.76627219 | 0.76627219 | NA | 0.766272189 | 0.766272189 | 0.784023669 | 0.553254438 | 0.62721893 | NA |
| Apparent | COAD | AGE | 0.590010406 | 0.67416753 | 0.67416753 | 0.597034339 | 0.674167534 | 0.663241415 | 0.728798127 | 0.590530697 | 0.68405307 | 0.590530697 |
| Apparent | ESCAD | AGE | 0.601108033 | 0.6800554 | 0.68282548 | 0.592797784 | 0.682825485 | 0.610803324 | 0.713296399 | 0.573407202 | 0.5166205 | 0.573407202 |
| Apparent | ESCSQ | AGE | 0.585470085 | 0.61965812 | 0.61965812 | 0.565527066 | 0.61965812 | 0.61965812 | 0.726495726 | 0.575498575 | 0.48717949 | 0.575498575 |
| Apparent | GBM | AGE | 0.677777778 | 0.71560847 | 0.71507937 | 0.627513228 | 0.715079365 | 0.705555556 | 0.738095238 | 0.612301587 | 0.68267196 | 0.612301587 |
| Apparent | HNSCC | AGE | 0.714365101 | 0.80494582 | 0.80494582 | 0.613086969 | 0.804945818 | 0.742706307 | 0.80869686 | 0.671158655 | 0.74576271 | 0.671158655 |
| Apparent | KICH | AGE | 0.828719723 | 0.83217993 | 0.83217993 | 0.541522491 | 0.832179931 | 0.832179931 | 0.873702422 | 0.709342561 | 0.85813149 | 0.761245675 |
| Apparent | KIRC | AGE | 0.657838983 | 0.81091102 | 0.81064619 | 0.576403602 | 0.810646186 | 0.762182203 | 0.800185381 | 0.551112288 | 0.7717161 | 0.724311441 |
| Apparent | KIRP | AGE | 0.688034188 | 0.76495726 | 0.76068376 | 0.688034188 | 0.760683761 | 0.746438746 | 0.811965812 | 0.494301994 | 0.71794872 | 0.705128205 |
| Apparent | LAML | AGE | 0.706597222 | 0.68315972 | 0.68315972 | 0.706597222 | 0.683159722 | 0.683159722 | 0.716145833 | 0.585069444 | 0.61545139 | 0.635416667 |
| Apparent | LGG | AGE | 0.766666667 | 0.88333333 | 0.88333333 | 0.85 | 0.883333333 | 0.883333333 | 0.881481481 | 0.792592593 | 0.87777778 | 0.944444444 |
| Apparent | UHC | AGE | 0.573275862 | 0.7567734 | 0.74692118 | 0.525862069 | 0.746921182 | 0.746921182 | 0.702586207 | 0.549261084 | 0.67426108 | 0.674876847 |
| Apparent | LUAD | AGE | 0.614197531 | 0.64351852 | 0.64351852 | 0.520061728 | 0.643518519 | 0.643518519 | 0.768518519 | 0.456790123 | 0.57407407 | 0.456790123 |
| Apparent | OV | AGE | 0.526511135 | 0.69379639 | 0.69379639 | 0.516967126 | 0.693796394 | 0.693796394 | 0.693796394 | 0.671792153 | 0.54003181 | 0.671792153 |
| Apparent | PAAD | AGE | 0.533333333 | 0.63684211 | 0.63684211 | 0.578947368 | 0.636842105 | 0.636842105 | 0.654385965 | 0.638596491 | 0.53333333 | 0.638596491 |
| Apparent | PCPG | AGE | 0.704294218 | 0.77104592 | 0.7684949 | 0.742772109 | 0.768494898 | 0.762117347 | 0.775935374 | 0.523384354 | 0.77827381 | 0.753401361 |
| Apparent | PRAD | AGE | 0.607053763 | 0.6852043 | 0.68733333 | 0.607053763 | 0.687333333 | 0.666989247 | 0.706860215 | 0.560451613 | 0.69178495 | 0.608924731 |
| Apparent | SARC | AGE | 0.749188897 | 0.81795242 | 0.78704037 | 0.798485941 | 0.787040375 | 0.787040375 | 0.80127974 | 0.692682048 | 0.79397981 | 0.805875991 |
| Apparent | SKCM | AGE | 0.624628198 | 0.62135634 | 0.62135634 | 0.489886972 | 0.621356336 | 0.621356336 | 0.679357525 | 0.483045806 | 0.53390839 | 0.483045806 |
| Apparent | STAD | AGE | 0.606119951 | 0.66119951 | 0.66119951 | 0.591921665 | 0.66119951 | 0.66119951 | 0.692839657 | 0.6000612 | 0.59461444 | 0.6000612 |
| Apparent | TGCT | AGE | 0.692763158 | 0.60164474 | 0.60164474 | 0.601973684 | 0.601644737 | 0.601644737 | 0.619407895 | 0.432894737 | 0.6 | 0.613157895 |
| Apparent | THCA | AGE | 0.664844509 | 0.77815841 | 0.77810982 | 0.664844509 | 0.778109815 | 0.777380952 | 0.814552964 | 0.518148688 | 0.74531098 | 0.774514091 |
| Apparent | THYM | AGE | 0.727650728 | 0.76923077 | 0.75502426 | 0.684684685 | 0.755024255 | 0.755024255 | 0.761607762 | 0.595980596 | 0.71067221 | 0.718641719 |
| Apparent | UCEC | AGE | 0.785123967 | 0.65289256 | 0.79752066 | 0.454545455 | 0.797520661 | 0.747933884 | 0.859504132 | 0.661157025 | 0.5785124 | 0.561983471 |
| Apparent | UCS | AGE | 0.633986928 | 0.57026144 | 0.57026144 | NA | 0.570261438 | 0.570261438 | 0.668300654 | 0.633986928 | 0.60947712 | NA |
| Apparent | UVM | AGE | 0.6775 | 0.69375 | 0.69375 | NA | 0.69375 | 0.69375 | 0.71875 | 0.29 | 0.58625 | NA |
| Apparent | Median | AGE | 0.646202655 | 0.70470243 | 0.72116638 | 0.594916062 | 0.721166383 | 0.699675975 | 0.747083795 | 0.574452889 | 0.62600317 | 0.637006579 |
| Apparent | Subset smoking | AGE | 0.661341746 | 0.70470243 | 0.72116638 | 0.594916062 | 0.721166383 | 0.699675975 | 0.747083795 | 0.58028401 | 0.64952425 | 0.637006579 |
| | median | | | | | | | | | | | |
| Apparent | Overall smoking | AGE | 0.646202655 | 0.70470243 | 0.72116638 | 0.586552325 | 0.721166383 | 0.699675975 | 0.747083795 | 0.574452889 | 0.62600317 | 0.612729741 |
| | median | | | | | | | | | | | |

Other Exposures Apparent (5%)

| type | tissue | factor | Best_NMF | LDA | Logit | Matched_NMF | NNLS_Logit_betas | NNLS_Logit_means | RF | Signature1 | SinglePeak | Unsupervised |
|---|---|---|---|---|---|---|---|---|---|---|---|---|
| Apparent | BLCA | AAcid | 0.890092879 | 0.95356037 | 1 | 0.890092879 | 1 | 1 | 1 | NA | NA | 0.964396285 |
| Apparent | ESCA | ALCOHOL | 0.773148148 | 0.9537037 | 0.94444444 | NA | 0.962962963 | 0.888888889 | 0.888888889 | NA | NA | NA |
| Apparent | HNSCC | ALCOHOL | 0.596774194 | 0.94239631 | 0.91935484 | NA | 0.535714286 | 0.535714286 | 0.950460829 | NA | NA | NA |
| Apparent | UHC | ALCOHOL | 0.613119835 | 0.91873278 | 0.92889118 | NA | 0.917527548 | 0.612086777 | 0.858126722 | NA | NA | NA |
| Apparent | CESC | APOBEC | 0.676772247 | 0.92850679 | 0.9546003 | 0.608144796 | 0.940120664 | 0.65520362 | 0.929864253 | NA | NA | 0.638612368 |
| Apparent | KIRC | APOBEC | 0.582369942 | 0.79190751 | 0.80419075 | NA | 0.812379576 | 0.627408478 | 0.924253372 | NA | NA | NA |
| Apparent | MESO | Asb* | 0.9375 | 0.94375 | 0.94375 | NA | 0.94375 | 0.94375 | 0.980681818 | NA | NA | NA |
| Apparent | COAD | BMI | 0.569592333 | 0.75973532 | 0.75045634 | NA | 0.739808336 | 0.60602373 | 0.888271981 | NA | NA | NA |
| Apparent | ESCA | BMI | 0.685960591 | 0.97413793 | 0.97475369 | NA | 0.96182266 | 0.52955665 | 0.919334975 | NA | NA | NA |
| Apparent | KIRP | BMI | 0.643548387 | 0.88145161 | 0.88467742 | NA | 0.875806452 | 0.838709677 | 0.953225806 | NA | NA | NA |
| Apparent | UCEC | BMI | 0.596869712 | 0.83432036 | 0.85279188 | NA | 0.846869712 | 0.5 | 0.944091935 | NA | NA | NA |
| Apparent | BRCA | BRCA | 0.706731177 | 0.92906324 | 0.9433866 | 0.732068585 | 0.942026522 | 0.852388643 | 0.96850561 | NA | NA | 0.67027417 |
| Apparent | OV | BRCA | 0.812738368 | 0.86498856 | 0.83524027 | 0.662852784 | 0.832951945 | 0.790236461 | 0.83409611 | NA | NA | 0.809687262 |
| Apparent | LIHC | HepB | 0.587575758 | 0.88636364 | 0.89 | NA | 0.89030303 | 0.699393939 | 0.874393939 | NA | NA | NA |
| Apparent | LIHC | HepC | 0.626282991 | 0.90065982 | 0.8914956 | NA | 0.846041056 | 0.677419355 | 0.954728739 | NA | NA | NA |
| Apparent | GBM | IDH | 0.714860515 | 0.91604077 | 0.91845494 | NA | 0.830874464 | 0.502145923 | 0.934683476 | NA | NA | NA |
| Apparent | LGG | IDH | 0.792294692 | 0.91197875 | 0.9210844 | NA | 0.866795433 | 0.516193564 | 0.955006381 | NA | NA | NA |
| Apparent | GBM | MGMT | 0.656996983 | 0.89030711 | 0.90237487 | NA | 0.902839019 | 0.837239886 | 0.939506459 | NA | NA | NA |
| Apparent | LGG | MGMT | 0.693452381 | 0.74891775 | 0.74891775 | NA | 0.748917749 | 0.748917749 | 0.761634199 | NA | NA | NA |
| Apparent | COAD | MSI | 0.912820513 | 0.99164292 | 0.98119658 | 0.968850902 | 0.981196581 | 0.981196581 | 0.991642925 | NA | NA | 0.967046534 |
| Apparent | STAD | MSI | 0.926793981 | 0.99962803 | 0.9857908 | 0.99963831 | NA | 0.997545008 | 0.998958488 | NA | NA | 0.999855324 |
| Apparent | UCEC | MSI | 0.933035714 | 0.99834656 | 0.99801587 | 0.97172619 | 0.998015873 | 0.998015873 | 1 | NA | NA | 1 |
| Apparent | STAD | POLD | 0.985042735 | 0.99973104 | 1 | NA | 1 | 0.997310382 | 1 | NA | NA | NA |
| Apparent | UCEC | POLD | 0.9375 | 0.99801587 | 0.99801587 | NA | 0.998015873 | 0.998015873 | 1 | NA | NA | NA |
| Apparent | BRCA | POLE | 0.66942689 | 0.81047463 | 0.82160393 | 0.586402266 | 0.821603928 | 0.722094926 | 0.890943808 | NA | NA | 0.423294835 |
| Apparent | COAD | POLE | 0.937660256 | 0.9775641 | 1 | 0.629807692 | 1 | 1 | 1 | NA | NA | 0.72275641 |
| Apparent | STAD | POLE | 0.945815058 | 0.97000368 | 0.94221568 | NA | NA | 0.999631947 | 0.998619801 | NA | NA | NA |
| Apparent | UCEC | POLE | 0.819047619 | 1 | 1 | 0.762698413 | 1 | 1 | 1 | NA | NA | 0.734126984 |
| Apparent | BLCA | SMOKING | 0.671283686 | 0.88953926 | 0.89901478 | 0.671283686 | 0.888554042 | 0.710402782 | 0.865807012 | NA | 0.64022023 | 0.683917705 |
| Apparent | CESC | SMOKING | 0.559580292 | 0.65264599 | 0.5959854 | NA | 0.543886861 | 0.587226277 | 0.757572993 | NA | 0.42810219 | NA |
| Apparent | ESCAD | SMOKING | 0.577639752 | 0.93540373 | 0.93664596 | NA | 0.950310559 | 0.737888199 | 0.913043478 | NA | 0.5826087 | NA |
| Apparent | ESCSQ | SMOKING | 0.56798959 | 0.83734548 | 0.82888744 | 0.453480807 | 0.803838647 | 0.575146389 | 0.833767079 | NA | 0.52635003 | 0.470071568 |
| Apparent | HNSCC | SMOKING | 0.750847868 | 0.86220123 | 0.87156815 | 0.755571705 | 0.874172319 | 0.779069767 | 0.910287468 | NA | 0.69533269 | 0.818213017 |
| Apparent | KIRP | SMOKING | 0.51264881 | 0.88020833 | 0.89583333 | 0.51264881 | 0.880580357 | 0.694940476 | 0.9609375 | NA | 0.60825893 | 0.625744048 |
| Apparent | LUAD | SMOKING | 0.845985173 | 0.88036304 | 0.93316832 | 0.883157565 | 0.900400754 | 0.910124941 | 0.948343942 | NA | 0.90980996 | 0.910619192 |
| Apparent | PAAD | SMOKING | 0.595192916 | 0.77925364 | 0.78810879 | NA | 0.854364326 | 0.549019608 | 0.924256799 | NA | 0.54854522 | NA |
| Apparent | SKCM | UV* | 0.922796441 | 0.94217172 | 0.95075758 | 0.888764646 | 0.95260101 | 0.972828283 | 0.963358586 | NA | NA | 0.949632943 |
| Apparent | Median | NA | 0.693452381 | 0.91604077 | 0.9210844 | 0.743820145 | 0.89030303 | 0.748917749 | 0.944091935 | NA | 0.59543381 | 0.771907123 |
| Apparent | Subset median | NA | 0.815892993 | 0.92878502 | 0.94707209 | 0.743820145 | 0.940120664 | 0.881256792 | 0.962148043 | NA | NA | 0.771907123 |
| Apparent | Subset smoking | SMOKING | 0.671283686 | 0.88020833 | 0.89583333 | 0.671283686 | 0.880580357 | 0.710402782 | 0.910287468 | NA | 0.64022023 | 0.683917705 |
| | median | | | | | | | | | | | |
| Apparent | Overall smoking | SMOKING | 0.586416334 | 0.87120478 | 0.88370074 | 0.506324405 | 0.877376338 | 0.702671629 | 0.911665473 | NA | 0.56730448 | 0.562872024 |
| | median | | | | | | | | | | | |

Age CV (5%)

| type | tissue | factor | Best_NMF | LDA | Logit | Matched_NMF | NNLS_Logit_betas | NNLS_Logit_means | RF | Signature1 | SinglePeak | Unsupervised |
|---|---|---|---|---|---|---|---|---|---|---|---|---|
| Cross-validated | ACC | AGE | 0.620966667 | 0.69406667 | 0.66246667 | NA | 0.661666667 | 0.7092 | 0.691833333 | NA | NA | NA |
| Cross-validated | BLCA | AGE | 0.617166811 | 0.71107092 | 0.72001032 | 0.481520779 | 0.720010317 | 0.708697186 | 0.730104113 | NA | NA | NA |
| Cross-validated | BRCA | AGE | 0.588515941 | 0.60245916 | 0.60319482 | 0.568264463 | 0.603194819 | 0.572849014 | 0.623053852 | NA | NA | NA |
| Cross-validated | CESC | AGE | 0.566670034 | 0.64217597 | 0.68817208 | 0.685258498 | 0.688172078 | 0.665546697 | 0.63831718 | NA | NA | NA |
| Cross-validated | CHOL | AGE | 0.476111111 | 0.77388889 | 0.77555556 | NA | 0.775555556 | 0.770555556 | 0.755555556 | NA | NA | NA |
| Cross-validated | COAD | AGE | 0.58820833 | 0.57815942 | 0.57478395 | 0.644243752 | 0.574783951 | 0.578253599 | 0.614713556 | NA | NA | NA |
| Cross-validated | ESCAD | AGE | 0.502 | 0.50466667 | 0.5 | 0.485666667 | 0.501666667 | 0.469 | 0.480333333 | NA | NA | NA |
| Cross-validated | ESCSQ | AGE | 0.531566667 | 0.52929048 | 0.52595714 | 0.565328571 | 0.525957143 | 0.507671429 | 0.493566667 | NA | NA | NA |
| Cross-validated | GBM | AGE | 0.61886731 | 0.66864333 | 0.66839905 | 0.61838224 | 0.668399045 | 0.670024753 | 0.693693445 | NA | NA | NA |
| Cross-validated | HNSCC | AGE | 0.728746392 | 0.68981699 | 0.68607351 | 0.63665737 | 0.68607351 | 0.683892635 | 0.717243173 | NA | NA | NA |
| Cross-validated | KICH | AGE | 0.838055556 | 0.65330556 | 0.65597222 | 0.608777778 | 0.655972222 | 0.703972222 | 0.715222222 | NA | NA | NA |
| Cross-validated | KIRC | AGE | 0.678399464 | 0.77688587 | 0.78030502 | 0.649175431 | 0.780305024 | 0.755497776 | 0.74695487 | NA | NA | NA |
| Cross-validated | KIRP | AGE | 0.728171429 | 0.7376381 | 0.73220952 | 0.728171429 | 0.732209524 | 0.740209524 | 0.734557143 | NA | NA | NA |
| Cross-validated | LAML | AGE | 0.5496 | 0.63563333 | 0.6353 | 0.5398 | 0.6353 | 0.666633333 | 0.551422222 | NA | NA | NA |
| Cross-validated | LGG | AGE | 0.722222222 | 0.85355556 | 0.84927778 | 0.834444444 | 0.849277778 | 0.825944444 | 0.8735 | NA | NA | NA |
| Cross-validated | UHC | AGE | 0.630956349 | 0.72851587 | 0.71864286 | 0.593833333 | 0.718642857 | 0.727880952 | 0.698007937 | NA | NA | NA |
| Cross-validated | LUAD | AGE | 0.419833333 | 0.45125 | 0.46308333 | 0.493333333 | 0.463083333 | 0.473083333 | 0.448083333 | NA | NA | NA |
| Cross-validated | OV | AGE | 0.456775794 | 0.62730467 | 0.62891578 | 0.495630952 | 0.628915785 | 0.619681217 | 0.621493827 | NA | NA | NA |
| Cross-validated | PAAD | AGE | 0.434138889 | 0.65838889 | 0.65672222 | 0.692305556 | 0.656722222 | 0.656722222 | 0.579805556 | NA | NA | NA |
| Cross-validated | PCPG | AGE | 0.656677778 | 0.72591237 | 0.72992904 | 0.729182828 | 0.72992904 | 0.745218939 | 0.741863889 | NA | NA | NA |
| Cross-validated | PRAD | AGE | 0.603191105 | 0.63751969 | 0.63574781 | 0.614716929 | 0.63574781 | 0.64823152 | 0.63824839 | NA | NA | NA |
| Cross-validated | SARC | AGE | 0.758524492 | 0.79250114 | 0.79140749 | 0.800618311 | 0.791407491 | 0.782575623 | 0.782031915 | NA | NA | NA |
| Cross-validated | SKCM | AGE | 0.638429123 | 0.60483311 | 0.58517262 | 0.445660935 | 0.594061508 | 0.607394841 | 0.620195216 | NA | NA | NA |
| Cross-validated | STAD | AGE | 0.578768782 | 0.64661173 | 0.64636173 | 0.596742936 | 0.647611731 | 0.655857102 | 0.65041323 | NA | NA | NA |
| Cross-validated | TGCT | AGE | 0.65756045 | 0.55216647 | 0.55216647 | 0.62656455 | 0.552166468 | 0.552166468 | 0.549913161 | NA | NA | NA |
| Cross-validated | THCA | AGE | 0.67245814 | 0.74836015 | 0.74783178 | 0.696432674 | 0.747831777 | 0.759577619 | 0.771587828 | NA | NA | NA |
| Cross-validated | THYM | AGE | 0.732011338 | 0.73762972 | 0.73930773 | 0.671088341 | 0.738593443 | 0.721929705 | 0.754272628 | NA | NA | NA |
| Cross-validated | UCEC | AGE | 0.628333333 | 0.66833333 | 0.67333333 | 0.348333333 | 0.67 | 0.65 | 0.625 | NA | NA | NA |
| Cross-validated | UCS | AGE | 0.483666667 | 0.42480556 | 0.41647222 | NA | 0.419805556 | 0.397805556 | 0.445305556 | NA | NA | NA |
| Cross-validated | UVM | AGE | 0.646305556 | 0.67861111 | 0.67861111 | NA | 0.678611111 | 0.678611111 | 0.631777778 | NA | NA | NA |
| Cross-validated | Median | AGE | 0.619916989 | 0.66336111 | 0.66543286 | 0.616549585 | 0.665032856 | 0.668329043 | 0.644365205 | NA | NA | NA |
| Cross-validated | Subset median | AGE | 0.623600322 | 0.65584722 | 0.66256063 | 0.616549585 | 0.662560634 | 0.666090015 | 0.644365205 | NA | NA | NA |
| Cross-validated | Overall median | AGE | 0.619916989 | 0.66336111 | 0.66543286 | 0.602760357 | 0.665032856 | 0.668329043 | 0.644365205 | NA | NA | NA |

Other Exposures Cross-Validation (5%)

| Type | tissue | factor | Best_NMF | LDA | Logit | Matched_NMF | NNLS_Logit_betas | NNLS_Logit_means | RF | Signature1 | SinglePeak | Unsupervised |
|---|---|---|---|---|---|---|---|---|---|---|---|---|
| Cross-validated | BLCA | AAcid | 0.900823924 | 0.94859595 | 0.96263103 | 0.934078195 | 0.959983975 | 0.979999455 | 0.997230392 | NA | NA | NA |
| Cross-validated | ESCA | ALCOHOL | 0.429722222 | 0.79444444 | 0.76333333 | NA | 0.776111111 | 0.537777778 | 0.838055556 | NA | NA | NA |
| Cross-validated | HNSCC | ALCOHOL | 0.540309524 | 0.88393651 | 0.86046032 | NA | 0.560214286 | 0.509166667 | 0.842349206 | NA | NA | NA |
| Cross-validated | UHC | ALCOHOL | 0.593067014 | 0.84724804 | 0.85247145 | NA | 0.832760024 | 0.652186173 | 0.821832655 | NA | NA | NA |
| Cross-validated | CESC | APOBEC | 0.642414743 | 0.89340815 | 0.94169964 | 0.625191031 | 0.934791505 | 0.624209846 | 0.889710983 | NA | NA | NA |
| Cross-validated | KIRC | APOBEC | 0.508318793 | 0.69826703 | 0.7117876 | NA | 0.705645293 | 0.627994419 | 0.841215789 | NA | NA | NA |
| Cross-validated | MESO | Asb* | 0.936992063 | 0.95929497 | 0.91134921 | NA | 0.909232804 | 0.910615079 | 0.948585979 | NA | NA | NA |
| Cross-validated | COAD | BMI | 0.509918686 | 0.75036104 | 0.75432404 | NA | 0.74678314 | 0.556597888 | 0.805663951 | NA | NA | NA |
| Cross-validated | ESCA | BMI | 0.660142857 | 0.94139048 | 0.93893968 | NA | 0.854126984 | 0.585069841 | 0.9104 | NA | NA | NA |
| Cross-validated | KIRP | BMI | 0.643020408 | 0.82332313 | 0.87274943 | NA | 0.868048753 | 0.826267574 | 0.904244898 | NA | NA | NA |
| Cross-validated | UCEC | BMI | 0.543204582 | 0.79259624 | 0.79995063 | NA | 0.797657233 | 0.527358531 | 0.881325989 | NA | NA | NA |
| Cross-validated | BRCA | BRCA | 0.707138959 | 0.88257263 | 0.8958253 | 0.705076898 | 0.877725086 | 0.827402831 | 0.947236057 | NA | NA | NA |
| Cross-validated | OV | BRCA | 0.79598898 | 0.81856945 | 0.79103983 | 0.737922445 | 0.795494084 | 0.775241733 | 0.81027886 | NA | NA | NA |
| Cross-validated | UHC | HepB | 0.512648409 | 0.80153666 | 0.79672446 | NA | 0.794404737 | 0.667174398 | 0.788709994 | NA | NA | NA |
| Cross-validated | UHC | HepC | 0.54616527 | 0.76805697 | 0.78378124 | NA | 0.777087324 | 0.697725531 | 0.844852709 | NA | NA | NA |
| Cross-validated | GBM | IDH | 0.718932271 | 0.91402419 | 0.92430051 | NA | 0.837869533 | 0.500425532 | 0.93758507 | NA | NA | NA |
| Cross-validated | LGG | IDH | 0.78981692 | 0.89885643 | 0.9071452 | NA | 0.836359764 | 0.700950293 | 0.948479735 | NA | NA | NA |
| Cross-validated | GBM | MGMT | 0.659302876 | 0.85676861 | 0.85996979 | NA | 0.85519465 | 0.794745829 | 0.915072586 | NA | NA | NA |
| Cross-validated | LGG | MGMT | 0.712933622 | 0.75147547 | 0.74939755 | NA | 0.749397547 | 0.747319625 | 0.739224387 | NA | NA | NA |
| Cross-validated | COAD | MSI | 0.958222478 | 0.95905945 | 0.96127324 | 0.947851012 | 0.965442063 | 0.982640656 | 0.971009938 | NA | NA | NA |
| Cross-validated | STAD | MSI | 0.956180366 | 0.99666866 | 0.96008308 | 0.998500114 | 0.979302584 | 0.995184412 | 0.99896893 | NA | NA | NA |
| Cross-validated | UCEC | MSI | 0.93743388 | 0.98660177 | 0.97469101 | 0.97435779 | 0.974691008 | 0.979018739 | 0.993460277 | NA | NA | NA |
| Cross-validated | STAD | POLD | 0.928667034 | 0.99320965 | 0.95884768 | NA | 0.96170482 | 0.995439793 | 0.997743497 | NA | NA | NA |
| Cross-validated | UCEC | POLD | 0.899027778 | 0.94547619 | 0.94809524 | NA | 0.97547619 | 0.990238095 | 0.98047619 | NA | NA | NA |
| Cross-validated | BRCA | POLE | 0.586189297 | 0.76336128 | 0.76911538 | 0.574040647 | 0.7697291 | 0.702234348 | 0.917948295 | NA | NA | NA |
| Cross-validated | COAD | POLE | 0.824664365 | 0.98611111 | 1 | 0.729909508 | 1 | 0.999652778 | 1 | NA | NA | NA |
| Cross-validated | STAD | POLE | 0.950729865 | 0.94326342 | 0.91240394 | NA | 0.958952185 | 0.99373984 | 0.998710757 | NA | NA | NA |
| Cross-validated | UCEC | POLE | 0.81869898 | 0.98280612 | 0.97744898 | 0.723664966 | 0.980306122 | 0.980306122 | 0.996122449 | NA | NA | NA |
| Cross-validated | BLCA | SMOKING | 0.604835049 | 0.86956502 | 0.86393174 | 0.654626096 | 0.857534289 | 0.7014081 | 0.820497197 | NA | NA | NA |
| Cross-validated | CESC | SMOKING | 0.532898402 | 0.55366447 | 0.54878214 | NA | 0.518490441 | 0.506370013 | 0.704938443 | NA | NA | NA |
| Cross-validated | ESCAD | SMOKING | 0.50725 | 0.88649603 | 0.8803373 | NA | 0.789400794 | 0.619666667 | 0.810960317 | NA | NA | NA |
| Cross-validated | ESCSQ | SMOKING | 0.443450697 | 0.82107143 | 0.81750469 | 0.525163781 | 0.779602934 | 0.587748918 | 0.84084139 | NA | NA | NA |
| Cross-validated | HNSCC | SMOKING | 0.751174232 | 0.85365719 | 0.86827177 | 0.74452894 | 0.868619311 | 0.773472203 | 0.860805238 | NA | NA | NA |
| Cross-validated | KIRP | SMOKING | 0.427135621 | 0.76639869 | 0.78816748 | 0.520120098 | 0.764123366 | 0.611937092 | 0.848280229 | NA | NA | NA |
| Cross-validated | LUAD | SMOKING | 0.854531001 | 0.86150447 | 0.91205215 | 0.886245807 | 0.899418592 | 0.909887707 | 0.933922819 | NA | NA | NA |
| Cross-validated | PAAD | SMOKING | 0.563984276 | 0.67212723 | 0.72299123 | NA | 0.764673932 | 0.55925747 | 0.887065773 | NA | NA | NA |
| Cross-validated | SKCM | UV* | 0.921960786 | 0.93954021 | 0.94968319 | 0.893159361 | 0.958016675 | 0.978354335 | 0.974175368 | NA | NA | NA |
| Cross-validated | Median | NA | 0.660142857 | 0.86956502 | 0.87274943 | 0.733915977 | 0.854126984 | 0.702234348 | 0.904244898 | NA | NA | NA |
| Cross-validated | Subset median | NA | 0.80734398 | 0.88799039 | 0.9268759 | 0.733915977 | 0.917105048 | 0.868645269 | 0.940579438 | NA | NA | NA |
| Cross-validated | Subset smoking | SMOKING | 0.604835049 | 0.85365719 | 0.86393174 | 0.654626096 | 0.857534289 | 0.7014081 | 0.848280229 | NA | NA | NA |
| | median | | | | | | | | | | | |
| Cross-validated | Overall smoking | SMOKING | 0.548441339 | 0.83736431 | 0.84071821 | 0.522641939 | 0.784501864 | 0.615801879 | 0.844560809 | NA | NA | NA |
| | median | | | | | | | | | | | |

Age Apparent (10%)

| type | tissue | factor | Best_NMF | LDA | Logit | Matched_NMF | NNLS_Logit_betas | NNLS_Logit_means | RF | Sig0.5ture1 | SinglePeak | Unsupervised |
|---|---|---|---|---|---|---|---|---|---|---|---|---|
| Apparent | ACC | AGE | 0.535652174 | 0.8 | 0.8 | NA | 0.8 | 0.777391304 | 0.795652174 | 0.471304348 | 0.74 | NA |
| Apparent | BLCA | AGE | 0.635629252 | 0.72130102 | 0.72130102 | 0.495748299 | 0.72130102 | 0.72130102 | 0.729804422 | 0.654336735 | 0.62478741 | 0.654336735 |
| Apparent | BRCA | AGE | 0.598032285 | 0.6116649 | 0.6116649 | 0.585342288 | 0.611664899 | 0.611664899 | 0.682832567 | 0.555190291 | 0.56808059 | 0.60466596 |
| Apparent | CESC | AGE | 0.643927649 | 0.70594315 | 0.73514212 | 0.643927649 | 0.735142119 | 0.705684755 | 0.714728682 | 0.56873385 | 0.61472868 | 0.56873385 |
| Apparent | CHOL | AGE | 0.49112426 | 0.76627219 | 0.76627219 | NA | 0.766272189 | 0.766272189 | 0.766272189 | 0.553254438 | 0.62721893 | NA |
| Apparent | COAD | AGE | 0.587669095 | 0.65816857 | 0.65816857 | 0.582726327 | 0.658168574 | 0.658168574 | 0.74726847 | 0.590530697 | 0.68405307 | 0.590530697 |
| Apparent | ESCAD | AGE | 0.573407202 | 0.65927978 | 0.6565097 | 0.548476454 | 0.656509695 | 0.628808864 | 0.663434903 | 0.573407202 | 0.5166205 | 0.573407202 |
| Apparent | ESCSQ | AGE | 0.586894587 | 0.64529915 | 0.64387464 | 0.586894587 | 0.643874644 | 0.613960114 | 0.677350427 | 0.575498575 | 0.48717949 | 0.575498575 |
| Apparent | GBM | AGE | 0.677777778 | 0.70357143 | 0.70357143 | 0.629365079 | 0.703571429 | 0.701719577 | 0.741137566 | 0.612301587 | 0.68267196 | 0.612301587 |
| Apparent | HNSCC | AGE | 0.718116143 | 0.81383718 | 0.81355932 | 0.636287858 | 0.813559322 | 0.739372048 | 0.823562101 | 0.671158655 | 0.74576271 | 0.671158655 |
| Apparent | KICH | AGE | 0.828719723 | 0.83217993 | 0.83217993 | 0.541522491 | 0.832179931 | 0.832179931 | 0.870242215 | 0.709342561 | 0.85813149 | 0.761245675 |
| Apparent | KIRC | AGE | 0.641551907 | 0.80058263 | 0.81064619 | 0.563426907 | 0.810646186 | 0.762711864 | 0.800052966 | 0.551112288 | 0.7717161 | 0.724311441 |
| Apparent | KIRP | AGE | 0.695156695 | 0.75356125 | 0.75356125 | 0.695156695 | 0.753561254 | 0.753561254 | 0.77991453 | 0.494301994 | 0.71794872 | 0.705128205 |
| Apparent | LAML | AGE | 0.419270833 | 0.68315972 | 0.68315972 | 0.419270833 | 0.683159722 | 0.683159722 | 0.722222222 | 0.585069444 | 0.61545139 | 0.635416667 |
| Apparent | LGG | AGE | 0.759259259 | 0.91481481 | 0.9037037 | 0.85 | 0.903703704 | 0.851851852 | 0.87962963 | 0.792592593 | 0.87777778 | 0.944444444 |
| Apparent | UHC | AGE | 0.571428571 | 0.75554187 | 0.75061576 | 0.594827586 | 0.750615764 | 0.746921182 | 0.769704433 | 0.549261084 | 0.67426108 | 0.674876847 |
| Apparent | LUAD | AGE | 0.657407407 | 0.62654321 | 0.62654321 | 0.484567901 | 0.62654321 | 0.62654321 | 0.765432099 | 0.456790123 | 0.57407407 | 0.456790123 |
| Apparent | OV | AGE | 0.528101803 | 0.69379639 | 0.69379639 | 0.511134677 | 0.693796394 | 0.693796394 | 0.693796394 | 0.671792153 | 0.54003181 | 0.671792153 |
| Apparent | PAAD | AGE | 0.649122807 | 0.63684211 | 0.63684211 | 0.549122807 | 0.636842105 | 0.636842105 | 0.707017544 | 0.638596491 | 0.53333333 | 0.638596491 |
| Apparent | PCPG | AGE | 0.704294218 | 0.76360544 | 0.76020408 | 0.742772109 | 0.760204082 | 0.758503401 | 0.760841837 | 0.523384354 | 0.77827381 | 0.753401361 |
| Apparent | PRAD | AGE | 0.606967742 | 0.66636559 | 0.66748387 | 0.606967742 | 0.667483871 | 0.664774194 | 0.651956989 | 0.560451613 | 0.69178495 | 0.608924731 |
| Apparent | SARC | AGE | 0.749188897 | 0.81542898 | 0.79596251 | 0.798485941 | 0.795962509 | 0.775775054 | 0.837959625 | 0.692682048 | 0.79397981 | 0.805875991 |
| Apparent | SKCM | AGE | 0.627602617 | 0.62135634 | 0.62135634 | 0.396490184 | 0.621356336 | 0.621356336 | 0.698691255 | 0.483045806 | 0.53390839 | 0.483045806 |
| Apparent | STAD | AGE | 0.631395349 | 0.66119951 | 0.66119951 | 0.631395349 | 0.66119951 | 0.66119951 | 0.688127295 | 0.6000612 | 0.59461444 | 0.6000612 |
| Apparent | TG CT | AGE | 0.692763158 | 0.60164474 | 0.60164474 | 0.601973684 | 0.601644737 | 0.601644737 | 0.617434211 | 0.432894737 | 0.6 | 0.613157895 |
| Apparent | THCA | AGE | 0.656948494 | 0.770724 | 0.770724 | 0.664941691 | 0.770724004 | 0.776311953 | 0.802040816 | 0.518148688 | 0.74531098 | 0.774514091 |
| Apparent | THYM | AGE | 0.727650728 | 0.74878725 | 0.73908524 | 0.684684685 | 0.739085239 | 0.759182259 | 0.776853777 | 0.595980596 | 0.71067221 | 0.718641719 |
| Apparent | UCEC | AGE | 0.702479339 | 0.65289256 | 0.79752066 | 0.446280992 | 0.797520661 | 0.747933884 | 0.805785124 | 0.661157025 | 0.5785124 | 0.561983471 |
| Apparent | UCS | AGE | 0.58496732 | 0.57026144 | 0.57026144 | NA | 0.570261438 | 0.570261438 | 0.609477124 | 0.633986928 | 0.60947712 | NA |
| Apparent | UVM | AGE | 0.675 | 0.69375 | 0.69375 | NA | 0.69375 | 0.69375 | 0.70125 | 0.29 | 0.58625 | NA |
| Apparent | Median | AGE | 0.642739778 | 0.69868391 | 0.71243622 | 0.590861087 | 0.712436224 | 0.703702166 | 0.744203018 | 0.574452889 | 0.62600317 | 0.637006579 |
| Apparent | Subset median | AGE | 0.646525228 | 0.69868391 | 0.71243622 | 0.590861087 | 0.712436224 | 0.703702166 | 0.744203018 | 0.58028401 | 0.64952425 | 0.637006579 |
| Apparent | Overall median | AGE | 0.642739778 | 0.69868391 | 0.71243622 | 0.584034307 | 0.712436224 | 0.703702166 | 0.744203018 | 0.574452889 | 0.62600317 | 0.612729741 |

Other Exposures Apparent (10%)

| type | tissue | factor | Best_NMF | LDA | Logit | Matched_NMF | NNLS_Logit_betas | NNLS_Logit_means | RF | Signature1 | SinglePeak | Unsupervised |
|---|---|---|---|---|---|---|---|---|---|---|---|---|
| Apparent | BLCA | AAcid | 0.854798762 | 0.86130031 | 0.8371517 | 0.854798762 | 0.90495356 | 0.983900929 | 1 | NA | NA | 0.964396285 |
| Apparent | ESCA | ALCOHOL | 0.643518519 | 0.94907407 | 0.95138889 | NA | 0.958333333 | 0.763888889 | 0.826388889 | NA | NA | NA |
| Apparent | HNSCC | ALCOHOL | 0.589861751 | 0.89861751 | 0.88248848 | NA | 0.804147465 | 0.617511521 | 0.965437788 | NA | NA | NA |
| Apparent | UHC | ALCOHOL | 0.603650138 | 0.86329201 | 0.88378099 | NA | 0.865530303 | 0.602789256 | 0.840564738 | NA | NA | NA |
| Apparent | CESC | APOBEC | 0.658823529 | 0.90739065 | 0.9239819 | 0.611764706 | 0.926696833 | 0.647360483 | 0.852036199 | NA | NA | 0.638612368 |
| Apparent | KIRC | APOBEC | 0.530708092 | 0.76710019 | 0.77480732 | NA | 0.744219653 | 0.669797688 | 0.903420039 | NA | NA | NA |
| Apparent | MESO | Asb* | 0.9375 | 0.91931818 | 0.91931818 | NA | 0.919318182 | 0.919318182 | 0.938068182 | NA | NA | NA |
| Apparent | COAD | BMI | 0.541831457 | 0.79099483 | 0.81480073 | NA | 0.801414664 | 0.563355643 | 0.826855796 | NA | NA | NA |
| Apparent | ESCA | BMI | 0.61637931 | 0.89408867 | 0.8953202 | NA | 0.86637931 | 0.593596059 | 0.905788177 | NA | NA | NA |
| Apparent | KIRP | BMI | 0.75 | 0.81451613 | 0.83870968 | NA | 0.848387097 | 0.819758065 | 0.893145161 | NA | NA | NA |
| Apparent | UCEC | BMI | 0.611745629 | 0.78666103 | 0.80738861 | NA | 0.803722504 | 0.505076142 | 0.898688663 | NA | NA | NA |
| Apparent | BRCA | BRCA | 0.716407775 | 0.86067664 | 0.87882523 | 0.666518122 | 0.879292758 | 0.839297858 | 0.948210643 | NA | NA | 0.67027417 |
| Apparent | OV | BRCA | 0.812738368 | 0.81998474 | 0.798627 | 0.663615561 | 0.802440885 | 0.789473684 | 0.845347063 | NA | NA | 0.809687262 |
| Apparent | UHC | HepB | 0.560757576 | 0.81909091 | 0.81848485 | NA | 0.816742424 | 0.65469697 | 0.798484848 | NA | NA | NA |
| Apparent | UHC | HepC | 0.635080645 | 0.72177419 | 0.83284457 | NA | 0.833944282 | 0.664956012 | 0.855571848 | NA | NA | NA |
| Apparent | GBM | IDH | 0.802843348 | 0.91335837 | 0.91201717 | NA | 0.836373391 | 0.504291845 | 0.899678112 | NA | NA | NA |
| Apparent | LGG | IDH | 0.787586659 | 0.87997103 | 0.88383403 | NA | 0.846514676 | 0.812265029 | 0.910616356 | NA | NA | NA |
| Apparent | GBM | MGMT | 0.660323354 | 0.86856966 | 0.87669219 | NA | 0.872746964 | 0.782470798 | 0.895451381 | NA | NA | NA |
| Apparent | LGG | MGMT | 0.70021645 | 0.74891775 | 0.74891775 | NA | 0.748917749 | 0.748917749 | 0.758387446 | NA | NA | NA |
| Apparent | COAD | MSI | 0.941120608 | 0.88528015 | 0.79430199 | 0.968660969 | 0.85660019 | 0.969230769 | 0.989268756 | NA | NA | 0.967046534 |
| Apparent | STAD | MSI | 0.933666088 | 0.9846749 | 0.92597828 | 0.999927662 | NA | 0.99657789 | 0.998288945 | NA | NA | 0.999855324 |
| Apparent | UCEC | MSI | 0.945767196 | 0.91997354 | 0.99041005 | 0.985780423 | 0.990410053 | 0.992063492 | 0.990244709 | NA | NA | 1 |
| Apparent | STAD | POLD | 0.936030983 | 0.99731038 | 1 | NA | 1 | 0.99704142 | 1 | NA | NA | NA |
| Apparent | UCEC | POLD | 0.903769841 | 0.99404762 | 0.91269841 | NA | 0.912698413 | 0.998015873 | 1 | NA | NA | NA |
| Apparent | BRCA | POLE | 0.664796252 | 0.78265139 | 0.80240044 | 0.530180867 | 0.802400436 | 0.689361702 | 0.862356792 | NA | NA | 0.423294835 |
| Apparent | COAD | POLE | 0.875 | 0.99070513 | 0.92964744 | 0.728685897 | 0.959775641 | 1 | 1 | NA | NA | 0.72275641 |
| Apparent | STAD | POLE | 0.945815058 | 0.97000368 | 0.94221568 | NA | NA | 0.999631947 | 0.998619801 | NA | NA | NA |
| Apparent | UCEC | POLE | 0.838888889 | 1 | 1 | 0.714285714 | 1 | 1 | 1 | NA | NA | 0.734126984 |
| Apparent | BLCA | SMOKING | 0.673109244 | 0.85395538 | 0.84775427 | 0.673109244 | 0.847058824 | 0.707794842 | 0.819559548 | NA | 0.64022023 | 0.683917705 |
| Apparent | CESC | SMOKING | 0.560538321 | 0.64114964 | 0.63567518 | NA | 0.522582117 | 0.522810219 | 0.729972628 | NA | 0.42810219 | NA |
| Apparent | ESCAD | SMOKING | 0.654037267 | 0.89440994 | 0.89192547 | NA | 0.894409938 | 0.628571429 | 0.888819876 | NA | 0.5826087 | NA |
| Apparent | ESCSQ | SMOKING | 0.572543917 | 0.81262199 | 0.81327261 | 0.405985686 | 0.761548471 | 0.529603123 | 0.833116461 | NA | 0.52635003 | 0.470071568 |
| Apparent | HNSCC | SMOKING | 0.759568798 | 0.88763727 | 0.90063792 | 0.765180879 | 0.899749677 | 0.770712209 | 0.833595769 | NA | 0.69533269 | 0.818213017 |
| Apparent | KIRP | SMOKING | 0.675967262 | 0.84672619 | 0.86011905 | 0.52046131 | 0.807291667 | 0.72172619 | 0.881696429 | NA | 0.60825893 | 0.625744048 |
| Apparent | LUAD | SMOKING | 0.878667641 | 0.86091466 | 0.9011669 | 0.886446695 | 0.904879774 | 0.909476662 | 0.942656766 | NA | 0.90980996 | 0.910619192 |
| Apparent | PAAD | SMOKING | 0.594560405 | 0.79190386 | 0.82258065 | NA | 0.852624921 | 0.71315623 | 0.868912081 | NA | 0.54854522 | NA |
| Apparent | SKCM | UV* | 0.905002674 | 0.90207071 | 0.91818182 | 0.825027955 | 0.931313131 | 0.970959596 | 0.964924242 | NA | NA | 0.949632943 |
| Apparent | Median | NA | 0.70021645 | 0.86856966 | 0.88248848 | 0.721485806 | 0.85660019 | 0.763888889 | 0.898688663 | NA | 0.59543381 | 0.771907123 |
| Apparent | Subset median | NA | 0.825813628 | 0.87329023 | 0.88973157 | 0.721485806 | 0.899749677 | 0.87438726 | 0.945433704 | NA | NA | 0.771907123 |
| Apparent | Subset smoking | SMOKING | 0.675967262 | 0.85395538 | 0.86011905 | 0.673109244 | 0.847058824 | 0.72172619 | 0.833595769 | NA | 0.64022023 | 0.683917705 |
| | median | | | | | | | | | | | |
| Apparent | Overall smoking | SMOKING | 0.663573255 | 0.85034078 | 0.85393666 | 0.510230655 | 0.849841872 | 0.710475536 | 0.851253925 | NA | 0.56730448 | 0.562872024 |
| | median | | | | | | | | | | | |

Age Cross-Validated (10%)

| type | tissue | factor | Best_NMF | LDA | Logit | Matched_NMF | NNLS_Logit_betas | NNLS_Logit_means | RF | Signature1 | SinglePeak | Unsupervised |
|---|---|---|---|---|---|---|---|---|---|---|---|---|
| Cross-validated | ACC | AGE | 0.574519048 | 0.68288016 | 0.68294683 | NA | 0.682946825 | 0.695997619 | 0.677542857 | NA | NA | NA |
| Cross-validated | BLCA | AGE | 0.647720274 | 0.73004766 | 0.72893149 | 0.489995563 | 0.728931494 | 0.718565332 | 0.727808405 | NA | NA | NA |
| Cross-validated | BRCA | AGE | 0.614161431 | 0.61052831 | 0.61083047 | 0.583118823 | 0.610830469 | 0.590651725 | 0.639621768 | NA | NA | NA |
| Cross-validated | CESC | AGE | 0.656680415 | 0.68225309 | 0.69877329 | 0.693029982 | 0.698773288 | 0.683083534 | 0.686296697 | NA | NA | NA |
| Cross-validated | CHOL | AGE | 0.515 | 0.71833333 | 0.71833333 | NA | 0.718333333 | 0.718333333 | 0.654444444 | NA | NA | NA |
| Cross-validated | COAD | AGE | 0.528622122 | 0.54679706 | 0.55294065 | 0.624598579 | 0.552940648 | 0.550545099 | 0.563112504 | NA | NA | NA |
| Cross-validated | ESCAD | AGE | 0.450083333 | 0.56341667 | 0.55911111 | 0.50525 | 0.559111111 | 0.540833333 | 0.534666667 | NA | NA | NA |
| Cross-validated | ESCSQ | AGE | 0.485142857 | 0.51900952 | 0.52847619 | 0.55027619 | 0.527142857 | 0.53327619 | 0.487219048 | NA | NA | NA |
| Cross-validated | GBM | AGE | 0.653904666 | 0.66336006 | 0.66422127 | 0.62620575 | 0.664221271 | 0.662888067 | 0.685370565 | NA | NA | NA |
| Cross-validated | HNSCC | AGE | 0.706410062 | 0.69315962 | 0.68853746 | 0.635974498 | 0.688840493 | 0.693242202 | 0.697412449 | NA | NA | NA |
| Cross-validated | KICH | AGE | 0.8425 | 0.78983333 | 0.78983333 | 0.617944444 | 0.789833333 | 0.799833333 | 0.775 | NA | NA | NA |
| Cross-validated | KIRC | AGE | 0.692228933 | 0.78105911 | 0.78891825 | 0.653249547 | 0.788918249 | 0.764047552 | 0.742943381 | NA | NA | NA |
| Cross-validated | KIRP | AGE | 0.739938095 | 0.70814762 | 0.70968095 | 0.712204762 | 0.709680952 | 0.715966667 | 0.720528571 | NA | NA | NA |
| Cross-validated | LAML | AGE | 0.561638095 | 0.64847619 | 0.65727619 | 0.551638095 | 0.65727619 | 0.65567619 | 0.610928571 | NA | NA | NA |
| Cross-validated | LGG | AGE | 0.6405 | 0.86588889 | 0.84116667 | 0.809 | 0.841166667 | 0.803166667 | 0.854666667 | NA | NA | NA |
| Cross-validated | LIHC | AGE | 0.617407407 | 0.68743122 | 0.70577116 | 0.596087963 | 0.705771164 | 0.704445767 | 0.683308201 | NA | NA | NA |
| Cross-validated | LUAD | AGE | 0.462916667 | 0.46533333 | 0.48916667 | 0.535083333 | 0.489166667 | 0.5065 | 0.43925 | NA | NA | NA |
| Cross-validated | OV | AGE | 0.512309444 | 0.62033995 | 0.61973389 | 0.506009059 | 0.619733886 | 0.621809644 | 0.599573312 | NA | NA | NA |
| Cross-validated | PAAD | AGE | 0.542416667 | 0.63416667 | 0.6215 | 0.64825 | 0.6215 | 0.624833333 | 0.585583333 | NA | NA | NA |
| Cross-validated | PCPG | AGE | 0.684682431 | 0.72387914 | 0.73304165 | 0.731034805 | 0.733041647 | 0.742699944 | 0.726150448 | NA | NA | NA |
| Cross-validated | PRAD | AGE | 0.598947215 | 0.64394413 | 0.64349867 | 0.591329496 | 0.643498671 | 0.657280487 | 0.64247725 | NA | NA | NA |
| Cross-validated | SARC | AGE | 0.75711987 | 0.78697231 | 0.78407881 | 0.802191324 | 0.784078807 | 0.792535659 | 0.781827517 | NA | NA | NA |
| Cross-validated | SKCM | AGE | 0.622791607 | 0.57033201 | 0.57033201 | 0.418247505 | 0.570332011 | 0.570193122 | 0.601539472 | NA | NA | NA |
| Cross-validated | STAD | AGE | 0.546411734 | 0.65021088 | 0.65021088 | 0.620354596 | 0.650210883 | 0.644162896 | 0.652493337 | NA | NA | NA |
| Cross-validated | TG CT | AGE | 0.665087302 | 0.56227381 | 0.56178175 | 0.616561508 | 0.561781746 | 0.566880952 | 0.585274802 | NA | NA | NA |
| Cross-validated | THCA | AGE | 0.660441709 | 0.76448582 | 0.76536298 | 0.674197388 | 0.765362982 | 0.764333006 | 0.775196822 | NA | NA | NA |
| Cross-validated | THYM | AGE | 0.676317725 | 0.71117421 | 0.7099619 | 0.678412698 | 0.709961905 | 0.740512169 | 0.718850529 | NA | NA | NA |
| Cross-validated | UCEC | AGE | 0.591666667 | 0.65611111 | 0.67611111 | 0.316666667 | 0.672777778 | 0.689444444 | 0.621111111 | NA | NA | NA |
| Cross-validated | UCS | AGE | 0.475027778 | 0.41177778 | 0.44177778 | NA | 0.441777778 | 0.408444444 | 0.444777778 | NA | NA | NA |
| Cross-validated | UVM | AGE | 0.6415 | 0.70116667 | 0.69516667 | NA | 0.695166667 | 0.699166667 | 0.649166667 | NA | NA | NA |
| Cross-validated | Median | AGE | 0.620099507 | 0.67280657 | 0.67952897 | 0.61914952 | 0.677862302 | 0.686263989 | 0.653468891 | NA | NA | NA |
| Cross-validated | Subset median | AGE | 0.631645803 | 0.65973558 | 0.67016619 | 0.61914952 | 0.668499525 | 0.672985801 | 0.667900769 | NA | NA | NA |
| Cross-validated | Overall median | AGE | 0.620099507 | 0.67280657 | 0.67952897 | 0.606324735 | 0.677862302 | 0.686263989 | 0.653468891 | NA | NA | NA |

Other Exposures Cross-Validated (10%)

| type | tissue | factor | Best_NMF | LDA | Logit | Matched NMF | NNLS_Logit_betas | NNLS_Logit_means | RF | Signature1 | SinglePeak | Unsupervised |
|---|---|---|---|---|---|---|---|---|---|---|---|---|
| Cross-validated | BLCA | AAcid | 0.903725754 | 0.83126305 | 0.90861624 | 0.909323449 | 0.907903632 | 0.992189945 | 1 | NA | NA | NA |
| Cross-validated | ESCA | ALCOHOL | 0.424666667 | 0.80788889 | 0.76288889 | NA | 0.722166667 | 0.574444444 | 0.782055556 | NA | NA | NA |
| Cross-validated | HNSCC | ALCOHOL | 0.522354497 | 0.73043981 | 0.73944279 | NA | 0.63188244 | 0.613475529 | 0.825248016 | NA | NA | NA |
| Cross-validated | UHC | ALCOHOL | 0.554455418 | 0.79647291 | 0.80077495 | NA | 0.781747324 | 0.590358522 | 0.788488574 | NA | NA | NA |
| Cross-validated | CESC | APOBEC | 0.601176836 | 0.84564804 | 0.87571154 | 0.61931539 | 0.879764934 | 0.637602522 | 0.849655583 | NA | NA | NA |
| Cross-validated | KIRC | APOBEC | 0.545962201 | 0.70725578 | 0.72693915 | NA | 0.718986481 | 0.636999562 | 0.874863791 | NA | NA | NA |
| Cross-validated | MESO | Asb* | 0.945670635 | 0.9538373 | 0.94792063 | NA | 0.946121693 | 0.928482804 | 0.954229497 | NA | NA | NA |
| Cross-validated | COAD | BMI | 0.516113319 | 0.73839959 | 0.73916569 | NA | 0.719921786 | 0.566866804 | 0.739136097 | NA | NA | NA |
| Cross-validated | ESCA | BMI | 0.643938492 | 0.89322222 | 0.89077381 | NA | 0.796559524 | 0.590357143 | 0.890428571 | NA | NA | NA |
| Cross-validated | KIRP | BMI | 0.697709733 | 0.78487412 | 0.82141394 | NA | 0.819683313 | 0.762189411 | 0.885854738 | NA | NA | NA |
| Cross-validated | UCEC | BMI | 0.532440251 | 0.78413256 | 0.78533259 | NA | 0.777406995 | 0.544087629 | 0.854642944 | NA | NA | NA |
| Cross-validated | BRCA | BRCA | 0.726189125 | 0.83412327 | 0.84848148 | 0.679601779 | 0.852882246 | 0.837853728 | 0.940529365 | NA | NA | NA |
| Cross-validated | OV | BRCA | 0.779991446 | 0.78037435 | 0.76773776 | 0.770713038 | 0.779448836 | 0.763722786 | 0.808636556 | NA | NA | NA |
| Cross-validated | UHC | HepB | 0.515693661 | 0.77662412 | 0.77575989 | NA | 0.765659137 | 0.673547902 | 0.767377529 | NA | NA | NA |
| Cross-validated | LIHC | HepC | 0.523444024 | 0.78339727 | 0.74981302 | NA | 0.761482593 | 0.690429759 | 0.811342593 | NA | NA | NA |
| Cross-validated | GBM | IDH | 0.727080796 | 0.90642046 | 0.89501526 | NA | 0.831550492 | 0.502564783 | 0.878435463 | NA | NA | NA |
| Cross-validated | LGG | IDH | 0.787125391 | 0.88255449 | 0.87897002 | NA | 0.827196079 | 0.639206559 | 0.912699043 | NA | NA | NA |
| Cross-validated | GBM | MGMT | 0.674698201 | 0.847976 | 0.85004088 | NA | 0.847367262 | 0.786999666 | 0.881131126 | NA | NA | NA |
| Cross-validated | LGG | MGMT | 0.713630203 | 0.76943309 | 0.76705936 | NA | 0.766069098 | 0.751602647 | 0.735820874 | NA | NA | NA |
| Cross-validated | COAD | MSI | 0.955370707 | 0.87677418 | 0.82713429 | 0.955495347 | 0.868981359 | 0.970629895 | 0.979853571 | NA | NA | NA |
| Cross-validated | STAD | MSI | 0.953151324 | 0.98783248 | 0.94099747 | 0.998538094 | 0.941558442 | 0.995304173 | 0.998239867 | NA | NA | NA |
| Cross-validated | UCEC | MSI | 0.943527783 | 0.96482797 | 0.96230635 | 0.99198372 | 0.963456678 | 0.986199043 | 0.985322127 | NA | NA | NA |
| Cross-validated | STAD | POLD | 0.927086208 | 0.91680428 | 0.99204444 | NA | 0.995834212 | 0.997855306 | 0.999067921 | NA | NA | NA |
| Cross-validated | UCEC | POLD | 0.8725 | 0.90357143 | 0.95166667 | NA | 0.9525 | 0.990952381 | 0.980535714 | NA | NA | NA |
| Cross-validated | BRCA | POLE | 0.633686757 | 0.73762873 | 0.70494221 | 0.582100599 | 0.693533571 | 0.698238365 | 0.883512685 | NA | NA | NA |
| Cross-validated | COAD | POLE | 0.752971435 | 0.98970721 | 0.99115105 | 0.830525421 | 0.994597598 | 0.998486486 | 0.999783784 | NA | NA | NA |
| Cross-validated | STAD | POLE | 0.950729865 | 0.94326342 | 0.91240394 | NA | 0.958952185 | 0.99373984 | 0.998710757 | NA | NA | NA |
| Cross-validated | UCEC | POLE | 0.762498488 | 0.97214286 | 0.97214286 | 0.754485828 | 0.972142857 | 0.972142857 | 0.998367347 | NA | NA | NA |
| Cross-validated | BLCA | SMOKING | 0.600783949 | 0.82475694 | 0.82345864 | 0.646157785 | 0.820741086 | 0.688619672 | 0.786677329 | NA | NA | NA |
| Cross-validated | CESC | SMOKING | 0.568110484 | 0.63113542 | 0.65288411 | NA | 0.602429397 | 0.532332716 | 0.713010881 | NA | NA | NA |
| Cross-validated | ESCAD | SMOKING | 0.590378968 | 0.8734127 | 0.82959921 | NA | 0.762785714 | 0.614460317 | 0.755079365 | NA | NA | NA |
| Cross-validated | ESCSQ | SMOKING | 0.460098232 | 0.81870809 | 0.83496688 | 0.468574143 | 0.769593566 | 0.521363165 | 0.821424133 | NA | NA | NA |
| Cross-validated | HNSCC | SMOKING | 0.756480544 | 0.83170192 | 0.8488077 | 0.749560806 | 0.855937317 | 0.768101165 | 0.847491077 | NA | NA | NA |
| Cross-validated | KIRP | SMOKING | 0.492380097 | 0.78516767 | 0.78369759 | 0.502989703 | 0.718827627 | 0.647765265 | 0.838436315 | NA | NA | NA |
| Cross-validated | LUAD | SMOKING | 0.843941368 | 0.84952076 | 0.86630973 | 0.887261331 | 0.855244263 | 0.908007745 | 0.924243732 | NA | NA | NA |
| Cross-validated | PAAD | SMOKING | 0.524265759 | 0.71613936 | 0.75783978 | NA | 0.785273202 | 0.581584315 | 0.842031471 | NA | NA | NA |
| Cross-validated | SKCM | UV* | 0.915968469 | 0.88877838 | 0.9178967 | 0.896815554 | 0.937495005 | 0.979617165 | 0.980605121 | NA | NA | NA |
| Cross-validated | Median | NA | 0.697709733 | 0.83170192 | 0.83496688 | 0.762599433 | 0.820741086 | 0.698238365 | 0.878435463 | NA | NA | NA |
| Cross-validated | Subset median | NA | 0.759489516 | 0.83988566 | 0.85755872 | 0.762599433 | 0.862459338 | 0.872930737 | 0.932386548 | NA | NA | NA |
| Cross-validated | Subset smoking | SMOKING | 0.600783949 | 0.82475694 | 0.83496688 | 0.646157785 | 0.820741086 | 0.688619672 | 0.838436315 | NA | NA | NA |
| | median | | | | | | | | | | | |
| Cross-validated | Overall smoking | SMOKING | 0.579244726 | 0.82173252 | 0.82652893 | 0.501494852 | 0.777433384 | 0.631112791 | 0.829930224 | NA | NA | NA |
| | median | | | | | | | | | | | |

Age Apparent (20%)

| type | tissue | factor | Best_NMF | LDA | Logit | Matched_NMF | NNLS_Logit_betas | NNLS_Logit_means | RF | Sig0.5ture1 | SinglePeak | Unsupervised |
|---|---|---|---|---|---|---|---|---|---|---|---|---|
| Apparent | ACC | AGE | 0.610434783 | 0.74695652 | 0.74695652 | NA | 0.746956522 | 0.743478261 | 0.766956522 | 0.471304348 | 0.74 | NA |
| Apparent | BLCA | AGE | 0.587159864 | 0.72937925 | 0.72130102 | 0.486819728 | 0.72130102 | 0.72130102 | 0.765093537 | 0.654336735 | 0.62478741 | 0.654336735 |
| Apparent | BRCA | AGE | 0.587863792 | 0.6116649 | 0.6116649 | 0.556945917 | 0.611664899 | 0.611664899 | 0.678838223 | 0.555190291 | 0.56808059 | 0.60466596 |
| Apparent | CESC | AGE | 0.626873385 | 0.72118863 | 0.7501292 | 0.712144703 | 0.750129199 | 0.741860465 | 0.714211886 | 0.56873385 | 0.61472868 | 0.56873385 |
| Apparent | CHOL | AGE | 0.49112426 | 0.58284024 | 0.58284024 | NA | 0.582840237 | 0.582840237 | 0.659763314 | 0.553254438 | 0.62721893 | NA |
| Apparent | COAD | AGE | 0.562955255 | 0.64047867 | 0.64047867 | 0.636706556 | 0.640478668 | 0.640478668 | 0.617065557 | 0.590530697 | 0.68405307 | 0.590530697 |
| Apparent | ESCAD | AGE | 0.549861496 | 0.64127424 | 0.64127424 | 0.601108033 | 0.641274238 | 0.641274238 | 0.680055402 | 0.573407202 | 0.5166205 | 0.573407202 |
| Apparent | ESCSQ | AGE | 0.605413105 | 0.63960114 | 0.63960114 | 0.605413105 | 0.63960114 | 0.61965812 | 0.574786325 | 0.575498575 | 0.48717949 | 0.575498575 |
| Apparent | GBM | AGE | 0.677777778 | 0.66732804 | 0.66732804 | 0.629365079 | 0.667328042 | 0.667328042 | 0.752380952 | 0.612301587 | 0.68267196 | 0.612301587 |
| Apparent | HNSCC | AGE | 0.726312865 | 0.75576549 | 0.75020839 | 0.612948041 | 0.750208391 | 0.778827452 | 0.718810781 | 0.671158655 | 0.74576271 | 0.671158655 |
| Apparent | KICH | AGE | 0.825259516 | 0.83217993 | 0.83217993 | 0.541522491 | 0.832179931 | 0.832179931 | 0.826989619 | 0.709342561 | 0.85813149 | 0.761245675 |
| Apparent | KIRC | AGE | 0.628972458 | 0.79528602 | 0.79528602 | 0.576800847 | 0.795286017 | 0.777277542 | 0.806541314 | 0.551112288 | 0.7717161 | 0.724311441 |
| Apparent | KIRP | AGE | 0.695156695 | 0.73361823 | 0.73361823 | 0.695156695 | 0.733618234 | 0.733618234 | 0.759259259 | 0.494301994 | 0.71794872 | 0.705128205 |
| Apparent | LAML | AGE | 0.706597222 | 0.68315972 | 0.68315972 | 0.706597222 | 0.683159722 | 0.683159722 | 0.710069444 | 0.585069444 | 0.61545139 | 0.635416667 |
| Apparent | LGG | AGE | 0.759259259 | 0.88518519 | 0.88518519 | 0.85 | 0.885185185 | 0.888888889 | 0.87037037 | 0.792592593 | 0.87777778 | 0.944444444 |
| Apparent | LIHC | AGE | 0.578817734 | 0.74938424 | 0.74692118 | 0.556650246 | 0.746921182 | 0.746921182 | 0.770935961 | 0.549261084 | 0.67426108 | 0.674876847 |
| Apparent | LUAD | AGE | 0.520061728 | 0.56481481 | 0.58950617 | 0.520061728 | 0.589506173 | 0.574074074 | 0.625 | 0.456790123 | 0.57407407 | 0.456790123 |
| Apparent | OV | AGE | 0.52757158 | 0.69379639 | 0.69379639 | 0.514316013 | 0.693796394 | 0.693796394 | 0.717656416 | 0.671792153 | 0.54003181 | 0.671792153 |
| Apparent | PAAD | AGE | 0.50877193 | 0.67719298 | 0.68421053 | 0.559649123 | 0.684210526 | 0.698245614 | 0.705263158 | 0.638596491 | 0.53333333 | 0.638596491 |
| Apparent | PCPG | AGE | 0.704294218 | 0.7442602 | 0.7442602 | 0.742772109 | 0.744260204 | 0.744260204 | 0.750637755 | 0.523384354 | 0.77827381 | 0.753401361 |
| Apparent | PRAD | AGE | 0.607053763 | 0.66348387 | 0.68182796 | 0.607053763 | 0.681827957 | 0.664752688 | 0.654451613 | 0.560451613 | 0.69178495 | 0.608924731 |
| Apparent | SARC | AGE | 0.749188897 | 0.78704037 | 0.78704037 | 0.798485941 | 0.787040375 | 0.787040375 | 0.79001442 | 0.692682048 | 0.79397981 | 0.805875991 |
| Apparent | SKCM | AGE | 0.636525877 | 0.62135634 | 0.62135634 | 0.405413444 | 0.621356336 | 0.621356336 | 0.674301011 | 0.483045806 | 0.53390839 | 0.483045806 |
| Apparent | STAD | AGE | 0.561321909 | 0.66119951 | 0.66119951 | 0.560097919 | 0.66119951 | 0.66119951 | 0.689412485 | 0.6000612 | 0.59461444 | 0.6000612 |
| Apparent | TGCT | AGE | 0.692763158 | 0.59407895 | 0.59407895 | 0.601973684 | 0.594078947 | 0.604605263 | 0.584868421 | 0.432894737 | 0.6 | 0.613157895 |
| Apparent | THCA | AGE | 0.656802721 | 0.77752672 | 0.77538873 | 0.665087464 | 0.775388727 | 0.777429543 | 0.810204082 | 0.518148688 | 0.74531098 | 0.774514091 |
| Apparent | THYM | AGE | 0.727650728 | 0.73908524 | 0.73839224 | 0.684684685 | 0.738392238 | 0.759182259 | 0.739085239 | 0.595980596 | 0.71067221 | 0.718641719 |
| Apparent | UCEC | AGE | 0.760330579 | 0.74380165 | 0.74380165 | 0.380165289 | 0.743801653 | 0.743801653 | 0.710743802 | 0.661157025 | 0.5785124 | 0.561983471 |
| Apparent | UCS | AGE | 0.588235294 | 0.57026144 | 0.64052288 | NA | 0.640522876 | 0.633986928 | 0.705882353 | 0.633986928 | 0.60947712 | NA |
| Apparent | UVM | AGE | 0.675 | 0.69375 | 0.69375 | NA | 0.69375 | 0.69375 | 0.725 | 0.29 | 0.58625 | NA |
| Apparent | Median | AGE | 0.627922921 | 0.6937732 | 0.6937732 | 0.603693395 | 0.693773197 | 0.696021004 | 0.715934151 | 0.574452889 | 0.62600317 | 0.637006579 |
| Apparent | Subset median | AGE | 0.632749168 | 0.70749251 | 0.70754871 | 0.603693395 | 0.707548707 | 0.709773317 | 0.715934151 | 0.58028401 | 0.64952425 | 0.637006579 |
| Apparent | Overall median | AGE | 0.627922921 | 0.6937732 | 0.6937732 | 0.58895444 | 0.693773197 | 0.696021004 | 0.715934151 | 0.574452889 | 0.62600317 | 0.612729741 |

Other Exposures Apparent (20%)

| type | tissue | factor | Best_NMF | LDA | Logit | Matched_NMF | NNLS_Logit_betas | NNLS_Logit_means | RF | Signature1 | SinglePeak | Unsupervised |
|---|---|---|---|---|---|---|---|---|---|---|---|---|
| Apparent | BLCA | AAcid | 0.906501548 | 0.83962848 | 0.82321981 | 0.877399381 | 0.830340557 | 0.978947368 | 0.988235294 | NA | NA | 0.964396285 |
| Apparent | ESCA | ALCOHOL | 0.708333333 | 0.77777778 | 0.78703704 | NA | 0.814814815 | 0.796296296 | 0.784722222 | NA | NA | NA |
| Apparent | HNSCC | ALCOHOL | 0.475806452 | 0.6359447 | 0.67741935 | NA | 0.571428571 | 0.516129032 | 0.705069124 | NA | NA | NA |
| Apparent | LIHC | ALCOHOL | 0.62172865 | 0.76515152 | 0.75223829 | NA | 0.741907713 | 0.583849862 | 0.758608815 | NA | NA | NA |
| Apparent | CESC | APOBEC | 0.665912519 | 0.82413273 | 0.83680241 | 0.621417798 | 0.850678733 | 0.649170437 | 0.809653092 | NA | NA | 0.638612368 |
| Apparent | KIRC | APOBEC | 0.532032755 | 0.63451349 | 0.62512042 | NA | 0.601637765 | 0.590799615 | 0.778540462 | NA | NA | NA |
| Apparent | MESO | Asb* | 0.9375 | 0.93636364 | 0.93636364 | NA | 0.936363636 | 0.936363636 | 0.876136364 | NA | NA | NA |
| Apparent | COAD | BMI | 0.567614846 | 0.75250989 | 0.75737755 | NA | 0.734408275 | 0.573243079 | 0.759925464 | NA | NA | NA |
| Apparent | ESCA | BMI | 0.523399015 | 0.82635468 | 0.83374384 | NA | 0.772783251 | 0.5 | 0.866995074 | NA | NA | NA |
| Apparent | KIRP | BMI | 0.745967742 | 0.80483871 | 0.79435484 | NA | 0.727016129 | 0.773387097 | 0.815322581 | NA | NA | NA |
| Apparent | UCEC | BMI | 0.604765933 | 0.74323181 | 0.74915398 | NA | 0.754230118 | 0.628454597 | 0.811689227 | NA | NA | NA |
| Apparent | BRCA | BRCA | 0.680332739 | 0.79696532 | 0.80597586 | 0.72073678 | 0.804615777 | 0.840232914 | 0.825527032 | NA | NA | 0.67027417 |
| Apparent | OV | BRCA | 0.808924485 | 0.76659039 | 0.74523265 | 0.671624714 | 0.759534706 | 0.5 | 0.647025172 | NA | NA | 0.809687262 |
| Apparent | UHC | HepB | 0.534772727 | 0.6519697 | 0.65242424 | NA | 0.654545455 | 0.672575758 | 0.746893939 | NA | NA | NA |
| Apparent | UHC | HepC | 0.589809384 | 0.82221408 | 0.81048387 | NA | 0.811217009 | 0.687316716 | 0.773826979 | NA | NA | NA |
| Apparent | GBM | IDH | 0.717274678 | 0.82859442 | 0.79801502 | NA | 0.739002146 | 0.5 | 0.716469957 | NA | NA | NA |
| Apparent | LGG | IDH | 0.756286 | 0.76753009 | 0.72789984 | NA | 0.71063705 | 0.559617839 | 0.826682303 | NA | NA | NA |
| Apparent | GBM | MGMT | 0.660787499 | 0.7964725 | 0.79832908 | NA | 0.800185658 | 0.798097006 | 0.78575849 | NA | NA | NA |
| Apparent | LGG | MGMT | 0.700757576 | 0.74891775 | 0.74891775 | NA | 0.748917749 | 0.748917749 | 0.753246753 | NA | NA | NA |
| Apparent | COAD | MSI | 0.977018044 | 0.81272555 | 0.80873694 | 0.90294397 | 0.831718898 | 0.96980057 | 0.981671415 | NA | NA | 0.967046534 |
| Apparent | STAD | MSI | 0.967592593 | 0.81684273 | 0.82443089 | 0.994140625 | 0.859023955 | 0.996354709 | 0.998735307 | NA | NA | 0.999855324 |
| Apparent | UCEC | MSI | 0.940806878 | 0.99537037 | 0.83994709 | 0.990410053 | 0.86359127 | 0.982142857 | 0.998842593 | NA | NA | 1 |
| Apparent | STAD | POLD | 0.847622863 | 0.99919311 | 0.91043572 | NA | 0.9345078 | 0.996234535 | 0.840909091 | NA | NA | NA |
| Apparent | UCEC | POLD | 0.906746032 | 0.64880952 | 0.88293651 | NA | 0.884920635 | 0.998015873 | 0.987103175 | NA | NA | NA |
| Apparent | BRCA | POLE | 0.648180431 | 0.66786688 | 0.63895254 | 0.584658967 | 0.66273868 | 0.68619749 | 0.773104201 | NA | NA | 0.423294835 |
| Apparent | COAD | POLE | 0.875 | 0.85865385 | 0.99903846 | 0.658333333 | 0.999038462 | 1 | 0.997435897 | NA | NA | 0.72275641 |
| Apparent | STAD | POLE | 0.954952485 | 0.83897681 | 0.84468163 | NA | 0.862716231 | 0.998527788 | 0.98951049 | NA | NA | NA |
| Apparent | UCEC | POLE | 0.844444444 | 1 | 1 | 0.785714286 | 1 | 1 | 1 | NA | NA | 0.734126984 |
| Apparent | BLCA | SMOKING | 0.585511446 | 0.77374674 | 0.77348595 | 0.672095045 | 0.755462185 | 0.686004057 | 0.774094465 | NA | 0.64022023 | 0.683917705 |
| Apparent | CESC | SMOKING | 0.55939781 | 0.60291971 | 0.56637774 | NA | 0.5 | 0.5 | 0.685127737 | NA | 0.42810219 | NA |
| Apparent | ESCAD | SMOKING | 0.611180124 | 0.79751553 | 0.76521739 | NA | 0.755279503 | 0.766459627 | 0.724223602 | NA | 0.5826087 | NA |
| Apparent | ESCSQ | SMOKING | 0.612882238 | 0.78724789 | 0.79407938 | 0.550748211 | 0.729342876 | 0.5 | 0.778464541 | NA | 0.52635003 | 0.470071568 |
| Apparent | HNSCC | SMOKING | 0.749535691 | 0.82263404 | 0.82247255 | 0.755410207 | 0.825056525 | 0.751776486 | 0.786195898 | NA | 0.69533269 | 0.818213017 |
| Apparent | KIRP | SMOKING | 0.513020833 | 0.77380952 | 0.83556548 | 0.513020833 | 0.849330357 | 0.723958333 | 0.819568452 | NA | 0.60825893 | 0.625744048 |
| Apparent | LUAD | SMOKING | 0.860995092 | 0.77015559 | 0.79632249 | 0.893703665 | 0.792609618 | 0.912187647 | 0.939268034 | NA | 0.90980996 | 0.910619192 |
| Apparent | PAAD | SMOKING | 0.589816572 | 0.7169513 | 0.70651486 | NA | 0.70113852 | 0.55597723 | 0.671252372 | NA | 0.54854522 | NA |
| Apparent | SKCM | UV* | 0.893966649 | 0.70060606 | 0.7809596 | 0.891876124 | 0.795782828 | 0.96040404 | 0.921540404 | NA | NA | 0.949632943 |
| Apparent | Median | NA | 0.700757576 | 0.78724789 | 0.79632249 | 0.738073493 | 0.792609618 | 0.748917749 | 0.809653092 | NA | 0.59543381 | 0.771907123 |
| Apparent | Subset median | NA | 0.826684465 | 0.80484543 | 0.81560474 | 0.738073493 | 0.827698541 | 0.876210281 | 0.873533718 | NA | NA | 0.771907123 |
| Apparent | Subset smoking | SMOKING | 0.612882238 | 0.77380952 | 0.79632249 | 0.672095045 | 0.792609618 | 0.723958333 | 0.786195898 | NA | 0.64022023 | 0.683917705 |
| | median | | | | | | | | | | | |
| Apparent | Overall smoking | SMOKING | 0.600498348 | 0.77377813 | 0.78378266 | 0.531884522 | 0.755370844 | 0.704981195 | 0.776279503 | NA | 0.56730448 | 0.562872024 |
| | median | | | | | | | | | | | |

Age Cross-Validated (20%)

| type | tissue | factor | Best_NMF | LDA | Logit | Matched_NMF | NNLS_Logit_betas | NNLS_Logit_means | RF | Signature1 | SinglePeak | Unsupervised |
|---|---|---|---|---|---|---|---|---|---|---|---|---|
| Cross-validated | ACC | AGE | 0.6034 | 0.65911508 | 0.65911508 | NA | 0.659115079 | 0.64459127 | 0.696947619 | NA | NA | NA |
| Cross-validated | BLCA | AGE | 0.560542424 | 0.70593304 | 0.69216367 | 0.499928644 | 0.692608117 | 0.696082864 | 0.702898449 | NA | NA | NA |
| Cross-validated | BRCA | AGE | 0.612730383 | 0.60568088 | 0.60613355 | 0.569087731 | 0.606217636 | 0.609979355 | 0.626722545 | NA | NA | NA |
| Cross-validated | CESC | AGE | 0.698665905 | 0.67335482 | 0.71002914 | 0.676590348 | 0.710279141 | 0.703201259 | 0.695814975 | NA | NA | NA |
| Cross-validated | CHOL | AGE | 0.554398148 | 0.62892512 | 0.63512731 | NA | 0.635127315 | 0.63744213 | 0.562210648 | NA | NA | NA |
| Cross-validated | COAD | AGE | 0.567054573 | 0.56296088 | 0.56547837 | 0.652345477 | 0.565478366 | 0.563560284 | 0.575126462 | NA | NA | NA |
| Cross-validated | ESCAD | AGE | 0.499492063 | 0.59626984 | 0.59426984 | 0.491603175 | 0.594269841 | 0.586269841 | 0.529027778 | NA | NA | NA |
| Cross-validated | ESCSQ | AGE | 0.537885714 | 0.54291429 | 0.51758095 | 0.562552381 | 0.518533333 | 0.540771429 | 0.4923 | NA | NA | NA |
| Cross-validated | GBM | AGE | 0.646858285 | 0.67417717 | 0.67449992 | 0.601324431 | 0.674499917 | 0.67291377 | 0.674352198 | NA | NA | NA |
| Cross-validated | HNSCC | AGE | 0.668899788 | 0.7047373 | 0.70686838 | 0.649250216 | 0.706868383 | 0.713166305 | 0.697572724 | NA | NA | NA |
| Cross-validated | KICH | AGE | 0.819666667 | 0.82855556 | 0.81266667 | 0.609555556 | 0.812666667 | 0.818 | 0.801833333 | NA | NA | NA |
| Cross-validated | KIRC | AGE | 0.675869608 | 0.75726399 | 0.75666903 | 0.637431868 | 0.756669025 | 0.744322394 | 0.746289655 | NA | NA | NA |
| Cross-validated | KIRP | AGE | 0.62197619 | 0.75280357 | 0.74994643 | 0.72952381 | 0.749946429 | 0.751946429 | 0.743375 | NA | NA | NA |
| Cross-validated | LAML | AGE | 0.5667 | 0.66194127 | 0.66073492 | 0.5367 | 0.660734921 | 0.675115873 | 0.610711111 | NA | NA | NA |
| Cross-validated | LGG | AGE | 0.708111111 | 0.86477778 | 0.89644444 | 0.824 | 0.896444444 | 0.906444444 | 0.877222222 | NA | NA | NA |
| Cross-validated | UHC | AGE | 0.628547619 | 0.64975397 | 0.65242063 | 0.605484127 | 0.652420635 | 0.6335 | 0.676355159 | NA | NA | NA |
| Cross-validated | LUAD | AGE | 0.428611111 | 0.45377778 | 0.45377778 | 0.565861111 | 0.453777778 | 0.433777778 | 0.428111111 | NA | NA | NA |
| Cross-validated | OV | AGE | 0.555694144 | 0.63014437 | 0.63507142 | 0.512488997 | 0.635071419 | 0.638142847 | 0.610252295 | NA | NA | NA |
| Cross-validated | PAAD | AGE | 0.559111111 | 0.61183333 | 0.61216667 | 0.707666667 | 0.612166667 | 0.61975 | 0.577555556 | NA | NA | NA |
| Cross-validated | PCPG | AGE | 0.683706094 | 0.74259066 | 0.74335348 | 0.742377145 | 0.74335348 | 0.752588695 | 0.728928386 | NA | NA | NA |
| Cross-validated | PRAD | AGE | 0.615229413 | 0.64932258 | 0.65011146 | 0.612353785 | 0.650111464 | 0.647967166 | 0.637256437 | NA | NA | NA |
| Cross-validated | SARC | AGE | 0.753010124 | 0.76988477 | 0.7680767 | 0.80586179 | 0.768076701 | 0.784333961 | 0.779707118 | NA | NA | NA |
| Cross-validated | SKCM | AGE | 0.612878968 | 0.57092857 | 0.56521429 | 0.44875496 | 0.566484127 | 0.563944444 | 0.606911706 | NA | NA | NA |
| Cross-validated | STAD | AGE | 0.582042028 | 0.64134135 | 0.6377611 | 0.627499247 | 0.638008015 | 0.634789269 | 0.63536753 | NA | NA | NA |
| Cross-validated | TGCT | AGE | 0.659009392 | 0.57854431 | 0.58156019 | 0.610812169 | 0.581560185 | 0.579544312 | 0.576799471 | NA | NA | NA |
| Cross-validated | THCA | AGE | 0.661717756 | 0.75657166 | 0.75533876 | 0.691038806 | 0.755338758 | 0.760412214 | 0.759098299 | NA | NA | NA |
| Cross-validated | THYM | AGE | 0.690251757 | 0.69128009 | 0.69721131 | 0.64931438 | 0.69721131 | 0.726876861 | 0.688193802 | NA | NA | NA |
| Cross-validated | UCEC | AGE | 0.552083333 | 0.640625 | 0.65972222 | 0.381944444 | 0.677083333 | 0.663194444 | 0.651041667 | NA | NA | NA |
| Cross-validated | UCS | AGE | 0.497444444 | 0.51252778 | 0.51661111 | NA | 0.516611111 | 0.512444444 | 0.497916667 | NA | NA | NA |
| Cross-validated | UVM | AGE | 0.571261905 | 0.66765476 | 0.66765476 | NA | 0.667654762 | 0.667654762 | 0.579571429 | NA | NA | NA |
| Cross-validated | Median | AGE | 0.612804676 | 0.65443452 | 0.65941865 | 0.611582977 | 0.659925 | 0.655580805 | 0.644149052 | NA | NA | NA |
| Cross-validated | Subset median | AGE | 0.618602802 | 0.65584762 | 0.66022857 | 0.611582977 | 0.667617419 | 0.668054107 | 0.662696932 | NA | NA | NA |
| Cross-validated | Overall median | AGE | 0.612804676 | 0.65443452 | 0.65941865 | 0.607519841 | 0.659925 | 0.655580805 | 0.644149052 | NA | NA | NA |

Other Exposures Cross-Validated (20%)

| type | tissue | factor | Best_NMF | LDA | Logit | Matched_NMF | NNLS_Logit_betas | NNLS_Logit_means | RF | Signature1 | SinglePeak | Unsupervised |
|---|---|---|---|---|---|---|---|---|---|---|---|---|
| Cross-validated | BLCA | AAcid | 0.887971309 | 0.7280731 | 0.76384577 | 0.928914193 | 0.791478479 | 0.965187001 | 0.930792693 | NA | NA | NA |
| Cross-validated | ESCA | ALCOHOL | 0.45150463 | 0.78263889 | 0.80648148 | NA | 0.788773148 | 0.63275463 | 0.746469907 | NA | NA | NA |
| Cross-validated | HNSCC | ALCOHOL | 0.392556349 | 0.64060635 | 0.55019524 | NA | 0.483452381 | 0.491025397 | 0.723809524 | NA | NA | NA |
| Cross-validated | LIHC | ALCOHOL | 0.55722389 | 0.70205052 | 0.70868596 | NA | 0.699325493 | 0.580243568 | 0.738151882 | NA | NA | NA |
| Cross-validated | CESC | APOBEC | 0.598692067 | 0.80146217 | 0.81847491 | 0.617283161 | 0.811750971 | 0.634813668 | 0.803595497 | NA | NA | NA |
| Cross-validated | KIRC | APOBEC | 0.53633751 | 0.59109595 | 0.60159829 | NA | 0.594570174 | 0.5131571 | 0.775106168 | NA | NA | NA |
| Cross-validated | MESO | Asb* | 0.932568543 | 0.89074844 | 0.88085823 | NA | 0.884457431 | 0.939112193 | 0.912184524 | NA | NA | NA |
| Cross-validated | COAD | BMI | 0.545186744 | 0.70717628 | 0.68822798 | NA | 0.666850144 | 0.561662098 | 0.669778499 | NA | NA | NA |
| Cross-validated | ESCA | BMI | 0.544666667 | 0.8352381 | 0.81178571 | NA | 0.777207341 | 0.546833333 | 0.817738095 | NA | NA | NA |
| Cross-validated | KIRP | BMI | 0.670406841 | 0.75360271 | 0.78302677 | NA | 0.779868039 | 0.77231064 | 0.855664826 | NA | NA | NA |
| Cross-validated | UCEC | BMI | 0.506145019 | 0.74461746 | 0.76153292 | NA | 0.753209412 | 0.530949731 | 0.774789454 | NA | NA | NA |
| Cross-validated | BRCA | BRCA | 0.691098126 | 0.71256229 | 0.77506445 | 0.675410545 | 0.768477686 | 0.847945953 | 0.833491461 | NA | NA | NA |
| Cross-validated | OV | BRCA | 0.816247518 | 0.69814089 | 0.64777538 | 0.789221664 | 0.667716632 | 0.53092572 | 0.611661484 | NA | NA | NA |
| Cross-validated | LIHC | HepB | 0.494499472 | 0.69198621 | 0.66767017 | NA | 0.658777902 | 0.644532408 | 0.659395957 | NA | NA | NA |
| Cross-validated | LIHC | HepC | 0.541244491 | 0.73115109 | 0.73341482 | NA | 0.753150634 | 0.597334258 | 0.759007038 | NA | NA | NA |
| Cross-validated | GBM | IDH | 0.741728023 | 0.73227204 | 0.72133923 | NA | 0.703680061 | 0.500879227 | 0.755594004 | NA | NA | NA |
| Cross-validated | LGG | IDH | 0.791074205 | 0.7816819 | 0.76714217 | NA | 0.703953185 | 0.585257753 | 0.812785326 | NA | NA | NA |
| Cross-validated | GBM | MGMT | 0.669443545 | 0.7869929 | 0.78468915 | NA | 0.778745084 | 0.79303369 | 0.769436717 | NA | NA | NA |
| Cross-validated | LGG | MGMT | 0.717749127 | 0.72654801 | 0.72326518 | NA | 0.723492455 | 0.733467203 | 0.723206124 | NA | NA | NA |
| Cross-validated | COAD | MSI | 0.967013936 | 0.77012354 | 0.80907043 | 0.939569543 | 0.83560639 | 0.968119658 | 0.984148932 | NA | NA | NA |
| Cross-validated | STAD | MSI | 0.953593049 | 0.80775667 | 0.82547074 | 0.999352582 | 0.863265631 | 0.995085867 | 0.996703209 | NA | NA | NA |
| Cross-validated | UCEC | MSI | 0.90572239 | 0.87652796 | 0.86245722 | 0.976540548 | 0.885317512 | 0.990889965 | 0.985929523 | NA | NA | NA |
| Cross-validated | STAD | POLD | 0.917821021 | 0.94148322 | 0.92258615 | NA | 0.941406114 | 0.993776634 | 0.880898685 | NA | NA | NA |
| Cross-validated | UCEC | POLD | 0.898401587 | 0.83857143 | 0.87059524 | NA | 0.886309524 | 0.992301587 | 0.982063492 | NA | NA | NA |
| Cross-validated | BRCA | POLE | 0.563037948 | 0.68824118 | 0.60865157 | 0.598047794 | 0.61720393 | 0.705011794 | 0.740302268 | NA | NA | NA |
| Cross-validated | COAD | POLE | 0.807521368 | 0.77410247 | 0.84262223 | 0.75190444 | 0.87752227 | 1 | 0.996222222 | NA | NA | NA |
| Cross-validated | STAD | POLE | 0.859097428 | 0.81746655 | 0.82179436 | NA | 0.854502205 | 0.998431132 | 0.98635011 | NA | NA | NA |
| Cross-validated | UCEC | POLE | 0.807402041 | 0.96399206 | 0.91047619 | 0.71065102 | 0.925486961 | 1 | 0.998722222 | NA | NA | NA |
| Cross-validated | BLCA | SMOKING | 0.568181812 | 0.77248381 | 0.75627442 | 0.659406741 | 0.745410612 | 0.68383884 | 0.75728234 | NA | NA | NA |
| Cross-validated | CESC | SMOKING | 0.554800971 | 0.52688809 | 0.54223654 | NA | 0.492854839 | 0.492953846 | 0.617941978 | NA | NA | NA |
| Cross-validated | ESCAD | SMOKING | 0.55022619 | 0.74486508 | 0.70204101 | NA | 0.673787037 | 0.581562169 | 0.691812831 | NA | NA | NA |
| Cross-validated | ESCSQ | SMOKING | 0.565768842 | 0.75509752 | 0.77109518 | 0.54783925 | 0.71827437 | 0.498 | 0.741529304 | NA | NA | NA |
| Cross-validated | HNSCC | SMOKING | 0.723819725 | 0.76139782 | 0.77030963 | 0.732077954 | 0.784037036 | 0.769028437 | 0.845737971 | NA | NA | NA |
| Cross-validated | KIRP | SMOKING | 0.502018358 | 0.68505741 | 0.68037214 | 0.499292729 | 0.660505619 | 0.530757805 | 0.818009941 | NA | NA | NA |
| Cross-validated | LUAD | SMOKING | 0.834751904 | 0.75116703 | 0.76641943 | 0.891679896 | 0.758664884 | 0.909354532 | 0.917972969 | NA | NA | NA |
| Cross-validated | PAAD | SMOKING | 0.542299915 | 0.66487306 | 0.6318205 | NA | 0.635648987 | 0.577596459 | 0.643515694 | NA | NA | NA |
| Cross-validated | SKCM | UV* | 0.919605213 | 0.78987378 | 0.81139628 | 0.899208005 | 0.859863689 | 0.985882919 | 0.950324215 | NA | NA | NA |
| Cross-validated | Median | NA | 0.670406841 | 0.75360271 | 0.76714217 | 0.741991197 | 0.758664884 | 0.68383884 | 0.803595497 | NA | NA | NA |
| Cross-validated | Subset median | NA | 0.807461704 | 0.76576068 | 0.77307982 | 0.741991197 | 0.787757758 | 0.878650243 | 0.88185547 | NA | NA | NA |
| Cross-validated | Subset smoking | SMOKING | 0.568181812 | 0.75509752 | 0.76641943 | 0.659406741 | 0.745410612 | 0.68383884 | 0.818009941 | NA | NA | NA |
| | median | | | | | | | | | | | |
| Cross-validated | Overall smoking | SMOKING | 0.560284907 | 0.74801606 | 0.72915771 | 0.523919625 | 0.696030704 | 0.579579314 | 0.749405822 | NA | NA | NA |
| | median | | | | | | | | | | | |

Age Apparent (25%)

| type | tissue | factor | Best_NMF | LDA | Logit | Matched_NMF | NNLS_Logit_betas | NNLS_Logit_means | RF | Signature1 | SinglePeak | Unsupervised |
|---|---|---|---|---|---|---|---|---|---|---|---|---|
| Apparent | ACC | AGE | 0.613913043 | 0.73478261 | 0.73478261 | NA | 0.734782609 | 0.734782609 | 0.782608696 | 0.471304348 | 0.74 | NA |
| Apparent | BLCA | AGE | 0.585034014 | 0.73107993 | 0.72130102 | 0.493622449 | 0.72130102 | 0.72130102 | 0.734481293 | 0.654336735 | 0.62478741 | 0.654336735 |
| Apparent | BRCA | AGE | 0.62078473 | 0.6116649 | 0.6116649 | 0.558784023 | 0.611664899 | 0.611664899 | 0.611346766 | 0.555190291 | 0.56808059 | 0.60466596 |
| Apparent | CESC | AGE | 0.621447028 | 0.74082687 | 0.74289406 | 0.720671835 | 0.742894057 | 0.704651163 | 0.735658915 | 0.56873385 | 0.61472868 | 0.56873385 |
| Apparent | CHOL | AGE | 0.49112426 | 0.76627219 | 0.76627219 | NA | 0.766272189 | 0.766272189 | 0.784023669 | 0.553254438 | 0.62721893 | NA |
| Apparent | COAD | AGE | 0.571800208 | 0.64047867 | 0.64047867 | 0.625130073 | 0.640478668 | 0.640478668 | 0.573361082 | 0.590530697 | 0.68405307 | 0.590530697 |
| Apparent | ESCAD | AGE | 0.58033241 | 0.59833795 | 0.57479224 | 0.58033241 | 0.574792244 | 0.574792244 | 0.581717452 | 0.573407202 | 0.5166205 | 0.573407202 |
| Apparent | ESCSQ. | AGE | 0.594729345 | 0.56481481 | 0.56481481 | 0.594729345 | 0.564814815 | 0.564814815 | 0.5997151 | 0.575498575 | 0.48717949 | 0.575498575 |
| Apparent | GBM | AGE | 0.677513228 | 0.60886243 | 0.60886243 | 0.627513228 | 0.608862434 | 0.608862434 | 0.686375661 | 0.612301587 | 0.68267196 | 0.612301587 |
| Apparent | HNSCC | AGE | 0.717421506 | 0.69574882 | 0.69574882 | 0.610725201 | 0.695748819 | 0.711030842 | 0.709919422 | 0.671158655 | 0.74576271 | 0.671158655 |
| Apparent | KICH | AGE | 0.828719723 | 0.83217993 | 0.83217993 | 0.544982699 | 0.832179931 | 0.832179931 | 0.832179931 | 0.709342561 | 0.85813149 | 0.761245675 |
| Apparent | KIRC | AGE | 0.61467161 | 0.80402542 | 0.80402542 | 0.570444915 | 0.804025424 | 0.774364407 | 0.805217161 | 0.551112288 | 0.7717161 | 0.724311441 |
| Apparent | KIRP | AGE | 0.686609687 | 0.73361823 | 0.73361823 | 0.686609687 | 0.733618234 | 0.733618234 | 0.778490028 | 0.494301994 | 0.71794872 | 0.705128205 |
| Apparent | LAML | AGE | 0.706597222 | 0.68315972 | 0.68315972 | 0.706597222 | 0.683159722 | 0.683159722 | 0.716145833 | 0.585069444 | 0.61545139 | 0.635416667 |
| Apparent | LGG | AGE | 0.766666667 | 0.86666667 | 0.88333333 | 0.85 | 0.883333333 | 0.883333333 | 0.868518519 | 0.792592593 | 0.87777778 | 0.944444444 |
| Apparent | UHC | AGE | 0.575123153 | 0.69704433 | 0.69704433 | 0.556034483 | 0.697044335 | 0.697044335 | 0.705665025 | 0.549261084 | 0.67426108 | 0.674876847 |
| Apparent | LUAD | AGE | 0.521604938 | 0.56481481 | 0.56481481 | 0.561728395 | 0.564814815 | 0.564814815 | 0.586419753 | 0.456790123 | 0.57407407 | 0.456790123 |
| Apparent | OV | AGE | 0.516967126 | 0.70652174 | 0.70652174 | 0.516967126 | 0.706521739 | 0.707051962 | 0.713679745 | 0.671792153 | 0.54003181 | 0.671792153 |
| Apparent | PAAD | AGE | 0.561403509 | 0.63684211 | 0.63684211 | 0.721052632 | 0.636842105 | 0.636842105 | 0.60877193 | 0.638596491 | 0.53333333 | 0.638596491 |
| Apparent | PCPG | AGE | 0.704294218 | 0.7442602 | 0.7442602 | 0.742772109 | 0.744260204 | 0.744260204 | 0.763392857 | 0.523384354 | 0.77827381 | 0.753401361 |
| Apparent | PRAD | AGE | 0.606967742 | 0.65004301 | 0.65004301 | 0.606967742 | 0.650043011 | 0.650043011 | 0.650688172 | 0.560451613 | 0.69178495 | 0.608924731 |
| Apparent | SARC | AGE | 0.749188897 | 0.78253425 | 0.78253425 | 0.798485941 | 0.782534247 | 0.789023071 | 0.789473684 | 0.692682048 | 0.79397981 | 0.805875991 |
| Apparent | SKCM | AGE | 0.634146341 | 0.62135634 | 0.62135634 | 0.37953599 | 0.621356336 | 0.621356336 | 0.668649613 | 0.483045806 | 0.53390839 | 0.483045806 |
| Apparent | STAD | AGE | 0.633170135 | 0.66119951 | 0.66119951 | 0.633170135 | 0.66119951 | 0.66119951 | 0.66119951 | 0.6000612 | 0.59461444 | 0.6000612 |
| Apparent | TGCT | AGE | 0.692763158 | 0.60164474 | 0.60164474 | 0.601973684 | 0.601644737 | 0.601644737 | 0.627302632 | 0.432894737 | 0.6 | 0.613157895 |
| Apparent | THCA | AGE | 0.714917396 | 0.73957726 | 0.73573858 | 0.714917396 | 0.735738581 | 0.781025267 | 0.80845481 | 0.518148688 | 0.74531098 | 0.774514091 |
| Apparent | THYM | AGE | 0.727650728 | 0.75051975 | 0.75190575 | 0.684684685 | 0.751905752 | 0.734580735 | 0.741857242 | 0.595980596 | 0.71067221 | 0.718641719 |
| Apparent | UCEC | AGE | 0.574380165 | 0.74380165 | 0.78099174 | 0.487603306 | 0.772727273 | 0.681818182 | 0.719008264 | 0.661157025 | 0.5785124 | 0.561983471 |
| Apparent | UCS | AGE | 0.633986928 | 0.57026144 | 0.57026144 | NA | 0.570261438 | 0.570261438 | 0.668300654 | 0.633986928 | 0.60947712 | NA |
| Apparent | UVM | AGE | 0.735 | 0.69375 | 0.69375 | NA | 0.69375 | 0.69375 | 0.72375 | 0.29 | 0.58625 | NA |
| Apparent | Median | AGE | 0.627308582 | 0.69639658 | 0.69639658 | 0.608846472 | 0.696396577 | 0.695397167 | 0.714912789 | 0.574452889 | 0.62600317 | 0.637006579 |
| Apparent | Subset median | AGE | 0.627308582 | 0.69639658 | 0.69639658 | 0.608846472 | 0.696396577 | 0.690102029 | 0.711799584 | 0.58028401 | 0.64952425 | 0.637006579 |
| Apparent | Overall median | AGE | 0.627308582 | 0.69639658 | 0.69639658 | 0.598351514 | 0.696396577 | 0.695397167 | 0.714912789 | 0.574452889 | 0.62600317 | 0.612729741 |

Other Exposures Apparent (25%)

| type | tissue | factor | Best_NMF | LDA | Logit | Matched_NMF | NNLS_Logit_betas | NNLS_Logit_means | RF | Signature1 | SinglePeak | Unsupervised |
|---|---|---|---|---|---|---|---|---|---|---|---|---|
| Apparent | BLCA | AAcid | 0.893188854 | 0.74365325 | 0.78266254 | 0.936842105 | 0.79380805 | 0.979566563 | 0.953560372 | NA | NA | 0.964396285 |
| Apparent | ESCA | ALCOHOL | 0.550925926 | 0.69907407 | 0.7337963 | NA | 0.782407407 | 0.689814815 | 0.777777778 | NA | NA | NA |
| Apparent | HNSCC | ALCOHOL | 0.433179724 | 0.64631336 | 0.72580645 | NA | 0.555299539 | 0.5 | 0.684331797 | NA | NA | NA |
| Apparent | UHC | ALCOHOL | 0.557248623 | 0.71849174 | 0.71229339 | NA | 0.699121901 | 0.58023416 | 0.728650138 | NA | NA | NA |
| Apparent | CESC | APOBEC | 0.65852187 | 0.77888386 | 0.81628959 | 0.630467572 | 0.793363499 | 0.638310709 | 0.77586727 | NA | NA | 0.638612368 |
| Apparent | KIRC | APOBEC | 0.532514451 | 0.62102601 | 0.61235549 | NA | 0.572133911 | 0.587909441 | 0.719773603 | NA | NA | NA |
| Apparent | MESO | Asb* | 0.9375 | 0.78295455 | 0.77727273 | NA | 0.782954545 | 0.8125 | 0.875568182 | NA | NA | NA |
| Apparent | COAD | BMI | 0.568223304 | 0.70033465 | 0.72254335 | NA | 0.711248859 | 0.555749924 | 0.713074232 | NA | NA | NA |
| Apparent | ESCA | BMI | 0.692118227 | 0.76477833 | 0.73706897 | NA | 0.76046798 | 0.610837438 | 0.806650246 | NA | NA | NA |
| Apparent | KIRP | BMI | 0.717741935 | 0.67580645 | 0.67419355 | NA | 0.7 | 0.5 | 0.769354839 | NA | NA | NA |
| Apparent | UCEC | BMI | 0.611604625 | 0.70135364 | 0.71192893 | NA | 0.699097575 | 0.513254371 | 0.709249859 | NA | NA | NA |
| Apparent | BRCA | BRCA | 0.665796622 | 0.78310949 | 0.7866797 | 0.67095323 | 0.791822509 | 0.840402924 | 0.798091635 | NA | NA | 0.67027417 |
| Apparent | OV | BRCA | 0.663996949 | 0.70823799 | 0.67124333 | 0.663996949 | 0.705949657 | 0.5 | 0.565789474 | NA | NA | 0.809687262 |
| Apparent | UHC | HepB | 0.525984848 | 0.68469697 | 0.67060606 | NA | 0.661515152 | 0.657575758 | 0.69280303 | NA | NA | NA |
| Apparent | UHC | HepC | 0.595857771 | 0.75843109 | 0.76008065 | NA | NA | 0.686583578 | 0.695747801 | NA | NA | NA |
| Apparent | GBM | IDH | 0.711641631 | 0.79425966 | 0.75643777 | NA | 0.699570815 | 0.5 | 0.614270386 | NA | NA | NA |
| Apparent | LGG | IDH | 0.795398889 | 0.7419722 | 0.73890249 | NA | 0.643724347 | 0.650536336 | 0.77856724 | NA | NA | NA |
| Apparent | GBM | MGMT | 0.660323354 | 0.75361646 | 0.7622805 | NA | 0.757677729 | 0.787034888 | 0.708091591 | NA | NA | NA |
| Apparent | LGG | MGMT | 0.700757576 | 0.74891775 | 0.74891775 | NA | 0.748917749 | 0.748917749 | 0.751893939 | NA | NA | NA |
| Apparent | COAD | MSI | 0.864197531 | 0.79012346 | 0.78252612 | 0.864197531 | 0.809401709 | 0.969990503 | 0.983855651 | NA | NA | 0.967046534 |
| Apparent | STAD | MSI | 0.959852431 | 0.76595745 | 0.76275852 | 0.998842593 | 0.811858354 | 0.996801071 | 0.999256063 | NA | NA | 0.999855324 |
| Apparent | UCEC | MSI | 0.946097884 | 0.7771164 | 0.75992063 | 0.963293651 | 0.783399471 | 0.962632275 | 0.998511905 | NA | NA | 1 |
| Apparent | STAD | POLD | 0.937900641 | 0.99677246 | 0.92576654 | NA | 0.924421732 | 0.995965573 | 0.78429263 | NA | NA | NA |
| Apparent | UCEC | POLD | 0.939484127 | 0.60119048 | 0.75992063 | NA | 0.762896825 | 0.996031746 | 0.994047619 | NA | NA | NA |
| Apparent | BRCA | POLE | 0.649487906 | 0.6466994 | 0.63022368 | 0.596971018 | 0.657392253 | 0.501036552 | 0.643371522 | NA | NA | 0.423294835 |
| Apparent | COAD | POLE | 0.812179487 | 0.75929487 | 0.76185897 | 0.601282051 | 0.819871795 | 1 | 1 | NA | NA | 0.72275641 |
| Apparent | STAD | POLE | 0.658991228 | 0.7603975 | 0.7298491 | NA | 0.803367685 | 0.998711815 | 0.840449025 | NA | NA | NA |
| Apparent | UCEC | POLE | 0.804761905 | 0.82460317 | 0.83253968 | 0.726984127 | 0.900793651 | 0.999206349 | 0.999206349 | NA | NA | 0.734126984 |
| Apparent | BLCA | SMOKING | 0.569487105 | 0.71492321 | 0.73193277 | 0.676789336 | 0.70831643 | 0.62103738 | 0.722138511 | NA | 0.64022023 | 0.683917705 |
| Apparent | CESC | SMOKING | 0.555565693 | 0.57559307 | 0.56637774 | NA | 0.5 | 0.5 | 0.663959854 | NA | 0.42810219 | NA |
| Apparent | ESCAD | SMOKING | 0.582608696 | 0.73043478 | 0.73913043 | NA | 0.737888199 | 0.730434783 | 0.707453416 | NA | 0.5826087 | NA |
| Apparent | ESCSQ | SMOKING | 0.575797007 | 0.74690956 | 0.74625895 | 0.404033832 | 0.6870527 | 0.5 | 0.767078725 | NA | 0.52635003 | 0.470071568 |
| Apparent | HNSCC | SMOKING | 0.75932655 | 0.7224241 | 0.7126938 | 0.761385659 | 0.723191214 | 0.768491602 | 0.817466085 | NA | 0.69533269 | 0.818213017 |
| Apparent | KIRP | SMOKING | 0.516369048 | 0.6547619 | 0.66071429 | 0.516369048 | 0.661830357 | 0.44047619 | 0.755952381 | NA | 0.60825893 | 0.625744048 |
| Apparent | LUAD | SMOKING | 0.837501305 | 0.74080622 | 0.75913484 | 0.892137413 | 0.780115512 | 0.912953795 | 0.926597124 | NA | 0.90980996 | 0.910619192 |
| Apparent | PAAD | SMOKING | 0.605154965 | 0.68880455 | 0.67741935 | NA | 0.664136622 | 0.558507274 | 0.632036686 | NA | 0.54854522 | NA |
| Apparent | SKCM | UV* | 0.947809811 | 0.75434343 | 0.76545455 | 0.836939083 | 0.848611111 | 0.960151515 | 0.895833333 | NA | NA | 0.949632943 |
| Apparent | Median | NA | 0.663996949 | 0.7419722 | 0.73913043 | 0.701886732 | 0.743402974 | 0.686583578 | 0.769354839 | NA | 0.59543381 | 0.771907123 |
| Apparent | Subset median | NA | 0.782044228 | 0.7506265 | 0.7608898 | 0.701886732 | 0.78761099 | 0.87667836 | 0.856649709 | NA | NA | 0.771907123 |
| Apparent | Subset smoking | SMOKING | 0.575797007 | 0.7224241 | 0.73193277 | 0.676789336 | 0.70831643 | 0.62103738 | 0.767078725 | NA | 0.64022023 | 0.683917705 |
| | median | | | | | | | | | | | |
| Apparent | Overall smoking | SMOKING | 0.579202851 | 0.71867365 | 0.72231329 | 0.508184524 | 0.697684565 | 0.589772327 | 0.739045446 | NA | 0.56730448 | 0.562872024 |
| | median | | | | | | | | | | | |

Age Cross-Validated (25%)

| type | tissue | factor | Best_NMF | LDA | Logit | Matched_NMF | NNLS_Logit_betas | NNLS_Logit_means | RF | Signature1 | SinglePeak | Unsupervised |
|---|---|---|---|---|---|---|---|---|---|---|---|---|
| Cross-validated | ACC | AGE | 0.641512698 | 0.68536825 | 0.69343492 | NA | 0.693434921 | 0.720346032 | 0.671644444 | NA | NA | NA |
| Cross-validated | BLCA | AGE | 0.530503105 | 0.72184624 | 0.72097503 | 0.504352783 | 0.720975031 | 0.720255334 | 0.721786988 | NA | NA | NA |
| Cross-validated | BRCA | AGE | 0.597488776 | 0.59962957 | 0.59960576 | 0.580654138 | 0.599605759 | 0.596100397 | 0.598366057 | NA | NA | NA |
| Cross-validated | CESC | AGE | 0.647620509 | 0.67318666 | 0.68541284 | 0.717960031 | 0.685698555 | 0.694536753 | 0.689949214 | NA | NA | NA |
| Cross-validated | CHOL | AGE | 0.476111111 | 0.77388889 | 0.77555556 | NA | 0.775555556 | 0.770555556 | 0.755555556 | NA | NA | NA |
| Cross-validated | COAD | AGE | 0.58439205 | 0.54584378 | 0.54032707 | 0.638453333 | 0.540327073 | 0.541154578 | 0.496464119 | NA | NA | NA |
| Cross-validated | ESCAD | AGE | 0.496861111 | 0.58858333 | 0.58858333 | 0.509555556 | 0.588583333 | 0.591916667 | 0.523277778 | NA | NA | NA |
| Cross-validated | ESCSQ | AGE | 0.498319048 | 0.48390476 | 0.48571429 | 0.589619048 | 0.485714286 | 0.508952381 | 0.508661905 | NA | NA | NA |
| Cross-validated | GBM | AGE | 0.646166631 | 0.63618578 | 0.61600396 | 0.624722398 | 0.61600396 | 0.62839357 | 0.635864762 | NA | NA | NA |
| Cross-validated | HNSCC | AGE | 0.632132564 | 0.67556502 | 0.67329662 | 0.621960726 | 0.673426492 | 0.695389777 | 0.669375815 | NA | NA | NA |
| Cross-validated | KICH | AGE | 0.849777778 | 0.81133333 | 0.78788889 | 0.685222222 | 0.792888889 | 0.819111111 | 0.796333333 | NA | NA | NA |
| Cross-validated | KIRC | AGE | 0.664324477 | 0.76654821 | 0.7561851 | 0.664269704 | 0.756185097 | 0.738814096 | 0.735286869 | NA | NA | NA |
| Cross-validated | KIRP | AGE | 0.624295238 | 0.68955238 | 0.70941905 | 0.717161905 | 0.708704762 | 0.703666667 | 0.732171429 | NA | NA | NA |
| Cross-validated | LAML | AGE | 0.5496 | 0.63563333 | 0.6353 | 0.5398 | 0.6353 | 0.666633333 | 0.551422222 | NA | NA | NA |
| Cross-validated | LGG | AGE | 0.726222222 | 0.88877778 | 0.89127778 | 0.849555556 | 0.887944444 | 0.883277778 | 0.869722222 | NA | NA | NA |
| Cross-validated | UHC | AGE | 0.634701587 | 0.64864034 | 0.66006944 | 0.670373016 | 0.660069444 | 0.660581614 | 0.653903968 | NA | NA | NA |
| Cross-validated | LUAD | AGE | 0.478 | 0.47275 | 0.49816667 | 0.566333333 | 0.494833333 | 0.470666667 | 0.408861111 | NA | NA | NA |
| Cross-validated | OV | AGE | 0.491186563 | 0.6343027 | 0.6343027 | 0.515598402 | 0.634302697 | 0.635413808 | 0.634285409 | NA | NA | NA |
| Cross-validated | PAAD | AGE | 0.507777778 | 0.58583333 | 0.5775 | 0.649166667 | 0.5775 | 0.574833333 | 0.479833333 | NA | NA | NA |
| Cross-validated | PCPG | AGE | 0.70173749 | 0.73984214 | 0.72796134 | 0.733036896 | 0.727961336 | 0.754980167 | 0.724617127 | NA | NA | NA |
| Cross-validated | PRAD | AGE | 0.600164342 | 0.62315464 | 0.62307552 | 0.608289934 | 0.623075522 | 0.621652708 | 0.62877019 | NA | NA | NA |
| Cross-validated | SARC | AGE | 0.741407195 | 0.77361751 | 0.77355057 | 0.803309 | 0.773639464 | 0.79414617 | 0.796901269 | NA | NA | NA |
| Cross-validated | SKCM | AGE | 0.612334506 | 0.59054473 | 0.58641775 | 0.443094697 | 0.58762987 | 0.600322511 | 0.57866342 | NA | NA | NA |
| Cross-validated | STAD | AGE | 0.551309442 | 0.63366918 | 0.63274413 | 0.638769558 | 0.632678769 | 0.612786471 | 0.607567067 | NA | NA | NA |
| Cross-validated | TGCT | AGE | 0.656273942 | 0.56539879 | 0.56539879 | 0.624913865 | 0.565398791 | 0.569710961 | 0.55743192 | NA | NA | NA |
| Cross-validated | THCA | AGE | 0.66839206 | 0.70097764 | 0.69658255 | 0.683454849 | 0.696582548 | 0.748949478 | 0.746020324 | NA | NA | NA |
| Cross-validated | THYM | AGE | 0.599306287 | 0.64225167 | 0.63800563 | 0.660300709 | 0.638005635 | 0.654661171 | 0.65408752 | NA | NA | NA |
| Cross-validated | UCEC | AGE | 0.711666667 | 0.72833333 | 0.75333333 | 0.425 | 0.75 | 0.73 | 0.723333333 | NA | NA | NA |
| Cross-validated | UCS | AGE | 0.483666667 | 0.42480556 | 0.41647222 | NA | 0.419805556 | 0.397805556 | 0.445305556 | NA | NA | NA |
| Cross-validated | UVM | AGE | 0.576261905 | 0.69789286 | 0.67789286 | NA | 0.677892857 | 0.692892857 | 0.564797619 | NA | NA | NA |
| Cross-validated | Median | AGE | 0.606249424 | 0.64544601 | 0.64903754 | 0.631683599 | 0.64903754 | 0.663607474 | 0.644884365 | NA | NA | NA |
| Cross-validated | Subset median | AGE | 0.618314872 | 0.63921872 | 0.63665282 | 0.631683599 | 0.636652817 | 0.657621392 | 0.644884365 | NA | NA | NA |
| Cross-validated | Overall median | AGE | 0.606249424 | 0.64544601 | 0.64903754 | 0.623341562 | 0.64903754 | 0.663607474 | 0.644884365 | NA | NA | NA |

Other Exposures Cross-Validated (25%)

| type | tissue | factor | Best_NMF | LDA | Logit | Matched_NMF | NNLS_Logit_betas | NNLS_Logit_means | RF | Signature1 | SinglePeak | Unsupervised |
|---|---|---|---|---|---|---|---|---|---|---|---|---|
| Cross-validated | BLCA | AAcid | 0.853415524 | 0.76086043 | 0.75484958 | 0.922084153 | 0.799133893 | 0.97615036 | 0.838270411 | NA | NA | NA |
| Cross-validated | ESCA | ALCOHOL | 0.366898148 | 0.71145833 | 0.68738426 | NA | 0.702025463 | 0.549537037 | 0.764236111 | NA | NA | NA |
| Cross-validated | HNSCC | ALCOHOL | 0.482525397 | 0.67277143 | 0.66178413 | NA | 0.556848413 | 0.476609524 | 0.755620635 | NA | NA | NA |
| Cross-validated | UHC | ALCOHOL | 0.545232021 | 0.6741559 | 0.67062075 | NA | 0.669262954 | 0.570116224 | 0.708983592 | NA | NA | NA |
| Cross-validated | CESC | APOBEC | 0.64039707 | 0.75183138 | 0.77558267 | 0.631997937 | 0.771250211 | 0.629976765 | 0.765537236 | NA | NA | NA |
| Cross-validated | KIRC | APOBEC | 0.526626053 | 0.60753075 | 0.60923763 | NA | 0.594728679 | 0.513004006 | 0.763767082 | NA | NA | NA |
| Cross-validated | MESO | Asb* | 0.93031746 | 0.8422108 | 0.79579678 | NA | 0.802902116 | 0.706291667 | 0.885181037 | NA | NA | NA |
| Cross-validated | COAD | BMI | 0.565842828 | 0.63814494 | 0.650479 | NA | 0.633859226 | 0.540709518 | 0.619848003 | NA | NA | NA |
| Cross-validated | ESCA | BMI | 0.577248016 | 0.76760317 | 0.76310317 | NA | 0.733242063 | 0.518809524 | 0.755696429 | NA | NA | NA |
| Cross-validated | KIRP | BMI | 0.596604205 | 0.665895 | 0.7072685 | NA | 0.715428451 | 0.554719611 | 0.71497035 | NA | NA | NA |
| Cross-validated | UCEC | BMI | 0.400389763 | 0.68208377 | 0.66640581 | NA | 0.660840042 | 0.509053943 | 0.7179244 | NA | NA | NA |
| Cross-validated | BRCA | BRCA | 0.679480853 | 0.76172047 | 0.77170856 | 0.662313155 | 0.775711923 | 0.826020938 | 0.79683041 | NA | NA | NA |
| Cross-validated | OV | BRCA | 0.791894644 | 0.64742102 | 0.62317131 | 0.751381761 | 0.655789908 | 0.496954023 | 0.506952503 | NA | NA | NA |
| Cross-validated | UHC | HepB | 0.50079654 | 0.67198144 | 0.64947486 | NA | 0.65032776 | 0.639810742 | 0.653682043 | NA | NA | NA |
| Cross-validated | UHC | HepC | 0.578200215 | 0.72204174 | 0.68347142 | NA | 0.709725806 | 0.615072433 | 0.681980939 | NA | NA | NA |
| Cross-validated | GBM | IDH | 0.744833928 | 0.73832244 | 0.72202831 | NA | 0.665995391 | 0.502083333 | 0.67539638 | NA | NA | NA |
| Cross-validated | LGG | IDH | 0.752872737 | 0.72759771 | 0.7256144 | NA | 0.673232092 | 0.609215281 | 0.764421101 | NA | NA | NA |
| Cross-validated | GBM | MGMT | 0.669368303 | 0.75452484 | 0.74751765 | NA | 0.746234014 | 0.775536537 | 0.737402676 | NA | NA | NA |
| Cross-validated | LGG | MGMT | 0.676041557 | 0.70522688 | 0.70222785 | NA | 0.704115773 | 0.726527189 | 0.731851482 | NA | NA | NA |
| Cross-validated | COAD | MSI | 0.914022007 | 0.79603712 | 0.73690594 | 0.956639912 | 0.789836081 | 0.971534088 | 0.976410878 | NA | NA | NA |
| Cross-validated | STAD | MSI | 0.948099767 | 0.77779815 | 0.78556657 | 0.996524529 | 0.826510371 | 0.995367287 | 0.997440774 | NA | NA | NA |
| Cross-validated | UCEC | MSI | 0.924969262 | 0.88679362 | 0.81721243 | 0.977319778 | 0.845158239 | 0.985163554 | 0.99357244 | NA | NA | NA |
| Cross-validated | STAD | POLD | 0.859019714 | 0.87150348 | 0.89575849 | NA | 0.919279372 | 0.998604501 | 0.792206909 | NA | NA | NA |
| Cross-validated | UCEC | POLD | 0.931666667 | 0.77833333 | 0.80800794 | NA | 0.863222222 | 0.990595238 | 0.992333333 | NA | NA | NA |
| Cross-validated | BRCA | POLE | 0.677190216 | 0.63809099 | 0.55034627 | 0.499601139 | 0.574034185 | 0.65941608 | 0.745659794 | NA | NA | NA |
| Cross-validated | COAD | POLE | 0.697209213 | 0.78529805 | 0.7914661 | 0.751356433 | 0.852579619 | 0.99965812 | 0.994993109 | NA | NA | NA |
| Cross-validated | STAD | POLE | 0.848812922 | 0.76928625 | 0.79783755 | NA | 0.840882968 | 0.997990245 | 0.891086022 | NA | NA | NA |
| Cross-validated | UCEC | POLE | 0.767543393 | 0.9209932 | 0.88703175 | 0.735653525 | 0.897923469 | 0.993238095 | 0.990066893 | NA | NA | NA |
| Cross-validated | BLCA | SMOKING | 0.560290513 | 0.69558726 | 0.70086577 | 0.660806465 | 0.679847597 | 0.648525653 | 0.697511281 | NA | NA | NA |
| Cross-validated | CESC | SMOKING | 0.534847483 | 0.54523646 | 0.56149354 | NA | 0.516021579 | 0.506014285 | 0.583155071 | NA | NA | NA |
| Cross-validated | ESCAD | SMOKING | 0.575963624 | 0.71819312 | 0.73060053 | NA | 0.721918651 | 0.534238095 | 0.653915344 | NA | NA | NA |
| Cross-validated | ESCSQ | SMOKING | 0.522603535 | 0.72919697 | 0.72075361 | 0.526170996 | 0.654224387 | 0.50717316 | 0.717378066 | NA | NA | NA |
| Cross-validated | HNSCC | SMOKING | 0.70946721 | 0.71296577 | 0.70969795 | 0.745648975 | 0.719205503 | 0.753710807 | 0.793511962 | NA | NA | NA |
| Cross-validated | KIRP | SMOKING | 0.557575091 | 0.62229624 | 0.63488981 | 0.512527081 | 0.61328694 | 0.552134108 | 0.678114474 | NA | NA | NA |
| Cross-validated | LUAD | SMOKING | 0.840233208 | 0.71787516 | 0.73014243 | 0.892731572 | 0.728786011 | 0.91019453 | 0.915371208 | NA | NA | NA |
| Cross-validated | PAAD | SMOKING | 0.57602188 | 0.64608525 | 0.6235223 | NA | 0.615117114 | 0.564759654 | 0.658821833 | NA | NA | NA |
| Cross-validated | SKCM | UV* | 0.915090917 | 0.73324921 | 0.76461558 | 0.891665643 | 0.83148562 | 0.965690019 | 0.937876544 | NA | NA | NA |
| Cross-validated | Median | NA | 0.676041557 | 0.72204174 | 0.72202831 | 0.748502704 | 0.715428451 | 0.639810742 | 0.755696429 | NA | NA | NA |
| Cross-validated | Subset median | NA | 0.738505301 | 0.74254029 | 0.74587776 | 0.748502704 | 0.773481067 | 0.868107734 | 0.81755041 | NA | NA | NA |
| Cross-validated | Subset smoking | SMOKING | 0.560290513 | 0.71296577 | 0.70969795 | 0.660806465 | 0.679847597 | 0.648525653 | 0.717378066 | NA | NA | NA |
| | median | | | | | | | | | | | |
| Cross-validated | Overall smoking | SMOKING | 0.568127069 | 0.70427652 | 0.70528186 | 0.519349038 | 0.667035992 | 0.558446881 | 0.687812877 | NA | NA | NA |
| | median | | | | | | | | | | | |

The “Subset median” AUC is the median AUC calculated only over the tissues where Alexandrov et al. found a signature for the given exposure.
The “Subset smoking median” was instead calculated by restricting the set of tissues to those where Alexandrov et al. detecetd smoking signatures.
To calculate the “Overall smoking median” AUC, whenever Alexandrov et al. methodology was not able to detect a smoking signature in a tissue, and therefore its intensities were not provided (NA), a 0.5 AUC was assigned for their methodology to the smoking signature for that tissue.
The “Subset median” AUC is the median AUC calculated only over the tissues where Alexandrov et al. found an age signature.
To calculate the “Overall median” AUC, whenever Alexandrov et al. methodology was not able to detect the age signature in a tissue, and therefore its intensities were not provided (NA), a 0.5 AUC was assigned to that signature for that tissue for their methodology.

What is claimed is:

1. A method for detecting an etiological factor of a disease in a subject having the disease, the method comprising:
receiving training data that includes data objects each recording i) a disease label, ii) at least one corresponding mutational signature, and iii) corresponding etiological tags;
generating a first set of features based on single nucleotide mutations;
generating a second set of features based on dinucleotide mutations;
training a machine learning model on the first set of features and on the second set of features;
generating, from the machine learning model, a classifier that is configured to:
operate by receiving a new-genomic-data-object, the new-genomic-data-object specific to the subject having the disease; and
generate, from the new-genomic-data-object, a etiological-classification for the new-genomic-data-object, the etiological-classification indicating a corresponding etiological factor that matches one of the etiological tags; and
receiving the subject's genome;
generating, from the subject's genome, a subject-genomic-data-object for the subject;
detecting an etiological factor for the subject by providing the subject-genomic-data-object to the classifier.

2. The method of claim 1, wherein the first set of features are possible substitutions of single nucleotides of a group consisting of C>A, C>G, C>T, T>A, T>C, and T>G.

3. The method of claim 2, wherein the first set of features are defined using a pyrimidine of the mutated Watson-Crick base pair.

4. The method of claim 1, the method further comprising generating a third set of features based on trinucleotide mutations;
wherein training the machine learning model further comprises training the machine learning model on the third set of features.

5. The method of claim 1, the method further comprising generating a fourth set of features based on all mutations;
wherein training the machine learning model further comprises training the machine learning model on the fourth set of features.

6. The method of claim 1, wherein training of the machine learning model comprises organizing the features into a partition tree that includes layers of nodes, each node representing a particular type of mutation and each child of the node representing possible mutations that are a type of mutation in the particular node.

7. The method of claim 6, the training of the machine learning model further comprises pruning the partition tree by removing a pruned node and all other nodes that are children of the pruned node.

8. The method of claim 7, the training of the machine learning model comprises:
selecting some, but not all, of the nodes as candidate nodes to be used for candidate testing; and
testing the candidate nodes to generate first-phase candidate nodes.

9. The method of claim 8, wherein training of the machine learning model further comprises:
generating second-phase candidates by:
for each particular first-phase candidate node, adjusting a value for each parent node that is also a first-phase candidate node, the adjustment being based on the particular first-phase candidate node;
selecting, as a second-phase candidate, a first-phase candidate with a remaining value above a threshold value.

10. The method of claim 9, wherein training of the machine learning model further comprises:
generating final candidates by:
combining second-phase candidates of training data that did have a particular tag with training data that did not have the particular tag.

11. The method of claim 1, wherein hypermethylation and hypomethylation are considered similarly and independently.

12. The method of claim 1, wherein the disease is a cancer.

13. A non-transitory computer-readable media containing instructions that, when executed by one or more processors, cause the one or more processors to perform operations comprising:
receiving training data that includes data objects each recording i) a disease label, ii) at least one corresponding mutational signature, and iii) corresponding etiological tags;
generating a first set of features based on single nucleotide mutations;
generating a second set of features based on dinucleotide mutations;
training a machine learning model on the first set of features and on the second set of features;
generating, from the machine learning model, a classifier that is configured to:
operate by receiving a new-genomic-data-object, the new-genomic-data-object specific to the subject having the disease; and
generate, from the new-genomic-data-object, a etiological-classification for the new-genomic-data-object, the etiological-classification indicating a corresponding etiological factor that matches one of the etiological tags; and
receiving the subject's genome;
generating, from the subject's genome, a subject-genomic-data-object for the subject;
detecting an etiological factor for the subject by providing the subject-genomic-data-object to the classifier.

14. The media of claim 13, wherein the first set of features are possible substitutions of single nucleotides of a group consisting of C>A, C>G, C>T, T>A, T>C, and T>G.

15. The media of claim 14, wherein the first set of features are defined using a pyrimidine of the mutated Watson-Crick base pair.

16. The media of claim 13, the operations further comprising generating a third set of features based on trinucleotide mutations;
wherein training the machine learning model further comprises training the machine learning model on the third set of features.

17. The media of claim 13, the operations further comprising generating a fourth set of features based on all mutations;
wherein training the machine learning model further comprises training the machine learning model on the fourth set of features.

18. The media of claim 13, wherein training of the machine learning model comprises organizing the features into a partition tree that includes layers of nodes, each node representing a particular type of mutation and each child of the node representing possible mutations that are a type of mutation in the particular node.

19. The media of claim 18, the training of the machine learning model further comprises pruning the partition tree by removing a pruned node and all other nodes that are children of the pruned node.

20. The media of claim 19, the training of the machine learning model comprises:
selecting some, but not all, of the nodes as candidate nodes to be used for candidate testing; and
testing the candidate nodes to generate first-phase candidate nodes.

21. The media of claim 20, wherein training of the machine learning model further comprises:
generating second-phase candidates by:
for each particular first-phase candidate node, adjusting a value for each parent node that is also a first-phase candidate node, the adjustment being based on the particular first-phase candidate node;
selecting, as a second-phase candidate, a first-phase candidate with a remaining value above a threshold value.

22. The media of claim 21, wherein training of the machine learning model further comprises:
generating final candidates by:
combining second-phase candidates of training data that did have a particular tag with training data that did not have the particular tag.

23. The media of claim 13, wherein hypermethylation and hypomethylation are considered similarly and independently.

24. The media of claim 13, wherein the disease is a cancer.

25. A system comprising:
one or more processors; and
a non-transitory computer-readable media containing instructions that, when executed by the one or more processors, cause the one or more processors to perform operations comprising:
receiving training data that includes data objects each recording i) a disease label, ii) at least one corresponding mutational signature, and iii) corresponding etiological tags;
generating a first set of features based on single nucleotide mutations;
generating a second set of features based on dinucleotide mutations;
training a machine learning model on the first set of features and on the second set of features;
generating, from the machine learning model, a classifier that is configured to:
operate by receiving a new-genomic-data-object, the new-genomic-data-object specific to the subject having the disease; and
generate, from the new-genomic-data-object, a etiological-classification for the new-genomic-data-object, the etiological-classification indicating a corresponding etiological factor that matches one of the etiological tags; and
receiving the subject's genome;
generating, from the subject's genome, a subject-genomic-data-object for the subject;
detecting an etiological factor for the subject by providing the subject-genomic-data-object to the classifier.
